(19) United States
(12) Patent Application Publication
(10) Pub. No.: US 2025/0257126 A1
(43) Pub. Date: Aug. 14, 2025

(54) THERAPEUTIC ANTIBODIES

(71) Applicants: CAMBRIDGE ENTERPRISE LIMITED, Cambridge (GB); RIGSHOSPITALET, Copenhagen (DK); SYDDANSK UNIVERSITET, Odense (DK)

(72) Inventors: Maike De La Roche, Cambridge (GB); Marc Andrew De La Roche, Cambridge (GB); Mikkel-Ole Skjodt, Copenhagen (DK); Karsten Skjodt, Odense (DK)

(21) Appl. No.: 18/842,730

(22) PCT Filed: Mar. 3, 2023
(86) PCT No.: PCT/GB2023/050512

§ 371 Date: Aug. 29, 2024

(30) Foreign Application Priority Data
Mar. 3, 2022 (GB) 2202990.4
Jun. 10, 2022 (GB) 2208544.3

(51) Int. Cl. C07K 16/28; A61K 39/00; A61K 40/11; A61K 40/15; A61K 40/31; A61K 40/42; A61K 47/68; A61P 35/00; C12N 5/0783; G01N 33/574

(52) U.S. Cl. CPC C07K 16/28; A61K 40/11; A61K 40/15; A61K 40/31; A61K 40/4202; A61K 47/68031; A61K 47/6849; A61P 35/00; C12N 5/0636; C12N 5/0646; G01N 33/57484; A61K 2039/505; C07K 2317/24; C07K 2317/34; C07K 2317/622; C07K 2317/77; C07K 2317/92; C07K 2319/03; C12N 2510/00; G01N 2333/726

(57) ABSTRACT

LGR5 binding agents, in particular antibodies or fragments thereof which bind to human LGR5, and the use of such binding agents in the treatment of disease, such as cancer and inflammatory disease, and the detection of LGR5.

### FIELD OF THE INVENTION

The invention relates to LGR5 binding agents, in particular antibodies or fragments and derivatives thereof which bind to LGR5, and the use of such binding agents in the treatment of disease, such as cancer and inflammatory disease, and the detection of LGR5.

### INTRODUCTION

The Wnt pathway is one of a cadre of cell-to-cell signalling pathways that coordinates development in metazoans and regulates stem cell function in adult tissues. Stimulation of the Wnt pathway is initiated by engagement of extracellular ligands, generally Wnts, by cognate Wnt receptors on the cell surface and transmission of the signal inside the cell. A subsequent series of molecular interactions culminates on the activation of a context-specific transcriptional program that mediates biological outputs.

Principal roles for the Wnt pathway in sustaining adult stem cell self-renewal and proliferation have been functionally established in a wide range of tissues including epithelial stem cell compartments along the gastrointestinal tract, liver, mammary gland, skin and the maturing B cell compartment. Mutational deregulation of Wnt pathway leading to aberrant target gene expression is one of the key drivers for cancer development in these and a number of other tissues. The oncogenic form of the Wnt pathway has received considerable attention in colorectal cancer (CRC), where somatic mutations de-regulating pathway activity, most commonly deactivating mutation in the tumour suppressor adenomatous polyposis coli (APC), are early and pervasive lesions in disease aetiology. In other epithelial malignancies, a spectrum of cancer driver mutations in other Wnt pathway regulatory components may be present; for instance, in the development of hepatocellular carcinoma, approximately half of the cases acquire gain-of-function mutations in p-catenin or inactivating mutations in AXIN1. In some epithelial cancers aberrant pathway activity and de-regulated target gene expression is a frequent characteristic despite the lack of mutations in the Wnt pathway and might be the consequence of convergent regulation by other cell signalling pathways.

Determining the gene expression signature endemic to cancer cells has enabled the identification of prognostic disease markers and provided a foundation for determining molecular vulnerabilities that may be exploited therapeutically. One notable study identified a cohort of Wnt target genes in a CRC cell line harbouring somatically inactivated APC. Leucine-rich repeat containing G-protein receptor 5 (LGR5) was identified as a gene target of oncogenic Wnt pathway activity and subsequent work established it as the prototypical stem cell marker in the murine and human intestinal epithelia, gastric epithelia, hair follicle, fetal mammary gland, nephrons in the developing kidney and the regenerating liver.

LGR5 is a member of a cell surface G P C R-like receptor family that includes LGR4 and LGR6. While LGR4 is broadly expressed in proliferative cell compartments throughout the body, LGR6 displays a more restricted expression to specific stem cell compartments that include sweat glands, interfollicular epidermis, and nail stem cells. The three LGR family proteins are co-receptors for R-spondin family proteins, with RNF43 or ZNRF3 being the other co-receptors. RNF43 and ZNRF3 are ubiquitin ligases that downregulate cell surface associated Wnt receptors. Occlusion of RNF43/ZNRF3 from Wnt receptors through recruitment to LGR-bound R-spondin serves to increase steady-state levels of Wnt pathway receptors at the cell surface and the responsiveness of pathway activity to stimulation with Wnt ligands.

LGR5 has attracted a great deal of therapeutic interest owing to its overexpression in cancers with oncogenic mutations de-regulating pathway activity. A plethora of studies have demonstrated increased LGR5 expression in CRC tumours and intestinal adenocarcinomas relative to adjacent normal intestinal tissue and have shown that LGR5 is associated with cells at the invasive front of tumours and metastatic cells. As a prognostic marker in CRC, LGR5 transcript levels in patient samples are associated with shorter overall survival and reduced disease-free survival.

Functionally, LGR5 sufficiency studies in CRC cell lines have ascribed roles in proliferative capacity, migration, chemosensitivity, colony formation and in vivo transplantation ability. Studies using human CRC organoid xenografts have found that the LGR5 expressing cell compartment is more proliferative than their non-expressing counterparts.

A number of other malignancies are characterized by increased LGR5 overexpression: basal cell carcinoma, glioblastoma—where upregulated LGR5 co-localizing with the cancer stem cell marker CD133 predicts poor prognosis, ovarian tumours, ER negative breast cancers—where high LGR5 expression during cancer development correlates worse prognosis and B cell malignancies.

Taken together, while LGR5 may have positive, opposing or functionally redundant roles in cancer development, LGR5 overexpression on the cell surface is a characteristic of various cancer types, providing a molecular handle for discriminating cancer cells from non-malignant tissues.

Immune therapies are a class of promising therapeutics, some of which harnessing the specificity and efficacy of antibody binding for recognition of cell surface proteins overexpressed on cancer cells. Indeed, antibodies are one of the best-selling classes of drugs today; five of the top ten best selling drugs are antibodies. The versatility and specificity of antibodies have found widespread use for detection and specific killing of cancer cells in a number of cancer types. Therapeutic antibodies include modalities like antibody-dependent cellular cytotoxicity (ADCC), antibody-drug conjugates (ADCs), chimeric antigen receptors (CARs), and bispecific immune cell engagers (e.g. Bispecific T cell Engagers, BiTEs).

Despite the clinical potential of an antibody to LGR5, there are only few, if any, that have undergone robust validation, hampering efforts in determining relative LGR5 protein levels in normal tissues and disease, examining cellular biology or developing immune therapies towards specific cancer types.

The aim of the present invention is to address the need for antibody-based treatments for use in the treatment of disease, particularly in the treatment of cancer and inflammatory disease, for example. The aim of the present invention is also to provide antibodies against LGR5 for use as a research tool and for use in the treatment and diagnosis of disease.

### SUMMARY OF THE INVENTION

The present invention relates to LGR5 binding agents. In particular, the present invention relates to antibodies which bind to human and cynomolgus (cyno) LGR5, wherein the antibody binds an epitope within amino acids 22-37 of LGR5 (SEQ ID NO.1) and related methods for treating disease, for example cancer and/or inflammatory disease, and identifying the presence of LGR5 in a biological sample. The invention is based, in part, on studies by the inventors in which they found that antibodies directed towards a specific epitope within amino acids 22-37 of LGR5 are highly specific, with high affinity and are taken up rapidly by LGR5 expressing cancer cells. The inventors have shown that the antibodies of the present invention are able to distinguish cells with high levels of LGR5 expression from cells with lower expression levels of LGR5. The antibodies of the present invention therefore represent excellent research tools as well as being particularly useful in therapy, such as the treatment of cancer, particularly cancers which express LGR5.

In one aspect, the invention relates to an antibody or fragment thereof which binds to human LGR5, wherein the antibody binds an epitope located within amino acids 22-37 of SEQ ID NO.1.

In one aspect, the invention relates to an antibody or fragment thereof which binds an epitope which comprises or consists of amino acids 22-37 of SEQ ID NO.1.

In one embodiment, the antibody is human or humanised. In one embodiment, the antibody is a murine antibody.

In one aspect the invention relates to an antibody or fragment thereof which binds to LGR5, wherein the V_{H} of the antibody comprises the following CDR1, CDR2 and CDR3:
a) a CDR1 of SEQ ID No. 2 or a sequence with at least 90% homology thereto, a CDR2 of SEQ ID No. 3 or a sequence with at least 90% homology thereto, a CDR3 of SEQ ID No. 4 or a sequence with at least 90% homology thereto; or
b) a CDR1 of SEQ ID No. 8 or a sequence with at least 90% homology thereto, a CDR2 of SEQ ID No. 9 or a sequence with at least 90% homology thereto, a CDR3 of SEQ ID No. 10 or a sequence with at least 90% homology thereto; or
c) a CDR1 of SEQ ID No. 14 or a sequence with at least 90% homology thereto, a CDR2 of SEQ ID No. 15 or a sequence with at least 90% homology thereto, a CDR3 of SEQ ID No. 16 or a sequence with at least 90% homology thereto; or
d) a CDR1 of SEQ ID No. 20 or a sequence with at least 90% homology thereto, a CDR2 of SEQ ID No. 21 or a sequence with at least 90% homology thereto, a CDR3 of SEQ ID No. 22 or a sequence with at least 90% homology thereto; or
e) a CDR1 of SEQ ID No. 26 or a sequence with at least 90% homology thereto, a CDR2 of SEQ ID No. 27 or a sequence with at least 90% homology thereto, a CDR3 of SEQ ID No. 28 or a sequence with at least 90% homology thereto; or
f) a CDR1 of SEQ ID No. 32 or a sequence with at least 90% homology thereto, a CDR2 of SEQ ID No. 33 or a sequence with at least 90% homology thereto, a CDR3 of SEQ ID No. 34 or a sequence with at least 90% homology thereto; or
g) a CDR1 of SEQ ID No. 38 or a sequence with at least 90% homology thereto, a CDR2 of SEQ ID No. 39 or a sequence with at least 90% homology thereto, a CDR3 of SEQ ID No. 40 or a sequence with at least 90% homology thereto; or
h) a CDR1 of SEQ ID No. 44 or a sequence with at least 90% homology thereto, a CDR2 of SEQ ID No. 45 or a sequence with at least 90% homology thereto, a CDR3 of SEQ ID No. 46 or a sequence with at least 90% homology thereto; or
i) a CDR1 of SEQ ID No. 50 or a sequence with at least 90% homology thereto, a CDR2 of SEQ ID No. 51 or a sequence with at least 90% homology thereto, a CDR3 of SEQ ID No. 52 or a sequence with at least 90% homology thereto; or
j) a CDR1 of SEQ ID No. 56 or a sequence with at least 90% homology thereto, a CDR2 of SEQ ID No. 57 or a sequence with at least 90% homology thereto, a CDR3 of SEQ ID No. 58 or a sequence with at least 90% homology thereto; or
k) a CDR1 of SEQ ID No. 62 or a sequence with at least 90% homology thereto, a CDR2 of SEQ ID No. 63 or a sequence with at least 90% homology thereto, a CDR3 of SEQ ID No. 64 or a sequence with at least 90% homology thereto; or
l) a CDR1 of SEQ ID No. 68 or a sequence with at least 90% homology thereto, a CDR2 of SEQ ID No. 69 or a sequence with at least 90% homology thereto, a CDR3 of SEQ ID No. 70 or a sequence with at least 90% homology thereto; or
m) a CDR1 of SE Q ID No. 74 or a sequence with at least 90% homology thereto, a CDR2 of SEQ ID No. 75 or a sequence with at least 90% homology thereto, a CDR3 of SEQ ID No. 76 or a sequence with at least 90% homology thereto.

In one aspect the invention relates to an antibody or fragment thereof which binds to LGR5, wherein the V_{L} of the antibody comprises the following CDR1, CDR2 and CDR3:
a) a CDR1 of SEQ ID No. 5 or a sequence with at least 90% homology thereto, a CDR2 of SEQ ID No. 6 or a sequence with at least 90% homology thereto, a CDR3 of SEQ ID No. 7 or a sequence with at least 90% homology thereto; or
b) a CDR1 of SE Q ID No. 11 or a sequence with at least 90% homology thereto, a CDR2 of SEQ ID No. 12 or a sequence with at least 90% homology thereto, a CDR3 of SEQ ID No. 13 or a sequence with at least 90% homology thereto; or
c) a CDR1 of SEQ ID No. 17 or a sequence with at least 90% homology thereto, a CDR2 of SEQ ID No. 18 or a sequence with at least 90% homology thereto, a CDR3 of SEQ ID No. 19 or a sequence with at least 90% homology thereto; or
d) a CDR1 of SEQ ID No. 23 or a sequence with at least 90% homology thereto, a CDR2 of SEQ ID No. 24 or a sequence with at least 90% homology thereto, a CDR3 of SEQ ID No. 25 or a sequence with at least 90% homology thereto; or
e) a CDR1 of SEQ ID No. 29 or a sequence with at least 90% homology thereto, a CDR2 of SEQ ID No. 30 or a sequence with at least 90% homology thereto, a CDR3 of SEQ ID No. 31 or a sequence with at least 90% homology thereto; or
f) a CDR1 of SEQ ID No. 35 or a sequence with at least 90% homology thereto, a CDR2 of SEQ ID No. 36 or a sequence with at least 90% homology thereto, a CDR3 of SEQ ID No. 37 or a sequence with at least 90% homology thereto; or
g) a CDR1 of SEQ ID No. 41 or a sequence with at least 90% homology thereto, a CDR2 of SEQ ID No. 42 or a sequence with at least 90% homology thereto, a CDR3 of SEQ ID No. 43 or a sequence with at least 90% homology thereto; or
h) a CDR1 of SEQ ID No. 47 or a sequence with at least 90% homology thereto, a CDR2 of SEQ ID No. 48 or a sequence with at least 90% homology thereto, a CDR3 of SEQ ID No. 49 or a sequence with at least 90% homology thereto; or
i) a CDR1 of SEQ ID No. 53 or a sequence with at least 90% homology thereto, a CDR2 of SEQ ID No. 54 or a sequence with at least 90% homology thereto, a CDR3 of SEQ ID No. 55 or a sequence with at least 90% homology thereto; or
j) a CDR1 of SEQ ID No. 59 or a sequence with at least 90% homology thereto, a CDR2 of SEQ ID No. 60 or a sequence with at least 90% homology thereto, a CDR3 of SEQ ID No. 61 or a sequence with at least 90% homology thereto; or
k) a CDR1 of SEQ ID No. 65 or a sequence with at least 90% homology thereto, a CDR2 of SEQ ID No. 66 or a sequence with at least 90% homology thereto, a CDR3 of SEQ ID No. 67 or a sequence with at least 90% homology thereto; or
l) a CDR1 of SEQ ID No. 71 or a sequence with at least 90% homology thereto, a CDR2 of SEQ ID No. 72 or a sequence with at least 90% homology thereto, a CDR3 of SEQ ID No. 73 or a sequence with at least 90% homology thereto; or
m) a CDR1 of SEQ ID No. 77 or a sequence with at least 90% homology thereto, a CDR2 of SEQ ID No. 78 or a sequence with at least 90% homology thereto, a CDR3 of SEQ ID No. 79 or a sequence with at least 90% homology thereto.

In one aspect the invention relates to an antibody or fragment thereof which binds to LGR5, the antibody comprising a V_{H} sequence selected from SEQ ID NOs: 80, 82, 84, 86, 88, 90, 92, 94, 96, 98, 100, 102, 104.

In one aspect the invention relates to an antibody or fragment thereof which binds to LGR5, the antibody comprising a V_{L} sequence selected from SEQ ID NOs: 81, 83, 85, 87, 89, 91, 93, 95, 97, 99, 101, 103, 105.

In one aspect the invention relates to an antibody or fragment thereof which binds to LGR5, the antibody comprising:
a) a V_{H} sequence of SEQ ID NO: 80 and a V_{L} sequence of SEQ ID NO: 81;
b) a V_{H} sequence of SEQ ID NO: 82 and a V_{L} sequence of SEQ ID NO: 83;
c) a V_{H} sequence of SEQ ID NO: 84 and a V_{L} sequence of SEQ ID NO: 85;
d) a V_{H} sequence of SEQ ID NO: 86 and a V_{L} sequence of SEQ ID NO: 87;
e) a V_{H} sequence of SEQ ID NO: 88 and a V_{L} sequence of SEQ ID NO: 89;
f) a V_{H} sequence of SEQ ID NO: 90 and a V_{L} sequence of SEQ ID NO: 91;
g) a V_{H} sequence of SEQ ID NO: 92 and a V_{L} sequence of SEQ ID NO: 93;
h) a V_{H} sequence of SEQ ID NO: 94 and a V_{L} sequence of SEQ ID NO: 95;
i) a V_{H} sequence of SEQ ID NO: 96 and a V_{L} sequence of SEQ ID NO: 97;
j) a V_{H} sequence of SEQ ID NO: 98 and a V_{L} sequence of SEQ ID NO: 99;
k) a V_{H} sequence of SEQ ID NO: 100 and a V_{L} sequence of SEQ ID NO: 101
l) a V_{H} sequence of SEQ ID NO: 102 and a V_{L} sequence of SEQ ID NO: 103; or
m) a V_{H} sequence of SEQ ID NO: 104 and a V_{L} sequence of SEQ ID NO: 105.

In one aspect the invention relates to an antibody or fragment thereof which binds to LGR5, the antibody comprising the combination of V_{H}, C_{H}, V_{L} and C_{L} SEQ ID NOS: 106 to 156 for the clones shown in Table 3 or a sequence with at least 70%, 80% or 90% homology thereto or a sequence of an antibody clone as shown in Table 3 or a sequence with at least 70%, 80% or 90% homology thereto.

In one embodiment, the antibody or fragment thereof binds to human LGR5. The may antibody or fragment may also bind to the corresponding sequence in cynomolgus (cyno) LGR5 (amino acids 22-37).

In embodiments the antibody or fragment thereof is a monoclonal antibody.

In embodiments the antibody or fragment thereof is a human, humanized, or chimeric antibody.

In embodiments the antibody or fragment thereof is capable of binding LGR5 with a Kd of less than around 4 nM.

In embodiments the fragment comprises a Fab, scFv or single domain antibody.

In embodiments, the antibody or fragment thereof is conjugated to a toxin, enzyme, radioisotope, label, therapeutic molecule or other chemical moiety.

In another aspect, the invention relates to an immunoconjugate comprising an antibody or fragment thereof according to the invention linked to a therapeutic agent.

The therapeutic agent may be a toxin, enzyme, radioisotope or other chemical moiety.

In another aspect, the invention relates to a pharmaceutical composition comprising an antibody or fragment thereof as described herein or an immunoconjugate as described herein and a pharmaceutical carrier.

In another aspect, the invention relates to a method for treating a cancer comprising administering a therapeutically effective amount of an antibody or fragment thereof as described herein, an immunoconjugate as described herein or a pharmaceutical composition as described herein.

In another aspect, the invention relates to the use of an antibody or fragment thereof as described herein, an immunoconjugate as described herein or a pharmaceutical composition as described herein in the manufacture of a medicament for the treatment of a cancer.

In another aspect, the invention relates to an antibody or fragment thereof as described herein, an immunoconjugate as described herein or a pharmaceutical composition as described herein for use as a medicament.

In another aspect, the invention relates to an antibody or fragment thereof as described herein, an immunoconjugate as described herein or a pharmaceutical composition as described herein for use in the treatment of a cancer.

In embodiments the cancer is an LGR5-positive cancer.

In embodiments the cancer overexpresses LGR5.

In embodiments the cancer is selected from cancer of the head or neck, uterine cancer, colorectal cancer, stomach cancer, carcinoma of the endometrium, cancer of the oesophagus, leukaemia, such as acute lymphoblastic leukaemia (ALL), liver cancer, such as hepatocellular carcinoma or pancreatic cancer.

In another aspect, the invention relates to an isolated nucleic acid molecule comprising a nucleotide sequence encoding an antibody or fragment thereof as described herein.

In another aspect, the invention relates to a vector comprising a nucleic acid as described herein.

In another aspect, the invention relates to a host cell comprising a nucleic acid as described herein or a vector as described herein.

The host cell may be a bacterial, viral or mammalian cell.

In yet another aspect, the invention relates to method of producing an antibody as described herein comprising culturing a host cell as described herein under conditions suitable for expression of the polynucleotide encoding the antibody and isolating the antibody.

In another aspect, the invention relates to a method of detecting LGR5 in a biological sample comprising contacting the biological sample with the antibody as described herein under conditions permissive for binding of the antibody to LGR5, and detecting whether a complex is formed between the antibody and LGR5.

In embodiments the biological sample may be a cancer sample selected from cancer of the head or neck, uterine cancer, colorectal cancer, stomach cancer, carcinoma of the endometrium, cancer of the oesophagus, leukaemia, such as acute lymphoblastic leukaemia (ALL), liver cancer, such as hepatocellular carcinoma or pancreatic cancer.

In another aspect, the invention relates to a kit comprising an antibody as described herein, an immunoconjugate as described herein, optionally with instructions for use, or a pharmaceutical composition as described herein.

In another aspect, the invention relates to a binding agent, e.g. and antibody or fragment thereof, that binds to essentially the same epitope as the antibody described herein or an antibody or fragment thereof that competes with one of the antibodies provided herein for binding to human or cyno LGR5.

In another aspect, the invention relates to an isolated synthetic or recombinant peptide comprising an epitope, the peptide consisting of residues 22 to 37 of SEQ ID NO: 1.

In another aspect, the invention relates to chimeric antigen receptor (CAR) comprising an antibody or fragment thereof as described herein.

In another aspect, the invention relates to a CAR comprising SEQ ID NO. 211 or a sequence with at least 70%, 80% or 90% homology thereto. In one embodiment, the CAR comprises the CDRs of clone 2.4

In another aspect, the invention relates to cell or cell population expressing such CAR.

In another aspect, the invention relates to such a cell or population of cells for use in adaptive immunotherapy.

In another aspect, the invention relates to a method for adaptive immunotherapy comprising administering such a cell or population of cells.

In another aspect, the invention relates to an immune cell engager, such as a bispecific T cell engager, i.e. a BiTE or a tri-Specific Killer Engager TriKE, comprising an antibody or fragment as described herein. In one embodiment, the BiTE or TriKE comprises the CDRs of clone 2.4

In another aspect, the invention relates to a BiTE comprising SEQ ID NO. 211 or a sequence with at least 70%, 80% or 90% homology thereto.

In another aspect, the invention relates to an isolated synthetic or recombinant peptide comprising an epitope, the peptide consisting of residues 22 to 37 of SEQ ID NO: 1.

In another aspect, the invention relates to a method of diagnosing or assessing progression of cancer comprising assessing expression of LGR5 and/or assessing protein levels of LGR5.

### FIGURES

The invention is further described in the following non-limiting figures.

FIG. 1. Validation of novel LGR5-specific antibodies. A. Western blot analysis of HEK293T lysates expressing LGR family transgenes probed with α-LGR5 hybridoma clone 2 (Table 1, antibody 2) and antibodies raised against HA and vinculin. B. Epitope mapping of a-LGR5 hybridoma clone 2 using fragments of the 100 amino acid antigen sequence traces the epitope to Fragment 1A, composed of the N-terminal of human LGR5 (amino acids 22-37). C. Immunofluorescence of HEK293T cells expressing eGFP-fused human LGR4-6 transgenes and cynomolgus LGR5 (in green) using fluorescently labelled (Alexa647) FL-α-LGR5 in red. D. Flow cytometric analysis of HEK293T cells expressing human LGR4 and LGR5 transgenes using FL-a-LGR5 for detection. E. Pre-incubation of FL-α-LGR5 with supra-stoichiometric amounts of the Fragment 1A peptide abrogates the signal in LGR5-eGFP expressing cells.

FIG. 2. Specificity of LGR5 antibodies generated in the study. A. Amino acid sequence of the human LGR5 antigen used for immunization and generation of α-LGR5. The sequence is annotated with the Fragments used in the RAD display system to localize the a-LGR5 epitope to Fragment 1A (see FIG. 1A). Below—annotation of amino acid sequences of the Fragments used for epitope mapping and structural/topological model showing the antigenic region (in red) within the extracellular domain of LGR5 (structural coordinates taken from (Peng eta)). B. Configuration of the LGR family transgenic constructs used for antibody validation. All expressed LGR proteins contain a common N-terminal hemagglutinin (HA) tag and fusion at the C-terminus to the vasopressin V2 receptor C-terminal tail followed by eGFP. C. Western blot analysis of HEK293T lysates expressing the human LGR family transgenes probed with α-LGR5 hybridoma clones 1, 3 and 4 and antibodies to HA and vinculin, as noted. Immune reactivity was also observed for hybridoma clone 12 (data not shown). No specific immune reactivity was observed when probing the western blots with the other 13 hybridoma clones. D. Sequence conservation amongst the α-LGR5 hybridoma clones within the complementary determining regions (CDRs). Conserved amino acids relative to α-LGR5 clone 1 for clones 2-4 are represented by a dash. Amino acid differences are shown with a closed circle. E. Epitope mapping for a-LGR5 hybridoma clones 1, 3 and 4, as in FIG. 1B, using Fragments delineated in FIG. 2A. F. Sequence alignment of the N-terminal 15 amino acids of human LGR5, corresponding to Fragment 1A, with the corresponding region in other LGR family members. Sequence alignments were based on the three invariant cysteine residues, boxed. Amino acid differences in the cyno sequence are in red. G. Wnt pathway reporter assays (SuperTopFlash assays) for HEK293T cells treated with Wnt3A ligand, R-spondin and either IgG1 or a-LGR5 at levels of approximately 10-fold molar excess over R-spondin. ns, no significant difference, determined by two-tailed t-test. H. Immune fluorescence using FI-α-LGR5 for HEK293T cells expressing transgenic LGR4 or LGR5, as in FIG. 1C.

FIG. 3. Census of healthy tissues and cancers for LGR5 expression levels. A. Quantitation of LGR5 expression levels on normal tissue from 27 fallopian tube samples (FT), 28 ovarian cancer cases (OvC) and 14 omentum cancer cases (OmC) from the Cambridge ovarian cancer TMA. Level of significance calculated using two-tailed t-test comparing ovarian cancer cases (OvC) and omentum cancer cases (OmC) with levels of LGR5 protein in fallopian tube (FT) samples. B. Quantitation of LGR5 expression levels in samples from the Cambridge Brain cancer TMA in 5 samples each of normal brain tissue (Brain), low grade glioma (LGG) and glioblastoma (GBB). Level of significance calculated using two-tailed t-test comparing either low grade glioma (LGG) or glioblastoma (GBM) cases with normal brain tissue.

FIG. 4. LGR5 expression analysis in cancers A. Normalised (log 2 median-centered) LGR5 gene expression levels are shown for each cancer type, ordered by median LGR5 gene expression (black point). The grey horizontal line indicates the median LGR5 expression across all cancer samples. Samples with lower or higher than median (grey line) LGR5 expression are shown in light or dark red, respectively. Cancer types for which more than 70% of samples had higher than median LGR5 expression were defined as “high LGR5 tumours” and are printed in bold. B. Comparison of LGR5 gene expression in tumour (red) vs. normal tissues (gray), where available, for high LGR5 tumours. The grey horizontal line indicates the median LGR5 expression across all pan-cancer tumours. Significance levels of the difference of cancer versus healthy tissue (Wilcoxon test) are indicated: **p 0.01, ***p 0.001, **** p 0.0001). C. Representative images for fallopian tube tissue (left panel set), ovarian cancers (middle panel set) and omentum cancer (right panel set). Arrows shown on one of the fallopian tube sample sets indicate very rare instances of epithelial cells containing LGR5 positive intacellular puncta. Inset white numbers refer to the relative corresponding level of LGR5 expression consistent with the scoring for the Cambridge ovarian cancer TMA. D. Relative expression levels of p-catenin in the fallopian tube, ovarian cancer and omentum cancer sample sets. Levels of significance between fallopian tube (FT) and either ovarian cancer (OvC) or omentum cancer (OmC) expression datasets were calculated by two-tailed t- test. E. Representative images for brain tissue (left panel set), G BM (middle panel set) and LGG (right panel set). Inset white numbers refer to LGR5 expression levels based on the criteria used for TMA scoring. F. Quantification of LGR5 protein expression in B cells, CD4+ T cells and CD8+ T cells from healthy donor PBMCs. There were no significant differences in cellular LGR5 expression (mean fluorescence intensity or % LGR5 expressing cells) in the presence or absence of a-LGR5 blocking peptide.

FIG. 5. Characterisation of LGR5 expression in NALM6 and LoVo cell lines. A. LGR5 transcript levels in LoVo and SW480 cell lines measured by RT-PCR using TBP as a housekeeping gene. B. LGR5 western blot analysis of lysates from 5 CRC cell lines. An antibody raised against vinculin was used as a loading control. C. Flow cytometric analysis of LoVo and SW480 cells with FI-a-LGR5 (red plots) or with the antibody pre-incubated with the Fragment 1A peptide (black plots). D. Relative LGR5 transcript levels in the NALM6, REH and 697 pre-B-ALL cell lines measured by RT-PCR using TBP as a housekeeping gene. E. Western blot analysis of LGR5 protein levels in lysates from the pre-B-ALL cell lines. F. LGR5 protein levels in the three pre-B-ALL cell lines, 697, REH and NALM6 analysed by flow cytometry.

FIG. 6. Intracellular localisation of LGR5. A. Detection of LGR5 in NALM6 and LoVo cell lines using immune fluorescence with FI-α-LGR5. B. Co-immune fluorescence detection of puncta decorated with either LGR5 and LAMP1 in LoVo cells using FI-a-LGR5 or an antibody raised against LAMP1.

FIG. 7: Rapid internalisation of cell surface LGR5

Murine clone 2 modified with a fluorescent tag was used (see clone 2 in Tables 1 and 2).
A. Time course of FL-α-LGR5 internalisation by LGR5-eGFP expressing HEK293T cells. Top right panel—enlarged image showing association of FL-α-LGR5 with the cell periphery after 5 minutes, followed by co-localisation with the internal LGR5-eGFP associated puncta within 30 minutes (middle right panel). Bottom right panel—no association or internalisation of FL-α-LGR5 for cells expressing LGR4-eGFP. Scale bar, 10 μM.
B. Time course of FI-α-LGR5v4 (red) internalisation by NALM6 cells detected by immunofluorescence. At the timepoints, fixed cells were probed with fluorescent (Alexa488) phalloidin (green) and Hoechst (blue). Bottom panels, FI-α-LGR5v4, substituted with FI-L-LGR5v6 (red) as isotype control. In the bottom right panel, the signal from the fluorescent Alexa488 phalloidin has been omitted. Scale bar, 10 μM.
C. Time course of FI-α-LGR5 (red) internalisation by LoVo cells. F-actin and nuclei were visualised by Alexa488-Phalloidin and Hoechst probes, respectively. For the two panels on the right, FI-α-LGR5 was pre-incubated with Frag1A. In the rightmost image, the signal from Alexa488 phalloidin has been omitted. Scale bars, 10 μM.

D. Flow cytometric detection of FI-α-LGR5 (red histograms) or fluorescent isotype control (grey histograms) association with NALM6 cells after 1 hour incubation at 4° C. (top panel) or 37° C. (bottom) panel. Numbers represent percentage of cells with detectable fluorescence.

E. Internalisation of FL-α-LGR5 by the c18 control or the c20 CRISP R/Cas9-LGR5 targeted LoVo cell lines monitored detected by flow cytometry.
F. Flow cytometric analysis of LoVo and NALM6 cells after 60 minutes incubation with FI-IgG1 (orange), and FI-α-H ER 2 (blue) with either FI-α-LGR5v4 (red).
G. Time course of FI-α-LGR5v4 and FI-α-HE R2 association with LoVo cells. Percent association was scored as fraction of cells associated with fluorescent signals from FI-□-HER2 (light grey bars) or FI-α-LGR5v4 (dark grey bars) at the indicated timepoints per field of view. Data points are average of 6 individual scoring experiments, each measuring at 80 to 200 cells over for 2 independent experiments. Error bars indicate SD amongst scoring experiments. There were no significant differences between FI-α-LGR5v4 or FI-α-HER2 association with LoVo cells or amongst any of the timepoints.
H. Time course of percent internalisation of FI-α-LGR5v4 and FI-α-H ER 2 by LoVo cells. Internalisation data scores 6 experimental counts of 80-200 cells over 2 independent experiments. Error bars indicate SD. Significant differences in internalisation between FI-α-LGR5v4 or FI-α-H ER 2 are at the *, p<0.001 level of significance, determined by two-tailed t-test, for the 5- to 180-minute timepoints.
I. Co-localisation between internalized FL-α-LGR5v4 puncta and markers of various intracellular compartments and IQGAP1 in LoVo cells. Co-localisation data were derived from automatic scoring of puncta in images of internalised FL-α-LGR5v4 and intracellular markers detected by indirect immunofluorescence and is the composite of a minimum of 200 cells over 2 individual experiments. Right—wheel graph indicating fractional association of internalised FL-α-LGR5v4 with specific intracellular vesicle markers in LoVo cells.
J. Co-localisation between internalized FL-α-LGR5 puncta and markers of various intracellular compartments and IQGAP1 in NALM6 cells, scoring as above. Right- wheel graph showing fractional association of internalised FL-α-LGR5v4 with specific intracellular vesicle markers in NALM6 cells.

FIG. 8. CRISPR/Cas9 targeting of exon 1 of LGR5. Sequencing of the LGR5 locus exon 1 indicates that clone 20 (c20) harbours an in-frame deletion of 7 amino acids of the coding region for the signal peptide.

FIG. 9. In vitro killing of LGR5-expressing cell lines by α-LGR5-ADC. A. NALM6 or REH cell lines were treated with α-LGR5-ADC and cell survival was determined after 72 hours. Cell killing data was fit to a non-linear EC50 shift model yielding EC50 values of 4 and 10 nM, respectively. As control, NALM6 cells were treated with the non-cleavable a-LGR5-ADC^{NC} which did not reduce the cell count after 72 hours, relative to non-treated controls. B. LoVo cells were treated with either α-LGR5-ADC or α-LGR5-ADC^{NC} as non-cell killing control. Modelling of a-LGR5-ADC mediated cell killing data yielded an EC50 of 9 nM. In this figure, murine clone 2 modified with a MMAE conjugate is used. The sequence is shown in Tables 1 and 2.

FIG. 10. Format and chemistry underpinning the IgG and α-LGR5-based ADCs used in the study (Walsh S J, Omarjee S, Galloway W R J D, Kwan T T L, Sore H F, Parker J S, Hyvönen M, Carroll J S, Spring D R. A general approach for the site-selective modification of native proteins, enabling the generation of stable and functional antibody-drug conjugates. Chem Sci. 2019; 10(3):694-700)

FIG. 11. In vivo targeting of NALM6 tumours with murine α-LGR5-ADC. A. Experimental design. B. NALM6 tumour burden measured by IVIS imaging over the course of the treatment with either a-LGR5-ADC or IgG1-ADC control. *p<0.01, **p<0.001 C. IVIS images of control and a-LGR5-ADC treated mice, dorsal view, at experimental endpoint. D. Spleen mass and absolute number of NALM6 tumour cells in the spleen at experimental endpoint. E. Relative number of NALM6 cells per gram blood in mice treated with a-LGR5-ADC or IgG1-ADC control. F. Number of NALM6 cells extracted from femurs of mice from the two treatment groups. (D-F) *p<0.001. In this figure, murine clone 2 modified with a MMAE conjugate is used. The sequence is shown in Tables 1 and 2.

FIG. 12. α-LGR5-ADC targeting of NALM6 tumours. A. H&E staining of tissue sections from IgG1-ADC (top row) and a-LGR5-ADC (bottom row) treated mice. No differences in gross morphology were observed, nor did we take any differences in the number of NALM6 cells in tissue between experimental groups except for spleen where we observed >20-fold more NALM6 cells (see inset) B. Immune fluorescence of intestinal epithelial sections from IgG1-ADC (top row) and α-LGR5-ADC treated (bottom row) mice using antibodies to Ki67 antigen and β-catenin. No differences in gross morphology of the intestinal epithelial monolayer were observed. C. Evaluation of β-catenin staining at the cell periphery juxtaposed to cell-cell contacts (left graph) and average number of Ki67 positive nuclei per confocal section of intestinal epithelial sections from IgG1-ADC (top row) and α-LGR5-ADC treated mice.

FIG. 13. In vivo targeting of NALM6 tumours with humanised α-LGR5v4-ADC. A. Experimental design. B. NALM6 tumour burden measured by IVIS imaging, dorsal view, over the course of the treatment with either a-LGR5v4-ADC or a-LGR5v4-ADC control. *p<0.01, **p<0.001 C. IVIS images of mice treated with α-LGR5v4-ADC or α-LGR5v6-ADC control at experimental endpoint. D. Spleen mass and absolute number of NALM6 cells in the spleen at experimental endpoint*, p<0.001. E. Number of NALM6 cells in the blood of mice treated with α-LGR5v4-ADC and α-LGR5v6-ADC control. F. Number of NALM6 cells in the bone marrow of α-LGR5v4-ADC and α-LGR5v6-ADC treated mice at experimental endpoint. (D-F) *p<0.001.

FIG. 14. α-LGR5v4 specifically binds human and cynoLGR5 and α-LGR5v4-ADC targeting of NALM6 tumours. A. Western blot analysis of lysates from transgenic murine, human and cyno LGR5 expressing HEK293T cells using antibodies to H A and the humanised antibodies α-LGR5v4 and a-LGR5v6. Note, humanised α-LGR5v6 has lost all binding to LGR5. B. Indirect immune fluorescence analysis of human and murine LGR family expressing HEK293T cells using the a-LGR5v4 antibody (in red), the Alexa488-coupled Phalloidin F-actin probe alongside eGFP fluorescence imaging. In the bottom row of images, LGR5-expressing HEK293T cells were stained with a-LGR5v6. C. H&E staining of tissue sections from a-LGR5v6-ADC (top row) and a-LGR5v4-ADC (bottom row) treated mice. No differences in gross morphology were observed, nor did we detect any differences in the number of NALM6 cells in tissue between experimental groups except for spleen where we observed >20-fold more NALM6 cells (see inset) D. Immune fluorescence of intestinal epithelial sections from α-LGR5v6-ADC (top row) and α-LGR5v4-ADC (bottom row) treated mice using antibodies to Ki67 antigen and β-catenin. No differences in gross morphology of the intestinal epithelial monolayer were observed. E. E valuation of β-catenin staining at the cell periphery juxtaposed to cell-cell contacts (left graph) and average number of Ki67 positive nuclei per confocal section of intestinal epithelial sections from IgG1-ADC (top row) and α-LGR5-ADC treated mice. (C-E) from experiment shown in FIG. 13.

FIG. 15. CAR cell killing. α-LGR5-CAR cell killing. HEK293 cells were transfected with human (A) and cynomolgus (B) LGR5 and served as target cells in killing assays. (C) α-LGR5 CAR NK92 cells form productive synapses with tumour target cells. (D) tumour cell killing of NALM6, R E H, and 647 ALL tumour cell lines at indicated effector:target ratios after 5 h.

FIG. 16. α-LGR5 Bispecific T cell engagers (BiTEs) are activating T cells and lead to efficient tumour cell destruction. (A) PBMCs were incubated with NALM6 tumour cells in the presence of α-LGR5 scFv control, α-LGR5LC or CL BiTE, respectively. CD4+ and CD8+ T cell activation was determined by flow cytometric analysis of CD69 and CD25 expression after 24 hours. (B) NALM6 tumour cells were co-cultured with cytotoxic CD8+ T cells generated from healthy donor PBMC in the presence of α-LGR5 scFv control, α-LGR5LC or CL BiTE, respectively. Tumour cell killing was assessed at an effector to target ratio of 5 to 1 after 5 hours.

FIG. 17. Analysis of LGR5 expression. A. LGR5 mRNA expression in NALM6 (ALL), LoVo (CRC) and HepG2 (HCC) human cell lines. RNA was extracted from cell lines at multiple timepoints and subjected qRT-PCR analysis of LGR5 using Taqman probes. TBP served as a housekeeping gene. B. Western blot analysis of LGR5 protein expression levels in HepG2 cells. HepG2 cells were lysed and 40pg of protein was loaded per well. Membranes were incubated with α-LGR5 antibody (19-24-1, (1:1000), 4° C., overnight) followed by incubation with a goat anti-mouse IgG-HRP ((1:15000), RT, 1 hr). Actin serves as a loading control.

FIG. 18. Sensitivity of CRC organoid lines expressing different levels of LGR5 to treatment with α-LGR5-ADC. A. Immunofluorescent imaging of CRC organoid lines 1 and 2 using the humanized antibody to LGR5 was used to quantify relative levels of the LGR5 protein. LGR5 protein expression was detectable in CRC organoid line 1 and was approximately 4-fold higher in CRC organoid 2. B. The presence of cleaved caspase 3 in CRC organoid lines 1 and 2 24 hours post α-LGR5-ADC treatment was used as a surrogate for cells undergoing apoptosis in response to treatment with α-LGR5-ADC. CRC organoid line 1 was less sensitive to treatment with α-LGR5-ADC than CRC organoid line 2; estimated EC50 levels for organoid cell apoptosis were 5 nM and 100 nM, respectively.

FIG. 19. α-LGR5-BiTEs lead to specific activation of human CD4⁺ and CD8⁺ T cells in the presence of LGR5⁺ tumour cells and induce efficient tumour cell destruction in vitro. A. CL (CD3/LGR5)—and LC (LGR5/CD3)-BiTE mediated activation of CD⁴⁺ T cells (left panel) and CD⁸⁺ T cells (right panel) in healthy donor PBMCs in the presence or absence of NALM6 tumour cells determined as percent cells with combined expression of CD25 and CD69 by flow cytometry after 24 hours. Control (ctrl), no addition of molecule, scFv refers to treatment with the α-LGR5^{scFv} fragment. Error bars represent SD for three independent activation assays using different healthy donor PBMCs. Significant differences in T cell activation in between addition of scFv and either the LC or CL BiTE molecules were determined by two-tailed t-test and shown at the **, p<0.001 level of significance. B. α-LGR5-BiTEs lead to efficient tumour cell destruction in vitro. NALM6 target cell killing by cytotoxic CD⁸⁺ T cells in the absence (ctrl) or with the addition of scFv or LC or CL BiTE s after 5 hours is shown. Data shown is from three donors and error bars represent SD. The effector to target cell ratios are 10:1 and 5:1, respectively. Significant differences in CD⁸⁺ T cell killing were determined by two-tailed t-test at the *, p<0.005 and **, p<0.001 level of significance.

FIG. 20. α-LGR5-CAR NK92 cells efficiently kill HEK293 cells overexpressing LGR5 and preferentially kill LGR5HIGH expressing tumour cells. A. α-LGR5-CAR NK92 cells efficiently kill HEK293 cells overexpressing LGR5. Cell killing activity of NK92 cells, or NK92 cells expressing α-LGR5-CAR-CD28- or α-LGR5-CAR-4-1BB. hLGR5-eGFP-overexpressing HEK293T target cells were incubated with effector NK92 cells at effector to target ratios of 2.5:1 and 10:1 for 5 h or 9 h, respectively. Error bars represent SD for three independent experiments. Significant differences in target cell killing were determined by two-tailed t-test and shown at *, p<0.05 and **, p<0.001. There were no significant differences in target cell killing between cellular ratios of 5:1 or 10:1. B. α-LGR5-CAR NK92 cells preferentially kill LGR5^{HIGH} expressing tumour cells. NALM 6 (left panel, LGR5^{HIGH}) and REH (right panel, LGR5^{LOW}) target cell killing activity of NK92 cells or NK92 cells expressing the α-LGR5-CAR-CD28 or the α-LGR5-CAR-4-1BB CARs with effector to target ratios of 2.5:1 and 10:1 after 12 hours incubation (top and bottom panels, respectively). Error bars represent SD for three independent experiments. Significant differences in target cell killing were determined by two-tailed t-test, at the *, p<0.05 and **, p<0.01.

FIG. 21 α-LGR5-CAR NK92 cells efficiently kill HepG2 tumour cells. HepG2 cells were pre-seeded 24 h prior to the assay in a 96-well plate. NK92 cells and α-LGR5-CAR NK92 cells were added to the target cells at a ratio of 5:1. Cell death was assessed using Apotracker Green and monitored for 15 h using the Incucyte SX5 (Sartorius). Error bars represent SEM of six different wells.

FIG. 22 α-LGR5-CAR NK92 cells kill NALM66 tumours in vivo. 1×10\hat{ }6 NALM6 cells were injected i.v. into NSG mice; on day 5, 7 and 10 the mice received either 10×10\hat{ }6 parental NK92 cells or α-LGR5-CAR NK92 cells i.v. Mice were taken on day 12, and Nalm6 cells were counted in the spleen. (Mean for Nalm6 count for NK92 cell treated NSG mice: 20664, for a-LGR5-CAR NK92 cell treated NSG mice: 11630). One outlier was removed after Grubbs' test.

FIG. 23 α-LGR5-CAR T cells efficiently kill HepG2 tumour cells. HepG2 cells were pre-seeded 24 h prior to the assay in a 96-well plate. Untransduced T cells and a-LGR5-CAR T cells were added to the target cells at a ratio of 5:1 (A) or 10:1 (B). Cell death was assessed using Apotracker Green and monitored for 15 h using the Incucyte SX5 (Sartorius). Error bars represent SEM of six different wells.

FIG. 24 α-LGR5-CAR T cells efficiently kill LoVo tumour cells. LoVo cells were pre-seeded 24 h prior to the assay in a 96-well plate. Untransduced T cells and α-LGR5-CAR T cells were added to the target cells at a ratio of 5:1 (A) or 10:1 (B). Cell death was assessed using Apotracker Green and monitored for 15 h using the Incucyte SX5 (Sartorius). Error bars represent SEM of six different wells.

FIG. 25 α-LGR5-CAR T cells kill NALM6 tumour cells in vivo. 1×10\hat{ }6 NALM 6-LucYFP cells were injected i.v. into NSG mice. On day 4 and 7, mice received 2.5×10\hat{ }6 untransduced T cells or α-LGR5-CAR T cells or 1×10\hat{ }6 a CD19-CAR T cells, respectively. A. IVIS imaging on day 10. NALM6 cell count in the bone marrow (taken from one tibia) of NSG mice on day 11 treated with untransduced T cells (negative control), α-LGR5-CAR T cells, or α-CD19-CAR T cells (positive control, benchmark). B. Mean for NALM6 count for untransduced T cell treated NSG mice: 22355, for α-LGR5-CAR T cell treated NSG mice: 6296, for α-CD19-CAR T cell treated NSG mice: 10

FIG. 26 α-LGR5-CAR-NK92 cells do not kill HEK cells overexpressing human LGR4, LGR6 or mouse Lgr5. α-LGR5-CAR NK92 cells do not target HEK293 cells overexpressing human LGR4, LGR6 and to much reduced degree mouse Lgr5. Cell killing activity of NK92 cells, or NK92 cells expressing a-LGR5-CAR-CD28- or a-LGR5-CAR-4-1BB. HEK293T target cells overexpressing the indicated proteins were incubated with effector NK92 cells at effector to target ratios of 2.5:1 and 10:1 for 9h. Error bars represent SD for three independent experiments.

FIG. 27 α-LGR5-CAR-T cells targeting of NALM6 cells. α-LGR5-CAR T cells efficiently kill NALM 6 tumour cells. NALM 6 tumour target cell killing activity of α-LGR5-41BB CAR-T cells at indicated effector to target ratios was assessed after 6 hours by VITAL assay. Error bars represent SD of three α-LGR5-CAR T cell batches generated from three different healthy donor PBMCs.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols 1 and 2, Ontermann and Dubel, eds., Springer-Verlag, Heidelberg (2010).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The inventors of the present invention have surprisingly identified antibodies which bind a specific epitope of human LGR5 within amino acids 22-37. As demonstrated in the examples, the antibodies are highly specific, with high affinity and are taken up rapidly by cells making the antibodies highly effective research tools as well as being extremely useful in therapy.

Leucine-rich repeat-containing G-protein receptor 5 (LGR5) has been characterised as a stem cell and cancer stem cell marker. Previous analyses of LGR5 transcript levels indicate high level expression discriminates malignancies such as colorectal cancer (CRC) and pre-B acute lymphoblastic leukaemia (pre-B ALL) from healthy tissues suggesting LGR5 protein expression may provide a molecular handle for prognosis and treatment. The inventors have developed highly specific, high affinity antibodies to the extracellular domain of human LGR5 45 (α-LGR5) that detect high LGR5 protein levels in colorectal cancer (CRC), hepatocellular carcinoma (HCC), and pre-B ALL. In contrast, there is low to undetectable levels of LGR5 protein in normal colon and rectal epithelia, liver, ovarian tissues, brain and immune cell types. LGR5 is rapidly internalised from the plasma membrane and trafficked to intracellular vesicular compartments including lysosomes.

The inventors have shown that treatment of high LGR5-expressing CRC and pre-B ALL cancer cell lines with an antibody-drug conjugate version of a-LGR5 (α-LGR5-ADC) lead to effective cell killing at nanomolar concentrations. Interventional treatment of pre-B ALL tumours with a-LGR5-ADC in vivo led to rapid tumour attrition. The inventors have demonstrated the therapeutic utility of humanised α-LGR5 by using the corresponding scFv fragment for the generation of α-LGR5 chimeric antigen receptors (CARs) and a Bispecific T cell Engager (BiTE). The inventors have demonstrated that α-LGR5-CAR-NK92 cells were effective at killing LGR5-expressing cells while α-LGR5/α-CD3 BiTEs induce T cell activation and killing of NALM6 cells by cytotoxic CD8+ T cells. Taken together, the invention establishes α-LGR5-based therapeutic modalities that effectively discriminate and target CRC, HCC and pre-B ALL tumour cells.

The inventors have also established elevated LGR5 expression as a characteristic of CRC, HCC and pre-B ALL. Importantly, their tissue census, both transcriptional and at the level of LGR5 protein, indicates that normal tissues harbour very low to undetectable LGR5 levels, paving the way for therapeutic targeting of malignancies that overexpress the protein. Indeed, our approach has established CRC, HCC and pre-B ALL as priority cancer targets for a-LGR5-based therapeutics and enables future studies to determine other targetable cancer types and stratifying LGR5 overexpression as a prognostic marker. This is particularly relevant to the assessment of LGR5 protein levels in HCC—high LGR5 protein expression may be used to further stratify the HCC subset with activating mutations in β-catenin, which are characterised by low T cell infiltration and thus referred to as immune deserts. The prediction is that this HCC subset will be refractory to both checkpoint inhibition and cellular therapies.

Indeed, recent reporting from the CheckMate 459 trial (NCT02576509) evaluating nivolumab (PD1 checkpoint inhibitor) versus sorafenib (small molecule kinase inhibitor) in HCC failed to meet its endpoint target of improved overall survival. However, the inventors propose that exclusion of the high LGR5 expressing patient cohort may prove to be important in the outcome of the trial. Hence, stratification of HCC patients with high levels of LGR5 using anti-LGR5 presents an intriguing biomarker opportunity that both reports the β-catenin mutant subset and supports the use of therapeutic molecules such as α-LGR5-ADC, where drug efficacy is not dependent on immune infiltration. Thus, the invention also relates to the use of LGR5 expression as a prognostic marker and related methods as described herein.

The invention therefore provides an antibody or fragment thereof which binds to human LGR5, wherein the antibody binds an epitope located within amino acids 22-37 of SEQ ID NO.1 or binds an epitope which comprises of consists of amino acids 22-37 of SEQ ID NO.1, immunoconjugates and pharmaceutical compositions comprising such antibodies, as well as isolated nucleic acid molecules, vectors and host cells for producing such antibodies. Also provided are methods of using the antibodies disclosed herein to detect human LGR5, methods of diagnosis and methods of treating disease, particularly cancer and/or inflammatory disease.

The antibodies of the present invention bind to LGR5 with high affinity and specificity. In addition, the inventors have shown that the antibodies of the present invention can be rapidly internalised by cells. Thus, antibodies as described herein are capable of binding to the extracellular domain of LGR5 and being internalised. The inventors also we also benchmarked to Trastuzumab and showed exceptionally quick internalisation (within 5 min) while only 40% of Teas is internalised after 3 hours. Moreover, when conjugated to a payload, the conjugated antibodies mediate effective cell killing at nanomolar concentrations.

These properties of the antibodies mean that they can be exploited therapeutically, for example in the treatment of cancer and/or inflammatory disease. For example, the antibodies can be used to bring a therapeutic molecule, for example a drug, into close proximity with LGR5. The antibodies of the present invention can therefore be used to deliver therapeutic molecules specifically to cancers, particularly those which express or overexpress LGR5. The inventors' studies have shown that the specific antibodies of the present invention display no off-target toxicities, further validating their use in therapy.

Furthermore, the antibodies of the present invention can be used as imaging agents, for example in methods for diagnosing cancer and/or inflammatory disease or other biomarker related methods, in addition to their use as highly effective research tools.

The antibodies and fragments of the present invention bind specifically to wildtype human LGR5 (UniProt Accession No. 075473). The amino acid sequence for wildtype human LGR5 is shown below (SEQ ID NO: 1).

MDTSRLGVLLSLPVLLQLATGGSSPRSGVLLRGCPTHCHCEPDGRMLLRV
DCSDLGLSELPSNLSVFTSYLDLSMNNISQLLPNPLPSLRFLEELRLAGN
ALTYIPKGAFTGLYSLKVLMLQNNQLRHVPTEALQNLRSLQSLRLDANHI
SYVPPSCFSGLHSLRHLWLDDNALTEIPVQAFRSLSALQAMTLALNKIHH
IPDYAFGNLSSLVVLHLHNNRIHSLGKKCFDGLHSLETLDLNYNNLDEFP
TAIRTLSNLKELGFHSNNIRSIPEKAFVGNPSLITIHFYDNPIQFVGRSA
FQHLPELRTLTLNGASQITEFPDLTGTANLESLTLTGAQISSLPQTVCNQ
LPNLQVLDLSYNLLEDLPSFSVCQKLQKIDLRHNEIYEIKVDTFQQLLSL
RSLNLAWNKIAIIHPNAFSTLPSLIKLDLSSNLLSSFPITGLHGLTHLKL
TGNHALQSLISSENFPELKVIEMPYAYQCCAFGVCENAYKISNQWNKGDN
SSMDDLHKKDAGMFQAQDERDLEDFLLDFEEDLKALHSVQCSPSPGPFKP
CEHLLDGWLIRIGVWTIAVLALTCNALVTSTVFRSPLYISPIKLLIGVIA
AVNMLTGVSSAVLAGVDAFTFGSFARHGAWWENGVGCHVIGFLSIFASES
SVFLLTLAALERGFSVKYSAKFETKAPFSSLKVIILLCALLALTMAAVPL
LGGSKYGASPLCLPLPFGEPSTMGYMVALILLNSLCFLMMTIAYTKLYCN
LDKGDLENIWDCSMVKHIALLLFTNCILNCPVAFLSFSSLINLTFISPEV
IKFILLVVVPLPACLNPLLYILFNPHFKEDLVSLRKQTYVWTRSKHPSLM
SINSDDVEKQSCDSTQALVTFTSSSITYDLPPSSVPSPAYPVTESCHLSS
VAFVPCL

Unless otherwise specified, the term LGR5 as used herein refers to human LGR5. The term LGR5 includes variants, isoforms and species homologs of human LGR5. The antibodies bind the extracellular domain. Antibodies that bind LGR5 are referenced herein as a-LGR5, aLGR5 or α-LGR5 or αLGR5. These terms are used interchangeably.

The antibodies of the present invention bind to LGR5. The term “binds” as used herein, means that the antibody binds the antigen with sufficient affinity such that the antibody is useful as a therapeutic agent in targeted a cell or tissue expressing the antigen. The binding reaction may be shown by standard methods, for example with reference to a negative control test using an antibody of unrelated specificity.

The invention provides antibodies, specifically humanised antibodies, that bind specifically to human LGR5. In other words, binding to the LGR5 antigen is measurably different from a non-specific interaction. As shown in the examples, the antibodies of the present invention that bind to human do not cross react with mouse LGR5 or human LGR4 or 6. Preferably the antibodies of the invention bind to human LGR5 and also bind to cyno LGR5.

The term “specific binding” or “specifically binds to” or is “specific for” a particular polypeptide or an epitope on a particular polypeptide target as used herein can be exhibited, for example, by a molecule having a Kd for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In one embodiment, the antibody or antibody fragment has a Kd of 0.7-3 nM. In one embodiment, the term “specific binding” refers to binding where a molecule binds to a particular polypeptide or epitope on a particular polypeptide without substantially binding to any other polypeptide or polypeptide epitope.

The term “antibody” broadly refers to any immunoglobulin (Ig) molecule, or antigen binding portion thereof, comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. The term “antibody” as used herein encompasses monoclonal antibodies, polyclonal antibodies, multispecific antibodies (e.g. bispecific antibodies) and antibody fragments, provided they retain the antigen-binding ability of the antibody. The term “antibody” as used herein also encompasses, for example, a T-cell Engager for example a Bispecific T-cell Engager (BiTE). As will be appreciated by the skilled person Bispecific T-cell Engagers (BiTEs) are fusion proteins including two scFvs of different antibodies wherein one of the scFvs binds to T cells via the CD3 receptor, and the other to a tumor cell via a tumor specific molecule.

In one embodiment, the BiTE or CAR described herein comprises a scFv as shown in SEQ ID NO: 211 or a sequence with at least sequence 70%, 80% or 90% homology thereto or a sequence having the CDRs of SEQ ID NO. 211. Thus, one embodiment, the BiTE comprises CDRs of clone 2.4, also numbered as SEQ ID NO. 212, 213 and 214 and the heavy chain CDRs of 215, 216 and 217. In one embodiment, the BiTE or CAR described herein comprises the VH and VL of clone 2.4 or a sequence having at least 90% sequence identity thereto. In embodiments, the antibody may comprise a monoclonal antibody. The term “monoclonal antibody” refers to an antibody obtained from a single close of cells or cell line. The individual antibodies are identical and/or bind the same epitope. Unlike polyclonal antibodies, which include different antibodies directed against different epitopes, each monoclonal antibody in a preparation is directed against a single epitope.

In a full-length antibody, each heavy chain is comprised of a heavy chain variable region or domain (abbreviated herein as HCVR) and a heavy chain constant region. The heavy chain constant region is comprised of three domains, C_{H}1, C_{H}2 and C_{H}3. Each light chain is comprised of a light chain variable region or domain (abbreviated herein as LCVR) and a light chain constant region. The light chain constant region is comprised of one domain, C_{L}.

The heavy chain and light chain variable regions can be further subdivided into regions of hypervariability, termed complementarity determining regions (CDR), interspersed with regions that are more conserved, termed framework regions (FR). Each heavy chain and light chain variable region is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “CDR” refers to the complementarity-determining region within antibody variable sequences. There are three CDRs in each of the variable regions of the heavy chain and the light chain, which are designated CDR1, CDR2 and CDR3, for each of the variable regions. The term “CDR set” refers to a group of three CDRs that occur in a single variable region capable of binding the antigen. The exact boundaries of these CDRs can be defined differently according to different systems known in the art.

The Kabat Complementarity Determining Regions (CDRs) are based on sequence variability and are the most commonly used (Kabat et al., (1971) Ann. N Y Acad. Sci. 190:382-391 and Kabat, et al., (1991) Sequences of Proteins of Immunological Interest, Fifth Edition, U.S.

Department of Health and Human Services, NIH Publication No. 91-3242). Chothia refers instead to the location of the structural loops (Chothia and Lesk J. Mol. Biol. 196:901-917 (1987)). The Kabat numbering system is generally used when referring to a residue in the variable domain (approximately residues 1-107 of the light chain and residues 1-113 of the heavy chain).

The system described by Kabat is used herein. The terms “Kabat numbering”, “Kabat definitions” and “Kabat labelling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion.

A chimeric antibody is a recombinant protein that contains the variable domains including the complementarity determining regions (CDRs) of an antibody derived from one species, such as a rodent antibody, while the constant domains of the antibody molecule are derived from those of a human antibody. For veterinary applications, the constant domains of the chimeric antibody may be derived from that of other species, such as a cat or dog.

A humanized antibody is a recombinant protein in which the CDRs from an antibody from one species; e.g., a rodent antibody, are transferred from the heavy and light variable chains of the rodent antibody into human heavy and light variable domains (e.g., framework region sequences). The constant domains of the antibody molecule are derived from those of a human antibody. In certain embodiments, a limited number of framework region amino acid residues from the parent (rodent) antibody may be substituted into the human antibody framework region sequences.

The term “antigen binding site” refers to the part of the antibody or antibody fragment that comprises the area that specifically binds to an antigen. An antigen binding site may be provided by one or more antibody variable domains. Preferably, an antigen binding site is comprised within the associated V_{H} and V_{L} of an antibody or antibody fragment.

An antibody fragment according to the invention is a functional portion of an antibody, for example a F(ab′)₂, Fab, Fv, sFv and the like. The term refers thus to an antigen binding fragment and is interchangeably used with antigen binding portion of an antibody. Functional fragments of a full-length antibody retain the target specificity of a full-length antibody. Recombinant functional antibody fragments, such as Fab (Fragment, antibody), scFv (single chain variable chain fragments) and single domain antibodies (dAbs) have therefore been used to develop therapeutics as an alternative to therapeutics based on mAbs. Derivatives of antibodies are also within the scope.

scFv fragments (˜25 kDa) consist of the two variable domains, V_{H} and V_{L}. Naturally, V_{H} and V_{L} domain are non-covalently associated via hydrophobic interaction and tend to dissociate. However, stable fragments can be engineered by linking the domains with a hydrophilic flexible linker to create a single chain Fv (scFv). In one embodiment, the scFv comprises SEQ ID NO: 211 or a sequence with at least sequence 70%, 80% or 90% homology thereto or a sequence having the CDRs of SEQ ID NO. 211. In one embodiment, the scFv comprises CDRs of SEQ ID NO. 212, 213 and 214 and the heavy chain CDRs of 215, 216 and 217.

The smallest antigen binding fragment is the single variable fragment, namely the V_{H} or V_{L} domain. Binding to a light chain/heavy chain partner respectively is not required for target binding. Such fragments are used in single domain antibodies. A single domain antibody (˜12 to 15 kDa) therefore consists of or comprises either the V_{H} or V_{L} domain.

As used herein, the term “homology” generally refers to the percentage of amino acid residues in a sequence that are identical with the residues of the reference polypeptide with which it is compared, after aligning the sequences and in some embodiments after introducing gaps, if necessary, to achieve the maximum percent homology, and not considering any conservative substitutions as part of the sequence identity. Thus, as used herein, the percent homology between two amino acid sequences is equivalent to the percent identity between the two sequences. Neither N- or C-terminal extensions, tags or insertions shall be construed as reducing identity or homology. Methods and computer programs for the alignment are well known. The percent identity or homology between two amino acid sequences can be determined using well known mathematical algorithms.

As used herein, when the term homology is used, this is interchangeably used with identity. Thus, when reference is made to sequence homology, these values also refer to sequence identity respectively.

Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps. Generally, default parameters are used, with a gap creation penalty equalling 12 and a gap extension penalty equalling 4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Altschul et al (1997) Nucl. Acids Res. 25 3389-3402) may be used. Sequence identity may be defined using the Bioedit, ClustalW algorithm. Alignments were performed using Snapgene and based on MUSCLE (Multiple Sequence Comparison by Log-Expectation) algorithms (Edgar (2004a) Nucleic Acids Res 32: 1792-7; Edgar (2004b) BMC Bioinformatics 5: 113.).

The present invention relates to an antibody or fragment thereof that binds to an epitope of LGR5 located within amino acids 22-37 of human LGR5 (SEQ ID NO:1).

Thus, the antibody of the present invention binds to an epitope of LGR5 which includes one or more of residues 22-37 of human LGR5 (SEQ ID NO:1). For example, the antibody of the present invention binds to an epitope of LGR5 which includes one or more, e.g. all of the following residues: G²², S²³, S²⁴, P²⁵, R²⁶, S²⁷, G²⁸, V²⁹, L³⁰, L³¹, R³², G³³, C³⁴, P³⁵, T³⁶, H³⁷.

The present invention also relates to an antibody that binds to an epitope which consists of amino acids 22-37 of LGR5 (SEQ ID NO:1).

Such an epitope is a linear epitope, as described fully below.

The term “epitope” or “antigenic determinant” refers to a site on the surface of an antigen (e.g., LGR5) to which an immunoglobulin, antibody or antibody fragment specifically binds.

Generally, an antigen has several or many different epitopes and reacts with many different antibodies. The term specifically includes linear epitopes and conformational epitopes.

Epitopes within protein antigens can be formed both from contiguous amino acids (usually a linear epitope) or non-contiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody or antibody fragment (i.e., epitope mapping) are well known in the art and include, for example, immunoblotting and immunoprecipitation assays, wherein overlapping or contiguous peptides from are tested for reactivity with a given antibody or antibody fragment.

In the present invention, the epitope was mapped by peptide mapping. In particular, the epitope was mapped by testing the reactivity of the antibody with overlapping peptide fragments and performing Western blot analysis.

In one embodiment, the invention relates to an antibody which binds LGR5, wherein the antibody comprises a V_{L} CDR3 sequence as shown in Table 1 below or a sequence with at least 60%, 70%, 80%, 90%, 95% or more sequence identity thereto. In one aspect, the invention relates to an antibody which binds LGR5, wherein the antibody comprises a V_{L} CDR3 sequence selected from SEQ ID NO. 7, 13, 19, 25, 31, 37, 43, 49, 55, 61, 67, 73, 79 or a sequence with at least 60%, 70%, 80%, 90%, 95% or more sequence homology to one of SEQ ID NO. 7, 13, 19, 25, 31, 37, 43, 49, 55, 61, 67, 73, 79. In one embodiment, said sequence homology is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In one embodiment, said sequence homology is at least 90%, 91%, 92%, 93%, 94%, 95%, 96%, 97%, 98% or 99%.

In one embodiment, the antibody has a V_{L} CDR3 sequence comprising or consisting of an amino acid sequence selected from SEQ ID NO. 7, 13, 19, 25, 31, 37, 43, 49, 55, 61, 67, 73, 79 or a sequence having at least at least 90%, or at least 95% homology thereto.

In one embodiment, the invention relates to an antibody which binds LGR5, wherein the antibody comprises a V_{L} CDR1 sequence as shown in Table 1 below or a sequence with at least 60%, 70%, 80%, 90%, 95% or more sequence identity thereto. In one aspect, the invention relates to an antibody which binds LGR5, wherein the antibody comprises a V_{L} C DR1 sequence selected from SEQ ID NO. 5, 11, 17, 23, 29, 35, 41, 47, 53, 59, 65, 71, 77 or a sequence with at least 60%, 70%, 80%, 90%, 95% or more sequence homology to one of SEQ ID NO. 5, 11, 17, 23, 29, 35, 41, 47, 53, 59, 65, 71, 77 In one embodiment, said sequence homology is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In one embodiment, said sequence homology is at least 90%, 91%, 92%, 93%, 94%, 95%, 96%, 97%, 98% or 99%.

In one embodiment, the antibody has a V_{L} CDR1 sequence comprising or consisting of an amino acid sequence selected from SEQ ID NO. 5, 11, 17, 23, 29, 35, 41, 47, 53, 59, 65, 71, 77 or a sequence having at least at least 90%, or at least 95% homology thereto.

In one embodiment, the invention relates to an antibody which binds LGR5, wherein the antibody comprises a V_{L} CDR2 sequence as shown in Table 1 below or a sequence with at least 60%, 70%, 80%, 90%, 95% or more sequence identity thereto. In one aspect, the invention relates to an antibody which binds LGR5, wherein the antibody comprises a V_{L} CDR2 sequence selected from SEQ ID NO. 6, 12, 18, 24, 30, 36, 42, 48, 54, 60, 66, 72, 78 or a sequence with at least 60%, 70%, 80%, 90%, 95% or more sequence homology to one of SEQ ID NO. 6, 12, 18, 24, 30, 36, 42, 48, 54, 60, 66, 72, 78. In one embodiment, said sequence homology is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In one embodiment, said sequence homology is at least 90%, 91%, 92%, 93%, 94%, 95%, 96%, 97%, 98% or 99%.

In one embodiment, the antibody has a V_{L} CDR2 sequence comprising or consisting of an amino acid sequence selected from SEQ ID NO. 6, 12, 18, 24, 30, 36, 42, 48, 54, 60, 66, 72, 78 or a sequence having at least at least 90%, or at least 95% homology thereto.

In one embodiment, the invention relates to an antibody which binds LGR5, wherein the antibody comprises a V_{H} CDR3 sequence as shown in Table 1 below or a sequence with at least 60%, 70%, 80%, 90%, 95% or more sequence identity thereto. In one aspect, the invention relates to an antibody which binds LGR5, wherein the antibody comprises a V_{H} CDR3 sequence selected from SEQ ID NO. 4, 10, 16, 22, 28, 34, 40, 46, 52, 58, 64, 70, 76 or a sequence with at least 60%, 70%, 80%, 90%, 95% or more sequence homology to one of SEQ ID NO. 4, 10, 16, 22, 28, 34, 40, 46, 52, 58, 64, 70, 76 In one embodiment, said sequence homology is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In one embodiment, said sequence homology is at least 90%, 91%, 92%, 93%, 94%, 95%, 96%, 97%, 98% or 99%.

In one embodiment, the antibody has a V_{H} CDR3 sequence comprising or consisting of an amino acid sequence selected from SEQ ID NO. 4, 10, 16, 22, 28, 34, 40, 46, 52, 58, 64, 70, 76 or a sequence having at least at least 90%, or at least 95% homology thereto.

In one embodiment, the invention relates to an antibody which binds LGR5, wherein the antibody comprises a V_{H} CDR1 sequence as shown in Table 1 below or a sequence with at least 60%, 70%, 80%, 90%, 95% or more sequence identity thereto. In one aspect, the invention relates to an antibody which binds LGR5, wherein the antibody comprises a V_{H} CDR1 sequence selected from SEQ ID NO. 2, 8, 14, 40, 26, 32, 38, 44, 50, 56, 62, 68, 74 or a sequence with at least 60%, 70%, 80%, 90%, 95% or more sequence homology to one of SEQ ID NO. 2, 8, 14, 40, 26, 32, 38, 44, 50, 56, 62, 68, 74. In one embodiment, said sequence homology is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In one embodiment, said sequence homology is at least 90%, 91%, 92%, 93%, 94%, 95%, 96%, 97%, 98% or 99%.

In one embodiment, the antibody has a V_{H} CDR1 sequence comprising or consisting of an amino acid sequence selected from SEQ ID NO. 2, 8, 14, 40, 26, 32, 38, 44, 50, 56, 62, 68, 74 or a sequence having at least at least 90%, or at least 95% homology thereto.

In one embodiment, the invention relates to an antibody which binds LGR5, wherein the antibody comprises a V_{H} CDR2 sequence as shown in Table 1 below or a sequence with at least 60%, 70%, 80%, 90%, 95% or more sequence identity thereto. In one aspect, the invention relates to an antibody which binds LGR5, wherein the antibody comprises a V_{H} CDR2 sequence selected from SEQ ID NO. 3, 9, 15, 21, 27, 33, 39, 45, 51, 57, 63, 69, 75 or a sequence with at least 60%, 70%, 80%, 90%, 95% or more sequence homology to one of SEQ ID NO. 3, 9, 15, 21, 27, 33, 39, 45, 51, 57, 63, 69, 75. In one embodiment, said sequence homology is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In one embodiment, said sequence homology is at least 90%, 91%, 92%, 93%, 94%, 95%, 96%, 97%, 98% or 99%.

In one embodiment, the antibody has a V_{H} CDR2 sequence comprising or consisting of an amino acid sequence selected from SEQ ID NO. 3, 9, 15, 21, 27, 33, 39, 45, 51, 57, 63, 69, 75 or a sequence having at least at least 90%, or at least 95% homology thereto.

In one embodiment, the antibody comprises a combination of V_{H} and V_{L} CDR1, 2 and 3 sequences selected from the sequences in Table 1. In one aspect, the invention also relates to an antibody which has combinations of V_{H} and V_{L} CDR1, CDR2 and CDR3 as shown for the clones in Table 1.

In one embodiment, the antibody comprises a set of V_{H} and V_{L} CDR1, 2 and 3 sequences selected from the sets of CDR1, 2 and 3 sequences as shown for the any of the clones in Table 1. In one embodiment, the antibody comprises V_{H} CDR1 having SEQ ID No. 2, V_{H} CDR2 having SEQ ID No. 3, V_{H} CDR3 having SEQ ID No. 4, V_{L} CDR1 having SEQ ID No. 5, V_{L} CDR2 having SEQ ID No. 6 and V_{L} CDR3 having SEQ ID No. 7. In one embodiment, the antibody comprises V_{H} CDR1 having SEQ ID No. 8, V_{H} CDR2 having SEQ ID No. 9, V_{H} CDR3 having SEQ ID No. 10, V_{L} CDR1 having SEQ ID No. 11, V_{L} CDR2 having SEQ ID No. 12 and V_{L} CDR3 having SEQ ID No. 13. In one embodiment, the antibody comprises V_{H} CDR1 having SEQ ID No. 14, V_{H} CDR2 having SEQ ID No. 15, V_{H} CDR3 having SEQ ID No. 16, V_{L} CDR1 having SEQ ID No. 17, V_{L} CDR2 having SEQ ID No. 18 and V_{L} CDR3 having SEQ ID No. 19. In one embodiment, the antibody comprises V_{H} CDR1 having SEQ ID No. 20, V_{H} CDR2 having SEQ ID No. 21, V_{H} CDR3 having SEQ ID No. 22, V_{L} CDR1 having SEQ ID No. 23, V_{L} CDR2 having SEQ ID No. 24 and V_{L} CDR3 having SEQ ID No. 25. In one embodiment, the antibody comprises V_{H} CDR1 having SEQ ID No. 26, V_{H} CDR2 having SEQ ID No. 27, V_{H} CDR3 having SEQ ID No. 28, V_{L} CDR1 having SEQ ID No. 29, V_{L} CDR2 having SEQ ID No. 30 and V_{L} CDR3 having SEQ ID No. 31. In one embodiment, the antibody comprises V_{H} CDR1 having SEQ ID No. 32, V_{H} CDR2 having SEQ ID No. 33, V_{H} CDR3 having SEQ ID No. 34, V_{L} CDR1 having SEQ ID No. 35, V_{L} CDR2 having SEQ ID No. 36 and V_{L} CDR3 having SEQ ID No. 37. In one embodiment, the antibody comprises V_{H} CDR1 having SEQ ID No. 38, V_{H} CDR2 having SEQ ID No. 39, V_{H} CDR3 having SEQ ID No. 40, V_{L} CDR1 having SEQ ID No. 41, V_{L} CDR2 having SEQ ID No. 42 and V_{L} CDR3 having SEQ ID No. 43. In one embodiment, the antibody comprises V_{H} CDR1 having SEQ ID No. 44, V_{H} CDR2 having SEQ ID No. 45, V_{H} CDR3 having SEQ ID No. 46, V_{L} CDR1 having SEQ ID No. 47, V_{L} CDR2 having SEQ ID No. 48 and V_{L} CDR3 having SEQ ID No. 49. In one embodiment, the antibody comprises V_{H} CDR1 having SEQ ID No. 50, V_{H} CDR2 having SEQ ID No. 51, V_{H} CDR3 having SEQ ID No. 52, V_{L} CDR1 having SEQ ID No. 53, V_{L} CDR2 having SEQ ID No. 54 and V_{L} CDR3 having SEQ ID No. 55. In one embodiment, the antibody comprises V_{H} CDR1 having SEQ ID No. 56, V_{H} CDR2 having SEQ ID No. 57, V_{H} CDR3 having SEQ ID No. 58, V_{L} CDR1 having SEQ ID No. 59, V_{L} CDR2 having SEQ ID No. 60 and V_{L} CDR3 having SEQ ID No. 61. In one embodiment, the antibody comprises V_{H} CDR1 having SEQ ID No. 62, V_{H} CDR2 having SEQ ID No. 63, V_{H} CDR3 having SEQ ID No. 64, V_{L} CDR1 having SEQ ID No. 65, V_{L} CDR2 having SEQ ID No. 66 and V_{L} CDR3 having SEQ ID No. 67. In one embodiment, the antibody comprises V_{H} CDR1 having SEQ ID No. 68, V_{H} CDR2 having SEQ ID No. 69, V_{H} CDR3 having SEQ ID No. 70, V_{L} CDR1 having SEQ ID No. 71, V_{L} CDR2 having SEQ ID No. 72 and V_{L} CDR3 having SEQ ID No. 73. In one embodiment, the antibody comprises V_{H} CDR1 having SEQ ID No. 74, V_{H} CDR2 having SEQ ID No. 75, V_{H} CDR3 having SEQ ID No. 76, V_{L} CDR1 having SEQ ID No. 77, V_{L} CDR2 having SEQ ID No. 78 and V_{L} CDR3 having SEQ ID No. 79.

In one aspect the invention provides an antibody or fragment thereof which binds to LGR5, wherein the V_{H} of the antibody comprises the following CDR1, CDR2 and CDR3:
a) a CDR1 of SEQ ID No. 2 or a sequence with at least 90% homology thereto, a CDR2 of SEQ ID No. 3 or a sequence with at least 90% homology thereto, a CDR3 of SEQ ID No. 4 or a sequence with at least 90% homology thereto; or
b) a CDR1 of SEQ ID No. 8 or a sequence with at least 90% homology thereto, a CDR2 of SEQ ID No. 9 or a sequence with at least 90% homology thereto, a CDR3 of SEQ ID No. 10 or a sequence with at least 90% homology thereto; or
c) a CDR1 of SEQ ID No. 14 or a sequence with at least 90% homology thereto, a CDR2 of SEQ ID No. 15 or a sequence with at least 90% homology thereto, a CDR3 of SEQ ID No. 16 or a sequence with at least 90% homology thereto; or
d) a CDR1 of SEQ ID No. 20 or a sequence with at least 90% homology thereto, a CDR2 of SEQ ID No. 21 or a sequence with at least 90% homology thereto, a CDR3 of SEQ ID No. 22 or a sequence with at least 90% homology thereto; or
e) a CDR1 of SEQ ID No. 26 or a sequence with at least 90% homology thereto, a CDR2 of SEQ ID No. 27 or a sequence with at least 90% homology thereto, a CDR3 of SEQ ID No. 28 or a sequence with at least 90% homology thereto; or
f) a CDR1 of SEQ ID No. 32 or a sequence with at least 90% homology thereto, a CDR2 of SEQ ID No. 33 or a sequence with at least 90% homology thereto, a CDR3 of SEQ ID No. 34 or a sequence with at least 90% homology thereto; or
g) a CDR1 of SEQ ID No. 38 or a sequence with at least 90% homology thereto, a CDR2 of SEQ ID No. 39 or a sequence with at least 90% homology thereto, a CDR3 of SEQ ID No. 40 or a sequence with at least 90% homology thereto; or
h) a CDR1 of SEQ ID No. 44 or a sequence with at least 90% homology thereto, a CDR2 of SEQ ID No. 45 or a sequence with at least 90% homology thereto, a CDR3 of SEQ ID No. 46 or a sequence with at least 90% homology thereto; or
i) a CDR1 of SEQ ID No. 50 or a sequence with at least 90% homology thereto, a CDR2 of SEQ ID No. 51 or a sequence with at least 90% homology thereto, a CDR3 of SEQ ID No. 52 or a sequence with at least 90% homology thereto; or
j) a CDR1 of SEQ ID No. 56 or a sequence with at least 90% homology thereto, a CDR2 of SEQ ID No. 57 or a sequence with at least 90% homology thereto, a CDR3 of SEQ ID No. 58 or a sequence with at least 90% homology thereto; or
k) a CDR1 of SEQ ID No. 62 or a sequence with at least 90% homology thereto, a CDR2 of SEQ ID No. 63 or a sequence with at least 90% homology thereto, a CDR3 of SEQ ID No. 64 or a sequence with at least 90% homology thereto; or
l) a CDR1 of SEQ ID No. 68 or a sequence with at least 90% homology thereto, a CDR2 of SEQ ID No. 69 or a sequence with at least 90% homology thereto, a CDR3 of SEQ ID No. 70 or a sequence with at least 90% homology thereto; or
m) a CDR1 of SEQ ID No. 74 or a sequence with at least 90% homology thereto, a CDR2 of SEQ ID No. 75 or a sequence with at least 90% homology thereto, a CDR3 of SEQ ID No. 76 or a sequence with at least 90% homology thereto.

In one aspect the invention provides an antibody or fragment thereof which binds to LGR5, wherein the V_{L} of the antibody comprises the following CDR1, CDR2 and CDR3:
a) a CDR1 of SEQ ID No. 5 or a sequence with at least 90% homology thereto, a CDR2 of SEQ ID No. 6 or a sequence with at least 90% homology thereto, a CDR3 of SEQ ID No. 7 or a sequence with at least 90% homology thereto; or
b) a CDR1 of SEQ ID No. 11 or a sequence with at least 90% homology thereto, a CDR2 of SEQ ID No. 12 or a sequence with at least 90% homology thereto, a CDR3 of SEQ ID No. 13 or a sequence with at least 90% homology thereto; or
c) a CDR1 of SEQ ID No. 17 or a sequence with at least 90% homology thereto, a CDR2 of SEQ ID No. 18 or a sequence with at least 90% homology thereto, a CDR3 of SEQ ID No. 19 or a sequence with at least 90% homology thereto; or
d) a CDR1 of SEQ ID No. 23 or a sequence with at least 90% homology thereto, a CDR2 of SEQ ID No. 24 or a sequence with at least 90% homology thereto, a CDR3 of SEQ ID No. 25 or a sequence with at least 90% homology thereto; or
e) a CDR1 of SEQ ID No. 29 or a sequence with at least 90% homology thereto, a CDR2 of SEQ ID No. 30 or a sequence with at least 90% homology thereto, a CDR3 of SEQ ID No. 31 or a sequence with at least 90% homology thereto; or
f) a CDR1 of SEQ ID No. 35 or a sequence with at least 90% homology thereto, a CDR2 of SEQ ID No. 36 or a sequence with at least 90% homology thereto, a CDR3 of SEQ ID No. 37 or a sequence with at least 90% homology thereto; or
g) a CDR1 of SEQ ID No. 41 or a sequence with at least 90% homology thereto, a CDR2 of SEQ ID No. 42 or a sequence with at least 90% homology thereto, a CDR3 of SEQ ID No. 43 or a sequence with at least 90% homology thereto; or
h) a CDR1 of SEQ ID No. 47 or a sequence with at least 90% homology thereto, a CDR2 of SEQ ID No. 48 or a sequence with at least 90% homology thereto, a CDR3 of SEQ ID No. 49 or a sequence with at least 90% homology thereto; or
i) a CDR1 of SEQ ID No. 53 or a sequence with at least 90% homology thereto, a CDR2 of SEQ ID No. 54 or a sequence with at least 90% homology thereto, a CDR3 of SEQ ID No. 55 or a sequence with at least 90% homology thereto; or
j) a CDR1 of SEQ ID No. 59 or a sequence with at least 90% homology thereto, a CDR2 of SEQ ID No. 60 or a sequence with at least 90% homology thereto, a CDR3 of SEQ ID No. 61 or a sequence with at least 90% homology thereto; or
k) a CDR1 of SEQ ID No. 65 or a sequence with at least 90% homology thereto, a CDR2 of SEQ ID No. 66 or a sequence with at least 90% homology thereto, a CDR3 of SEQ ID No. 67 or a sequence with at least 90% homology thereto; or
l) a CDR1 of SEQ ID No. 71 or a sequence with at least 90% homology thereto, a CDR2 of SEQ ID No. 72 or a sequence with at least 90% homology thereto, a CDR3 of SEQ ID No. 73 or a sequence with at least 90% homology thereto; or
m) a CDR1 of SEQ ID No. 77 or a sequence with at least 90% homology thereto, a CDR2 of SEQ ID No. 78 or a sequence with at least 90% homology thereto, a CDR3 of SEQ ID No. 79 or a sequence with at least 90% homology thereto.

TABLE 1
CDR sequences of antibodies of the invention

| | V_{H} CDR1 | V_{H} CDR2 | V_{H} CDR3 | V_{L} CDR1 | V_{L} CDR2 | V_{L} CDR3 |
|---|---|---|---|---|---|---|
| Name | sequence | sequence | sequence | sequence | sequence | sequence |
| 1 | SEQ ID | SEQ ID | SEQ ID | SEQ ID | SEQ ID NO: | SEQ ID NO: |
| | NO: 2 | NO: 3 | NO: 4 | NO: 5 | 6 | 7 |
| | SYWMQ | EIDPSDYY | SLSGYVD | RASQDIRN | YRSRLQS | QQGNTLPP |
| | | TNYNQKFK | Y | RLN | | T |
| | | G | | | | |
| 2 | SEQ ID | SEQ ID | SEQ ID | SEQ ID | SEQ ID NO: | SEQ ID NO: |
| | NO: 8 | NO: 9 | NO: 10 | NO: 11 | 12 | 13 |
| | NYWMQ | EIDPSDSY | SLSGYVD | RASQDISN | YRSRLHS | QQGNSLPP |
| | | TNYNQKFK | Y | RLN | | T |
| | | G | | | | |
| 3 | SEQ ID | SEQ ID NO: | SEQ ID | SEQ ID | SEQ ID NO: | SEQ ID NO: |
| | NO: 14 | 15 | NO: 16 | NO: 17 | 18 | 19 |
| | SYWMQ | EIDPSDSY | SLSGYVD | RASQDISN | YRSRLQS | QQGNTLPP |
| | | SNYNQKFK | Y | RLN | | T |
| | | G | | | | |
| 4 | SEQ ID | SEQ ID NO: | SEQ ID | SEQ ID | SEQ ID NO: | SEQ ID NO: |
| | NO: 20 | 21 | NO: 22 | NO: 23 | 24 | 25 |
| | DYWMQ | EIDPSDSY | SLSGSVD | RASQDISN | YRSRLQS | QQGNTLPP |
| | | TNYNQKFQ | Y | RLN | | T |
| | | G | | | | |
| 12 | SEQ ID | SEQ ID NO: | SEQ ID | SEQ ID | SEQ ID NO: | SEQ ID NO: |
| | NO: 26 | 27 | NO: 28 | NO: 29 | 30 | 31 |
| | DYYMN | DINPNNGG | GYLFDY | KSSQSLLN | FASTRES | QQHYSTPL |
| | | TIYNQKFK | | SSNQKNY | | T |
| | | G | | LA | | |
| 2.1 | SEQ ID | SEQ ID NO: | SEQ ID | SEQ ID | SEQ ID NO: | SEQ ID NO: |
| | NO: 32 | 33 | NO: 34 | NO: 35 | 36 | 37 |
| | NYWMQ | EIDPSDSY | SLSGYVD | RASQDISN | YRSRLHS | QQGNSLPP |
| | | TNYNQKFQ | Y | RLN | | T |
| | | G | | | | |
| 2.2 | SEQ ID | SEQ ID NO: | SEQ ID | SEQ ID | SEQ ID NO: | SEQ ID NO: |
| | NO: 38 | 39 | NO: 40 | NO: 41 | 42 | 43 |
| | NYWMQ | EIDPSDSY | SLSGYVD | RASQDISN | YRSRLHS | QQGNSLPP |
| | | TNYNQKFQ | Y | RLN | | T |
| | | G | | | | |
| 2.3 | SEQ ID | SEQ ID NO: | SEQ ID | SEQ ID | SEQ ID NO: | SEQ ID NO: |
| | NO: 44 | 45 | NO: 46 | NO: 47 | 48 | 49 |
| | NYWMQ | EIDPSDSY | SLSGYVD | RASQDISN | YRSRLHS | QQGNSLPP |
| | | TNYNQKFQ | Y | RLN | | T |
| | | G | | | | |
| 2.4 | SEQ ID | SEQ ID | SEQ ID | SEQ ID | SEQ ID NO: | SEQ ID NO: |
| | NO: 50 | NO: 51 | NO: 52 | NO: 53 | 54 | 55 |
| | NYWMQ | EIDPSDSY | SLSGYVD | RASQDISN | YRSRRHT | QQGNSLPP |
| | | TNYNQKFQ | Y | RLN | | T |
| | | G | | | | |
| 2.9 | SEQ ID | SEQ ID NO: | SEQ ID | SEQ ID | SEQ ID NO: | SEQ ID NO: |
| | NO: 56 | 57 | NO: 58 | NO: 59 | 60 | 61 |
| | NYWMQ | EIDPSDSY | SLSGYVD | RASQDISN | YRSRLHS | QQGNSLPP |
| | | TNYNQGFT | Y | RLN | | T |
| | | G | | | | |
| 2.11 | SEQ ID | SEQ ID NO: | SEQ ID | SEQ ID | SEQ ID NO: | SEQ ID NO: |
| | NO: 62 | 63 | NO: 64 | NO: 65 | 66 | 67 |
| | NYWMQ | EIDPSDSY | SLSGYVD | RASQDISN | YRSRLHS | QQGNSLPP |
| | | TNYNQGFT | Y | RLN | | T |
| | | G | | | | |
| 2.12 | SEQ ID | SEQ ID NO: | SEQ ID | SEQ ID | SEQ ID NO: | SEQ ID NO: |
| | NO: 68 | 69 | NO: 70 | NO: 71 | 72 | 73 |
| | NYWMQ | EIDPSDSY | SLSGYVD | RASQDISN | YRSRRHT | QQGNSLPP |
| | | TNYNQGFT | Y | RLN | | T |
| | | G | | | | |
| 2.16 | SEQ ID | SEQ ID | SEQ ID | SEQ ID | SEQ ID NO: | SEQ ID NO: |
| | NO: 74 | NO: 75 | NO: 76 | NO: 77 | 78 | 79 |
| | NYWMN | EIDPSDSY | SLSGYVD | RASQDISN | YRSRRHT | QQGNSLPP |
| | | TNYNQGFT | Y | RLN | | T |
| | | G | | | | |

In Table 1, the clones 1, 2, 3, 4 and 12 are mouse antibodies and clones 2.1, 2.2, 2.3, 2.4, 2.9, 2.11, 2.12 and 2.16 are humanized antibodies based on clone 2.

In embodiments in which the antibodies are humanized antibodies, the antibodies may comprise CDR1, CDR2 and CDR3 sequences as shown in Table 1 for clones 2.1, 2.2, 2.3, 2.4, 2.9, 2.11, 2.12 and 2.16 or sequences with at least 90% homology thereto. In one embodiment, the CDR1, CDR2 and CDR3 sequences as shown in Table 1 for clones 2.1, 2.2, 2.3, 2.4, 2.9, 2.11, 2.12 and 2.16 but include 1, 2 or 3 amino acid modifications, e.g. a substitution, deletion or insertion.

In one embodiment, the antibody provided comprise CDR1, CDR2 and CDR3 sequences as shown in Table 1 for Clone 2.4. Clone 2.4 is also referred to herein as α-LGR5v4, α-LGR5 variant 4 or human variant 4, see e.g. Table 5.

In one embodiment, the invention relates to an antibody or fragment thereof which binds human LGR5, wherein the antibody comprises a V_{H} sequence selected from those shown in Table 2 below or a sequence with at least 60%, 70%, 80%, 90%, 95% or more sequence identity thereto. In one aspect, the invention relates to an antibody which binds LGR5, wherein the antibody comprises a V_{H} sequence selected from SEQ ID NOs: 80, 82, 84, 86, 88, 90, 92, 94, 96, 98, 100, 102, 104 or a sequence with at least 60%, 70%, 80%, 90%, 95% or more sequence homology to one of SEQ ID NOs: 80, 82, 84, 86, 88, 90, 92, 94, 96, 98, 100, 102, 104. In one embodiment, said sequence homology is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In one embodiment, said sequence homology is at least 90%, 91%, 92%, 93%, 94%, 95%, 96%, 97%, 98% or 99%.

In one embodiment, the invention relates to an antibody which binds LGR5, wherein the antibody comprises a V_{L} sequence selected from those shown in Table 2 below or a sequence with at least 60%, 70%, 80%, 90%, 95% or more sequence identity thereto. In one aspect, the invention relates to an antibody which binds LGR5, wherein the antibody comprises a V_{L} sequence selected from SEQ ID Nos. 81, 83, 85, 87, 89, 91, 93, 95, 97, 99, 101, 103, 105 or a sequence with at least 60%, 70%, 80%, 90%, 95% or more sequence homology to one of SEQ ID Nos. 81, 83, 85, 87, 89, 91, 93, 95, 97, 99, 101, 103, 105. In one embodiment, said sequence homology is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In one embodiment, said sequence homology is at least 90%, 91%, 92%, 93%, 94%, 95%, 96%, 97%, 98% or 99%.

The invention also provides an antibody which binds to LGR5, the antibody comprising the V_{H} and V_{L} sequences shown in Table 2. Thus, the invention provides antibodies which bind to LGR5, and comprise:
a) a V_{H} sequence of SEQ ID NO: 80 and a V_{L} sequence of SEQ ID NO: 81;
b) a V_{H} sequence of SEQ ID NO: 82 and a V_{L} sequence of SEQ ID NO: 83;
c) a V_{H} sequence of SEQ ID NO: 84 and a V_{L} sequence of SEQ ID NO: 85;
d) a V_{H} sequence of SEQ ID NO: 86 and a V_{L} sequence of SEQ ID NO: 87;
e) a V_{H} sequence of SEQ ID NO: 88 and a V_{L} sequence of SEQ ID NO: 89;
f) a V_{H} sequence of SEQ ID NO: 90 and a V_{L} sequence of SEQ ID NO: 91;
g) a V_{H} sequence of SEQ ID NO: 92 and a V_{L} sequence of SEQ ID NO: 93;
h) a V_{H} sequence of SEQ ID NO: 94 and a V_{L} sequence of SEQ ID NO: 95;
i) a V_{H} sequence of SEQ ID NO: 96 and a V_{L} sequence of SEQ ID NO: 97;
j) a V_{H} sequence of SEQ ID NO: 98 and a V_{L} sequence of SEQ ID NO: 99;
k) a V_{H} sequence of SEQ ID NO: 100 and a V_{L} sequence of SEQ ID NO: 101
l) a V_{H} sequence of SEQ ID NO: 102 and a V_{L} sequence of SEQ ID NO: 103; or
m) a V_{H} sequence of SEQ ID NO: 104 and a V_{L} sequence of SEQ ID NO: 105.

In one embodiment, the antibody comprises comprise V_{H} and V_{L} sequences as shown in Table 2 for clone 2.4.

TABLE 2
V_{H} and V_{L} sequences of antibodies of the invention

| Name | V_{H} | V_{L} |
|---|---|---|
| 1 | SEQ ID NO: 80 | SEQ ID NO: 81 |
| | QVQLQQPGAELVKPGASVKLSCK | DIQMTQSTSSLSASLGDRVTINCRA |
| | ASGYTFTSYWMQWVKQRPGQGL | SQDIRNRLNWYQQKPDGTVKLLISY |
| | EWIGEIDPSDYYTNYNQKFKGKAT | RSRLQSGVPSRFSGSGSGTEYSLTI |
| | LTVDTSSSTAYMQLSSLTSEDSAV | SNLEQEDIATYFCQQGNTLPPTFG |
| | YYCARSLSGYVDYWGQGTTLTVS | GGTKLEVR |
| | S | |
| 2 | SEQ ID NO: 82 | SEQ ID NO: 83 |
| | QVQLQQPGAELVKPGASVKLSCK | DIQMTQTTSSLSASLGDRVTISCRA |
| | ASGYTFTNYWMQWVKQRPGQGL | SQDISNRLNWYQQKPDGTVKLLISY |
| | EWIGEIDPSDSYTNYNQKFKGKAT | RSRLHSGVPSRFSGSGSGTDYSLTI |
| | LTVDTSSSTAYMQLSSLTSEDSAV | SNLEQEDIATYFCQQGNSLPPTFG |
| | YYCARSLSGYVDYWGQGTTLTVS | GGTKLEVR |
| | S | |
| 3 | SEQ ID NO: 84 | SEQ ID NO: 85 |
| | QVQLQQPGAELVKPGASVKLSCK | DIQMTQTTSSLSASLGDRVTISCRA |
| | ASGYTFTSYWMQWVKQRPGQGL | SQDISNRLNWYQQKPDGTVKLLISY |
| | EWIGEIDPSDSYSNYNQKFKGKAT | RSRLQSGVPSRFSGSGSGTDYSLT |
| | LTVDTSSSTAYMQLSSLTSEDSAV | ISNLEEEDIATYFCQQGNTLPPTFG |
| | YYCARSLSGYVDYWGQGTTLTVS | GGTKLEVR |
| | S | |
| 4 | SEQ ID NO: 86 | SEQ ID NO: 87 |
| | QVQLQQPGAELVKPGASVKLSCK | DIQMTQTTSSLSASLGDRVTISCRA |
| | ASGYTFTDYWMQWVKQRPGQGL | SQDISNRLNWYQQKPDGTVKLLISY |
| | EWIGEIDPSDSYTNYNQKFQGKAT | RSRLQSGVPSRFSGSGSGTDYSLT |
| | LTVDTSSSTAYIQLSSLTSDDSAVY | ITNLEQEDIATYFCQQGNTLPPTFG |
| | YCARSLSGSVDYWGQGTTLTVSS | GGTKLEVR |
| 12 | SEQ ID NO: 88 | SEQ ID NO: 89 |
| | EVQLQQSGPELVKPGASVKISCKA | DIVMTQSPSSLAMSVGQKVTMSCK |
| | SGYTFTDYYMNWVKQSHGKSLEW | SSQSLLNSSNQKNYLAWYQQKPG |
| | IGDINPNNGGTIYNQKFKGKATLTV | QSPKLLVYFASTRESGVPDRFIGSG |
| | DKSSSTAYMELRSLTSEDSAVYYC | SGTDFTLTISSVQAEDLAAYFCQQH |
| | ASGYLFDYWGPGTTLTVSS | YSTPLTFGAGTKLELK |
| 2.1 | SEQ ID NO: 90 | SEQ ID NO: 91 |
| | QVQLVQSGAEVKKPGASVKVSCK | DIQMTQSPSSLSASVGDRVTITCRA |
| | ASGYTFTNYWMQWVRQAPGQGL | SQDISNRLNWYQQKPGKAVKLLISY |
| | EWIGEIDPSDSYTNYNQKFQGRVT | RSRLHSGVPSRFSGSGSGTDYTLTI |
| | LTVDTSTSTAYMELSSLRSEDTAV | SSLQPEDFATYFCQQGNSLPPTFG |
| | YYCARSLSGYVDYWGQGTTVTVS | GGTKLEIK |
| | S | |
| 2.2 | SEQ ID NO: 92 | SEQ ID NO: 93 |
| | QVQLVQSGAEVKKPGASVKVSCK | DIQMTQSPSSLSASVGDRVTITCRA |
| | ASGYTFTNYWMQWVRQAPGQGL | SQDISNRLNWYQQKPGKAVKLLISY |
| | EWIGEIDPSDSYTNYNQKFQGRVT | RSRLHSGVPSRFSGSGSGTDYTLTI |
| | LTVDTSTSTAYMELSSLRSEDTAV | SSLQPEDFATYYCQQGNSLPPTFG |
| | YYCARSLSGYVDYWGQGTTVTVS | GGTKLEIK |
| | S | |
| 2.3 | SEQ ID NO: 94 | SEQ ID NO: 95 |
| | QVQLVQSGAEVKKPGASVKVSCK | DIVMTQSPATLSLSPGERATLSCRA |
| | ASGYTFTNYWMQWVRQAPGQGL | SQDISNRLNWYQQKPGQAVRLLIS |
| | EWIGEIDPSDSYTNYNQKFQGRVT | YRSRLHSGVPARFSGSGSGTDYTL |
| | LTVDTSTSTAYMELSSLRSEDTAV | TISSLEPEDFAVYFCQQGNSLPPTF |
| | YYCARSLSGYVDYWGQGTTVTVS | GGGTKLEIK |
| | S | |
| 2.4 | SEQ ID NO: 96 | SEQ ID NO: 97 |
| | QVQLVQSGAEVKKPGASVKVSCK | EIVMTQSPATLSLSPGERATLSCRA |
| | ASGYTFTNYWMQWVRQAPGQGL | SQDISNRLNWYQQKPGQAVRLLIS |
| | EWIGEIDPSDSYTNYNQKFQGRVT | YRSRRHTGIPARFSGSGSGTDYTL |
| | LTVDTSTSTAYMELSSLRSEDTAV | TISSLEPEDFAVYYCQQGNSLPPTF |
| | YYCARSLSGYVDYWGQGTTVTVS | GGGTKLEIK |
| | S | |
| 2.9 | SEQ ID NO: 98 | SEQ ID NO: 99 |
| | QVQLVQSGSELKKPGASVKVSCK | DIQMTQSPSSLSASVGDRVTITCRA |
| | ASGYTFTNYWMQWVRQAPGQGL | SQDISNRLNWYQQKPGKAVKLLISY |
| | EWIGEIDPSDSYTNYNQGFTGRFV | RSRLHSGVPSRFSGSGSGTDYTLTI |
| | LSVDTSVSTAYLQISSLKAEDTAVY | SSLQPEDFATYFCQQGNSLPPTFG |
| | YCARSLSGYVDYWGQGTTVTVSS | GGTKLEIK |
| 2.11 | SEQ ID NO: 100 | SEQ ID NO: 101 |
| | QVQLVQSGSELKKPGASVKVSCK | DIQMTQSPSSLSASVGDRVTITCRA |
| | ASGYTFTNYWMQWVRQAPGQGL | SQDISNRLNWYQQKPGKAVKLLISY |
| | EWIGEIDPSDSYTNYNQGFTGRFV | RSRLHSGVPSRFSGSGSGTDYTLTI |
| | LSVDTSVSTAYLQISSLKAEDTAVY | SSLQPEDFATYYCQQGNSLPPTFG |
| | YCARSLSGYVDYWGQGTTVTVSS | GGTKLEIK |
| 2.12 | SEQ ID NO: 102 | SEQ ID NO: 103 |
| | QVQLVQSGSELKKPGASVKVSCK | DIVMTQSPATLSLSPGERATLSCRA |
| | ASGYTFTNYWMQWVRQAPGQGL | SQDISNRLNWYQQKPGQAVRLLIS |
| | EWIGEIDPSDSYTNYNQGFTGRFV | YRSRLHSGVPARFSGSGSGTDYTL |
| | LSVDTSVSTAYLQISSLKAEDTAVY | TISSLEPEDFAVYFCQQGNSLPPTF |
| | YCARSLSGYVDYWGQGTTVTVSS | GGGTKLEIK |
| 2.16 | SEQ ID NO: 104 | SEQ ID NO: 105 |
| | QVQLVQSGSELKKPGASVKVSCK | EIVMTQSPATLSLSPGERATLSCRA |
| | ASGYTFTNYWMNWVRQAPGQGL | SQDISNRLNWYQQKPGQAVRLLIS |
| | EWMGEIDPSDSYTNYNQGFTGRF | YRSRRHTGIPARFSGSGSGTDYTL |
| | VFSVDTSVSTAYLQISSLKAEDTAV | TISSLEPEDFAVYYCQQGNSLPPTF |
| | YYCARSLSGYVDYWGQGTTVTVS | GGGTKLEIK |
| | S | |

In Table 2, clones 1, 2, 3, 4 and 12 are murine antibodies and clones 2.1, 2.2, 2.3, 2.4, 2.9, 2.11, 2.12 and 2.16 are humanized antibodies.

In embodiments in which the antibodies are humanized antibodies, the antibodies may comprise V_{H} and V_{L} sequences as shown in Table 2 for clones 2.1, 2.2, 2.3, 2.4, 2.9, 2.11, 2.12 and 2.16 or sequences with at least 90% homology thereto.

In one embodiment, the V_{H} and V_{L} sequences as shown in Table 2 for clones 2.1, 2.2, 2.3, 2.4, 2.9, 2.11, 2.12 and 2.16 but include 1,2, 3, 4, 5, 6, 7, 8, 9 or 10 amino acid modifications, e.g. a substitution, deletion or insertion.

The antibody may comprise a CH2 domain. The CH2 domain is preferably located at the N-terminus of the CH3 domain, as in the case in a human IgG molecule. The CH2 domain of the antibody is preferably the CH2 domain of human IgG1, IgG2, IgG3, or IgG4, more preferably the CH2 domain of human IgG1. The sequences of human IgG domains are known in the art.

The antibody may comprise an immunoglobulin hinge region, or part thereof, at the N-terminus of the CH2 domain. The immunoglobulin hinge region allows the two CH2-CH3 domain sequences to associate and form a dimer. Preferably, the hinge region, or part thereof, is a human IgG1, IgG2, IgG3 or IgG4 hinge region, or part thereof. More preferably, the hinge region, or part thereof, is an IgG1 hinge region, or part thereof.

The sequence of the CH3 domain is not particularly limited. Preferably, the CH3 domain is a human immunoglobulin G domain, such as a human IgG1, IgG2, IgG3, or IgG4 CH3 domain, most preferably a human IgG1 CH3 domain.

An antibody of the invention may comprise a human IgG1, IgG2, IgG3, or IgG4 constant region. The sequences of human IgG1, IgG2, IgG3, or IgG4 CH3 domains are known in the art. An antibody of the invention may comprise a non-human IgG constant region, e.g., a rabbit IgG 1 constant region.

In another embodiment, the antibody comprises or consists of a polypeptide sequence as shown for any one of the antibody clones in Table 3 or a sequence with at least 60%, 70%, 80%, 90%, 95% or more sequence homology thereto. Thus, the antibody comprises or consists of an amino acid sequence selected from the combination of V_{H}, C_{H}, V_{L} and C_{L} sequences for the clones shown in Table 3 or a sequence with at least 60%, 70%, 80%, 90%, 95% or more sequence homology thereto. In one embodiment, said sequence homology is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Thus, the invention provides antibodies which bind to LGR5, and comprise:
a) a V_{H} sequence of SEQ ID NO: 106; a C_{H} sequence of SEQ ID NO:107; a V_{L} sequence of SEQ ID NO: 108; and a C_{L} sequence of SEQ ID NO: 109;
b) a V_{H} sequence of SEQ ID NO: 110; a C_{H} sequence of SEQ ID NO:111; a V_{L} sequence of SEQ ID NO: 112; and a C_{L} sequence of SEQ ID NO: 113;
c) a V_{H} sequence of SEQ ID NO: 114; a C_{H} sequence of SEQ ID NO:115; a V_{L} sequence of SEQ ID NO: 116; and a C_{L} sequence of SEQ ID NO: 117;
d) a V_{H} sequence of SEQ ID NO: 118; a C_{H} sequence of SEQ ID NO:119; a V_{L} sequence of SEQ ID NO: 120; and a C_{L} sequence of SEQ ID NO: 121;
e) a V_{H} sequence of SEQ ID NO: 122; a C_{H} sequence of SEQ ID NO:123; a V_{L} sequence of SEQ ID NO: 124; and a C_{L} sequence of SEQ ID NO: 125;
f) a V_{H} sequence of SEQ ID NO: 126; a C_{H} sequence of SEQ ID NO:127; a V_{L} sequence of SEQ ID NO: 128; and a C_{L} sequence of SEQ ID NO: 129;
g) a V_{H} sequence of SEQ ID NO: 130; a C_{H} sequence of SEQ ID NO:131; a V_{L} sequence of SEQ ID NO: 132; and a C_{L} sequence of SEQ ID NO: 133;
h) a V_{H} sequence of SEQ ID NO: 134; a C_{H} sequence of SEQ ID NO:135; a V_{L} sequence of SEQ ID NO: 136; and a C_{L} sequence of SEQ ID NO: 137;
i) a V_{H} sequence of SEQ ID NO: 138; a C_{H} sequence of SEQ ID NO:139; a V_{L} sequence of SEQ ID NO: 140; and a C_{L} sequence of SEQ ID NO: 141;
j) a V_{H} sequence of SEQ ID NO: 142; a C_{H} sequence of SEQ ID NO:143; a V_{L} sequence of SEQ ID NO: 144; and a C_{L} sequence of SEQ ID NO: 145;
k) a V_{H} sequence of SEQ ID NO: 146; a C_{H} sequence of SEQ ID NO:147; a V_{L} sequence of SEQ ID NO: 148; and a C_{L} sequence of SEQ ID NO: 149;
l) a V_{H} sequence of SEQ ID NO: 150; a C_{H} sequence of SEQ ID NO:151; a V_{L} sequence of SEQ ID NO: 152; and a C_{L} sequence of SEQ ID NO: 153; or
m) a V_{H} sequence of SEQ ID NO: 154; a C_{H} sequence of SEQ ID NO:155; a V_{L} sequence of SEQ ID NO: 156; and a C_{L} sequence of SEQ ID NO: 157.

TABLE 3
Full sequences of antibodies of the invention

| | V_{H} | C_{H} | V_{L} | C_{L} |
|---|---|---|---|---|
| 1 | SEQ ID NO: 106 | SEQ ID NO: 107 | SEQ ID NO: 108 | SEQ ID NO: 109 |
| | QVQLQQPGAEL | AKTTPPSVYPLAPGSAAQTNSMV | DIQMTQSTSSL | RADAAPTVSIF |
| | VKPGASVKLSCK | TLGCLVKGYFPEPVTVTWNSGSL | SASLGDRVTIN | PPSSEQLTSG |
| | ASGYTFTSYWM | SSGVHTFPAVLQSDLYTLSSSVTV | CRASQDIRNRL | GASVVCFLNNF |
| | QWVKQRPGQGL | PSSTWPSQTVTCNVAHPASSTKV | NWYQQKPDGT | YPKDINVKWKI |
| | EWIGEIDPSDYY | DKKIVPRDCGCKPCICTVPEVSSV | VKLLISYRSRL | DGSERQNGVL |
| | TNYNQKFKGKA | FIFPPKPKDVLTITLTPKVTCVVVDI | QSGVPSRFSG | NSWTDQDSKD |
| | TLTVDTSSSTAY | SKDDPEVQFSWFVDDVEVHTAQT | SGSGTEYSLTI | STYSMSSTLTL |
| | MQLSSLTSEDSA | KPREEQINSTFRSVSELPIMHQD | SNLEQEDIATY | TKDEYERHNS |
| | VYYCARSLSGYV | WLNGKEFKCRVNSAAFPAPIEKTI | FCQQGNTLPP | YTCEATHKTST |
| | DYWGQGTTLTV | SKTKGRPKAPQVYTIPPPKEQMA | TFGGGTKLEV | SPIVKSFNRNE |
| | SS | KDKVSLTCMITNFFPEDITVEWQW | R | C |
| | | NGQPAENYKNTQPIMDTDGSYFV | | |
| | | YSKLNVQKSNWEAGNTFTCSVLH | | |
| | | EGLHNHHTEKSLSHSPGK | | |
| 2 | SEQ ID NO: 110 | SEQ ID NO: 111 | SEQ ID NO: 112 | SEQ ID NO: 113 |
| | QVQLQQPGAEL | AKTTPPSVYPLAPGCGDTTGSSV | DIQMTQTTSSL | RADAAPTVSIF |
| | VKPGASVKLSCK | TLGCLVKGYFPESVTVTWNSGSL | SASLGDRVTIS | PPSSEQLTSG |
| | ASGYTFTNYWM | SSSVHTFPALLQSGLYTMSSSVTV | CRASQDISNRL | GASVVCFLNNF |
| | QWVKQRPGQGL | PSSTWPSQTVTCSVAHPASSTTV | NWYQQKPDGT | YPKDINVKWKI |
| | EWIGEIDPSDSY | DKKLEPSGPISTINPCPPCKECHK | VKLLISYRSRL | DGSERQNGVL |
| | TNYNQKFKGKA | CPAPNLEGGPSVFIFPPNIKDVLMI | HSGVPSRFSG | NSWTDQDSKD |
| | TLTVDTSSSTAY | SLTPKVTCVVVDVSEDDPDVRIS | SGSGTDYSLTI | STYSMSSTLTL |
| | MQLSSLTSEDSA | WFVNNVEVHTAQTQTHREDYNS | SNLEQEDIATY | TKDEYERHNS |
| | VYYCARSLSGYV | TIRVVSALPIQHQDWMSGKEFKC | FCQQGNSLPP | YTCEATHKTST |
| | DYWGQGTTLTV | KVNNKDLPSPIERTISKIKGLVRAP | TFGGGTKLEV | SPIVKSFNRNE |
| | SS | QVYILPPPAEQLSRKDVSLTCLVV | R | C |
| | | GFNPGDISVEWTSNGHTEENYKD | | |
| | | TAPVLDSDGSYFIYSKLDIKTSKW | | |
| | | EKTDSFSCNVRHEGLKNYYLKKTI | | |
| | | SRSPGK | | |
| 3 | SEQ ID NO: 114 | SEQ ID NO: 115 | SEQ ID NO: 116 | SEQ ID NO: 117 |
| | QVQLQQPGAEL | AKTTPPSVYPLAPGSAAQTNSMV | DIQMTQTTSSL | RADAAPTVSIF |
| | VKPGASVKLSCK | TLGCLVKGYFPEPVTVTWNSGSL | SASLGDRVTIS | PPSSEQLTSG |
| | ASGYTFTSYWM | SSGVHTFPAVLQSDLYTLSSSVTV | CRASQDISNRL | GASVVCFLNNF |
| | QWVKQRPGQGL | PSSTWPSQTVTCNVAHPASSTKV | NWYQQKPDGT | YPKDINVKWKI |
| | EWIGEIDPSDSY | DKKIVPRDCGCKPCICTVPEVSSV | VKLLISYRSRL | DGSERQNGVL |
| | SNYNQKFKGKA | FIFPPKPKDVLTITLTPKVTCVVVDI | QSGVPSRFSG | NSWTDQDSKD |
| | TLTVDTSSSTAY | SKDDPEVQFSWFVDDVEVHTAQT | SGSGTDYSLTI | STYSMSSTLTL |
| | MQLSSLTSEDSA | KPREEQINSTFRSVSELPIMHQD | SNLEEEDIATY | TKDEYERHNS |
| | VYYCARSLSGYV | WLNGKEFKCRVNSAAFPAPIEKTI | FCQQGNTLPP | YTCEATHKTST |
| | DYWGQGTTLTV | SKTKGRPKAPQVYTIPPPKEQMA | TFGGGTKLEV | SPIVKSFNRNE |
| | SS | KDKVSLTCMITNFFPEDITVEWQW | R | C |
| | | NGQPAENYKNTQPIMDTDGSYFV | | |
| | | YSKLNVQKSNWEAGNTFTCSVLH | | |
| | | EGLHNHHTEKSLSHSPGK | | |
| 4 | SEQ ID NO: 118 | SEQ ID NO: 119 | SEQ ID NO: 120 | SEQ ID NO: 121 |
| | QVQLQQPGAEL | AKTTPPSVYPLAPGCGDTTGSSV | DIQMTQTTSSL | RADAAPTVSIF |
| | VKPGASVKLSCK | TLGCLVKGYFPESVTVTWNSGSL | SASLGDRVTIS | PPSSEQLTSG |
| | ASGYTFTDYWM | SSSVHTFPALLQSGLYTMSSSVTV | CRASQDISNRL | GASVVCFLNNF |
| | QWVKQRPGQGL | PSSTWPSQTVTCSVAHPASSTTV | NWYQQKPDGT | YPKDINVKWKI |
| | EWIGEIDPSDSY | DKKLEPSGPISTINPCPPCKECHK | VKLLISYRSRL | DGSERQNGVL |
| | TNYNQKFQGKA | CPAPNLEGGPSVFIFPPNIKDVLMI | QSGVPSRFSG | NSWTDQDSKD |
| | TLTVDTSSSTAYI | SLTPKVTCVVVDVSEDDPDVRIS | SGSGTDYSLTI | STYSMSSTLTL |
| | QLSSLTSDDSAV | WFVNNVEVHTAQTQTHREDYNS | TNLEQEDIATY | TKDEYERHNS |
| | YYCARSLSGSV | TIRVVSALPIQHQDWMSGKEFKC | FCQQGNTLPP | YTCEATHKTST |
| | DYWGQGTTLTV | KVNNKDLPSPIERTISKIKGLVRAP | TFGGGTKLEV | SPIVKSFNRNE |
| | SS | QVYILPPPAEQLSRKDVSLTCLVV | R | C |
| | | GFNPGDISVEWTSNGHTEENYKD | | |
| | | TAPVLDSDGSYFIYSKLDIKTSKW | | |
| | | EKTDSFSCNVRHEGLKNYYLKKTI | | |
| | | SRSPGK | | |
| 12 | SEQ ID NO: 122 | SEQ ID NO: 123 | SEQ ID NO: 124 | SEQ ID NO: 125 |
| | EVQLQQSGPEL | AKTTAPSVYPLAPVCGGTTGSSV | DIVMTQSPSSL | RADAAPTVSIF |
| | VKPGASVKISCK | TLGCLVKGYFPEPVTLTWNSGSL | AMSVGQKVTM | PPSSEQLTSG |
| | ASGYTFTDYYM | SSGVHTFPALLQSGLYTLSSSVTV | SCKSSQSLLNS | GASVVCFLNNF |
| | NWVKQSHGKSL | TSNTWPSQTITCNVAHPASSTKV | SNQKNYLAWY | YPKDINVKWKI |
| | EWIGDINPNNGG | DKKIEPRVPITQNPCPPLKECPPC | QQKPGQSPKL | DGSERQNGVL |
| | TIYNQKFKGKAT | AAPDLLGGPSVFIFPPKIKDVLMIS | LVYFASTRESG | NSWTDQDSKD |
| | LTVDKSSSTAYM | LSPMVTCVVVDVSEDDPDVQISW | VPDRFIGSGSG | STYSMSSTLTL |
| | ELRSLTSEDSAV | FVNNVEVHTAQTQTHREDYNSTL | TDFTLTISSVQ | TKDEYERHNS |
| | YYCASGYLFDY | RVVSALPIQHQDWMSGKEFKCKV | AEDLAAYFCQ | YTCEATHKTST |
| | WGPGTTLTVSS | NNRALPSPIEKTISKPRGPVRAPQ | QHYSTPLTFGA | SPIVKSFNRNE |
| | | VYVLPPPAEEMTKKEFSLTCMITG | GTKLELK | C |
| | | FLPAEIAVDWTSNGRTEQNYKNT | | |
| | | ATVLDSDGSYFMYSKLRVQKSTW | | |
| | | ERGSLFACSVVHEGLHNHLTTKTI | | |
| | | SRSLGK | | |
| 2.1 | SEQ ID NO: 126 | SEQ ID NO: 127 | SEQ ID NO: 128 | SEQ ID NO: 129 |
| | QVQLVQSGAEV | ASTKGPSVFPLAPSSKSTSGGTA | DIQMTQSPSSL | RTVAAPSVFIF |
| | KKPGASVKVSC | ALGCLVKDYFPEPVTVSWNSGAL | SASVGDRVTIT | PPSDEQLKSG |
| | KASGYTFTNYW | TSGVHTFPAVLQSSGLYSLSSVVT | CRASQDISNRL | TASVVCLLNNF |
| | MQWVRQAPGQ | VPSSSLGTQTYICNVNHKPSNTKV | NWYQQKPGKA | YPREAKVQWK |
| | GLEWIGEIDPSD | DKKVEPKSCDKTHTCPPCPAPEL | VKLLISYRSRL | VDNALQSGNS |
| | SYTNYNQKFQG | LGGPSVFLFPPKPKDTLMISRTPE | HSGVPSRFSG | QESVTEQDSK |
| | RVTLTVDTSTST | VTCVVVDVSHEDPEVKFNWYVD | SGSGTDYTLTI | DSTYSLSSTLT |
| | AYMELSSLRSED | GVEVHNAKTKPREEQYNSTYRVV | SSLQPEDFATY | LSKADYEKHKV |
| | TAVYYCARSLSG | SVLTVLHQDWLNGKEYKCKVSNK | FCQQGNSLPP | YACEVTHQGL |
| | YVDYWGQGTTV | ALPAPIEKTISKAKGQPREPQVYT | TFGGGTKLEIK | SSPVTKSFNR |
| | TVSS | LPPSRDELTKNQVSLTCLVKGFYP | | GEC |
| | | SDIAVEWESNGQPENNYKTTPPV | | |
| | | LDSDGSFFLYSKLTVDKSRWQQG | | |
| | | NVFSCSVMHEALHNHYTQKSLSL | | |
| | | SPGK | | |
| 2.2 | SEQ ID NO: 130 | SEQ ID NO: 131 | SEQ ID NO: 132 | SEQ ID NO: 133 |
| | QVQLVQSGAEV | ASTKGPSVFPLAPSSKSTSGGTA | DIQMTQSPSSL | RTVAAPSVFIF |
| | KKPGASVKVSC | ALGCLVKDYFPEPVTVSWNSGAL | SASVGDRVTIT | PPSDEQLKSG |
| | KASGYTFTNYW | TSGVHTFPAVLQSSGLYSLSSVVT | CRASQDISNRL | TASVVCLLNNF |
| | MQWVRQAPGQ | VPSSSLGTQTYICNVNHKPSNTKV | NWYQQKPGKA | YPREAKVQWK |
| | GLEWIGEIDPSD | DKKVEPKSCDKTHTCPPCPAPEL | VKLLISYRSRL | VDNALQSGNS |
| | SYTNYNQKFQG | LGGPSVFLFPPKPKDTLMISRTPE | HSGVPSRFSG | QESVTEQDSK |
| | RVTLTVDTSTST | VTCVVVDVSHEDPEVKFNWYVD | SGSGTDYTLTI | DSTYSLSSTLT |
| | AYMELSSLRSED | GVEVHNAKTKPREEQYNSTYRVV | SSLQPEDFATY | LSKADYEKHKV |
| | TAVYYCARSLSG | SVLTVLHQDWLNGKEYKCKVSNK | YCQQGNSLPP | YACEVTHQGL |
| | YVDYWGQGTTV | ALPAPIEKTISKAKGQPREPQVYT | TFGGGTKLEIK | SSPVTKSFNR |
| | TVSS | LPPSRDELTKNQVSLTCLVKGFYP | | GEC |
| | | SDIAVEWESNGQPENNYKTTPPV | | |
| | | LDSDGSFFLYSKLTVDKSRWQQG | | |
| | | NVFSCSVMHEALHNHYTQKSLSL | | |
| | | SPGK | | |
| 2.3 | SEQ ID NO: 134 | SEQ ID NO: 135 | SEQ ID NO: 136 | SEQ ID NO: 137 |
| | QVQLVQSGAEV | ASTKGPSVFPLAPSSKSTSGGTA | DIVMTQSPATL | RTVAAPSVFIF |
| | KKPGASVKVSC | ALGCLVKDYFPEPVTVSWNSGAL | SLSPGERATLS | PPSDEQLKSG |
| | KASGYTFTNYW | TSGVHTFPAVLQSSGLYSLSSVVT | CRASQDISNRL | TASVVCLLNNF |
| | MQWVRQAPGQ | VPSSSLGTQTYICNVNHKPSNTKV | NWYQQKPGQ | YPREAKVQWK |
| | GLEWIGEIDPSD | DKKVEPKSCDKTHTCPPCPAPEL | AVRLLISYRSR | VDNALQSGNS |
| | SYTNYNQKFQG | LGGPSVFLFPPKPKDTLMISRTPE | LHSGVPARFS | QESVTEQDSK |
| | RVTLTVDTSTST | VTCVVVDVSHEDPEVKFNWYVD | GSGSGTDYTL | DSTYSLSSTLT |
| | AYMELSSLRSED | GVEVHNAKTKPREEQYNSTYRVV | TISSLEPEDFA | LSKADYEKHKV |
| | TAVYYCARSLSG | SVLTVLHQDWLNGKEYKCKVSNK | VYFCQQGNSL | YACEVTHQGL |
| | YVDYWGQGTTV | ALPAPIEKTISKAKGQPREPQVYT | PPTFGGGTKLE | SSPVTKSFNR |
| | TVSS | LPPSRDELTKNQVSLTCLVKGFYP | IK | GEC |
| | | SDIAVEWESNGQPENNYKTTPPV | | |
| | | LDSDGSFFLYSKLTVDKSRWQQG | | |
| | | NVFSCSVMHEALHNHYTQKSLSL | | |
| | | SPGK | | |
| 2.4 | SEQ ID NO: 138 | SEQ ID NO: 139 | SEQ ID NO: 140 | SEQ ID NO: 141 |
| | QVQLVQSGAEV | ASTKGPSVFPLAPSSKSTSGGTA | EIVMTQSPATL | RTVAAPSVFIF |
| | KKPGASVKVSC | ALGCLVKDYFPEPVTVSWNSGAL | SLSPGERATLS | PPSDEQLKSG |
| | KASGYTFTNYW | TSGVHTFPAVLQSSGLYSLSSVVT | CRASQDISNRL | TASVVCLLNNF |
| | MQWVRQAPGQ | VPSSSLGTQTYICNVNHKPSNTKV | NWYQQKPGQ | YPREAKVQWK |
| | GLEWIGEIDPSD | DKKVEPKSCDKTHTCPPCPAPEL | AVRLLISYRSR | VDNALQSGNS |
| | SYTNYNQKFQG | LGGPSVFLFPPKPKDTLMISRTPE | RHTGIPARFSG | QESVTEQDSK |
| | RVTLTVDTSTST | VTCVVVDVSHEDPEVKFNWYVD | SGSGTDYTLTI | DSTYSLSSTLT |
| | AYMELSSLRSED | GVEVHNAKTKPREEQYNSTYRVV | SSLEPEDFAVY | LSKADYEKHKV |
| | TAVYYCARSLSG | SVLTVLHQDWLNGKEYKCKVSNK | YCQQGNSLPP | YACEVTHQGL |
| | YVDYWGQGTTV | ALPAPIEKTISKAKGQPREPQVYT | TFGGGTKLEIK | SSPVTKSFNR |
| | TVSS | LPPSRDELTKNQVSLTCLVKGFYP | | GEC |
| | | SDIAVEWESNGQPENNYKTTPPV | | |
| | | LDSDGSFFLYSKLTVDKSRWQQG | | |
| | | NVFSCSVMHEALHNHYTQKSLSL | | |
| | | SPGK | | |
| 2.9 | SEQ ID NO: 142 | SEQ ID NO: 143 | SEQ ID NO: 144 | SEQ ID NO: 145 |
| | QVQLVQSGSEL | ASTKGPSVFPLAPSSKSTSGGTA | DIQMTQSPSSL | RTVAAPSVFIF |
| | KKPGASVKVSC | ALGCLVKDYFPEPVTVSWNSGAL | SASVGDRVTIT | PPSDEQLKSG |
| | KASGYTFTNYW | TSGVHTFPAVLQSSGLYSLSSVVT | CRASQDISNRL | TASVVCLLNNF |
| | MQWVRQAPGQ | VPSSSLGTQTYICNVNHKPSNTKV | NWYQQKPGKA | YPREAKVQWK |
| | GLEWIGEIDPSD | DKKVEPKSCDKTHTCPPCPAPEL | VKLLISYRSRL | VDNALQSGNS |
| | SYTNYNQGFTG | LGGPSVFLFPPKPKDTLMISRTPE | HSGVPSRFSG | QESVTEQDSK |
| | RFVLSVDTSVST | VTCVVVDVSHEDPEVKFNWYVD | SGSGTDYTLTI | DSTYSLSSTLT |
| | AYLQISSLKAED | GVEVHNAKTKPREEQYNSTYRVV | SSLQPEDFATY | LSKADYEKHKV |
| | TAVYYCARSLSG | SVLTVLHQDWLNGKEYKCKVSNK | FCQQGNSLPP | YACEVTHQGL |
| | YVDYWGQGTTV | ALPAPIEKTISKAKGQPREPQVYT | TFGGGTKLEIK | SSPVTKSFNR |
| | TVSS | LPPSRDELTKNQVSLTCLVKGFYP | | GEC |
| | | SDIAVEWESNGQPENNYKTTPPV | | |
| | | LDSDGSFFLYSKLTVDKSRWQQG | | |
| | | NVFSCSVMHEALHNHYTQKSLSL | | |
| | | SPGK | | |
| 2.11 | SEQ ID NO: 146 | SEQ ID NO: 147 | SEQ ID NO: 148 | SEQ ID NO: 149 |
| | QVQLVQSGSEL | ASTKGPSVFPLAPSSKSTSGGTA | DIVMTQSPATL | RTVAAPSVFIF |
| | KKPGASVKVSC | ALGCLVKDYFPEPVTVSWNSGAL | SLSPGERATLS | PPSDEQLKSG |
| | KASGYTFTNYW | TSGVHTFPAVLQSSGLYSLSSVVT | CRASQDISNRL | TASVVCLLNNF |
| | MQWVRQAPGQ | VPSSSLGTQTYICNVNHKPSNTKV | NWYQQKPGQ | YPREAKVQWK |
| | GLEWIGEIDPSD | DKKVEPKSCDKTHTCPPCPAPEL | AVRLLISYRSR | VDNALQSGNS |
| | SYTNYNQGFTG | LGGPSVFLFPPKPKDTLMISRTPE | LHSGVPARFS | QESVTEQDSK |
| | RFVLSVDTSVST | VTCVVVDVSHEDPEVKFNWYVD | GSGSGTDYTL | DSTYSLSSTLT |
| | AYLQISSLKAED | GVEVHNAKTKPREEQYNSTYRVV | TISSLEPEDFA | LSKADYEKHKV |
| | TAVYYCARSLSG | SVLTVLHQDWLNGKEYKCKVSNK | VYFCQQGNSL | YACEVTHQGL |
| | YVDYWGQGTTV | ALPAPIEKTISKAKGQPREPQVYT | PPTFGGGTKLE | SSPVTKSFNR |
| | TVSS | LPPSRDELTKNQVSLTCLVKGFYP | IK | GEC |
| | | SDIAVEWESNGQPENNYKTTPPV | | |
| | | LDSDGSFFLYSKLTVDKSRWQQG | | |
| | | NVFSCSVMHEALHNHYTQKSLSL | | |
| | | SPGK | | |
| 2.12 | SEQ ID NO: 150 | SEQ ID NO: 151 | SEQ ID NO: 152 | SEQ ID NO: 153 |
| | QVQLVQSGSEL | ASTKGPSVFPLAPSSKSTSGGTA | EIVMTQSPATL | RTVAAPSVFIF |
| | KKPGASVKVSC | ALGCLVKDYFPEPVTVSWNSGAL | SLSPGERATLS | PPSDEQLKSG |
| | KASGYTFTNYW | TSGVHTFPAVLQSSGLYSLSSVVT | CRASQDISNRL | TASVVCLLNNF |
| | MQWVRQAPGQ | VPSSSLGTQTYICNVNHKPSNTKV | NWYQQKPGQ | YPREAKVQWK |
| | GLEWIGEIDPSD | DKKVEPKSCDKTHTCPPCPAPEL | AVRLLISYRSR | VDNALQSGNS |
| | SYTNYNQGFTG | LGGPSVFLFPPKPKDTLMISRTPE | RHTGIPARFSG | QESVTEQDSK |
| | RFVLSVDTSVST | VTCVVVDVSHEDPEVKFNWYVD | SGSGTDYTLTI | DSTYSLSSTLT |
| | AYLQISSLKAED | GVEVHNAKTKPREEQYNSTYRVV | SSLEPEDFAVY | LSKADYEKHKV |
| | TAVYYCARSLSG | SVLTVLHQDWLNGKEYKCKVSNK | YCQQGNSLPP | YACEVTHQGL |
| | YVDYWGQGTTV | ALPAPIEKTISKAKGQPREPQVYT | TFGGGTKLEIK | SSPVTKSFNR |
| | TVSS | LPPSRDELTKNQVSLTCLVKGFYP | | GEC |
| | | SDIAVEWESNGQPENNYKTTPPV | | |
| | | LDSDGSFFLYSKLTVDKSRWQQG | | |
| | | NVFSCSVMHEALHNHYTQKSLSL | | |
| | | SPGK | | |
| 2.16 | SEQ ID NO: 154 | SEQ ID NO: 155 | SEQ ID NO: 156 | SEQ ID NO: 157 |
| | QVQLVQSGSEL | ASTKGPSVFPLAPSSKSTSGGTA | EIVMTQSPATL | RTVAAPSVFIF |
| | KKPGASVKVSC | ALGCLVKDYFPEPVTVSWNSGAL | SLSPGERATLS | PPSDEQLKSG |
| | KASGYTFTNYW | TSGVHTFPAVLQSSGLYSLSSVVT | CRASQDISNRL | TASVVCLLNNF |
| | MNWVRQAPGQ | VPSSSLGTQTYICNVNHKPSNTKV | NWYQQKPGQ | YPREAKVQWK |
| | GLEWMGEIDPS | DKKVEPKSCDKTHTCPPCPAPEL | AVRLLISYRSR | VDNALQSGNS |
| | DSYTNYNQGFT | LGGPSVFLFPPKPKDTLMISRTPE | RHTGIPARFSG | QESVTEQDSK |
| | GRFVFSVDTSVS | VTCVVVDVSHEDPEVKFNWYVD | SGSGTDYTLTI | DSTYSLSSTLT |
| | TAYLQISSLKAE | GVEVHNAKTKPREEQYNSTYRVV | SSLEPEDFAVY | LSKADYEKHKV |
| | DTAVYYCARSLS | SVLTVLHQDWLNGKEYKCKVSNK | YCQQGNSLPP | YACEVTHQGL |
| | GYVDYWGQGTT | ALPAPIEKTISKAKGQPREPQVYT | TFGGGTKLEIK | SSPVTKSFNR |
| | VTVSS | LPPSRDELTKNQVSLTCLVKGFYP | | GEC |
| | | SDIAVEWESNGQPENNYKTTPPV | | |
| | | LDSDGSFFLYSKLTVDKSRWQQG | | |
| | | NVFSCSVMHEALHNHYTQKSLSL | | |
| | | SPGK | | |

In Table 3, clones 1, 2, 3, 4 and 12 are murine antibodies and clones 2.1, 2.2, 2.3, 2.4, 2.9, 2.11, 2.12 and 2.16 are humanized antibodies.

In embodiments in which the antibodies are humanized antibodies, the antibodies may comprise sequences as shown in Table 3 for clones 2.1, 2.2, 2.3, 2.4, 2.9, 2.11, 2.12 and 2.16 or a sequence with at least 90% homology thereto.

In some embodiments, the invention provides an antibody that is a variant of any of the above antibodies or fragments having one or more amino acid modifications, e.g. substitutions, deletions, additions, insertions or other modifications, and which retains a biological function of the antibody. Thus, variant antibodies can be sequence engineered. Modifications include at least one substitution, deletion or insertion of one or more codons encoding the antibody or polypeptide that results in a change in the amino acid sequence as compared with the native antibody or polypeptide. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence. A variant of an antibody described herein has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence homology to the non-variant molecule, preferably at least 95%, 96%, 97%, 98% or 99% sequence homology.

In one embodiment, the modification is a conservative sequence modification. As used herein, the term “conservative sequence modifications” is intended to referto amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the functions set forth in (c) through (I) above) using the functional assays described herein.

In some embodiments, the invention provides an antibody that is a variant of an antibody selected from those shown in Table 3 that comprises one or more sequence modifications and has improvements in one or more of a property such as binding affinity, specificity, thermostability, expression level, effector function, glycosylation, reduced immunogenicity, or solubility as compared to the unmodified antibody. Sequence modifications may reside in the CDR1, CDR2 and/or CDR3 or in one or more of the framework regions.

An antibody of the invention may comprise a human IgG Fc with effector function.

Fc receptors (FcRs) are key immune regulatory receptors connecting the antibody mediated (humoral) immune response to cellular effector functions. Receptors for all classes of immunoglobulins have been identified, including FcyR (IgG), FcsRI (IgE), FcaRI (IgA), FcpR (IgM) and FcbR (IgD). There are three classes of receptors for human IgG found on leukocytes: CD64 (FcyRI), CD32 (FcyRIIa, FcyRIIb and FcyRIIc) and CD16 (FcyRIIIa and FcyRIIIb). FcyRI is classed as a high affinity receptor (nanomolar range Kd) while FcyRII and FcyRIII are low to intermediate affinity (micromolar range Kd).

In antibody dependent cellular cytotoxicity (ADCC), FcvRs on the surface of effector cells (natural killer cells, macrophages, monocytes and eosinophils) bind to the F c region of an IgG which itself is bound to a target cell. Upon binding a signalling pathway is triggered which results in the secretion of various substances, such as lytic enzymes, perforin, granzymes and tumour necrosis factor, which mediate in the destruction of the target cell. The level of ADCC effector function various for IgG subtypes. Although this is dependent on the allotype and specific FcvR in simple terms ADCC effector function is high for human IgG1 and IgG3, and low for IgG2 and IgG4. See below for IgG subtype variation in effector functions, ranked in decreasing potency.

FcyRs bind to IgG asymmetrically across the hinge and upper CH2 region. Knowledge of the binding site has resulted in engineering efforts to modulate IgG effector functions Antibodies of the invention may have an Fc with effector function, with enhanced effector function or with reduced effector function.

The potency of antibodies can be increased by enhancement of the ability to mediate cellular cytotoxicity functions, such as antibody-dependent cell-mediated cytotoxicity (ADCC) and antibody-dependent cell-mediated phagocytosis (ADC P). A number of mutations within the F c domain have been identified that either directly or indirectly enhance binding of Fc receptors and significantly enhance cellular cytotoxicity: the mutations S239D/A330L/I332E (“3M”), F243L or G236A. Alternatively enhancement of effector function can be achieved by modifying the glycosylation of the Fc domain, FcyRs interact with the carbohydrates on the CH2 domain and the glycan composition has a substantial effect on effector function activity. Afucosylated (non-fucosylated) antibodies, exhibit greatly enhanced ADCC activity through increased binding to FcyRIIIa.

Activation of ADCC and CDC may be desirable for some therapeutic antibodies, however, in some embodiments, an antibody that does not activate effector functions is preferred. Due to their lack of effector functions, IgG4 antibodies are the preferred IgG subclass for receptor blocking without cell depletion. However, IgG4 molecules can exchange half molecules in a dynamic process termed Fab-arm exchange. This phenomenon can occur between therapeutic antibodies and endogenous IgG4. The S228P mutation has been shown to prevent this recombination process allowing the design of IgG4 antibodies with a reduced propensity for Fab-arm exchange. Fc engineering approaches have been used to determine the key interaction sites for the IgG1 Fc domain with Fey receptors and C1 q and then mutate these positions to reduce or abolish binding. Through alanine scanning the binding site of C1 q to a region covering the hinge and upper CH2 of the F c domain was identified. The CH2 domain of an antibody or fragment of the invention may comprise one or more mutations to decrease or abrogate binding of the CH2 domain to one or more Fey receptors, such as FcyRI, FcyRIIa, FcyRIIb, FcyRIII and/or to complement. CH2 domains of human IgG domains normally bind to Fey receptors and complement, decreased binding to Fey receptors is expected to decrease antibody-dependent cell-mediated cytotoxicity (ADCC) and decreased binding to complement is expected to decrease the complement-dependent cytotoxicity (CDC) activity of the antibody molecule. Mutations to decrease or abrogate binding of the CH2 domain to one or more Fey receptors and/or complement are known in the art. An antibody molecule of the invention may comprise an Fc with modifications K322A/L234A/L235A or L234F/L235E/P331S (“TM”), which almost completely abolish FcyR and C1q binding. An antibody molecule of the invention may comprise a CH2 domain, wherein the CH2 domain comprises alanine residues at EU positions 234 and 235 (positions 1.3 and 1.2 by IMGT numbering) (“LALA mutation”). Furthermore, complement activation and ADCC can be decreased by mutation of Pro329 (position according to EU numbering), e.g., to either P 329A or P 329G. The antibody molecule of the invention may comprise a CH2 domain, wherein the CH2 domain comprises alanine residues at EU positions 234 and 235 (positions 1.3 and 1.2 by IMGT numbering) and an alanine (LALA-PA) or glycine (LALA-PG) at EU position 329 (position 114 by IMGT numbering). Additionally or alternatively an antibody molecule of the invention may comprise an alanine, glutamine or glycine at EU position 297 (position 84.4 by IMGT numbering).

Modification of glycosylation on asparagine 297 of the Fc domain, which is known to be required for optimal FcR interaction may confer a loss of binding to FcRs; a loss of binding to FcRs has been observed in N297 point mutations. An antibody molecule of the invention may comprise an Fc with an N297A, N297G or N297Q mutation. An antibody molecule of the invention with an aglycosyl Fc domain may be obtained by enzymatic deglycosylation, by recombinant expression in the presence of a glycosylation inhibitor, or following the expression of Fc domains in bacteria.

IgG naturally persists for a prolonged period in the serum due to FcRn-mediated recycling, giving it a typical half-life of approximately 21 days. Half-life can be extended by engineering the pH-dependent interaction of the Fc domain with FcRn to increase affinity at pH 6.0 while retaining minimal binding at pH 7.4. The T250Q/M428L variant, conferred an approximately 2-fold increase in IgG half-life (assessed in rhesus monkeys), while the M252Y/S254T/T256E variant (“YTE”), gave an approximately 4-fold increase in IgG half-life (assessed in cynomolgus monkeys). Extending half-life may allow the possibility of decreasing administration frequency, while maintaining or improving efficacy.

The antibodies of the invention preferably have Kd value of less than around 4 nM, less than around 3 nM, less than around 3 nM, less than around 2 nM, less than around 1 nM, as shown in the examples.

The term “Kd” refers to the “equilibrium dissociation constant” and refers to the value obtained in a titration measurement at equilibrium, or by dividing the dissociation rate constant (Koff) by the association rate constant (Kon). “Ka” refers to the affinity constant. The association rate constant, the dissociation rate constant and the equilibrium dissociation constant are used to represent the binding affinity of an antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used.

In embodiments, the antibody according to the invention has a Kd value in the nanomolar range.

The invention provides an antigen-binding protein, such as an antibody or antigen binding fragment thereof, capable of competing with an antibody of the invention described herein (e.g., comprising a set of HCDR and LCDRs, defined by Kabat nomenclature, and/or VH and VL amino acid sequence of any one of clones in Tables 1 to 3) for binding to an epitope within an isolated peptide comprising or consisting of residues 22-37 (SEQ ID NO: 1).

Competition assays include cell-based and cell-free binding assays including an immunoassay such as ELISA, HTRF; flow cytometry; fluorescent microvolume assay technology (FMAT) assay, Mirrorball, high content imaging based fluorescent immunoassays, radioligand binding assays, bio-layer interferometry (BLI), surface plasmon resonance (SPR) and thermal shift assays.

An antibody that binds to the same epitope as, or an epitope overlapping with, a reference antibody refers to an antibody that blocks binding of the reference antibody to its binding partner (e.g., an antigen or “target”) in a competition assay by 50% or more, and/or conversely, the reference antibody blocks binding of the antibody to its binding partner in a competition assay by 50% or more. Such antibodies are said to compete for binding to an epitope of interest.

The present invention further provides an isolated nucleic acid encoding an antibody of the present invention. Nucleic acid may include DNA and/or RNA.

In one aspect, the invention thus also relates to nucleic acid sequences comprising or consisting of a sequence selected from the combination of V_{H}, C_{H}, V_{L} and C_{L} sequences for the clones shown in Table 4. The nucleic acid sequences in Table 4 encode the antibodies shown in Table 3.

Thus, the invention provides antibodies or fragments thereof which bind to LGR5, and comprise the following nucleic acid sequences:
a) a V_{H} sequence of SEQ ID NO: 158; a C_{H} sequence of SEQ ID NO:159; a V_{L} sequence of SEQ ID NO: 160; and a C_{L} sequence of SEQ ID NO: 161;
b) a V_{H} sequence of SEQ ID NO: 162; a C_{H} sequence of SEQ ID NO:163; a V_{L} sequence of SEQ ID NO: 164; and a C_{L} sequence of SEQ ID NO: 165;
c) a V_{H} sequence of SEQ ID NO: 166; a C_{H} sequence of SEQ ID NO:167; a V_{L} sequence of SEQ ID NO: 168; and a C_{L} sequence of SEQ ID NO: 169;
d) a V_{H} sequence of SEQ ID NO: 170; a C_{H} sequence of SEQ ID NO:171; a V_{L} sequence of SEQ ID NO: 172; and a C_{L} sequence of SEQ ID NO: 173;
e) a V_{H} sequence of SEQ ID NO: 174; a C_{H} sequence of SEQ ID NO:175; a V_{L} sequence of SEQ ID NO: 176; and a C_{L} sequence of SEQ ID NO: 177;
f) a V_{H} sequence of SEQ ID NO: 178; a C_{H} sequence of SEQ ID NO:179; a V_{L} sequence of SEQ ID NO: 180; and a C_{L} sequence of SEQ ID NO: 181;
g) a V_{H} sequence of SEQ ID NO: 182; a C_{H} sequence of SEQ ID NO:183; a V_{L} sequence of SEQ ID NO: 184; and a C_{L} sequence of SEQ ID NO: 185;
h) a V_{H} sequence of SEQ ID NO: 186; a C_{H} sequence of SEQ ID NO:187; a V_{L} sequence of SEQ ID NO: 188; and a C_{L} sequence of SEQ ID NO: 189;
i) a V_{H} sequence of SEQ ID NO: 190; a C_{H} sequence of SEQ ID NO:191; a V_{L} sequence of SEQ ID NO: 192; and a C_{L} sequence of SEQ ID NO: 193;
j) a V_{H} sequence of SEQ ID NO: 194; a C_{H} sequence of SEQ ID NO:195; a V_{L} sequence of SEQ ID NO: 196; and a C_{L} sequence of SEQ ID NO: 197;
k) a V_{H} sequence of SEQ ID NO: 198; a C_{H} sequence of SEQ ID NO:199; a V_{L} sequence of SEQ ID NO: 200; and a C_{L} sequence of SEQ ID NO: 201;
l) a V_{H} sequence of SEQ ID NO: 202; a C_{H} sequence of SEQ ID NO:203; a V_{L} sequence of SEQ ID NO: 204; and a C_{L} sequence of SEQ ID NO: 205; or
m) a V_{H} sequence of SEQ ID NO: 206; a C_{H} sequence of SEQ ID NO:207; a V_{L} sequence of SEQ ID NO: 208; and a C_{L} sequence of SEQ ID NO: 209.

In one embodiment, the nucleic acid is SEQ ID NO. 218.

TABLE 4
Nucleic acid sequences encoding antibodies of the invention as shown in Table 3

| | V_{H} | C_{H} | V_{L} | C_{L} |
|---|---|---|---|---|
| 1 | SEQ ID NO: 158 | SEQ ID NO: 159 | SEQ ID NO: 160 | SEQ ID NO: |
| | CAGGTCCAAC | GCCAAAACGACACCCCCATCTGTC | GATATCCAGA | 160 |
| | TGCAGCAGCC | TATCCACTGGCCCCTGGATCTGCT | TGACACAGAG | CGGGCTGAT |
| | TGGGGCTGAG | GCCCAAACTAACTCCATGGTGACC | TACATCCTCC | GCTGCACCA |
| | CTTGTGAAGC | CTGGGATGCCTGGTCAAGGGCTAT | CTGTCTGCCT | ACTGTATCC |
| | CTGGGGCTTC | TTCCCTGAGCCAGTGACAGTGACC | CTCTGGGAGA | ATCTTCCCA |
| | AGTGAAACTG | TGGAACTCTGGATCCCTGTCCAGC | CAGAGTCACC | CCATCCAGT |
| | TCCTGCAAGG | GGTGTGCACACCTTCCCAGCTGTC | ATCAATTGCA | GAGCAGTTA |
| | CTTCTGGCTA | CTGCAGTCTGACCTCTACACTCTGA | GGGCAAGTCA | ACATCTGGA |
| | CACCTTCACC | GCAGCTCAGTGACTGTCCCCTCCA | GGACATTAGA | GGTGCCTCA |
| | AGCTACTGGA | GCACCTGGCCCAGCCAGACCGTCA | AATCGTTTAAA | GTCGTGTGC |
| | TGCAGTGGGT | CCTGCAACGTTGCCCACCCGGCCA | CTGGTATCAG | TTCTTGAACA |
| | AAAACAGAGG | GCAGCACCAAGGTGGACAAGAAAA | CAGAAACCAG | ACTTCTACC |
| | CCTGGACAGG | TTGTGCCCAGGGATTGTGGTTGTAA | ATGGAACTGT | CCAAAGACA |
| | GCCTTGAGTG | GCCTTGCATATGTACAGTCCCAGAA | TAAACTCCTG | TCAATGTCA |
| | GATCGGAGAG | GTATCATCTGTCTTCATCTTCCCCC | ATCAGCTACA | AGTGGAAGA |
| | ATTGATCCTTC | CAAAGCCCAAGGATGTGCTCACCA | GATCAAGATT | TTGATGGCA |
| | TGATTACTATA | TTACTCTGACTCCTAAGGTCACGTG | ACAGTCAGGA | GTGAACGAC |
| | CTAACTACAAT | TGTTGTGGTAGACATCAGCAAGGAT | GTCCCATCAA | AAAATGGCG |
| | CAAAAGTTCA | GATCCCGAGGTCCAGTTCAGCTGG | GGTTCAGTGG | TCCTGAACA |
| | AGGGCAAGGC | TTTGTAGATGATGTGGAGGTGCACA | CAGTGGGTCT | GTTGGACTG |
| | CACATTGACT | CAGCTCAGACGAAACCCCGGGAGG | GGCACAGAGT | ATCAGGACA |
| | GTAGACACAT | AGCAGATCAACAGCACTTTCCGTTC | ATTCTCTCACC | GCAAAGACA |
| | CCTCCAGCAC | AGTCAGTGAACTTCCCATCATGCAC | ATTAGCAACC | GCACCTACA |
| | AGCCTACATG | CAGGACTGGCTCAATGGCAAGGAG | TGGAGCAAGA | GCATGAGCA |
| | CAGCTCAGCA | TTCAAATGCAGGGTCAACAGTGCA | AGACATTGCC | GCACCCTCA |
| | GCCTGACATC | GCTTTCCCTGCCCCCATCGAGAAA | ACTTATTTTTG | CGTTGACCA |
| | TGAGGACTCT | ACCATCTCCAAAACCAAAGGCAGA | CCAACAGGGC | AGGACGAGT |
| | GCGGTCTATT | CCGAAGGCTCCACAGGTGTACACC | AATACGCTTC | ATGAACGAC |
| | ACTGTGCAAG | ATTCCACCTCCCAAGGAGCAGATG | CTCCCACGTT | ATAACAGCT |
| | ATCCCTCTCA | GCCAAGGATAAAGTCAGTCTGACC | CGGAGGGGG | ATACCTGTG |
| | GGCTACGTTG | TGCATGATAACAAACTTCTTCCCTG | GACCAAGCTG | AGGCCACTC |
| | ACTACTGGGG | AAGACATTACTGTGGAGTGGCAGT | GAAGTGAGA | ACAAGACAT |
| | CCAAGGCACC | GGAATGGGCAGCCAGCGGAGAACT | | CAACTTCAC |
| | ACTCTCACAG | ACAAGAACACTCAGCCCATCATGG | | CCATTGTCA |
| | TCTCCTCA | ACACAGATGGCTCTTACTTCGTCTA | | AGAGCTTCA |
| | | CAGCAAGCTCAATGTGCAGAAGAG | | ACAGGAATG |
| | | CAACTGGGAGGCAGGAAATACTTT | | AGTGT |
| | | CACCTGCTCTGTGTTACATGAGGG | | |
| | | CCTGCACAACCACCATACTGAGAA | | |
| | | GAGCCTCTCCCACTCTCCTGGTAAA | | |
| 2 | SEQ ID NO: 162 | SEQ ID NO: 163 | SEQ ID NO: 164 | SEQ ID NO: |
| | CAAGTCCAAC | GCCAAAACAACACCCCCATCAGTCT | GATATCCAGA | 165 |
| | TGCAGCAGCC | ATCCACTGGCCCCTGGGTGTGGAG | TGACACAGAC | CGGGCTGAT |
| | TGGGGCTGAG | ATACAACTGGTTCCTCTGTGACTCT | TACATCCTCC | GCTGCACCA |
| | CTTGTGAAGC | GGGATGCCTGGTCAAGGGCTACTT | CTGTCTGCCT | ACTGTATCC |
| | CTGGGGCTTC | CCCTGAGTCAGTGACTGTGACTTG | CTCTGGGAGA | ATCTTCCCA |
| | AGTGAAGCTG | GAACTCTGGATCCCTGTCCAGCAG | CAGAGTCACC | CCATCCAGT |
| | TCCTGCAAGG | TGTGCACACCTTCCCAGCTCTCCTG | ATCAGTTGCA | GAGCAGTTA |
| | CTTCTGGCTA | CAGTCTGGACTCTACACTATGAGCA | GGGCAAGTCA | ACATCTGGA |
| | CACCTTCACC | GCTCAGTGACTGTCCCCTCCAGCA | GGACATTAGC | GGTGCCTCA |
| | AACTACTGGA | CCTGGCCAAGTCAGACCGTCACCT | AATCGTTTAAA | GTCGTGTGC |
| | TGCAGTGGGT | GCAGCGTTGCTCACCCAGCCAGCA | CTGGTATCAG | TTCTTGAACA |
| | AAAACAGAGG | GCACCACGGTGGACAAAAAACTTG | CAGAAACCAG | ACTTCTACC |
| | CCTGGACAGG | AGCCCAGCGGGCCCATTTCAACAA | ATGGAACTGT | CCAAAGACA |
| | GCCTTGAGTG | TCAACCCCTGTCCTCCATGCAAGG | TAAACTCCTG | TCAATGTCA |
| | GATCGGAGAG | AGTGTCACAAATGCCCAGCTCCTAA | ATCAGCTACA | AGTGGAAGA |
| | ATTGATCCTTC | CCTCGAGGGTGGACCATCCGTCTT | GATCAAGATT | TTGATGGCA |
| | TGATAGTTATA | CATCTTCCCTCCAAATATCAAGGAT | ACACTCAGGA | GTGAACGAC |
| | CTAACTACAAT | GTACTCATGATCTCCCTGACACCCA | GTCCCATCAA | AAAATGGCG |
| | CAAAAGTTCA | AGGTCACGTGTGTGGTGGTGGATG | GGTTCAGTGG | TCCTGAACA |
| | AGGGCAAGGC | TGAGCGAGGATGACCCAGACGTCC | CAGTGGGTCT | GTTGGACTG |
| | CACATTGACT | GGATCAGCTGGTTTGTGAACAACG | GGCACAGATT | ATCAGGACA |
| | GTAGACACAT | TGGAAGTACACACAGCTCAGACAC | ATTCTCTCACC | GCAAAGACA |
| | CCTCCAGCAC | AAACCCATAGAGAGGATTACAACAG | ATTAGCAACC | GCACCTACA |
| | AGCCTACATG | TACTATCCGGGTGGTCAGTGCCCT | TGGAGCAAGA | GCATGAGCA |
| | CAGCTCAGCA | CCCCATCCAGCACCAGGACTGGAT | AGACATTGCC | GCACCCTCA |
| | GCCTGACATC | GAGTGGCAAGGAGTTCAAATGCAA | ACTTATTTTTG | CGTTGACCA |
| | TGAGGACTCT | GGTCAACAACAAAGACCTCCCATCA | CCAACAGGGT | AGGACGAGT |
| | GCGGTCTATT | CCCATCGAGAGAACCATCTCAAAAA | AATTCGCTTC | ATGAACGAC |
| | ACTGTGCAAG | TTAAAGGGCTAGTCAGAGCTCCAC | CTCCCACGTT | ATAACAGCT |
| | ATCCCTCTCA | AAGTATACATCTTGCCGCCACCAGC | CGGAGGGGG | ATACCTGTG |
| | GGCTACGTTG | AGAGCAGTTGTCCAGGAAAGATGT | GACCAAGCTG | AGGCCACTC |
| | ACTACTGGGG | CAGTCTCACTTGCCTGGTCGTGGG | GAAGTGAGA | ACAAGACAT |
| | CCAAGGCACC | CTTCAACCCTGGAGACATCAGTGT | | CAACTTCAC |
| | ACTCTCACAG | GGAGTGGACCAGCAATGGGCATAC | | CCATTGTCA |
| | TCTCCTCA | AGAGGAGAACTACAAGGACACCGC | | AGAGCTTCA |
| | | ACCAGTCCTGGACTCTGACGGTTC | | ACAGGAATG |
| | | TTACTTCATATACAGCAAGCTCGAT | | AGTGT |
| | | ATAAAAACAAGCAAGTGGGAGAAAA | | |
| | | CAGATTCCTTCTCATGCAACGTGAG | | |
| | | ACACGAGGGTCTGAAAAATTACTAC | | |
| | | CTGAAGAAGACCATCTCCCGGTCT | | |
| | | CCGGGTAAA | | |
| 3 | SEQ ID NO: 166 | SEQ ID NO: 167 | SEQ ID NO: 168 | SEQ ID NO: |
| | CAGGTCCAAC | GCCAAAACGACACCCCCATCTGTC | GATATCCAGA | 169 |
| | TGCAGCAGCC | TATCCACTGGCCCCTGGATCTGCT | TGACACAGAC | CGGGCTGAT |
| | TGGGGCTGAG | GCCCAAACTAACTCCATGGTGACC | TACATCCTCC | GCTGCACCA |
| | CTTGTGAAGC | CTGGGATGCCTGGTCAAGGGCTAT | CTGTCTGCCT | ACTGTATCC |
| | CTGGGGCTTC | TTCCCTGAGCCAGTGACAGTGACC | CTCTGGGAGA | ATCTTCCCA |
| | AGTGAAGCTG | TGGAACTCTGGATCCCTGTCCAGC | CAGAGTCACC | CCATCCAGT |
| | TCCTGCAAGG | GGTGTGCACACCTTCCCAGCTGTC | ATCAGTTGCA | GAGCAGTTA |
| | CTTCTGGCTA | CTGCAGTCTGACCTCTACACTCTGA | GGGCAAGTCA | ACATCTGGA |
| | CACCTTCACC | GCAGCTCAGTGACTGTCCCCTCCA | GGACATTAGC | GGTGCCTCA |
| | AGCTACTGGA | GCACCTGGCCCAGCCAGACCGTCA | AATCGTTTAAA | GTCGTGTGC |
| | TGCAGTGGGT | CCTGCAACGTTGCCCACCCGGCCA | CTGGTATCAG | TTCTTGAACA |
| | AAAACAGAGG | GCAGCACCAAGGTGGACAAGAAAA | CAGAAACCAG | ACTTCTACC |
| | CCTGGACAGG | TTGTGCCCAGGGATTGTGGTTGTAA | ATGGAACTGT | CCAAAGACA |
| | GCCTTGAGTG | GCCTTGCATATGTACAGTCCCAGAA | TAAACTCCTG | TCAATGTCA |
| | GATCGGAGAG | GTATCATCTGTCTTCATCTTCCCCC | ATCAGCTACA | AGTGGAAGA |
| | ATTGATCCTTC | CAAAGCCCAAGGATGTGCTCACCA | GATCAAGATT | TTGATGGCA |
| | TGATAGCTATA | TTACTCTGACTCCTAAGGTCACGTG | ACAGTCAGGA | GTGAACGAC |
| | GTAACTACAAT | TGTTGTGGTAGACATCAGCAAGGAT | GTCCCATCAA | AAAATGGCG |
| | CAAAAGTTCA | GATCCCGAGGTCCAGTTCAGCTGG | GGTTCAGTGG | TCCTGAACA |
| | AGGGCAAGGC | TTTGTAGATGATGTGGAGGTGCACA | CAGTGGGTCT | GTTGGACTG |
| | CACATTGACT | CAGCTCAGACGAAACCCCGGGAGG | GGCACAGATT | ATCAGGACA |
| | GTAGACACAT | AGCAGATCAACAGCACTTTCCGTTC | ATTCTCTCACC | GCAAAGACA |
| | CCTCCAGCAC | AGTCAGTGAACTTCCCATCATGCAC | ATTAGCAACC | GCACCTACA |
| | AGCCTACATG | CAGGACTGGCTCAATGGCAAGGAG | TGGAGGAAGA | GCATGAGCA |
| | CAGCTCAGCA | TTCAAATGCAGGGTCAACAGTGCA | AGACATTGCC | GCACCCTCA |
| | GCCTGACATC | GCTTTCCCTGCCCCCATCGAGAAA | ACCTATTTTTG | CGTTGACCA |
| | TGAGGACTCT | ACCATCTCCAAAACCAAAGGCAGA | CCAACAGGGT | AGGACGAGT |
| | GCGGTCTATT | CCGAAGGCTCCACAGGTGTACACC | AATACCCTTC | ATGAACGAC |
| | ACTGTGCAAG | ATTCCACCTCCCAAGGAGCAGATG | CTCCCACGTT | ATAACAGCT |
| | ATCCCTCTCA | GCCAAGGATAAAGTCAGTCTGACC | CGGAGGGGG | ATACCTGTG |
| | GGCTACGTTG | TGCATGATAACAAACTTCTTCCCTG | GACCAAGCTG | AGGCCACTC |
| | ACTACTGGGG | AAGACATTACTGTGGAGTGGCAGT | GAAGTGAGA | ACAAGACAT |
| | CCAAGGCACC | GGAATGGGCAGCCAGCGGAGAACT | | CAACTTCAC |
| | ACTCTCACAG | ACAAGAACACTCAGCCCATCATGG | | CCATTGTCA |
| | TCTCCTCA | ACACAGATGGCTCTTACTTCGTCTA | | AGAGCTTCA |
| | | CAGCAAGCTCAATGTGCAGAAGAG | | ACAGGAATG |
| | | CAACTGGGAGGCAGGAAATACTTT | | AGTGT |
| | | CACCTGCTCTGTGTTACATGAGGG | | |
| | | CCTGCACAACCACCATACTGAGAA | | |
| | | GAGCCTCTCCCACTCTCCTGGTAAA | | |
| 4 | SEQ ID NO: 170 | SEQ ID NO: 171 | SEQ ID NO: 172 | SEQ ID NO: |
| | CAGGTCCAAC | GCCAAAACAACACCCCCATCAGTCT | GATATCCAGA | 173 |
| | TGCAGCAGCC | ATCCACTGGCCCCTGGGTGTGGAG | TGACACAGAC | CGGGCTGAT |
| | TGGGGCTGAG | ATACAACTGGTTCCTCTGTGACTCT | TACTTCCTCC | GCTGCACCA |
| | CTTGTGAAGC | GGGATGCCTGGTCAAGGGCTACTT | CTGTCTGCCT | ACTGTATCC |
| | CTGGGGCTTC | CCCTGAGTCAGTGACTGTGACTTG | CTCTGGGAGA | ATCTTCCCA |
| | AGTGAAGCTG | GAACTCTGGATCCCTGTCCAGCAG | CAGAGTCACC | CCATCCAGT |
| | TCCTGCAAGG | TGTGCACACCTTCCCAGCTCTCCTG | ATCAGTTGCA | GAGCAGTTA |
| | CTTCTGGCTA | CAGTCTGGACTCTACACTATGAGCA | GGGCAAGTCA | ACATCTGGA |
| | CACCTTCACC | GCTCAGTGACTGTCCCCTCCAGCA | GGACATTAGC | GGTGCCTCA |
| | GACTACTGGA | CCTGGCCAAGTCAGACCGTCACCT | AATCGTTTAAA | GTCGTGTGC |
| | TGCAGTGGGT | GCAGCGTTGCTCACCCAGCCAGCA | CTGGTATCAG | TTCTTGAACA |
| | AAAACAGAGG | GCACCACGGTGGACAAAAAACTTG | CAGAAACCAG | ACTTCTACC |
| | CCTGGACAGG | AGCCCAGCGGGCCCATTTCAACAA | ATGGAACTGT | CCAAAGACA |
| | GCCTTGAGTG | TCAACCCCTGTCCTCCATGCAAGG | TAAACTCCTG | TCAATGTCA |
| | GATCGGAGAG | AGTGTCACAAATGCCCAGCTCCTAA | ATCAGCTACA | AGTGGAAGA |
| | ATTGATCCTTC | CCTCGAGGGTGGACCATCCGTCTT | GATCAAGATT | TTGATGGCA |
| | TGATAGCTATA | CATCTTCCCTCCAAATATCAAGGAT | ACAGTCAGGA | GTGAACGAC |
| | CTAACTACAAT | GTACTCATGATCTCCCTGACACCCA | GTCCCATCAA | AAAATGGCG |
| | CAAAAGTTCC | AGGTCACGTGTGTGGTGGTGGATG | GGTTCAGTGG | TCCTGAACA |
| | AGGGCAAGGC | TGAGCGAGGATGACCCAGACGTCC | CAGTGGGTCT | GTTGGACTG |
| | CACATTGACT | GGATCAGCTGGTTTGTGAACAACG | GGCACAGATT | ATCAGGACA |
| | GTAGACACAT | TGGAAGTACACACAGCTCAGACAC | ATTCTCTCACC | GCAAAGACA |
| | CCTCCAGCAC | AAACCCATAGAGAGGATTACAACAG | ATTACCAACCT | GCACCTACA |
| | AGCCTACATC | TACTATCCGGGTGGTCAGTGCCCT | GGAGCAAGAA | GCATGAGCA |
| | CAGCTCAGCA | CCCCATCCAGCACCAGGACTGGAT | GACATTGCCA | GCACCCTCA |
| | GCCTGACATC | GAGTGGCAAGGAGTTCAAATGCAA | CTTATTTTTGC | CGTTGACCA |
| | TGACGACTCT | GGTCAACAACAAAGACCTCCCATCA | CAACAGGGTA | AGGACGAGT |
| | GCGGTCTATT | CCCATCGAGAGAACCATCTCAAAAA | ATACGCTTCC | ATGAACGAC |
| | ACTGTGCAAG | TTAAAGGGCTAGTCAGAGCTCCAC | TCCCACGTTC | ATAACAGCT |
| | ATCCCTCTCA | AAGTATACATCTTGCCGCCACCAGC | GGAGGGGGG | ATACCTGTG |
| | GGCTCCGTTG | AGAGCAGTTGTCCAGGAAAGATGT | ACCAAGCTGG | AGGCCACTC |
| | ACTACTGGGG | CAGTCTCACTTGCCTGGTCGTGGG | AAGTGAGA | ACAAGACAT |
| | CCAAGGCACC | CTTCAACCCTGGAGACATCAGTGT | | CAACTTCAC |
| | ACTCTCACAG | GGAGTGGACCAGCAATGGGCATAC | | CCATTGTCA |
| | TCTCCTCA | AGAGGAGAACTACAAGGACACCGC | | AGAGCTTCA |
| | | ACCAGTCCTGGACTCTGACGGTTC | | ACAGGAATG |
| | | TTACTTCATATACAGCAAGCTCGAT | | AGTGT |
| | | ATAAAAACAAGCAAGTGGGAGAAAA | | |
| | | CAGATTCCTTCTCATGCAACGTGAG | | |
| | | ACACGAGGGTCTGAAAAATTACTAC | | |
| | | CTGAAGAAGACCATCTCCCGGTCT | | |
| | | CCGGGTAAA | | |
| 12 | SEQ ID NO: 174 | SEQ ID NO: 175 | SEQ ID NO: 176 | SEQ ID NO: |
| | GAGGTCCAGC | GCCAAAACAACAGCCCCATCGGTC | GACATTGTGA | 177 |
| | TGCAACAATC | TATCCACTGGCCCCTGTGTGTGGA | TGACACAGTC | CGGGCTGAT |
| | TGGACCTGAG | GGTACAACTGGCTCCTCGGTGACT | TCCATCCTCC | GCTGCACCA |
| | CTGGTGAAGC | CTAGGATGCCTGGTCAAGGGTTATT | CTGGCTATGT | ACTGTATCC |
| | CTGGGGCTTC | TCCCTGAGCCAGTGACCTTGACCT | CAGTAGGACA | ATCTTCCCA |
| | AGTGAAGATA | GGAACTCTGGATCCCTGTCCAGTG | GAAGGTCACT | CCATCCAGT |
| | TCCTGTAAGG | GTGTGCACACCTTCCCAGCTCTCCT | ATGAGCTGCA | GAGCAGTTA |
| | CTTCTGGATA | GCAGTCTGGCCTCTACACCCTCAG | AGTCCAGTCA | ACATCTGGA |
| | CACGTTCACT | CAGCTCAGTGACTGTAACCTCGAA | GAGCCTTTTA | GGTGCCTCA |
| | GACTACTACA | CACCTGGCCCAGCCAGACCATCAC | AATAGTAGCA | GTCGTGTGC |
| | TGAACTGGGT | CTGCAATGTGGCCCACCCGGCAAG | ATCAAAAGAA | TTCTTGAACA |
| | GAAGCAGAGC | CAGCACCAAAGTGGACAAGAAAATT | CTATTTGGCC | ACTTCTACC |
| | CATGGAAAGA | GAGCCCAGAGTGCCCATAACACAG | TGGTACCAGC | CCAAAGACA |
| | GCCTTGAGTG | AACCCCTGTCCTCCACTCAAAGAGT | AGAAACCAGG | TCAATGTCA |
| | GATTGGAGAT | GTCCCCCATGCGCAGCTCCAGACC | ACAGTCTCCT | AGTGGAAGA |
| | ATTAATCCTAA | TCTTGGGTGGACCATCCGTCTTCAT | AAACTTCTGG | TTGATGGCA |
| | CAATGGTGGT | CTTCCCTCCAAAGATCAAGGATGTA | TATACTTTGCA | GTGAACGAC |
| | ACTATCTACAA | CTCATGATCTCCCTGAGCCCCATG | TCCACTAGGG | AAAATGGCG |
| | CCAGAAGTTC | GTCACATGTGTGGTGGTGGATGTG | AATCTGGGGT | TCCTGAACA |
| | AAGGGCAAGG | AGCGAGGATGACCCAGACGTCCAG | CCCTGATCGC | GTTGGACTG |
| | CCACATTGAC | ATCAGCTGGTTTGTGAACAACGTG | TTCATAGGCA | ATCAGGACA |
| | TGTAGACAAG | GAAGTACACACAGCTCAGACACAA | GTGGATCTGG | GCAAAGACA |
| | TCCTCCAGCA | ACCCATAGAGAGGATTACAACAGTA | GACAGATTTC | GCACCTACA |
| | CAGCCTACAT | CTCTCCGGGTGGTCAGTGCCCTCC | ACTCTTACCAT | GCATGAGCA |
| | GGAGCTCCGC | CCATCCAGCACCAGGACTGGATGA | CAGCAGTGTG | GCACCCTCA |
| | AGCCTGACAT | GTGGCAAGGAGTTCAAATGCAAGG | CAGGCTGAAG | CGTTGACCA |
| | CTGAGGACTC | TCAACAACAGAGCCCTCCCATCCC | ACCTGGCAGC | AGGACGAGT |
| | TGCAGTCTATT | CCATCGAGAAAACCATCTCAAAACC | TTACTTCTGTC | ATGAACGAC |
| | ACTGTGCAAG | CAGAGGGCCAGTAAGAGCTCCACA | AGCAACATTAT | ATAACAGCT |
| | CGGATACCTC | GGTATATGTCTTGCCTCCACCAGCA | AGCACTCCGC | ATACCTGTG |
| | TTTGACTACTG | GAAGAGATGACTAAGAAAGAGTTCA | TCACGTTCGG | AGGCCACTC |
| | GGGCCCAGG | GTCTGACCTGCATGATCACAGGCTT | TGCTGGGACC | ACAAGACAT |
| | CACCACTCTC | CTTACCTGCCGAAATTGCTGTGGAC | AAGCTGGAGC | CAACTTCAC |
| | ACAGTCTCCT | TGGACCAGCAATGGGCGTACAGAG | TGAAA | CCATTGTCA |
| | CA | CAAAACTACAAGAACACCGCAACA | | AGAGCTTCA |
| | | GTCCTGGACTCTGATGGTTCTTACT | | ACAGGAATG |
| | | TCATGTACAGCAAGCTCAGAGTACA | | AGTGT |
| | | AAAGAGCACTTGGGAAAGAGGAAG | | |
| | | TCTTTTCGCCTGCTCAGTGGTCCAC | | |
| | | GAGGGTCTGCACAATCACCTTACG | | |
| | | ACTAAGACCATCTCCCGGTCTCTG | | |
| | | GGTAAA | | |
| 2.1 | SEQ ID NO: 178 | SEQ ID NO: 179 | SEQ ID NO: 180 | SEQ ID NO: |
| | CAAGTGCAGC | GCCAGCACCAAAGGTCCATCCGTG | GACATTCAGA | 181 |
| | TGGTGCAGAG | TTTCCGCTCGCCCCGTCCTCAAAGT | TGACACAGAG | AGAACTGTG |
| | CGGGGCCGA | CGACCTCCGGAGGCACTGCCGCCC | CCCTAGCAGC | GCCGCGCC |
| | GGTGAAGAAG | TGGGCTGCCTTGTCAAGGACTATTT | CTGAGCGCAA | GTCAGTGTT |
| | CCCGGCGCAA | CCCCGAACCTGTCACGGTGTCCTG | GCGTGGGCGA | TATCTTCCCT |
| | GCGTGAAGGT | GAACAGCGGCGCCCTGACTTCCGG | CAGAGTGACC | CCATCGGAT |
| | GAGCTGCAAG | AGTGCACACCTTCCCCGCCGTCCT | ATCACCTGCA | GAACAGCTT |
| | GCAAGCGGCT | GCAATCCAGCGGCCTGTACTCACT | GAGCAAGCCA | AAGTCCGGC |
| | ACACCTTCAC | GTCATCCGTTGTGACTGTCCCGTC | AGACATCAGC | ACGGCGTCT |
| | CAACTACTGG | GTCCAGCCTGGGAACCCAAACCTA | AACAGACTGA | GTGGTCTGC |
| | ATGCAGTGGG | CATTTGCAACGTGAATCACAAACCA | ACTGGTATCA | CTGCTCAAT |
| | TGAGACAAGC | TCGAATACCAAGGTCGATAAGAAAG | GCAGAAGCCC | AACTTTTACC |
| | CCCCGGCCAA | TCGAGCCGAAGTCATGCGACAAGA | GGCAAGGCCG | CTAGGGAAG |
| | GGCCTGGAGT | CTCACACCTGTCCGCCTTGCCCAG | TGAAGCTGCT | CTAAAGTCC |
| | GGATCGGCGA | CCCCAGAACTGCTCGGCGGCCCTT | GATCAGCTAC | AATGGAAAG |
| | GATCGACCCT | CGGTGTTTTTGTTTCCGCCGAAGCC | AGAAGCAGAC | TGGATAACG |
| | AGCGACAGCT | GAAGGACACTCTGATGATCTCACG | TGCACAGCGG | CCCTGCAGT |
| | ACACCAACTA | CACTCCAGAGGTGACTTGCGTGGT | CGTGCCTAGC | CAGGAAACA |
| | CAATCAGAAG | GGTCGATGTTTCGCACGAGGACCC | AGATTCAGCG | GCCAGGAAT |
| | TTCCAAGGCA | GGAAGTGAAATTCAACTGGTATGTC | GCAGCGGCAG | CGGTTACCG |
| | GAGTGACCCT | GACGGGGTGGAAGTGCATAATGCC | CGGCACCGAC | AACAGGACA |
| | GACCGTGGAC | AAGACGAAGCCGAGGGAGGAACAG | TACACCCTGA | GCAAGGACA |
| | ACAAGCACAA | TACAACTCCACCTACAGAGTGGTTT | CCATCAGCAG | GCACTTACT |
| | GCACCGCCTA | CAGTCCTTACCGTCCTCCATCAAGA | CCTGCAGCCT | CCTTGTCGT |
| | CATGGAGCTG | TTGGCTGAACGGAAAGGAGTACAA | GAGGACTTCG | CGACTCTTA |
| | AGCAGCCTGA | ATGTAAGGTGTCGAACAAAGCGTT | CCACCTACTT | CTCTGAGCA |
| | GAAGCGAGGA | GCCGGCCCCTATCGAAAAGACTAT | CTGTCAGCAA | AGGCCGATT |
| | CACCGCCGTG | CAGCAAGGCCAAAGGACAGCCGCG | GGCAACAGCC | ACGAGAAGC |
| | TACTACTGCG | GGAGCCGCAAGTGTACACCCTCCC | TGCCCCCCAC | ACAAGGTCT |
| | CTAGAAGCCT | GCCTTCGCGGGACGAGCTGACCAA | CTTCGGCGGC | ACGCCTGCG |
| | GAGCGGCTAC | GAATCAGGTGTCCCTTACTTGCCTG | GGCACCAAGC | AGGTCACCC |
| | GTGGACTACT | GTGAAGGGATTCTACCCCTCGGAT | TCGAGATCAA | ATCAGGGAC |
| | GGGGCCAAG | ATCGCAGTCGAATGGGAATCGAAT | G | TCTCGTCCC |
| | GCACCACCGT | GGACAGCCAGAAAACAACTACAAG | | CGGTGACCA |
| | GACCGTCTCG | ACCACTCCCCCGGTGCTCGACTCC | | AATCCTTCAA |
| | AGC | GACGGTTCCTTCTTCCTGTACTCGA | | TAGAGGCGA |
| | | AGCTGACCGTGGACAAATCACGCT | | ATGC |
| | | GGCAGCAGGGAAACGTGTTTAGCT | | |
| | | GCAGCGTGATGCATGAGGCGCTGC | | |
| | | ATAATCACTACACCCAGAAGTCACT | | |
| | | CTCGCTCAGCCCAGGGAAG | | |
| 2.2 | SEQ ID NO: 182 | SEQ ID NO: 183 | SEQ ID NO: 184 | SEQ ID NO: |
| | CAAGTGCAGC | GCCAGCACCAAAGGTCCATCCGTG | GACATTCAGA | 185 |
| | TGGTGCAGAG | TTTCCGCTCGCCCCGTCCTCAAAGT | TGACACAGAG | AGAACTGTG |
| | CGGGGCCGA | CGACCTCCGGAGGCACTGCCGCCC | CCCTAGCAGC | GCCGCGCC |
| | GGTGAAGAAG | TGGGCTGCCTTGTCAAGGACTATTT | CTGAGCGCAA | GTCAGTGTT |
| | CCCGGCGCAA | CCCCGAACCTGTCACGGTGTCCTG | GCGTGGGCGA | TATCTTCCCT |
| | GCGTGAAGGT | GAACAGCGGCGCCCTGACTTCCGG | CAGAGTGACC | CCATCGGAT |
| | GAGCTGCAAG | AGTGCACACCTTCCCCGCCGTCCT | ATCACCTGCA | GAACAGCTT |
| | GCAAGCGGCT | GCAATCCAGCGGCCTGTACTCACT | GAGCAAGCCA | AAGTCCGGC |
| | ACACCTTCAC | GTCATCCGTTGTGACTGTCCCGTC | AGACATCAGC | ACGGCGTCT |
| | CAACTACTGG | GTCCAGCCTGGGAACCCAAACCTA | AACAGACTGA | GTGGTCTGC |
| | ATGCAGTGGG | CATTTGCAACGTGAATCACAAACCA | ACTGGTATCA | CTGCTCAAT |
| | TGAGACAAGC | TCGAATACCAAGGTCGATAAGAAAG | GCAGAAGCCC | AACTTTTACC |
| | CCCCGGCCAA | TCGAGCCGAAGTCATGCGACAAGA | GGCAAGGCCG | CTAGGGAAG |
| | GGCCTGGAGT | CTCACACCTGTCCGCCTTGCCCAG | TGAAGCTGCT | CTAAAGTCC |
| | GGATCGGCGA | CCCCAGAACTGCTCGGCGGCCCTT | GATCAGCTAC | AATGGAAAG |
| | GATCGACCCT | CGGTGTTTTTGTTTCCGCCGAAGCC | AGAAGCAGAC | TGGATAACG |
| | AGCGACAGCT | GAAGGACACTCTGATGATCTCACG | TGCACAGCGG | CCCTGCAGT |
| | ACACCAACTA | CACTCCAGAGGTGACTTGCGTGGT | CGTGCCTAGC | CAGGAAACA |
| | CAATCAGAAG | GGTCGATGTTTCGCACGAGGACCC | AGATTCAGCG | GCCAGGAAT |
| | TTCCAAGGCA | GGAAGTGAAATTCAACTGGTATGTC | GCAGCGGCAG | CGGTTACCG |
| | GAGTGACCCT | GACGGGGTGGAAGTGCATAATGCC | CGGCACCGAC | AACAGGACA |
| | GACCGTGGAC | AAGACGAAGCCGAGGGAGGAACAG | TACACCCTGA | GCAAGGACA |
| | ACAAGCACAA | TACAACTCCACCTACAGAGTGGTTT | CCATCAGCAG | GCACTTACT |
| | GCACCGCCTA | CAGTCCTTACCGTCCTCCATCAAGA | CCTGCAGCCT | CCTTGTCGT |
| | CATGGAGCTG | TTGGCTGAACGGAAAGGAGTACAA | GAGGACTTCG | CGACTCTTA |
| | AGCAGCCTGA | ATGTAAGGTGTCGAACAAAGCGTT | CCACCTACTA | CTCTGAGCA |
| | GAAGCGAGGA | GCCGGCCCCTATCGAAAAGACTAT | CTGTCAGCAA | AGGCCGATT |
| | CACCGCCGTG | CAGCAAGGCCAAAGGACAGCCGCG | GGCAACAGCC | ACGAGAAGC |
| | TACTACTGCG | GGAGCCGCAAGTGTACACCCTCCC | TGCCCCCCAC | ACAAGGTCT |
| | CTAGAAGCCT | GCCTTCGCGGGACGAGCTGACCAA | CTTCGGCGGC | ACGCCTGCG |
| | GAGCGGCTAC | GAATCAGGTGTCCCTTACTTGCCTG | GGCACCAAGC | AGGTCACCC |
| | GTGGACTACT | GTGAAGGGATTCTACCCCTCGGAT | TCGAGATCAA | ATCAGGGAC |
| | GGGGCCAAG | ATCGCAGTCGAATGGGAATCGAAT | G | TCTCGTCCC |
| | GCACCACCGT | GGACAGCCAGAAAACAACTACAAG | | CGGTGACCA |
| | GACCGTCTCG | ACCACTCCCCCGGTGCTCGACTCC | | AATCCTTCAA |
| | AGC | GACGGTTCCTTCTTCCTGTACTCGA | | TAGAGGCGA |
| | | AGCTGACCGTGGACAAATCACGCT | | ATGC |
| | | GGCAGCAGGGAAACGTGTTTAGCT | | |
| | | GCAGCGTGATGCATGAGGCGCTGC | | |
| | | ATAATCACTACACCCAGAAGTCACT | | |
| | | CTCGCTCAGCCCAGGGAAG | | |
| 2.3 | SEQ ID NO: 186 | SEQ ID NO: 187 | SEQ ID NO: 188 | SEQ ID NO: |
| | CAAGTGCAGC | GCCAGCACCAAAGGTCCATCCGTG | GACATCGTAA | 189 |
| | TGGTGCAGAG | TTTCCGCTCGCCCCGTCCTCAAAGT | TGACACAGAG | AGAACTGTG |
| | CGGGGCCGA | CGACCTCCGGAGGCACTGCCGCCC | CCCCGCGACC | GCCGCGCC |
| | GGTGAAGAAG | TGGGCTGCCTTGTCAAGGACTATTT | CTGAGCCTGT | GTCAGTGTT |
| | CCCGGCGCAA | CCCCGAACCTGTCACGGTGTCCTG | CCCCCGGTGA | TATCTTCCCT |
| | GCGTGAAGGT | GAACAGCGGCGCCCTGACTTCCGG | GAGAGCTACC | CCATCGGAT |
| | GAGCTGCAAG | AGTGCACACCTTCCCCGCCGTCCT | CTGAGCTGCA | GAACAGCTT |
| | GCAAGCGGCT | GCAATCCAGCGGCCTGTACTCACT | GAGCAAGCCA | AAGTCCGGC |
| | ACACCTTCAC | GTCATCCGTTGTGACTGTCCCGTC | AGACATCAGC | ACGGCGTCT |
| | CAACTACTGG | GTCCAGCCTGGGAACCCAAACCTA | AACAGACTGA | GTGGTCTGC |
| | ATGCAGTGGG | CATTTGCAACGTGAATCACAAACCA | ACTGGTATCA | CTGCTCAAT |
| | TGAGACAAGC | TCGAATACCAAGGTCGATAAGAAAG | GCAGAAGCCC | AACTTTTACC |
| | CCCCGGCCAA | TCGAGCCGAAGTCATGCGACAAGA | GGCCAAGCCG | CTAGGGAAG |
| | GGCCTGGAGT | CTCACACCTGTCCGCCTTGCCCAG | TGAGACTGCT | CTAAAGTCC |
| | GGATCGGCGA | CCCCAGAACTGCTCGGCGGCCCTT | GATCAGCTAC | AATGGAAAG |
| | GATCGACCCT | CGGTGTTTTTGTTTCCGCCGAAGCC | AGAAGCAGAC | TGGATAACG |
| | AGCGACAGCT | GAAGGACACTCTGATGATCTCACG | TGCACAGCGG | CCCTGCAGT |
| | ACACCAACTA | CACTCCAGAGGTGACTTGCGTGGT | CGTGCCCGCT | CAGGAAACA |
| | CAATCAGAAG | GGTCGATGTTTCGCACGAGGACCC | AGATTCAGCG | GCCAGGAAT |
| | TTCCAAGGCA | GGAAGTGAAATTCAACTGGTATGTC | GCAGCGGCAG | CGGTTACCG |
| | GAGTGACCCT | GACGGGGTGGAAGTGCATAATGCC | CGGCACCGAC | AACAGGACA |
| | GACCGTGGAC | AAGACGAAGCCGAGGGAGGAACAG | TACACCCTGA | GCAAGGACA |
| | ACAAGCACAA | TACAACTCCACCTACAGAGTGGTTT | CCATCAGCAG | GCACTTACT |
| | GCACCGCCTA | CAGTCCTTACCGTCCTCCATCAAGA | CCTGGAGCCT | CCTTGTCGT |
| | CATGGAGCTG | TTGGCTGAACGGAAAGGAGTACAA | GAGGACTTCG | CGACTCTTA |
| | AGCAGCCTGA | ATGTAAGGTGTCGAACAAAGCGTT | CCGTGTACTT | CTCTGAGCA |
| | GAAGCGAGGA | GCCGGCCCCTATCGAAAAGACTAT | CTGTCAGCAA | AGGCCGATT |
| | CACCGCCGTG | CAGCAAGGCCAAAGGACAGCCGCG | GGCAACAGCC | ACGAGAAGC |
| | TACTACTGCG | GGAGCCGCAAGTGTACACCCTCCC | TGCCCCCCAC | ACAAGGTCT |
| | CTAGAAGCCT | GCCTTCGCGGGACGAGCTGACCAA | CTTCGGCGGC | ACGCCTGCG |
| | GAGCGGCTAC | GAATCAGGTGTCCCTTACTTGCCTG | GGCACCAAGC | AGGTCACCC |
| | GTGGACTACT | GTGAAGGGATTCTACCCCTCGGAT | TCGAGATCAA | ATCAGGGAC |
| | GGGGCCAAG | ATCGCAGTCGAATGGGAATCGAAT | G | TCTCGTCCC |
| | GCACCACCGT | GGACAGCCAGAAAACAACTACAAG | | CGGTGACCA |
| | GACCGTCTCG | ACCACTCCCCCGGTGCTCGACTCC | | AATCCTTCAA |
| | AGC | GACGGTTCCTTCTTCCTGTACTCGA | | TAGAGGCGA |
| | | AGCTGACCGTGGACAAATCACGCT | | ATGC |
| | | GGCAGCAGGGAAACGTGTTTAGCT | | |
| | | GCAGCGTGATGCATGAGGCGCTGC | | |
| | | ATAATCACTACACCCAGAAGTCACT | | |
| | | CTCGCTCAGCCCAGGGAAG | | |
| 2.4 | SEQ ID NO: 190 | SEQ ID NO: 191 | SEQ ID NO: 192 | SEQ ID NO: |
| | CAAGTGCAGC | GCCAGCACCAAAGGTCCATCCGTG | GAGATCGTAA | 193 |
| | TGGTGCAGAG | TTTCCGCTCGCCCCGTCCTCAAAGT | TGACACAGAG | AGAACTGTG |
| | CGGGGCCGA | CGACCTCCGGAGGCACTGCCGCCC | CCCCGCGACC | GCCGCGCC |
| | GGTGAAGAAG | TGGGCTGCCTTGTCAAGGACTATTT | CTGAGCCTGT | GTCAGTGTT |
| | CCCGGCGCAA | CCCCGAACCTGTCACGGTGTCCTG | CCCCCGGTGA | TATCTTCCCT |
| | GCGTGAAGGT | GAACAGCGGCGCCCTGACTTCCGG | GAGAGCTACC | CCATCGGAT |
| | GAGCTGCAAG | AGTGCACACCTTCCCCGCCGTCCT | CTGAGCTGCA | GAACAGCTT |
| | GCAAGCGGCT | GCAATCCAGCGGCCTGTACTCACT | GAGCAAGCCA | AAGTCCGGC |
| | ACACCTTCAC | GTCATCCGTTGTGACTGTCCCGTC | AGACATCAGC | ACGGCGTCT |
| | CAACTACTGG | GTCCAGCCTGGGAACCCAAACCTA | AACAGACTGA | GTGGTCTGC |
| | ATGCAGTGGG | CATTTGCAACGTGAATCACAAACCA | ACTGGTATCA | CTGCTCAAT |
| | TGAGACAAGC | TCGAATACCAAGGTCGATAAGAAAG | GCAGAAGCCC | AACTTTTACC |
| | CCCCGGCCAA | TCGAGCCGAAGTCATGCGACAAGA | GGCCAAGCCG | CTAGGGAAG |
| | GGCCTGGAGT | CTCACACCTGTCCGCCTTGCCCAG | TGAGACTGCT | CTAAAGTCC |
| | GGATCGGCGA | CCCCAGAACTGCTCGGCGGCCCTT | GATCAGCTAC | AATGGAAAG |
| | GATCGACCCT | CGGTGTTTTTGTTTCCGCCGAAGCC | AGAAGCAGAA | TGGATAACG |
| | AGCGACAGCT | GAAGGACACTCTGATGATCTCACG | GACACACCGG | CCCTGCAGT |
| | ACACCAACTA | CACTCCAGAGGTGACTTGCGTGGT | CATCCCCGCT | CAGGAAACA |
| | CAATCAGAAG | GGTCGATGTTTCGCACGAGGACCC | AGATTCAGCG | GCCAGGAAT |
| | TTCCAAGGCA | GGAAGTGAAATTCAACTGGTATGTC | GCAGCGGCAG | CGGTTACCG |
| | GAGTGACCCT | GACGGGGTGGAAGTGCATAATGCC | CGGCACCGAC | AACAGGACA |
| | GACCGTGGAC | AAGACGAAGCCGAGGGAGGAACAG | TACACCCTGA | GCAAGGACA |
| | ACAAGCACAA | TACAACTCCACCTACAGAGTGGTTT | CCATCAGCAG | GCACTTACT |
| | GCACCGCCTA | CAGTCCTTACCGTCCTCCATCAAGA | CCTGGAGCCT | CCTTGTCGT |
| | CATGGAGCTG | TTGGCTGAACGGAAAGGAGTACAA | GAGGACTTCG | CGACTCTTA |
| | AGCAGCCTGA | ATGTAAGGTGTCGAACAAAGCGTT | CCGTGTACTA | CTCTGAGCA |
| | GAAGCGAGGA | GCCGGCCCCTATCGAAAAGACTAT | CTGTCAGCAA | AGGCCGATT |
| | CACCGCCGTG | CAGCAAGGCCAAAGGACAGCCGCG | GGCAACAGCC | ACGAGAAGC |
| | TACTACTGCG | GGAGCCGCAAGTGTACACCCTCCC | TGCCCCCCAC | ACAAGGTCT |
| | CTAGAAGCCT | GCCTTCGCGGGACGAGCTGACCAA | CTTCGGCGGC | ACGCCTGCG |
| | GAGCGGCTAC | GAATCAGGTGTCCCTTACTTGCCTG | GGCACCAAGC | AGGTCACCC |
| | GTGGACTACT | GTGAAGGGATTCTACCCCTCGGAT | TCGAGATCAA | ATCAGGGAC |
| | GGGGCCAAG | ATCGCAGTCGAATGGGAATCGAAT | G | TCTCGTCCC |
| | GCACCACCGT | GGACAGCCAGAAAACAACTACAAG | | CGGTGACCA |
| | GACCGTCTCG | ACCACTCCCCCGGTGCTCGACTCC | | AATCCTTCAA |
| | AGC | GACGGTTCCTTCTTCCTGTACTCGA | | TAGAGGCGA |
| | | AGCTGACCGTGGACAAATCACGCT | | ATGC |
| | | GGCAGCAGGGAAACGTGTTTAGCT | | |
| | | GCAGCGTGATGCATGAGGCGCTGC | | |
| | | ATAATCACTACACCCAGAAGTCACT | | |
| | | CTCGCTCAGCCCAGGGAAG | | |
| 2.9 | SEQ ID NO: 194 | SEQ ID NO: 195 | SEQ ID NO: 196 | SEQ ID NO: |
| | CAAGTGCAGC | GCCAGCACCAAAGGTCCATCCGTG | GACATTCAGA | 197 |
| | TGGTGCAGAG | TTTCCGCTCGCCCCGTCCTCAAAGT | TGACACAGAG | AGAACTGTG |
| | CGGCAGCGAG | CGACCTCCGGAGGCACTGCCGCCC | CCCTAGCAGC | GCCGCGCC |
| | CTGAAGAAGC | TGGGCTGCCTTGTCAAGGACTATTT | CTGAGCGCAA | GTCAGTGTT |
| | CCGGCGCAAG | CCCCGAACCTGTCACGGTGTCCTG | GCGTGGGCGA | TATCTTCCCT |
| | CGTGAAGGTG | GAACAGCGGCGCCCTGACTTCCGG | CAGAGTGACC | CCATCGGAT |
| | AGCTGCAAGG | AGTGCACACCTTCCCCGCCGTCCT | ATCACCTGCA | GAACAGCTT |
| | CAAGCGGCTA | GCAATCCAGCGGCCTGTACTCACT | GAGCAAGCCA | AAGTCCGGC |
| | CACCTTCACC | GTCATCCGTTGTGACTGTCCCGTC | AGACATCAGC | ACGGCGTCT |
| | AACTACTGGA | GTCCAGCCTGGGAACCCAAACCTA | AACAGACTGA | GTGGTCTGC |
| | TGCAGTGGGT | CATTTGCAACGTGAATCACAAACCA | ACTGGTATCA | CTGCTCAAT |
| | GAGACAAGCC | TCGAATACCAAGGTCGATAAGAAAG | GCAGAAGCCC | AACTTTTACC |
| | CCCGGCCAAG | TCGAGCCGAAGTCATGCGACAAGA | GGCAAGGCCG | CTAGGGAAG |
| | GCCTGGAGTG | CTCACACCTGTCCGCCTTGCCCAG | TGAAGCTGCT | CTAAAGTCC |
| | GATCGGCGAG | CCCCAGAACTGCTCGGCGGCCCTT | GATCAGCTAC | AATGGAAAG |
| | ATCGACCCTA | CGGTGTTTTTGTTTCCGCCGAAGCC | AGAAGCAGAC | TGGATAACG |
| | GCGACAGCTA | GAAGGACACTCTGATGATCTCACG | TGCACAGCGG | CCCTGCAGT |
| | CACCAACTAC | CACTCCAGAGGTGACTTGCGTGGT | CGTGCCTAGC | CAGGAAACA |
| | AACCAAGGCT | GGTCGATGTTTCGCACGAGGACCC | AGATTCAGCG | GCCAGGAAT |
| | TCACCGGCAG | GGAAGTGAAATTCAACTGGTATGTC | GCAGCGGCAG | CGGTTACCG |
| | ATTCGTGCTG | GACGGGGTGGAAGTGCATAATGCC | CGGCACCGAC | AACAGGACA |
| | AGCGTGGACA | AAGACGAAGCCGAGGGAGGAACAG | TACACCCTGA | GCAAGGACA |
| | CAAGCGTGAG | TACAACTCCACCTACAGAGTGGTTT | CCATCAGCAG | GCACTTACT |
| | CACCGCCTAC | CAGTCCTTACCGTCCTCCATCAAGA | CCTGCAGCCT | CCTTGTCGT |
| | CTGCAGATCA | TTGGCTGAACGGAAAGGAGTACAA | GAGGACTTCG | CGACTCTTA |
| | GCAGCCTGAA | ATGTAAGGTGTCGAACAAAGCGTT | CCACCTACTT | CTCTGAGCA |
| | GGCCGAGGAC | GCCGGCCCCTATCGAAAAGACTAT | CTGTCAGCAA | AGGCCGATT |
| | ACCGCCGTGT | CAGCAAGGCCAAAGGACAGCCGCG | GGCAACAGCC | ACGAGAAGC |
| | ACTACTGCGC | GGAGCCGCAAGTGTACACCCTCCC | TGCCCCCCAC | ACAAGGTCT |
| | TAGAAGCCTG | GCCTTCGCGGGACGAGCTGACCAA | CTTCGGCGGC | ACGCCTGCG |
| | AGCGGCTACG | GAATCAGGTGTCCCTTACTTGCCTG | GGCACCAAGC | AGGTCACCC |
| | TGGACTACTG | GTGAAGGGATTCTACCCCTCGGAT | TCGAGATCAA | ATCAGGGAC |
| | GGGCCAAGGC | ATCGCAGTCGAATGGGAATCGAAT | G | TCTCGTCCC |
| | ACCACCGTGA | GGACAGCCAGAAAACAACTACAAG | | CGGTGACCA |
| | CCGTCTCGAG | ACCACTCCCCCGGTGCTCGACTCC | | AATCCTTCAA |
| | C | GACGGTTCCTTCTTCCTGTACTCGA | | TAGAGGCGA |
| | | AGCTGACCGTGGACAAATCACGCT | | ATGC |
| | | GGCAGCAGGGAAACGTGTTTAGCT | | |
| | | GCAGCGTGATGCATGAGGCGCTGC | | |
| | | ATAATCACTACACCCAGAAGTCACT | | |
| | | CTCGCTCAGCCCAGGGAAG | | |
| 2.11 | SEQ ID NO: 198 | SEQ ID NO: 199 | SEQ ID NO: 200 | SEQ ID NO: |
| | CAAGTGCAGC | GCCAGCACCAAAGGTCCATCCGTG | GACATCGTAA | 201 |
| | TGGTGCAGAG | TTTCCGCTCGCCCCGTCCTCAAAGT | TGACACAGAG | AGAACTGTG |
| | CGGCAGCGAG | CGACCTCCGGAGGCACTGCCGCCC | CCCCGCGACC | GCCGCGCC |
| | CTGAAGAAGC | TGGGCTGCCTTGTCAAGGACTATTT | CTGAGCCTGT | GTCAGTGTT |
| | CCGGCGCAAG | CCCCGAACCTGTCACGGTGTCCTG | CCCCCGGTGA | TATCTTCCCT |
| | CGTGAAGGTG | GAACAGCGGCGCCCTGACTTCCGG | GAGAGCTACC | CCATCGGAT |
| | AGCTGCAAGG | AGTGCACACCTTCCCCGCCGTCCT | CTGAGCTGCA | GAACAGCTT |
| | CAAGCGGCTA | GCAATCCAGCGGCCTGTACTCACT | GAGCAAGCCA | AAGTCCGGC |
| | CACCTTCACC | GTCATCCGTTGTGACTGTCCCGTC | AGACATCAGC | ACGGCGTCT |
| | AACTACTGGA | GTCCAGCCTGGGAACCCAAACCTA | AACAGACTGA | GTGGTCTGC |
| | TGCAGTGGGT | CATTTGCAACGTGAATCACAAACCA | ACTGGTATCA | CTGCTCAAT |
| | GAGACAAGCC | TCGAATACCAAGGTCGATAAGAAAG | GCAGAAGCCC | AACTTTTACC |
| | CCCGGCCAAG | TCGAGCCGAAGTCATGCGACAAGA | GGCCAAGCCG | CTAGGGAAG |
| | GCCTGGAGTG | CTCACACCTGTCCGCCTTGCCCAG | TGAGACTGCT | CTAAAGTCC |
| | GATCGGCGAG | CCCCAGAACTGCTCGGCGGCCCTT | GATCAGCTAC | AATGGAAAG |
| | ATCGACCCTA | CGGTGTTTTTGTTTCCGCCGAAGCC | AGAAGCAGAC | TGGATAACG |
| | GCGACAGCTA | GAAGGACACTCTGATGATCTCACG | TGCACAGCGG | CCCTGCAGT |
| | CACCAACTAC | CACTCCAGAGGTGACTTGCGTGGT | CGTGCCCGCT | CAGGAAACA |
| | AACCAAGGCT | GGTCGATGTTTCGCACGAGGACCC | AGATTCAGCG | GCCAGGAAT |
| | TCACCGGCAG | GGAAGTGAAATTCAACTGGTATGTC | GCAGCGGCAG | CGGTTACCG |
| | ATTCGTGCTG | GACGGGGTGGAAGTGCATAATGCC | CGGCACCGAC | AACAGGACA |
| | AGCGTGGACA | AAGACGAAGCCGAGGGAGGAACAG | TACACCCTGA | GCAAGGACA |
| | CAAGCGTGAG | TACAACTCCACCTACAGAGTGGTTT | CCATCAGCAG | GCACTTACT |
| | CACCGCCTAC | CAGTCCTTACCGTCCTCCATCAAGA | CCTGGAGCCT | CCTTGTCGT |
| | CTGCAGATCA | TTGGCTGAACGGAAAGGAGTACAA | GAGGACTTCG | CGACTCTTA |
| | GCAGCCTGAA | ATGTAAGGTGTCGAACAAAGCGTT | CCGTGTACTT | CTCTGAGCA |
| | GGCCGAGGAC | GCCGGCCCCTATCGAAAAGACTAT | CTGTCAGCAA | AGGCCGATT |
| | ACCGCCGTGT | CAGCAAGGCCAAAGGACAGCCGCG | GGCAACAGCC | ACGAGAAGC |
| | ACTACTGCGC | GGAGCCGCAAGTGTACACCCTCCC | TGCCCCCCAC | ACAAGGTCT |
| | TAGAAGCCTG | GCCTTCGCGGGACGAGCTGACCAA | CTTCGGCGGC | ACGCCTGCG |
| | AGCGGCTACG | GAATCAGGTGTCCCTTACTTGCCTG | GGCACCAAGC | AGGTCACCC |
| | TGGACTACTG | GTGAAGGGATTCTACCCCTCGGAT | TCGAGATCAA | ATCAGGGAC |
| | GGGCCAAGGC | ATCGCAGTCGAATGGGAATCGAAT | G | TCTCGTCCC |
| | ACCACCGTGA | GGACAGCCAGAAAACAACTACAAG | | CGGTGACCA |
| | CCGTCTCGAG | ACCACTCCCCCGGTGCTCGACTCC | | AATCCTTCAA |
| | C | GACGGTTCCTTCTTCCTGTACTCGA | | TAGAGGCGA |
| | | AGCTGACCGTGGACAAATCACGCT | | ATGC |
| | | GGCAGCAGGGAAACGTGTTTAGCT | | |
| | | GCAGCGTGATGCATGAGGCGCTGC | | |
| | | ATAATCACTACACCCAGAAGTCACT | | |
| | | CTCGCTCAGCCCAGGGAAG | | |
| 2.12 | SEQ ID NO: 202 | SEQ ID NO: 203 | SEQ ID NO: 204 | SEQ ID NO: |
| | CAAGTGCAGC | GCCAGCACCAAAGGTCCATCCGTG | GAGATCGTAA | 205 |
| | TGGTGCAGAG | TTTCCGCTCGCCCCGTCCTCAAAGT | TGACACAGAG | AGAACTGTG |
| | CGGCAGCGAG | CGACCTCCGGAGGCACTGCCGCCC | CCCCGCGACC | GCCGCGCC |
| | CTGAAGAAGC | TGGGCTGCCTTGTCAAGGACTATTT | CTGAGCCTGT | GTCAGTGTT |
| | CCGGCGCAAG | CCCCGAACCTGTCACGGTGTCCTG | CCCCCGGTGA | TATCTTCCCT |
| | CGTGAAGGTG | GAACAGCGGCGCCCTGACTTCCGG | GAGAGCTACC | CCATCGGAT |
| | AGCTGCAAGG | AGTGCACACCTTCCCCGCCGTCCT | CTGAGCTGCA | GAACAGCTT |
| | CAAGCGGCTA | GCAATCCAGCGGCCTGTACTCACT | GAGCAAGCCA | AAGTCCGGC |
| | CACCTTCACC | GTCATCCGTTGTGACTGTCCCGTC | AGACATCAGC | ACGGCGTCT |
| | AACTACTGGA | GTCCAGCCTGGGAACCCAAACCTA | AACAGACTGA | GTGGTCTGC |
| | TGCAGTGGGT | CATTTGCAACGTGAATCACAAACCA | ACTGGTATCA | CTGCTCAAT |
| | GAGACAAGCC | TCGAATACCAAGGTCGATAAGAAAG | GCAGAAGCCC | AACTTTTACC |
| | CCCGGCCAAG | TCGAGCCGAAGTCATGCGACAAGA | GGCCAAGCCG | CTAGGGAAG |
| | GCCTGGAGTG | CTCACACCTGTCCGCCTTGCCCAG | TGAGACTGCT | CTAAAGTCC |
| | GATCGGCGAG | CCCCAGAACTGCTCGGCGGCCCTT | GATCAGCTAC | AATGGAAAG |
| | ATCGACCCTA | CGGTGTTTTTGTTTCCGCCGAAGCC | AGAAGCAGAA | TGGATAACG |
| | GCGACAGCTA | GAAGGACACTCTGATGATCTCACG | GACACACCGG | CCCTGCAGT |
| | CACCAACTAC | CACTCCAGAGGTGACTTGCGTGGT | CATCCCCGCT | CAGGAAACA |
| | AACCAAGGCT | GGTCGATGTTTCGCACGAGGACCC | AGATTCAGCG | GCCAGGAAT |
| | TCACCGGCAG | GGAAGTGAAATTCAACTGGTATGTC | GCAGCGGCAG | CGGTTACCG |
| | ATTCGTGCTG | GACGGGGTGGAAGTGCATAATGCC | CGGCACCGAC | AACAGGACA |
| | AGCGTGGACA | AAGACGAAGCCGAGGGAGGAACAG | TACACCCTGA | GCAAGGACA |
| | CAAGCGTGAG | TACAACTCCACCTACAGAGTGGTTT | CCATCAGCAG | GCACTTACT |
| | CACCGCCTAC | CAGTCCTTACCGTCCTCCATCAAGA | CCTGGAGCCT | CCTTGTCGT |
| | CTGCAGATCA | TTGGCTGAACGGAAAGGAGTACAA | GAGGACTTCG | CGACTCTTA |
| | GCAGCCTGAA | ATGTAAGGTGTCGAACAAAGCGTT | CCGTGTACTA | CTCTGAGCA |
| | GGCCGAGGAC | GCCGGCCCCTATCGAAAAGACTAT | CTGTCAGCAA | AGGCCGATT |
| | ACCGCCGTGT | CAGCAAGGCCAAAGGACAGCCGCG | GGCAACAGCC | ACGAGAAGC |
| | ACTACTGCGC | GGAGCCGCAAGTGTACACCCTCCC | TGCCCCCCAC | ACAAGGTCT |
| | TAGAAGCCTG | GCCTTCGCGGGACGAGCTGACCAA | CTTCGGCGGC | ACGCCTGCG |
| | AGCGGCTACG | GAATCAGGTGTCCCTTACTTGCCTG | GGCACCAAGC | AGGTCACCC |
| | TGGACTACTG | GTGAAGGGATTCTACCCCTCGGAT | TCGAGATCAA | ATCAGGGAC |
| | GGGCCAAGGC | ATCGCAGTCGAATGGGAATCGAAT | G | TCTCGTCCC |
| | ACCACCGTGA | GGACAGCCAGAAAACAACTACAAG | | CGGTGACCA |
| | CCGTCTCGAG | ACCACTCCCCCGGTGCTCGACTCC | | AATCCTTCAA |
| | C | GACGGTTCCTTCTTCCTGTACTCGA | | TAGAGGCGA |
| | | AGCTGACCGTGGACAAATCACGCT | | ATGC |
| | | GGCAGCAGGGAAACGTGTTTAGCT | | |
| | | GCAGCGTGATGCATGAGGCGCTGC | | |
| | | ATAATCACTACACCCAGAAGTCACT | | |
| | | CTCGCTCAGCCCAGGGAAG | | |
| 2.16 | SEQ ID NO: 206 | SEQ ID NO: 207 | SEQ ID NO: 208 | SEQ ID NO: |
| | CAAGTGCAGC | GCCAGCACCAAAGGTCCATCCGTG | GAGATCGTAA | 209 |
| | TGGTGCAGAG | TTTCCGCTCGCCCCGTCCTCAAAGT | TGACACAGAG | AGAACTGTG |
| | CGGCAGCGAG | CGACCTCCGGAGGCACTGCCGCCC | CCCCGCGACC | GCCGCGCC |
| | CTGAAGAAGC | TGGGCTGCCTTGTCAAGGACTATTT | CTGAGCCTGT | GTCAGTGTT |
| | CCGGCGCAAG | CCCCGAACCTGTCACGGTGTCCTG | CCCCCGGTGA | TATCTTCCCT |
| | CGTGAAGGTG | GAACAGCGGCGCCCTGACTTCCGG | GAGAGCTACC | CCATCGGAT |
| | AGCTGCAAGG | AGTGCACACCTTCCCCGCCGTCCT | CTGAGCTGCA | GAACAGCTT |
| | CAAGCGGCTA | GCAATCCAGCGGCCTGTACTCACT | GAGCAAGCCA | AAGTCCGGC |
| | CACCTTCACC | GTCATCCGTTGTGACTGTCCCGTC | AGACATCAGC | ACGGCGTCT |
| | AACTACTGGA | GTCCAGCCTGGGAACCCAAACCTA | AACAGACTGA | GTGGTCTGC |
| | TGAACTGGGT | CATTTGCAACGTGAATCACAAACCA | ACTGGTATCA | CTGCTCAAT |
| | GAGACAAGCC | TCGAATACCAAGGTCGATAAGAAAG | GCAGAAGCCC | AACTTTTACC |
| | CCCGGCCAAG | TCGAGCCGAAGTCATGCGACAAGA | GGCCAAGCCG | CTAGGGAAG |
| | GCCTGGAGTG | CTCACACCTGTCCGCCTTGCCCAG | TGAGACTGCT | CTAAAGTCC |
| | GATGGGCGAG | CCCCAGAACTGCTCGGCGGCCCTT | GATCAGCTAC | AATGGAAAG |
| | ATCGACCCTA | CGGTGTTTTTGTTTCCGCCGAAGCC | AGAAGCAGAA | TGGATAACG |
| | GCGACAGCTA | GAAGGACACTCTGATGATCTCACG | GACACACCGG | CCCTGCAGT |
| | CACCAACTAC | CACTCCAGAGGTGACTTGCGTGGT | CATCCCCGCT | CAGGAAACA |
| | AACCAAGGCT | GGTCGATGTTTCGCACGAGGACCC | AGATTCAGCG | GCCAGGAAT |
| | TCACCGGCAG | GGAAGTGAAATTCAACTGGTATGTC | GCAGCGGCAG | CGGTTACCG |
| | ATTCGTGTTCA | GACGGGGTGGAAGTGCATAATGCC | CGGCACCGAC | AACAGGACA |
| | GCGTGGACAC | AAGACGAAGCCGAGGGAGGAACAG | TACACCCTGA | GCAAGGACA |
| | AAGCGTGAGC | TACAACTCCACCTACAGAGTGGTTT | CCATCAGCAG | GCACTTACT |
| | ACCGCCTACC | CAGTCCTTACCGTCCTCCATCAAGA | CCTGGAGCCT | CCTTGTCGT |
| | TGCAGATCAG | TTGGCTGAACGGAAAGGAGTACAA | GAGGACTTCG | CGACTCTTA |
| | CAGCCTGAAG | ATGTAAGGTGTCGAACAAAGCGTT | CCGTGTACTA | CTCTGAGCA |
| | GCCGAGGACA | GCCGGCCCCTATCGAAAAGACTAT | CTGTCAGCAA | AGGCCGATT |
| | CCGCCGTGTA | CAGCAAGGCCAAAGGACAGCCGCG | GGCAACAGCC | ACGAGAAGC |
| | CTACTGCGCT | GGAGCCGCAAGTGTACACCCTCCC | TGCCCCCCAC | ACAAGGTCT |
| | AGAAGCCTGA | GCCTTCGCGGGACGAGCTGACCAA | CTTCGGCGGC | ACGCCTGCG |
| | GCGGCTACGT | GAATCAGGTGTCCCTTACTTGCCTG | GGCACCAAGC | AGGTCACCC |
| | GGACTACTGG | GTGAAGGGATTCTACCCCTCGGAT | TCGAGATCAA | ATCAGGGAC |
| | GGCCAAGGCA | ATCGCAGTCGAATGGGAATCGAAT | G | TCTCGTCCC |
| | CCACCGTGAC | GGACAGCCAGAAAACAACTACAAG | | CGGTGACCA |
| | CGTCTCGAGC | ACCACTCCCCCGGTGCTCGACTCC | | AATCCTTCAA |
| | | GACGGTTCCTTCTTCCTGTACTCGA | | TAGAGGCGA |
| | | AGCTGACCGTGGACAAATCACGCT | | ATGC |
| | | GGCAGCAGGGAAACGTGTTTAGCT | | |
| | | GCAGCGTGATGCATGAGGCGCTGC | | |
| | | ATAATCACTACACCCAGAAGTCACT | | |
| | | CTCGCTCAGCCCAGGGAAG | | |

In one embodiment, the nucleic acid sequence has at least 60%, 70%, 80%, 90%, 95% or more sequence homology to one of the sequences selected from Table 4. In one embodiment, said sequence homology is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%.

A nucleic acid according to the present invention may comprise DNA or RNA and may be wholly or partially synthetic or recombinantly produced. Reference to a nucleotide sequence as set out herein encompasses a DNA molecule with the specified sequence and encompasses an RNA molecule with the specified sequence in which U is substituted for T, unless context requires otherwise.

Furthermore, the invention relates to a nucleic acid construct comprising at least one nucleic acid as defined above. The construct may be in the form of a plasmid, vector, transcription or expression cassette.

The invention also relates to an isolated recombinant host cell comprising one or more nucleic acid constructs as described above. The host cell may be a bacterial, viral, mammalian or other suitable host cell. In one embodiment, the cell is an E. coli cell. In another embodiment, the cell is a yeast cell. In another embodiment, the cell is a Chinese Hamster Ovary (CHO) cell.

The invention also provides a method of producing an antibody as described herein, wherein the method comprises culturing the host cell under conditions suitable for expression of the polynucleotide encoding the antibody and isolating the antibody.

In one embodiment, the antibodies of the invention are conjugated to a toxin, enzyme, radioisotope, label, therapeutic agent or other chemical moiety.

In embodiments, the antibodies are conjugated to (or labelled with) a detectable or functional label. A label can be any molecule that produces or can be induced to produce a signal, including but not limited to fluorophores, fluorescers, radiolabels, enzymes, chemiluminescers, a nuclear magnetic resonance active label or photosensitizers. Thus, the binding may be detected and/or measured by detecting fluorescence or luminescence, radioactivity, enzyme activity or light absorbance.

The invention provides an immunoconjugate comprising an antibody of the invention conjugated to at least one therapeutic and/or diagnostic agent.

In embodiments, the therapeutic agent may be a toxin, enzyme, radioisotope or other chemical moiety. In one embodiment, the therapeutic agent comprises a toxin, for example a cytotoxic radionuclide, chemical toxin or protein toxin.

The present invention thus relates to an antibody-drug conjugate (ADC) comprising (i) the antibody or antigen-binding fragment thereof as described herein, (ii) a payload, such as a toxin with cytostatic activity capable of killing a cell, and (iii) a payload-antibody linker moiety which is covalently bound to the antibody or antigen-binding fragment thereof. Linker groups for attaching biologically active moieties are well-known in the art.

The payload may be a cytotoxin. In another embodiment, the payload may be an immune-modulating molecule, e.g. a TLR8 or TLR7 agonist or a STING agonist (Gingrich J. How the Next Generation Antibody Drug Conjugates Expands Beyond Cytotoxic Payloads for Cancer Therapy—J. ADC. Apr. 7, 2020).

Examples of toxins which may be conjugated to the antibody are well known in the art and include, for example auristatins, for example monomethyl auristatin E (MMAE) and monomethyl auristatin F (MMAF), or duocarmycins, calicheamicins, pyrrolobenzodiazepines and SN-38. In embodiments, the immunoconjugate comprises an antibody linked to MMAE.

MMAE is a synthetic antimitotic antineoplastic agent. MMAE is 100-1000 times more potent than doxorubicin but its toxicity is such that it cannot be used as a drug itself. However, it has been used as part of an antibody-drug conjugate (ADC) where MMAE is linked to an antibody that recognises a specific marker expressed in cancer cells and directs MMAE to the cancer cell.

The structure linking the antibody to MMAE may comprise an attachment group (maleimide (aml) and caproic acid (cap)), a spacer (paraaminobenzoic acid) and a cathepsin-cleavable linker (amino acids valine (Val) and citrulline (Cit)). The structure that connects MMAE to the antibody may be cleaved by cathespsin once the ADC has bound to the targeted cancer cell antigen and entered the cancer cell, after which the ADC releases the toxic MMAE and activates the potent anti-mitotic mechanism. ADCs reduce adverse systemic effects of highly potent cytotoxic agents.

MMAF is a synthetic antineoplastic agent which is used in some experimental anti-cancer ADCs as the toxic entity. The structure linking the antibody to MMAF may comprise maleimide and caproic acid.

Monomethyl auristatin E (MMAE, desmethyl-auristatin E) is a synthetic antimitotic, antineoplastic agent The IUPAC name for MMAE is (S)-N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide.

Monomethyl auristatin E or MMAE is 100-1000 times more potent than doxorubicin, but its toxicity is such that cannot be used as a drug itself. However, it has been used as part of an antibody-drug conjugate or ADC, wherein MMAE is linked to a monoclonal antibody (mAb) that recognizes a specific marker expressed in cancer cells and directs MMAE to the cancer cell.

As MMAE is toxic, it has been used as a therapeutic only when conjugated to a monoclonal antibody (mAb) to target the MMAE to cancer cells. In the International Nonproprietary Names for MMAE-mAb-conjugates, the name “vedotin” denotes MMAE plus its linking structure to the antibody. The structure linking the targeting mAb to MMAE may comprise an attachment group (maleimide (mal) and caproic acid (cap)), a spacer (paraaminobenzoic acid) and a cathepsin-cleavable linker (amino acids valine (Val) and citrulline (Cit)).

The tether that connects MMAE to the monoclonal antibody is stable in extracellular fluid, but is cleaved by cathepsin once the antibody-drug-conjugate has bound to the targeted cancer cell antigen and entered the cancer cell, after which the ADC releases the toxic M MAE and activates the potent anti-mitotic mechanism. Antibody-drug conjugates enhance the antitumor effects of antibodies and reduce adverse systemic effects of highly potent cytotoxic agents.

Monomethyl auristatin F (MMAF, desmethyl-auristatin F) is a synthetic antineoplastic agent. The IUPAC name for MMAF is (S)-2-((2R,3R)-3-((S)-1-((3R,4S,5S)-4-((S)-N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-phenylpropanoic acid MMAF is the toxic payload used in some experimental anti-cancer antibody-drug conjugates such as vorsetuzumab mafodotin and SGN-CD19A. In International Nonproprietary Names for MMAF-antibody-conjugates, the name mafodotin refers to MMAF plus its attachment structure to the antibody. The attachment group may consist of maleimide and caproic acid.

Auristatins and their use as components of ADC are reviewed by Maderna and Leverett in “Recent Advances in the Development of New Auristatins: Structural Modifications and Application in Antibody Drug Conjugates”; Mol. Pharmaceutics, 2015, 12 (6), pp 1798-1812 Mendelsohn et al., “Investigation of Hydrophilic Auristatin Derivatives for Use in Antibody Drug Conjugates”. Bioconjugate Chem., Article ASAP DOI: 10.1021/acs.bioconjchem.6b00530, Publication Date (Web): Jan. 6, 2017 describe derivatives of the natural product dolastatin 10 containing pyridines and other basic amines, which were examined to assess more hydrophilic auristatin derivatives would be sufficiently potent for use in ADC. A pyridine derivative, monomethyl auristatin PYE, showed the greatest potency when tested in vivo.

MMAE may be conjugated to the antigen-binding moiety via a valine-citrulline (vc) linker (vc-MMAE). MMAF is conjugated to the antigen-binding moiety via a maleimidocaproyl linker (mc-MMAF) using HiPEG™ technology (WO 2009/047500). Thus, in one embodiment, D is MMAE or a derivative thereof conjugated to the antigen- binding moiety via a valine-citrulline (vc) linker (vc-M MAE). In another embodiment D is MMAF or a derivative thereof conjugated to the antigen-binding moiety via a maleimidocaproyl linker (mc-MMAF). In another embodiment L-D is vedotin or mafodotin.

The immunoconjugate, compositions and methods of the invention may feature an auristatin which is either monomethylaunstatin E (MMAE) or monomethylaunstatin F (MMAF) or a derivative thereof.

Immunoconjugates in which the antibody is conjugated to a toxin can be used to mediate specific cell killing of LGR5 expressing cancer cells without systemic toxicity.

A diagnostic agent may be a detectable or functional label, as described above.

In another aspect of the present invention, there is provided a pharmaceutical composition comprising an antibody according to the present invention or an immunoconjugate according to the invention and, optionally, a pharmaceutically acceptable carrier.

An antibody, immunoconjugate, or pharmaceutical composition of the invention can be administered by any convenient route, including but not limited to oral, topical, parenteral, sublingual, rectal, vaginal, ocular, intranasal, pulmonary, intradermal, intravitrial, intramuscular, intraperitoneal, intravenous, subcutaneous, intracerebral, transdermal, transmucosal, by inhalation, or topical, particularly to the ears, nose, eyes, or skin or by inhalation.

Parenteral administration includes, for example, intravenous, intramuscular, intraarterial, intraperitoneal, intranasal, rectal, intravesical, intradermal, topical or subcutaneous administration. Preferably, the compositions are administered parenterally.

The pharmaceutically acceptable carrier or vehicle can be particulate, so that the compositions are, for example, in tablet or powder form. The term “carrier” refers to a diluent, adjuvant or excipient, with which a drug antibody conjugate of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the antibody of the present invention or compositions and pharmaceutically acceptable carriers are sterile. Water is a preferred carrier when the drug antibody conjugates of the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The pharmaceutical composition of the invention can be in the form of a liquid, e.g., a solution, emulsion or suspension. The liquid can be useful for delivery by injection, infusion (e.g., IV infusion) or subcutaneously.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e. g. a gelatin capsule), it can contain, in addition to materials of the above type, a liquid carrier such as polyethylene glycol, cyclodextrin or a fatty oil.

The composition can be in the form of a liquid, e. g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units.

In specific embodiments, it can be desirable to administer the composition locally to the area in need of treatment, or by intravenous injection or infusion.

The amount of the antibody of the present invention that is effective/active in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances. Factors like age, body weight, sex, diet, time of administration, rate of excretion, condition of the host, drug combinations, reaction sensitivities and severity of the disease shall be taken into account.

Typically, the amount is at least about 0.01% of an antibody of the present invention by weight of the composition. W hen intended for oral administration, this amount can be varied to range from about 0.1% to about 80% by weight of the composition. P referred oral compositions can comprise from about 4% to about 50% of the antibody of the present invention by weight of the composition.

Preferred compositions of the present invention are prepared so that a parenteral dosage unit contains from about 0.01% to about 2% by weight of the antibody of the present invention.

For administration by injection, the composition can comprise from about typically about 0.1 mg/kg to about 250 mg/kg of the animal's body weight, preferably, between about 0.1 mg/kg and about 20 mg/kg of the animal's body weight, and more preferably about 1 mg/kg to about 10 mg/kg of the animal's body weight. In one embodiment, the composition is administered at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks.

The invention provides methods of treating or diagnosing disease, for example cancer, in a subject, e.g., a mammal (e.g. human patient), comprising administering an effective amount of an antibody, immunoconjugate or pharmaceutical composition of the present invention to the subject. The invention provides methods of diagnosing disease, for example cancer, by assessing LGR5 expression, e.g. in a target tissue. The method is described in detail herein.

As used herein, “treat”, “treating” or “treatment” means inhibiting or relieving a disease or disorder. For example, treatment can include a postponement of development of the symptoms associated with a disease or disorder, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the subjects, e.g., human patients, being treated. Many medical treatments are effective for some, but not all, patients that undergo the treatment.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

As used herein, the term “effective amount” means an amount of an antibody, that when administered alone or in combination with an additional therapeutic agent to a cell, tissue, or subject, is effective to achieve the desired therapeutic or prophylactic effect under the conditions of administration

The invention also relates to an antibody, immunoconjugate or pharmaceutical composition of the invention for use in the treatment or prevention of a disease.

In another aspect, the invention relates to an antibody, immunoconjugate or pharmaceutical composition of the invention for use in the treatment or prevention of cancer.

In another aspect, the invention relates to the use of an antibody, immunoconjugate or pharmaceutical composition of the invention in the treatment or prevention of a disease.

In another aspect, the invention relates to the use of an antibody, immunoconjugate or pharmaceutical composition of the invention in the manufacture of a medicament for the treatment or prevention of cancer.

In an aspect of the invention the antibodies may be used as part of a CAR-T cell for the treatment of cancer. The CAR-T cell may comprise a chimeric antigen receptors (CAR) which comprises an antibody of the invention i.e. an antibody or fragment thereof which binds to LGR5.

Adoptive cellular therapy (ACT) has received much attention as a technique for cancer treatment. One therapeutic approach of ACT involves genetic engineering of T cells to express chimeric antigen receptors (CARs) on the surface of T cells to enable targeting of specific tumours. Once the CAR is expressed in T cells, the CAR modified T cell (CAR-T or CAR- T cell) acquires properties that include antigen-specific recognition, activation and proliferation and the cells thus act as “living drugs”. The purpose of expressing a CAR in a T cell is therefore to redirect immune reactivity of the cell to a chosen target. Furthermore, CARs with different strength and signalling can also modulate T cell expansion as well as alter the strength of T cell activation.

CARs are synthetic receptors typically consisting of a targeting/binding moiety that is associated with one or more signaling domains in a single fusion molecule. The binding moiety of a CAR typically consists of an antigen-binding domain of a single-chain antibody (scFv) comprising paired antibody light chain and heavy chain variable domains (V_{L} and V_{H}) that are fused into a single polypeptide chain via a short flexible linker. The scFv retains the same specificity and a similar affinity as the full antibody from which it was derived and is capable of binding to the specific target of interest. In addition to an extracellular antigen-binding domain CARs also comprise a transmembrane domain and signaling molecules such as costimulatory endodomains and CD3(chain.

CARs combine antigen-specificity and T cell activating properties in a single fusion molecule. First generation CARs typically included the cytoplasmic region of the CD3zeta or Fc receptor γ chain as their signalling domain. First generation CARs have been tested in phase I clinical studies in patients with ovarian cancer, renal cancer, lymphoma, and neuroblastoma, where they have induced modest responses (reviewed in Sadelain et al., Curr Opin Immunol, 21 (2): 215-223, 2009). Second generation CARs, which contain the signalling domains of both CD28 and CD3zeta, provide dual signalling to direct combined activating and co-stimulatory signals. Third generation CARs are more complex with three or more signalling domains.

The terms “Chimeric antigen receptor” or “CAR” or “CARs” as used herein refer to engineered receptors, which graft an antigen specificity onto cells (for example T cells such as naive T cells, central memory T cells, effector memory T cells or combination thereof) thus combining the antigen binding properties of the antigen binding domain with the lytic capacity and self renewal of T cells. CARs are also known as artificial T cell receptors, chimeric T cell receptors or chimeric immunoreceptors. The term “antigen binding domain or “antigen-specific targeting domain” as used herein refers to the region of the CAR which targets and binds to specific antigens as explained above. When a CAR is expressed in a host cell, this domain forms the extracellular domain (ectodomain).

A skilled person would know that a CAR may comprise additional elements.

A skilled person would also know that such elements of a CAR (other than antigen-specific targeting domain described herein) are well known in the art. Thus, the invention is not limited to specific domains of the CAR in addition to the antigen-specific targeting domain described herein.

As mentioned above, the first generation CARs have been tested in various phase I clinical studies in patients with cancer. Second generation CARs and third generation CARs have also been described are more complex with three or more signalling domains (reviewed in Sadelain et al., Curr Opin Immunol, 21 (2): 215-223, 2009, Sterner, R. C., Sterner, R. M. CAR-T cell therapy: current limitations and potential strategies. Blood Cancer J. 11, 69, 2021). CARs are also described in US2004043401, WO2019200007 and WO2021108613, all incorporated herein by reference.

For example, the CAR of the invention may comprise a molecule of the general formula: LGR5 binding scFv—transmembrane domain- Intracellular signaling domain. Exemplary domains transmembrane domains and intracellular signaling domains are listed below and are known in the art.

The present invention may therefore also provide a chimeric antigen receptor comprising an antibody or fragment thereof, e.g. a scFv, of the invention. In one embodiment, the CAR may be labelled. In one embodiment, the CAR comprises a scFv as shown in SEQ ID NO: 211 or a sequence with 70%, 80 or 90% sequence identity thereto as encoded by SEQ ID NO: 218 or a sequence with 70%, 80 or 90% sequence identity thereto. In one embodiment, the CAR comprises a scFv that comprises the CDRs of clone 2.4, also numbered herein as SEQ ID NO. 212, 213 and 214 and the heavy chain CDRs of 215, 216 and 217. In one embodiment, the CAR comprises a scFv that comprises the VH and VL of clone 2.4 or a sequence with at least 90% sequence identity thereto.

The invention also relates to a cell expressing a CAR of the invention, for example an immune cell, for example wherein the immune cell is selected from the group consisting of a T cell, a Natural Killer (NK) cell, a cytotoxic T lymphocyte (CTL), tumor infiltrating lymphocyte (TIL), TCR-expressing cell, dendritic cell, or NK-T cell and a regulatory T cell. The cell may be an autologous T cell or an allogeneic T cell.

The invention also relates to a cell or population of cells comprising a CAR as described herein. The invention also relates to a population of cells comprising a CAR as described herein for use in adaptive immunotherapy, e.g. to treat cancer. The invention also relates to a method for treating, e.g. for adoptive adaptive immunotherapy for treating a cancer comprising administering a population of cells comprising a CAR as described herein.

The invention also relates to a method for stimulating a T cell-mediated immune response to a target cell population or tissue in a subject, the method comprising administering to the subject an effective amount of a cell or cell population comprising a CAR as described herein.

The invention also relates to an ex vivo method for generating a population of cells for use in adaptive immunotherapy comprising transforming said cell with a nucleic acid encoding a CAR, e.g. SEQ ID NO. 218 or a sequence with 70%, 80 or 90% sequence identity thereto.

The invention also relates to method of making a population expressing a CAR, the method comprising:
(i) contacting a population of cells (for example, T cells, for example, T cells isolated from a frozen or fresh leukapheresis product) with an agent that stimulates a CD3/TCR complex and/or an agent that stimulates a costimulatory molecule on the surface of the cells; (ii) contacting the population of cells (for example, T cells) with the nucleic acid molecule of SEQ ID NO. 218 or a sequence with 70%, 80 or 90% sequence identity thereto, thereby providing a population of cells (for example, T cells) comprising the nucleic acid molecule, and (iii) harvesting the population of cells (for example, T cells) for storage (for example, reformulating the population of cells in cryopreservation media) or administration.

As will be appreciated by the skilled person, the term “chimeric antigen receptor” or “CAR” refers to a recombinant molecule containing an extracellular recognition domain, a transmembrane region and an intracellular signaling domain.

The extracellular domain comprises a ligand specific for a target tumour antigen, for example an antibody or fragment thereof which binds to LGR5, as described herein.

The extracellular domain is tethered to a transmembrane region. The transmembrane region may be selected from the transmembrane region(s) of the alpha, beta or zeta chain of the T-cell receptor, PD-1, 4-1BB, OX40, ICOS, CTLA-4, LAG3, 2B4, BTLA4, TIM-3, TIGIT, SIRPA, CD28, CD3 epsilon, CD3zeta, CD45, CD4, CD5, CD8, CD9, CD16, CD22, CD33, CD37, CD64, CD80, CD86, CD134, CD137 or CD154, for example.

The intracellular (cytoplasmic) domain of the CAR can provide activation of at least one of the normal effector functions of the immune cell. The CAR of the invention may thus further comprise an intracellular signaling domain. An “intracellular signaling domain”, “cytoplasmic domain” or “endodomain” is the domain that transmits activation signals to T cells and directs the cell to perform its specialized function.

An “intracellular signaling domain,” as the term is used herein, refers to an intracellular portion of a molecule. The intracellular signaling domain generates a signal that promotes an immune effector function of the CAR containing cell, e.g., a CART cell or CAR-expressing NK cell.

Examples of immune effector function, e.g., in a CART cell or CAR-expressing NK cell, include cytolytic activity and helper activity, including the secretion of cytokines.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

The intracellular signaling domain contains a signaling domain, for example CD28, OX40 and/or CD3zeta. The intracellular signaling domain may comprise a co-stimulatory domain the costimulatory domain is a signaling region of CD28, CD8, OX40, 4-1BB, CD2, CD7, CD27, CD30, CD40, programmed death-1 (PD-1), inducible T cell co-stimulator (ICOS), lymphocyte function- associated antigen-1 (LFA-1 (CD1 la/CDl8), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM1l, B7-H3, CDS, ICAM-I, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, IT GAD, CD1 Id, ITGAE, CD 103, ITGAL, CD1 la, LFA-I, ITGAM, CD1 lb, ITGAX, CD1 Ic, ITGB1, CD29, ITGB2, CD 18, LFA-I, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Lyl08), SLAM (SLAMF1, CD 150, IPO-3), BLAME (SLAMF8), SELPLG (CD162), LTBR, LAT, GADS, SLP-76, PAG/Cbp, CDI9a, a ligand that specifically binds with CD83, or any combination thereof.

In an aspect of the invention the antibodies may be used as part of a Bispecific T-cell Engager (BiTE) for the treatment of cancer. The BiTE may comprise an antibody of the invention, i.e. an antibody or fragment thereof which binds to LGR5.

The present invention may therefore also provide a BiTE comprising an antibody of the invention.

As will be appreciated by the skilled person, the term “Bispecific T-cell Engager” or “BiTE” As refers to fusion proteins including two scFvs of different antibodies wherein one of the scFvs binds to T cells via the CD3 receptor, and the other to a tumor cell via a tumor specific molecule (e.g. an antibody or fragment thereof which binds to LGR5). The CD3 molecule non-covalently associates with a T cell receptor (TCR) and participates in antigen-specific signal transduction which activates T cells and initiates redirected lysis of the tumour cell by the T cell.

In one embodiment, the BiTE comprises a scFv as shown in SEQ ID NO: 211 or a sequence with 70%, 80 or 90% sequence identity thereto or one or more CDR of SEQ ID NO: 211.

The cancer may be a solid or non-solid tumor. For example, the cancer may be selected from cancer of the head or neck, uterine cancer, colorectal cancer, stomach cancer, carcinoma of the endometrium, cancer of the esophagus, leukemia, such as acute lymphoblastic leukemia (ALL), liver cancer, such as hepatocellular carcinoma or pancreatic cancer.

In one embodiment, the tumour is a solid tumour. Solid tumours which may be treated include colorectal carcinoma, for example. Some examples of such tumours include epidermoid tumours, squamous tumours, such as head and neck tumours, colorectal tumours.

In one embodiment, the tumour is a non-solid tumour. Examples of non-solid tumours include leukemia.

In one embodiment, the cancer is selected from colorectal cancer (CRC), hepatocellular carcinoma (HCC), and pre-B ALL.

In one embodiment, the cancer is identified as a LGR5 positive cancer. The term “LGR5 positive cancer” as used herein, means a cancer whose cells express LGR5. The antibody and pharmaceutical compositions of the present invention are particularly useful for the treatment of cancers that express abnormally high levels of LGR5, for example cancers which overexpress LGR5. The term “overexpress” as used herein means that the cells express more LGR5 than that observed in normal, non-cancerous cells. The cancer cells may express 5, 10, 20, 30, 40, 50, 60, 70, 80, 90% more LGR5 than that observed in normal, non-cancerous cells.

The skilled person will be well aware how to assess whether a cancer is LGR5 positive and/or overexpresses LGR5. Methods include for example, using fluorescence in-situ hybridization, immunohistochemistry approaches, flow cytometry, RT-PCR.

The present inventors have found that by utilising the antibodies of the present invention, a toxic payload, in the form of an antibody drug conjugate (ADC) can be delivered to LGR5-expressing cancer cells to kill those cells without systemic toxicity.

In one embodiment, the cancer is locally advanced, unresectable, metastatic, or recurrent cancer.

In one embodiment, the cancer has progressed after another treatment, for example chemotherapy.

The antibody, immunoconjugate or pharmaceutical composition of the invention may be administered as the sole active ingredient or in combination with one or more other therapeutic agents. A therapeutic agent is a compound or molecule which is useful in the treatment of a disease. Examples of therapeutic agents include antibodies, antibody fragments, drugs, toxins, nucleases, hormones, immunomodulators, pro-apoptotic agents, anti-angiogenic agents, boron compounds, photoactive agents or dyes and radioisotopes. An antibody molecule includes a full antibody or fragment thereof (e.g., a Fab, F(ab′)2, Fv, a single chain Fv fragment (scFv) or a single domain antibody, for example a V_{H} domain) or antibody mimetic protein.

In one embodiment, the treatment is used in combination with an existing therapy or therapeutic agent, for example an existing anti-cancer therapy. Thus, in another aspect, the invention also relates to a combination therapy comprising administration of an antibody, immunoconjugate or pharmaceutical composition of the invention and an anti-cancer therapy.

The anti-cancer therapy may include a therapeutic agent or radiation therapy and includes gene therapy, viral therapy, RNA therapy bone marrow transplantation, nanotherapy, targeted anti-cancer therapies or oncolytic drugs. Examples of other therapeutic agents include other checkpoint inhibitors, antineoplastic agents, immunogenic agents, attenuated cancerous cells, tumor antigens, antigen presenting cells such as dendritic cells pulsed with tumor-derived antigen or nucleic acids, immune stimulating cytokines (e.g., IL-2, IFNa2, GM-CSF), targeted small molecules and biological molecules (such as components of signal transduction pathways, e.g. modulators of tyrosine kinases and inhibitors of receptor tyrosine kinases, and agents that bind to tumor- specific antigens, including E G F R antagonists), an anti-inflammatory agent, a cytotoxic agent, a radiotoxic agent, or an immunosuppressive agent and cells transfected with a gene encoding an immune stimulating cytokine (e.g., GM-CSF), chemotherapy. In one embodiment, the antibody is used in combination with surgery.

In a specific embodiment of the present invention, the composition is administered concurrently with a chemotherapeutic agent or with radiation therapy. In another specific embodiment, the chemotherapeutic agent or radiation therapy is administered prior or subsequent to administration of the composition of the present invention, preferably at least an hour, five hours, 12 hours, a day, a week, a month, more preferably several months (e. g. up to three months), prior or subsequent to administration of composition of the present invention.

In some embodiments, the antibody, immunoconjugate or pharmaceutical composition of the invention may be administered with two or more therapeutic agents.

The antibody, immunoconjugate or pharmaceutical composition of the invention may be administered at the same time or at a different time as the other therapy or therapeutic compound or therapy, e.g., simultaneously, separately or sequentially.

In another aspect, the invention relates to an antibody, immunoconjugate or pharmaceutical composition of the invention for use in the treatment or prevention of inflammatory disease.

In another aspect, the invention relates to the use of an antibody, immunoconjugate or pharmaceutical composition of the invention in the manufacture of a medicament for the treatment or prevention of inflammatory disease.

In another aspect, the invention relates to a method for treating an inflammatory disease comprising administering a therapeutically effective amount of an antibody as described herein, an immunoconjugate as described herein or a pharmaceutical composition as described herein.

As will be appreciated by the skilled person the term “inflammatory disease” refers to a disease in which tissues are inflamed, with, for example, increased recruitment of white blood cells to a tissue, which may cause swelling, pain and loss of function. Inflammatory diseases include, for example, arthritis, asthma, Crohn's disease, colitis, dermatitis, irritable bowel syndrome.

Notably, chronic inflammation is often a precursor to cancer. Inflammatory diseases also include autoimmune diseases and other inflammatory disorders such as myositis, ankylosing spondylitis, vasculitis.

In embodiments, the inflammatory disease is an inflammatory disease that expresses abnormally high levels of LGR5, for example inflammatory disease which overexpress LGR5.

The term “overexpress” as used herein means that the cells express more LGR5 than that observed in normal cells not suffering from inflammatory disease. The cells may express 5, 10, 20, 30, 40, 50, 60, 70, 80, 90% more LGR5 than that observed in normal cells. The skilled person will be well aware how to assess whether an inflammatory disease overexpresses LGR5. Methods include for example, using fluorescence in-situ hybridization, immunohistochemistry approaches, flow cytometry, RT-PCR.

Without wish to be bound by theory, the inventors hypothesize that chronically inflamed tissues result in the upregulation of stem cell pathways and overexpression of LGR5. By targeting LGR5, using the antibodies of the present invention, the inventors can therefore target cells affected by inflammatory disease.

The present inventors have found that by utilising the antibodies of the present invention, a toxic payload, in the form of an antibody drug conjugate (ADC) can be delivered to LGR5-expressing cells to kill those cells without systemic toxicity.

In another aspect, the invention provides a kit comprising an antibody, immunoconjugate or a pharmaceutical composition as described herein.

In another aspect, the invention provides a kit for the treatment or prevention of a disease and/or for detecting LGR5 for diagnosis, prognosis or monitoring disease comprising an antibody or immunoconjugate of the invention. Such a kit may contain other components, packaging, instructions, or material to aid in the detection of LGR5 protein. The kit may include a labeled antibody of the invention as described above and one or more compounds for detecting the label.

The invention in another aspect provides an antibody of the invention packaged in lyophilized form or packaged in an aqueous medium.

The invention also relates to an antibody as described herein with reference to the figures and examples.

In another aspect, antibodies of the invention are used for non-therapeutic purposes, such as diagnostic tests and assays. A method for detecting the presence of LGR5 in a biological sample comprises contacting said sample with an antibody according to the invention under conditions permissive for binding of the antibody to LGR5, and detecting whether a complex is formed between the antibody and LGR5.

The inventors have shown that the antibodies of the present invention bind with high affinity and specificity to cancer cells expressing LGR5. Therefore, the antibodies of the present invention may be used in a method of detecting cancer is a sample from a subject.

The invention therefore provides a method of detecting cancer in a biological sample from a subject, the method comprising contacting the sample with an antibody according to the invention under conditions permissive for binding of the antibody to LGR5, and detecting whether a complex is formed between the antibody and LGR5. The method is particularly useful in detecting a cancer which is positive for LGR5 or overexpresses LGR5.

The invention therefore also provides a method of detecting inflammatory disease in a biological sample from a subject, the method comprising contacting the sample with an antibody according to the invention under conditions permissive for binding of the antibody to LGR5, and detecting whether a complex is formed between the antibody and LGR5. The method is particularly useful in detecting an inflammatory disease which overexpresses LGR5.

The methods may be carried out in vivo, in vitro or ex vivo.

Modifications of antibodies for diagnostic purposes are well known in the art. For example, antibodies may be modified with a ligand group such as biotin, or a detectable marker group such as a fluorescent group, a radioisotope, or an enzyme. Compounds of the invention can be labelled using conventional techniques. Suitable detectable labels include but are not limited to fluorophores, chromophores, radioactive atoms, electron-dense reagents, enzymes, and ligands having specific binding partners.

The biological sample, may include, for example, a tissue sample, blood and blood components (e.g. serum), mucus, saliva, urine, vomit, faeces, sweat, semen, vaginal secretion, tears, pus, sputum or pleural fluid. Preferably the biological sample is a blood sample or tissue sample. As will be appreciated by the skilled person, the method of detecting cancer may therefore be in vitro method for detecting cancer in a subject, the method being carried out on a sample provided from a subject.

There is also provided a method of detecting cancer in a biological sample from a subject, the method comprising contacting the sample with an antibody according to the invention under conditions permissive for binding of the antibody to LGR5, and detecting whether a complex is formed between the antibody and LGR5. The method is particularly useful in detecting a cancer which is positive for LGR5 or overexpresses LGR5. If a complex if formed between the antibody and LGR5 this maybe indicative that cancer is present in the subject. In such cases, the method may further comprise administering to the subject a cancer treatment. In embodiments, the treatment may comprise administering the antibody, immunoconjugate or pharmaceutical composition of the invention, particularly immunoconjugates or pharmaceutical compositions wherein the antibody is conjugated to a therapeutic agent such as a toxin, enzyme, radioisotope or other chemical moiety (as described above).

There is also provided a method of detecting inflammatory disease in a biological sample from a subject, the method comprising contacting the sample with an antibody according to the invention under conditions permissive for binding of the antibody to LGR5, and detecting whether a complex is formed between the antibody and LGR5. The method is particularly useful in detecting an inflammatory disease which overexpresses LGR5. If a complex if formed between the antibody and LGR5 this may be indicative that inflammatory disease may be present in the subject. In such cases, the method may further comprise administering to the subject a treatment. In embodiments, the treatment may comprise administering the antibody, immunoconjugate or pharmaceutical composition of the invention, particularly immunoconjugates or pharmaceutical compositions wherein the antibody is conjugated to a therapeutic agent such as a toxin, enzyme, radioisotope or other chemical moiety (as described above).

In a preferred embodiment, the present invention relates to an in vitro method of detecting an epitope of the invention in a sample, wherein the method comprises incubating an antigen binding protein of the invention with a sample of interest, and determining binding of the antigen-binding protein to an epitope of the invention present in the sample, wherein binding of the antigen-binding protein indicates the presence of an epitope of the invention in the sample. Methods for detecting binding of an antigen-binding protein to its target antigen are known in the art and include ELISA, ICC, IHC, immunofluorescence, western blot, IP, SPR and flow cytometry. The sample of interest may be a sample obtained from an individual. The individual may be human. Samples include, but are not limited to, tissue such as brain tissue, cerebro-spinal fluid (CSF), primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, saliva, sputum, tears, perspiration, mucus, tumour lysates, and tissue culture medium, tissue extracts such as homogenized tissue, tumour tissue, cellular extracts, and combinations thereof.

Following incubation, antigen-binding protein to antigen binding, e.g., antibody to antigen binding, is detected using an appropriate detection system. The method of detection can be direct or indirect, and may generate a fluorescent or chromogenic signal. Direct detection involves the use of primary antibodies that are directly conjugated to a label. Indirect detection methods employ a labelled secondary antibody raised against the primary antigen-binding protein, e.g., antibody, host species. Indirect methods may include amplification steps to increase signal intensity. Commonly used labels for the visualization (i.e., detection) of antigen-binding protein—antigen (e.g., antibody—epitope) interactions include fluorophores and enzymes that convert soluble substrates into insoluble, chromogenic end products.

The term “detecting” is used herein in the broadest sense to include both qualitative and quantitative measurements of a target molecule. Detecting includes identifying the mere presence of the target molecule in a sample as well as determining whether the target molecule is present in the sample at detectable levels. Detecting may be direct or indirect.

Suitable detectable labels which may be conjugated to antigen-binding proteins, such as antibodies are known in the art and include radioisotopes such as iodine-125, iodine-131, yttrium-90, indium-1 11 and technetium-99; fluorochromes, such as fluorescein, rhodamine, phycoerythrin, Texas Red and cyanine dye derivatives for example, Cy7, Alexa750 and Alexa Fluor 647; chromogenic dyes, such as diaminobenzidine; latex beads; enzyme labels such as horseradish peroxidase; phospho or laser/fluorescent dyes with spectrally isolated absorption or emission characteristics; electro-chemiluminescent labels, such as SULFO-TAG which may be detected via stimulation with electricity in an appropriate chemical environment; and chemical moieties, such as biotin, which may be detected via binding to a specific cognate detectable moiety, e.g. labelled avidin or streptavidin.

In another aspect, the invention relates to an isolated synthetic or recombinant peptide comprising an epitope, the peptide consisting of residues 22 to 37 of SEQ ID NO: 1. This may be used in method for immunization to identify and select antibodies. Thus, methods of immunization using the fragment are also contemplated.

Further. the invention relates to the use of LGR5 expression as a prognostic marker.

Thus, the invention relates to a method of diagnosing or assessing progression of cancer comprising assessing expression of LGR5 and/or protein levels of LGR5. Assessing expression includes measuring expression using routine methods. Proteins levels can be assessed using the antibodies of the invention.

In one embodiment, the method may further comprise comparing the level of expression to a threshold level.

In one embodiment, the threshold level is the level of expression in normal tissue.

In one embodiment, the method may further comprise selecting a treatment if the level of expression is above the threshold.

In one embodiment, the method may further comprise administering said treatment.

In one embodiment, the treatment is an antibody or antibody fragment as described herein.

In one embodiment, the cancer is CRC, HCC and pre-B ALL.

In one embodiment, the cancer is HCC and the method may further comprise assessing the presence of mutations in β-catenin.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety, including references to gene accession numbers and references to patent publications.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The invention is further described in the non-limiting examples.

### EXAMPLES

The present inventors undertook significant experimentation to develop the antibodies of the present invention and in doing so have established antibodies with high binding affinity and specificity which may be used as both a research tool and for therapeutic purposes.

### Materials and Methods

### Plasmid Constructs

The plasmid for hLGR5-eGFP expression plasmid has been previously described. All other LGR transgenes used in the study were constructed by direct replacement of the LGR5 coding sequence with PCR amplicons from the corresponding LGR -family coding sequence by either Gibson assembly (New England Biolabs) or restriction enzyme cloning. LGR-family coding sequences were sourced as follows: hLGR4 (HG15689), Sino Biological; mLGR4 (MR219497) Origene; mLGR5 (MR219702) Origene; and hLGR6(LGR6_OHu16329D) GenScript.

Sequencing of antibody hybridoma clones and generation of plasmids encoding heavy and light chains of the humanised α-LGR5 antibodies (α-LGR5v4 and α-LGR5v6) and the scFv fragment were obtained from Absolute Antibody.

Preparation of Proteins from Bacterial Expression

The coding sequence for the N-terminal 100 amino acids of human LGR5 lacking the signal peptide was inserted into the pGEX-4T1 bacterial expression vector and expressed in E. coli XL1 blue. The expressed GST-fusion protein was absorbed to glutathione-Sepharose 4B (Sigma-Aldrich) followed by extensive washing of the column with phosphate buffered saline (PBS) and elution with 20 mM glutathione. The eluate was digested, overnight with 2 U of thrombin protease (Sigma) and the LGR5 N-terminal fragment was resolved in PBS on a Superdex 75 10/300 gel filtration column on an ÄKTA pure system.

Bacterial expression plasmids for RAD display of LGR5 N-terminal fragments were generated by Gibson assembly, expressed in bacteria and purified by heat denaturation and Ni-Sepharose affinity chromatography as previously described (Rossmann et al).

### Mammalian Cell Lines

Cell lines were purchased from the European Collection of Cell Cultures and have been authenticated by short tandem repeat (STR) DNA profiling. Upon receipt, cell lines were frozen, and individual aliquots were taken into culture, typically for analysis within <10 passages. Cells were grown in a humidified incubator at 37° C. and 5% CO2 and tested mycoplasma negative (MycoProbe® Mycoplasma Detection Kit, R&D systems). HEK293T cells, and the colorectal cancer cell lines LoVo, SW480, HT29, HCT116, CaCo and DLD1, were maintained in DMEM medium supplemented with 10% heat-inactivated FCS (Gibco) and 100 U/ml penicillin/streptomycin (Gibco). pre-B ALL cell lines NALM6, REH, 697, RS4-11, HAL-01, NALM16, SupB15 KOP N8, and MHH-CALL2 were maintained in RPMI-1640 (Gibco) medium supplemented with 10% heat-inactivated FCS (Gibco) and 100 U/ml penicillin/streptomycin (Gibco).

### Cellular Assays and Manipulation

Indirect immunofluorescence, western blot analysis, flow cytometry and TopFlash assays have been previously described. For immunofluorescence, detection was carried out by confocal spinning disc microscopy using an Andor Dragonfly 500 (Oxford Instruments). Images were processed using Imaris software (Bitplane/Oxford Instruments). Flow cytometry was carried out with a BD LSR Fortessa or BD LSR Symphony cell analyzer using the BD FACSDiva software (BD Biosciences Inc.). Quantitative real-time PCR (qRT-PCR) has been previously described and used Taqman probes specific for human LGR5 (Life Technology, Hs00969422_m1) and for TBP (Life Technology, Hs00427620_m1) as control housekeeping gene. The Prism software package was used to graph data sets from TopFlash assays and qRT-PCR experiments and for statistical analysis using two-tailed Students t-test.

For overexpression of LGR family proteins, HEK 293T cells were transfected with the corresponding plasmids using lipofectamine 2000 (Life Technology) according to manufacturer's recommendations. Cells were transfected overnight and recovered in culture media for an additional 16 hours prior to immunofluorescence, western blot or flow cytometry.

Generation of a-LGR5v4 and a-LGR5v6 was carried out by transfection of HEK293T 4×T175 flasks with 15 μg of each encoding plasmid (Absolute Antibody). Media containing antibodies was collected at 2- and 4-days post-transfection and subject to Protein G purification. The fluorescent versions of a-LGR5 (FI-α-LGR5), α-LGR5v4 (FI-α-LGR5v4) and α-LGR5v6 (FI-α-LGR5v6) were generated using the Dylight TM650 Antibody labelling kit (Thermo Fisher Scientific). Fluorescent Trastuzumab (FI-α-HER2) was generated using the Dylight TM550 Antibody labelling kit, respectively. In some controls for flow cytometry and immunofluorescence, FI-α-LGR5 was pre-incubated with supra-stoichiometric amounts of RAD-displayed Frag1A or Frag1B, or the Frag1A peptide (Cambridge Peptides) at a molar ratio of 10:1.

Antibody internalisation assays were carried out by incubation of cells with 20 μg/ml of FI-α-LGR5 or FI-a-HER2 for various timepoints, followed by fixation in 4% paraformaldehyde and immunofluorescence. Images derived from z-stacks of cells were analysed for FI-a-LGR5v4 and fluorescent signals from antibodies against various cellular markers, fluorescent phalloidin to visualise cortical actin, and Hoechst 33342 to visualise nuclei. Images were processed and analysed used Arivis Vision 4D software. Image segmentation to delineate fluorescent features: whole cells, delineated by cortical F-actin, nuclei and puncta) in 3D was carried out using Blob Finder. The Arivis Vision 4D software was then used to classify puncta associated with the cell membrane or within the cells and to determine the degree of co-localisation between LGR5 and compartment-specific markers—co-localisation is defined as >50% overlap with LGR5 puncta.

For the internalisation kinetics experiments, images were analysed using Arivis Vision 4D software, combined with a deep-learning segmentation using Cellpose. Two regions were defined: cell outer membrane, and cell cytoplasm. Segmented dotted signal corresponding to FI-α-LGR5 or FI-α-LGR5v4, as well as segments-like signal corresponding to FI-□-HER2, were classified according to their location within these two regions. Ratiometric analysis was performed to quantify the internalisation of both markers at the various time points.

### TCGA Data Mining for LGR5 Expression in Cancers

Publicly available gene expression data (RNAseq V2) from The Cancer Genome Atlas (TCGA; https://www.cancer.gov/about-nci/organization/ccg/research/structural-genomics/tcga) were downloaded using Firebrowse (http://firebrowse.org/). Gene-level read counts were quantile normalised using Voom and (log 2 median-centred) LGR5 gene expression was determined for each sample. Tumour subtypes for which more than 70% of samples had higher than pan-cancer median LGR5 expression were triaged as “high LGR5 tumours”.

Generation of a-LGR5 Antibodies

The fusion was made from splenocytes of NMRI mice or S P R D rats (Taconic), which were SC immunized twice at a 14-day interval with 30 mg of the LGR5 N-terminal fragment glutaraldehyde coupled to diphtheria toxoid. The antigen was administered with the GERBU Pa adjuvant according to the manufacturer's recommendations. Four days prior to the fusion, the animals received an IV injection boost of 15 mg antigen administered with adrenaline.

Fusions and screenings were essentially performed as described before with the mouse SP2 myeloma cell line as the fusion partner.

Purification of immunoglobin fractions was carried out by absorption of 1 L of the hybridoma supernatants to 3 ml packed volume of Protein G fast flow Sepharose equilibrated in PBS.

After extensive washing of the column with PBS, the immunoglobin fraction was eluted with 100 mM glycine, pH 2.7 and immediately neutralization with 200 mM Tris pH 8.0.

### Western Blotting

Western blots of HEK293t, LoVo and NALM6 lysates was carried out with 40 μg of protein. Western blots of RAD displayed peptides were carried out with 2.5, 12 and 60 ng of purified protein.

### Wnt Signaling Reporter Assay

TopFlash assays were performed as previously reported (de la Roche et al).

### DNA Sequencing of the CDR3 Region of Anti-LGR5 Mouse Monoclonal Antibodies

Outsourced to Absolute Antibody.

### Humanisation of Anti-LGR5 Mouse Monoclonal Antibodies

Outsourced to Absolute Antibody.

### Cell Culture

All cells were grown in a humidified incubator at 37° C. and 5% CO₂. Colorectal cancer cell lines, LoVo, SW480, HT29, HCT116, CaCo and DLD1 were maintained in DMEM medium supplemented with 10% heat-inactivated FCS (Gibco) and 100 U/ml penicillin/streptomycin (Gibco). HEK 293T were maintained in DMEM medium supplemented with 10% heat-inactivated FCS (Gibco) without 100 U/ml penicillin/streptomycin (Gibco). Colo205 colorectal cancer cell line and pre-B-ALL cell lines, NALM6, REH and 697, were maintained in resuspended in RPM I-1640 (Gibco) medium supplemented with 10% heat-inactivated FCS (Gibco) and 100 U/ml penicillin/streptomycin (Gibco).

Quantification of LGR5 Expression at mRNA Level

Cells harvested for RNA extraction were washed twice in ice-cold PBS and snap-freeze as dry pellets for storage at −80° C. freezer. Total RNA was extracted following manufacturer's instructions using Single cell RNA Purification kit (Geneflow) or PureLink RNA Mini kit (Life Tech) according to the number of cells acquired. Quantitative RT-PCR (qRT-PCR) was carried out using the One-Step qRT-PCR Kit (Thermo Fisher SuperScript III Platinum) with Taqman probes specific for human LGR5 (Life Technology, Hs00969422_ml) and for TBP (Life Technology, Hs00427620_ml) as control housekeeping gene. Each experiment included a non-template water control for the assurance of clean reaction background. The non-RT control substituting SuperScript Platinum III RT with Platinum Taq DNA Polymerase (Life Technology, cat no. 10966018) was performed to control for non-specificity of RT amplifying genomic DNA. Expression level of gene transcript was calculated by the ΔCt method. The cycle threshold (Ct) value from the gene of interest was subtracted from the housekeeping gene and transformed with a factor of 2\hat{ }(˜ΔCt) to give the fold expression relative to the housekeeping gene.

Patient Samples and Immunofluorescence Detection of LGR5 and f-Catenin

All human tissue biopsies used in the study were paraformaldehyde-fixed paraffin embedded and probed with α-LGR5 and an antibody to β-catenin and visualised by fluorescent secondary antibodies to mouse labelled with Alexa 488 and rabbit labelled with Alexa 555. All immune stained samples were imaged using the PhenoImager HT™ Automated Quantitative Pathology Imaging System (Akoya Biosciences). Scoring of all human biopsies for simultaneous LGR5 and a-catenin expression was performed by an individual blind to the provenance of the samples and graded from no expression—0 to high expression—3 for all sample sets. The Prism software package was used for plotting of LGR5 or β-catenin expression levels for all biopsy sample sets and for determining statistical differences using two-tailed students t-test. Unless otherwise notes, all relevant legal and ethical guidelines of the Addenbrooke's Hospital (Cambridge, UK) were followed for collection of samples and provision for the present study. Informed consent for research application was obtained from all subjects.

LGR5 expression in individual colorectal cancer cases and adjacent healthy tissue were determined for biopsies provided by Dr Olivier Giger(OG; (IRAS: 162057). Within these tissue samples, regions were annotated as normal, dysplastic or invasive tissue from consecutive H&E sections. LGR5 and β-catenin protein levels were additionally determined in CRC by immunofluorescence using the Bern CRC sample set, provided by Dr Inti Zlobec. The Bern CRC sample set is a highly annotated tumour microarray (TMA) consisting of 160 individual cases in duplicate with determined phenotypic feature—gender, age, tumour stage, therapeutic intervention, and MSI status. Biopsies used in the construction of the TMA were collected under ethics 2020-00498 granted by the Ethical Committee of the Canton of Bern, Switzerland. All relevant guidelines of the Institute of Pathology, University of Bern, Canton of Bern, Switzerland were followed for construction of the TMA.

The Cambridge HCC TMA consists of 104 human liver samples and was collected by Drs Sarah Aitken and Matthew Hoare with informed consent from Addenbrooke's Hospital, Cambridge, UK, according to procedures approved by the East of England Local Research Ethics Committee (16/NI/0196 and 20/EE/0109). Liver samples classified as healthy were obtained from resections from females with inflammatory adenoma of the liver (2 individuals) or focal nodular hyperplasia (2 individuals) or from a male with an HNF1 a-inactivated adenoma. All biopsies of healthy liver tissue were taken from patients between the ages of 25 and 36.

High grade serous ovarian carcinoma (HGSOC) samples comprising the Cambridge ovarian cancer TMA were provided by Prof James Brenton. Tumour samples were obtained from patients enrolled in the Cambridge Translational Cancer Research Ovarian Study 04 (CTCROV04, short OV04) study approved by the Institutional Ethics Committee (REC08/H0306/61). Samples were processed following standardised operating protocols as outlined in the OV04 study design. Tissue quality was assessed using haematoxylin and eosin (H&E) sections, and high purity regions were selected for tissue microarray (TMA) generation (using 0.1 cm cores). The TMA consisted of healthy fallopian tube (FT; 27 samples), 28 ovarian cancer cases (OvC) and 14 omentum cancer cases (OmC).

The Cambridge brain cancer TMA consists of 5 samples of healthy brain tissue, 5 from low grade glioma and 5 from glioblastoma that were collected via the ICARUS biorepository, Addenbrooke's Hospital, according to approved local research ethics (LREC 18/EE/0172).

Sections of PDAC and healthy pancreas were provided by Dr Eva Serrao and were obtained from the Cambridge University Hospital Human Tissue bank, Cambridge, UK, according to procedures approved by the Cambridge South Research Ethics Committee (REC18/EE/0227). Primary haematological malignancy samples used in this study were provided by Blood Cancer UK Childhood Leukaemia Cell Bank (LREC 16/SW/0219) and Cambridge Blood and Stem Cell Biobank (LREC 18/EE/0199) as well as the Blood Cancer UK Biobank (REC16/SW/0219) according to procedures approved by the South West-Central Bristol Research Ethics Committee.

Buffy Coats from healthy donors were acquired from NHS Blood and Transplant (Cambridge) under appropriate ethics (Research into Altered Lymphocyte Function in Health and Disease, REC reference: 17/YH/0304). PBMCs were isolated using SepMate PBMC Isolation Tubes (Stemcell Technologies) and B cells and CD8+ T cells were isolated from these using the human CD19 Microbeads (Miltenyi Biotec) or the human CD8+ isolation kit (Miltenyi Biotec).

### ADC Generation and in Vitro Killing Assays.

All antibodies and IgG1 (Sigma-Aldrich) were coupled to MMAE through a divinyl pyrimidine bridging linker inserted within the heavy chain-light chain disulphide linkages for precise drug-to-antibody ratio of 4. F or in vitro killing assays, LoVo target cells were seeded into opaque 96-well plate for overnight, allowing settlement before treatment using ADCs with cleavable linker or non-cleavable control linker at doses of 30, 10, 3, 1, 0.3, and 0.1 nM for 3 days. NALM 6 and REH target cells were seeded into opaque 96-well plate and treated directly by ADCs with cleavable linker or non-cleavable control linker at doses of 30, 10, 3, 1, 0.3, and 0.1 nM for 3 days. On day 3, cell viability was evaluated by CellTiter-Glo 2.0 Cell Viability Assay (Promega) according to manufacturer's instructions. Bioluminescence was measured using a CLARIOStar(BMG Labtech).

The efficacy of α-LGR5-ADC in targeting CRC was tested in CRC organoid models from the de la Roche laboratory biobank. For killing assays, organoid models 1 and 2 were treated with a concentration range of α-LGR5-ADC from 1 to 100 nM. Organoid killing was evaluated after 24 hours by the percentage of cleaved caspase 3 (CC3) expressing cells.

### Flow Cytometric Analysis for Internalisation of LGR5

Cells were blocked with Human TruStain fcX (Biolegend) for 15 mins and then incubated with FI-labeled a-LGR5 mAbs with or without blocking by epitope peptides (synthesised by Cambridge Peptides) at a ratio of 10:1 (peptide:mAb). Incubation was performed at 37° C. (surface and internalised LGR5) or 4° C. (surface LGR5). For cell surface staining, cells were washed twice with ice-cold PBS and incubated for 10 mins light protected at room temperature with Fixable Viability dye eFlour780 (eBioscience). Cells were then washed once with FACS buffer, made with PBS (Gibco), 3% FCS (Biosera), 0.05% Sodium Azide (Sigma), and 2 mM EDTA (Sigma). Cells were then incubated with fluorophore-conjugated antibodies at the appropriate dilution in the presence of Human TruStain fcX (Biolegend) for 30 mins at 4° C. protected from light. Cells were washed twice with FACS buffer and flow cytometric analyses were conducted on a BD Fortessa or Symphony cell analyser, and data was analysed with FlowJo software (Tree Star Inc., version 10.4).

### Immunofluorescence (IF) Microscopy

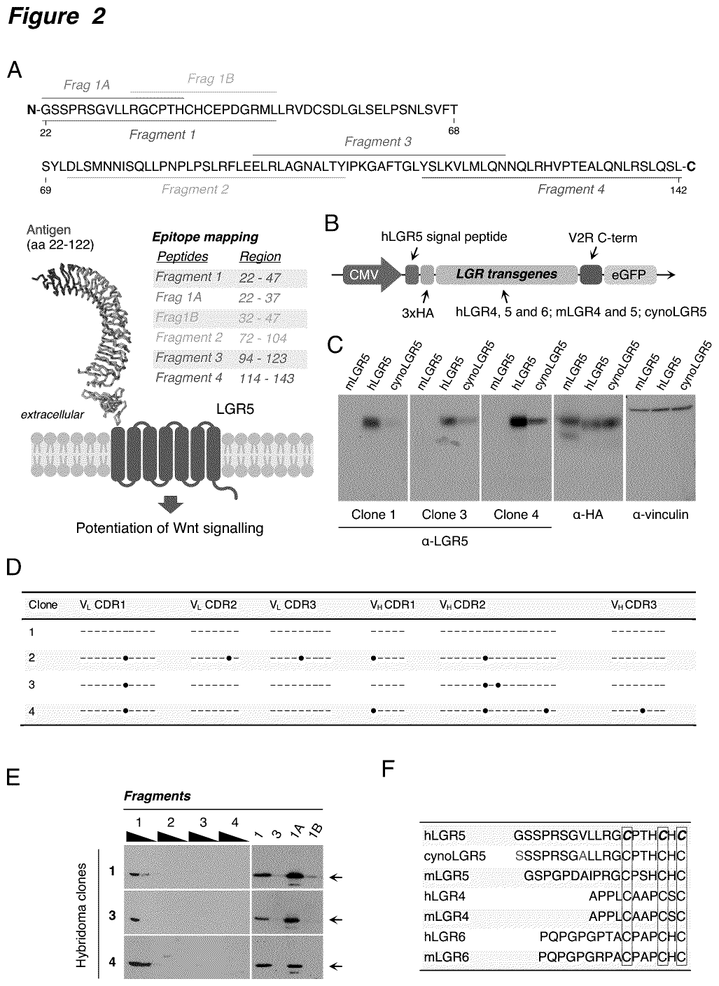

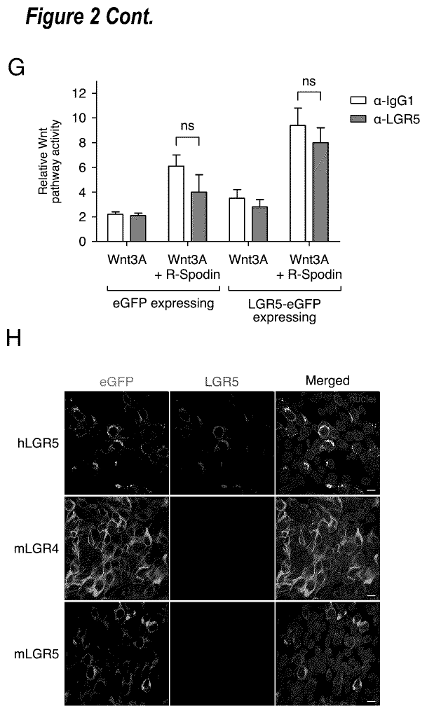

For the overexpression of different LGRs from different species (human, mouse and macaque), HEK 293T cells were cultured on top of glass coverslip and grown in culture for overnight before transfection with various expression vectors (FIG. 2B) using lipofectamine 2000 (Life Technology) according to manufacturer's instructions. Transfected cells were further incubated for overnight to allow transgenes to be expressed before subjected to IF staining and microscopy.

LoVo cells were seeded on top of glass coverslip and grown in culture for 2 days before being stained for IF Microscopy. NALM6 cells were washed with RPMI medium without FCS twice, resuspended and seeded on top of glass slides for 8 minutes at 37° C. in incubator, allowing firm adherence for further IF staining and microscopy.

To visualise internalisation, NALM6 cells were incubated in full medium in a 37° C. incubator with FI-conjugated anti-LGR5 mAbs or FI-conjugated anti-LGR5 mAbs blocked by epitope peptides as negative control for 5, 15, 30 and 60 minutes. Then NALM6 cells were washed with RPMI medium without FCS twice, resuspended and seeded on top of glass slides for 8 minutes at 37° C. in an incubator. For LoVo, cells were seeded on top of glass coverslip and grown in culture for 2 days, then incubated in full medium in a 37° C. incubator with FI-conjugated anti-LGR5 mAbs or FI-conjugated anti-LGR5 mAbs blocked by epitope peptides as negative control for 5, 15, 30 and 60 minutes for further IF staining and microscopy.

Cells were washed twice with PBS then fixed with 4% PFA (CN Technical Services) for 10 mins at room temperature. Slides were washed 5 times with PBS and blocked with blocking buffer including 1% bovine serum albumin (BSA, Sigma) and 0.1% TritonX-100 (Alfa Aesar) in PBS for 30 mins at room temperature. Blocking buffer was aspirated and slides were stained with primary Abs in blocking buffer for 1 hours at room temperature and then washed 5 times with blocking buffer. Secondary antibodies (anti-mouse Alexa488 (Thermo) and Alexa647 AffiniPure Donkey Anti-Human IgG (H+L) (Jackson ImmunoResearch)) with or without FI-conjugated Phalloidin (Alexa488 or Alexa555, Thermo) in blocking buffer were added onto slides for 30-minutes at room temperature. After incubation, slides were washed 5 times with blocking buffer and stained with Hoechst33342(Invitrogen) prepared in blocking buffer for 5 mins, light protected at room temperature. Slides were washed 5 times with blocking buffer and mounted with ProLong Diamond Antifade Mountant (Fisher). Excess mounting fluid was wiped off and slides were allowed to set at room temperature and light protected overnight before imaging. Confocal spinning disc microscopy was performed on an Andor Dragonfly 500 (Oxford Instruments). Images were processed using Imaris software (Bitplane/Oxford Instruments).

### Generation of ADCs

All antibodies and IgG 1 were coupled to MMAE using a previously reported protocol (Walsh et al).

Cytotoxicity of ADCs Against CRC and preB ALL Cell Lines in Vitro

LoVo target cells were seeded into opaque 96-well plate for overnight, allowing settlement before treatment using ADCs with cleavable linker or non-cleavable control linker at doses of 30, 10, 3, 1, 0.3, and 0.1 nM for 3 days. NALM6 and REH cells were seeded into opaque 96-well plate and treated directly by ADCs with cleavable linker or non-cleavable control linker at doses of 30, 10, 3, 1, 0.3, and 0.1 nM for 3 days. On day 3, cell viability was evaluated by CellTiter-Glo® 2.0 Cell Viability Assay (Promega) according to manufacturer's instructions. Bioluminescence was measured by CLARIOStar (BMG Labtech).

In Vivo Therapeutic Efficacy of Mouse and Humanised ADCs Against Human NALM6 preB ALL Cells in an Immunodeficient NSG Mouse Model

NSG mice (NOD scid gamma; strain NOD.Cg-Prkdc^{scid} Il2rg^{tm1wjl}Szj) were purchased from Charles River UK Ltd (Margate, UK) and housed under specific pathogen-free conditions at the University of Cambridge, CRUK Cambridge Institute in accordance with UK Home Office regulations. NALM6 cells were transduced to stably expressing LucEYFP reporter.

Immunodeficient NSG mice were engrafted with NALM6 cells by intravenous (i.v) injection and monitored for weight loss and imaged using IVIS imaging in a 2-3 day interval. (FIGS. 11A and 13A). For IVIS imaging, mice were given D-luciferin at dose of 150 mg/kg by i.p 10 minutes prior to imaging by IVIS2000 under general anaesthesia with isofluorane. Mice were randomised into control and treatment groups according to the bioluminescence signal detected on day 5 after engraftment. Treatment with mIgG-ADC or α-LGR5-ADC at 5 mg/kg was carried out by i.v injection starting on day 6 for 4 times every other day. Treatment with a-LGR5v6-ADC (control) or α-LGR5v4-ADC at 5 mg/kg was carried out by i.v injection on days 6 and 7.At the experimental endpoint on day 20, all animals were euthanised in accordance with Schedule 1 of the Animals (Scientific Procedures) Act 1986. Spleen, blood, heart, kidney, lung, liver, small intestine, femur and tibia were collected at the experimental endpoint for histology and/or FACS.

Single cell suspensions prepared from spleen, blood, and bone marrow of femur and tibia were stained with eflour780 fixable live/dead, followed by fluorescence-conjugated antibody against human CD19 for identification of NALM6 cells (CD19+EYFP+) by FACS analysis. Absolute number of NAML6 cells was quantified by the use of AccuCheck Counting Beads (Thermo).

Whole heart, kidney, lung, liver, small intestine, and part of spleen were fixed with paraformaldehyde, dehydrated by 70% ethanol, then embedded in paraffin blocks. Tissue sections were stained by hematoxylin and eosin (H&E) for evaluating general cellular structures of different tissues. Small intestine sections were stained by fluorescence-conjugated antibody against Ki67 and β-catenin.

### Engineering of LoVo and NALM6 Cell Lines

The LGR5 and LGR4 genes were targeted by CRISP R-Cas9 using the following guide RNAs (gRNAs) targeting the first exon of both gene-LGR5-5′-GCTGCTGCAGCTGGCGACCGG-3′; (SEQ ID No. 219) LGR4-5′-GCGCGGCGCCGCCTCTCTGCG-3′. (SEQ ID No. 220)

The DNA sequence corresponding to the gRNAs were integrated into the pSpCas9(BB)-2A-GFP (Addgene plasmid 48138). LoVo and NALM6 cell lines were transfected with the gRNA-bearing pSpCas9(BB)-2A-GFP plasmids and the next day, single GFP expressing cells were seeding into wells of a 96-well plate. Clonal targeted cells lines were confirmed by PCR amplification of the targeted region and sequencing.

The luciferase transgene was introduced into LoVo and NALM6 cell lines by transduction with the lentiviral vector lenti-luc.

### Patient Samples

CRC patient sections—CRC tumour samples were obtained under ethics. Sections of the tumour were assessed by a histopathologist (O.T.G.) to identify areas of normal colon epithelia, dysplastic epithelial growth and cancer.

Bern TMA—All relevant guidelines of the Institute of Pathology, University of Bern, Canton of Bern, Switzerland were followed for the study. The informed consent was obtained from all subjects or, if subjects are under 18, from a parent and/or legal guardian. At the time of ngTMA construction, similar tissue types, i.e. normal epithelium, tumor epithelium or tumor stroma, were pooled onto the same TMA recipient block, therefore prior knowledge with regard to the expected content of the tissue punch was available.

### Binding Affinity Determination

Binding affinities were determined by bio-layer interferometry measurements using the Octet platform. For svFv-α-LGR5, Kd values were derived for scFv-α-LGR5 absorbed to Ni-NTA biosensors from changes in interference measurements with the addition of the α-LGR5 epitope peptide N-SSPRSGVLLRGCPTHCHC-C(SEQ ID NO: 210).

Preparation of scFv-α-LGR5

An expression plasmid containing the 6×His-tagged scFv-α-LGR5 transgene was transfected into HEK293T cells and the protein purified from the conditioned media using Ni-NTA-Sepharose. Purified protein was dialyzed in phosphate-buffered saline (pH 7.2).

### Cambridge Liver Cancer TMA

Liver and liver cancer samples ethic are under Rec—20/NI/0109 to M.H.

### Bite Production, T Cell Activation and Killing Assay

Expression plasmids containing the coding region for transgenic BiTE versions of α-LGR5^{scFv} fused to the scFv antibody fragment of CD3E were constructed in both orientations: either with LGR5scFv at the N-terminus, LGR5^{scFv}-CD3ε^{scFv} (LC BiTE), or with CD3ε^{scFv} at the N-terminus (CL BiTE). The CL and LC BITEs were engineered to include an N-terminal signal sequence and FLAG epitope tag. Both BiTEs were purified from conditioned media of transfected HEK293T cells using HiTrap Protein L column (Cytiva) chromatography.

T cell activation assays were initiated by adding 3.7 nM CL or LC BiTEs or a-LGR5scFv as a negative control to a mixture of 10⁶ PBMCs and 10⁶ NALM6 cells. After 24-hour incubation, cells were stained with eflour780 fixable live/dead dye, followed by fluorescence-conjugated antibodies against CD4, CD8, CD25 and CD69. Flow cytometry was used to assess expression of the T cell activation markers CD25 and CD69 on CD4+ and CD8+ T cells.

BiTE-mediated killing assays were carried out using cytotoxic CD8+ T cells. For this, CD8+ T cells were isolated from PBMCs as described and stimulated for 72 h with 25 μL/mL ImmunoCult™ Human CD3/CD28/CD2 T Cell Activator (STEMCELL). CD⁸⁺ T cells were cultured in TexMACS media (Miltenyi Biotec) supplemented with 100 U/ml human IL-2 (Miltenyi Biotec) and 100 U/ml Penicillin/Streptomycin (Gibco). Cells were restimulated at 1×106 cells/mL concentration for 48 h on day 8-10 with 1 μg/mL plate-bound anti-CD3E antibody (clone UCHT1, Biolegend). BiTE-mediated killing assays were carried out on day 14-16 of culture. NALM6 target cells were labelled with CellVue membrane dye (CellVue Claret Far Red Fluorescent Cell Linker Kit protocol; Sigma-Aldrich) and 6.25×10⁴ or 1.25×10⁵ cytotoxic CD8+ T cells were used at indicated effector to target rations. After 6 hours of co-culture at 37° C., cells were labelled with eFluor780 fixable live/dead dye (ThermoFisher) and NALM6 cells were analysed for viability by flow cytometry.

### α-LGR5 CAR-NK92 and CAR-T Cell Production

The α-CD19-CAR lentiviral plasmid was a kind gift from Dr John James (University of Warwick) and contained the coding region for the α-CD19 scFv domain (clone FM363) fused to the CD8 stalk and transmembrane regions, a 4-1BB or CD28 intracellular costimulatory domain, respectively, and mScarlet. The humanized a-LGR5 scFv fragment (Absolute Antibody) was used to replace the α-CD19 scFv domain in the α-CD19-CAR lentiviral plasmid (pHR-SIN) using restriction digest and Gibson Assembly. PHR-SIN backbone was cut with BamHI and MIul and the following PCR primers were used:

TABLE 6

| | |
|---|---|
| Forward Primer to amplify LGR5 scFv: | Gacagactgagtcgcccgggacgcgtcccaccatgccgctgc |
| LGR5scFv_F1 | (SEQ ID NO. 221) |
| Reverse Primer to amplify LGR5 scFv: | Gcgtcgtggtgctgctcacggtcacgg |
| LGR5scFv_R1 | (SEQ ID NO. 222) |
| Forward Primer to amplify CAR: | Cgtgagcagcaccacgacgccagcgc |
| CAR_F1 | (SEQ ID NO. 223) |
| Reverse Primer to amplify CAR: | Caccatggtggcgaccggtggatcccgagggggcagggc |
| CAR_R1 | (SEQ ID NO. 224) |

Lentiviruses were produced using HEK293T cells. For the transfection, 1.5 mL of OptiMEM (Gibco, cat no. 31985070) were mixed with 30 μL of 1 mM HEPES (in-house) and 42 μL of TransIT-293 Transfection Reagent (Geneflow, cat no. E7-0026) and incubated at RT for 5 mins. In parallel, 6 μg of the desired lentiviral plasmid, was mixed with 4pg of the lentiviral packaging plasmid pCMV8.91 and 4pg of the lentiviral envelope plasmid pMDG. Next, the OptiMEM/TransIT solution was gently added and mixed with the lentiviral plasmids. After 15 min incubation at RT, 13 ml of pre-warmed complete DMEM was added and the mixture added to HEK293T cells.

After 48 h and 72 h, respectively, the viral supernatant was harvested, centrifuged at 250 g for 5 min, before being filtered through a 0.45 μm filter into 30 ml conical ultracentrifuge tubes. Viral supernatant was ultracentrifuged using a SW-28 rotor (Beckmann) at 25000 g for 90 min at 4° C. After the spin the supernatant was discarded, and the virus pellet was gently resuspended in 200-300 μl PBS containing 1% heat-inactivated FCS by pipetting several times over the pellet without creating air bubbles or dislodging the pellet. Resuspended, concentrated virus was stored in aliquots at −80° C.

NK92 cells were transduced with an MOI of 10. For this, the virus stock was mixed with 6 μg/ml protamine sulfate and added to NK92 cells, that had been washed and seeded at 0.2×10⁶ cells the previous day. Spinfection was carried out at 1800 rpm for 15 min at 32° C. mScarlet expression was assessed after 48-72 h and NK92 cells were sorted form Scarlet. >95% Scarlet-positive NK92 cells were used for all in vitro and in vivo assays.

CD8+ T cells were isolated from PBMCs as described above and stimulated with 25 μL/mL ImmunoCult™ Human CD3/CD28/CD2 T Cell Activator (STEMCELL). CD8+ T cells were cultured in TexMACS media (Miltenyi Biotec) supplemented with 100 U/ml human IL-2 (Miltenyi Biotec) and 100 U/ml Penicillin/Streptomycin (Gibco). 24 h later CD8 T cells were transduced with an MOI of 5 and used between day 15-20 post stimulation. α-LGR5-CAR+CD8 T cells were sorted for in vitro killing assays with purities routinely >90%.

a-LGR 5 CAR-NK and CAR-T Cell Killing Assays

NALM6, REH or HEK293T cells overexpressing hLGR5-eGFP, mLGR5-eGFP or cLGR5-eGFP were used as target cells in the VITAL killing assay and were pre-loaded with CellVue membrane dye (CellVue Claret Far Red Fluorescent Cell Linker Kit protocol; Sigma-Aldrich). a-LGR5 CAR-NK and CAR-T cells or respective untransduced control cells were added at indicated effector to target ratios. At designated timepoints percent target cell killing was assessed by flow cytometry.

LoVo and HepG2 cells were used as target cells in incucyte killing assays. For this, tumour cells were pre-seeded 24 h prior to the assay in a 96-well plate. α-LGR5 CAR-NK and CAR-T cells or respective untransduced control cells were added at indicated effector to target ratios. Cell death was assessed using Apotracker Green (Biolegend, 1:200) and monitored over 15h using the Incucyte SX5 (Sartorius).

### Results

### Generation and Validation of Antibodies Against LGR5

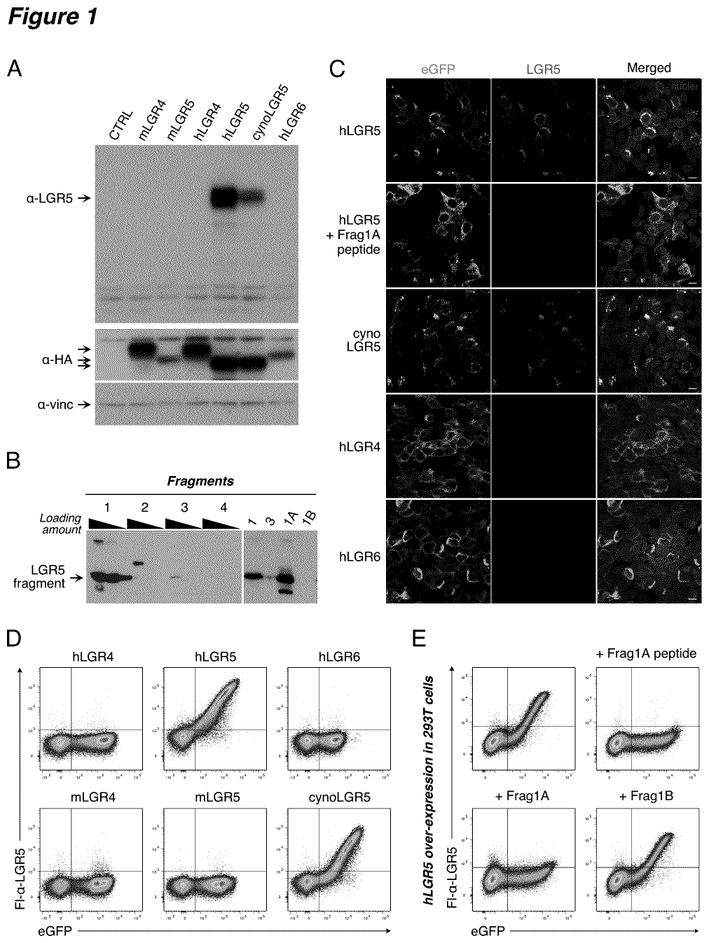

For monoclonal antibody production the inventors immunized mice with the N-terminal 100 amino acids of the human LGR5 extracellular domain (FIG. 2A). Coupling of the antigen to diphtheria toxin was necessary to initiate effective immune responses. Fusion reactions led to the generation of 18 hybridoma clones. To functionally test the clones the inventors generated transgenic versions of human and murine LGR family members: all human and murine LGR4-6 transgenes contain a common N-terminal influenza hemagglutinin (HA) epitope tag and a C-terminal extension consisting of the fusion to the vasopressin V2 receptor C-terminal tail for enhanced protein stability that is in turn fused to eGFP (FIG. 2B). The inventors also created a version of human LGR5 with Gly1Ser and Val8Ala substitutions to match the corresponding cynomolgus LGR5 N-terminus (cynoLGR5). The N-terminal 100 amino acid residues of cynoLGR5 are otherwise identical to the human protein. Western blot analysis of lysates derived from HEK293T cells expressing the various LGR transgenes demonstrated specific immunoreactivity of hybridoma clones 1, 2, 3, and 4 towards human and cynomolgus LGR5 (FIG. 1A, FIG. 2C). None of the hybridoma clones were reactive towards murine LGR5 protein, nor the human or murine LGR4 and LGR6 proteins. Moreover, the inventors did not observe any specific immunoreactivity of the hybridoma clones towards HEK293T cells, as these cells do not express endogenous LGR5 in the absence of pathway activity.

All a-LGR5 Antibodies Bind a Common Epitope at the LGR5 N-Terminus

Upon sequencing, the inventors found that the complementary determining regions (CDRs) of the light and heavy chains of the four a-LGR5 antibodies were highly conserved, displaying only four variable amino acid positions (FIG. 2D). To determine whether the a-LGR5 clones bind to a common epitope, the inventors designed four overlapping fragments of approximately 35 amino acids in length of the 100 amino acid antigen (Fragment 1-4) whose sequences were unique to the human but not murine LGR5 protein (FIG. 2A). The inventors expressed the individual fragments as RAD-display fusion constructs and purified them from expressing bacteria using standard protocols. Western blot analysis indicated that all four a-LGR5 clones bound specifically to Fragment 1 (FIG. 1B; FIG. 2E). The inventors further narrowed down the epitope to Fragment 1A, a 15 amino acid sequence at the N-terminus of the LGR5 protein that was bound by all four a-LGR5 clones (FIG. 1B, FIG. 2E). None of the clones bound to the adjacent LGR5-fragment 1B that contains 5 overlapping amino acid residues. Notably, the sequence of the Fragment 1A epitope diverges substantially in sequence from the corresponding region in human LGR4/6 and mouse LGR4/5 and by two residues in the corresponding cynoLGR5 sequence (FIG. 2F) explaining why all of the hybridoma clones are specific to the human and cyno LGR5.

Binding affinities between the a-LGR5 clones and the RAD-Fragment 1 fusion were determined by bio-layer interferometry measurements using the Octet platform. Owing to the bidentate binding of the two arms of the antibodies and high binding affinities displayed by the LGR5 antibodies, accurate determination of the Kd values required inclusion of 10 μM of the Fragment 1 peptide. The inventors observed high affinity binding between the captured Fragment 1A and the original murine clones 1 and 2 with Kd values of 0.76 and 1.1 nM, respectively (Table 5). No detectable binding was observed between all LGR5 antibodies and captured Fragment 1B.

TABLE 5
Binding affinity values for murine, humanized
and ADC-modified a-LGR5 antibodies

Kd (nM)

| Antibody | Fragment 1A | Fragment 1B |
|---|---|---|
| α-LGR 5 clone 1 | 0.76 ± 0.006 | ND |
| α-LGR 5 clone 2 | 1.1 ± 0.005 | ND |
| α-LGR 5 clone 3 | 1.0 ± 0.005 | ND |
| α-LGR 5 clone 4 | 1.4 ± 0.007 | ND |
| IgG2 | ND | ND |
| α-LGR 5 humanized | 3.4 ± 0.019 | ND |
| α-LGR 5 variant 4 | 2.0 ± 0.017 | ND |
| α-LGR 5 variant 6 | ND | ND |
| α-LGR 5 variant 12 | 2.0 ± 0.017 | ND |
| IgG2-ADC | ND | ND |
| α-LGR 5 clone 2-ADC | 2.7 ± 0.028 | ND |
| α-LGR 5 variant 4-ADC | 2.0 ± 0.017 | ND |
| α-LGR 5 variant 6-ADC | ND | ND |
| scFv fragment | 0.77 ± 0.029 | ND |

ND - not detectable; below the differential light pathlength threshold value of the 1 nm used for the interference measurements

The humanised variant 4 corresponds to clone 2.4 as in Tables 1 and 2. The α-LGR5 humanised is the direct transposition of the murine clone 2 variable domain sequences into the human IgG scaffold. The variable domain protein sequences of this humanised clone match the murine clone 2.

Humanized variant 6 (α-LGR5v6) is an antibody also generated by the inventors, but this has lost all reactivity and did not bind to either human or cyno LGR5 proteins (FIG. 14A) or the LGR5 epitope. This variant was used as a negative control for the experiments.

Clones 1, 2, 3 and 4 in table 5 are the murine antibodies 1, 2, 3 and 4 as in Tables 1 and 2.

Because of the proximity of the α-LGR5 epitope on LGR5 to the binding site for its R-spondin family ligands, the inventors sought to determine whether antibody binding interfered with the ability of R-spondin1 to potentiate Wnt pathway activity using the TopFlash assay. The inventors treated HEK293T cells expressing LGR5-eGFP or eGFP controls with Wnt and R-spondin1, in the presence of greater than 10-fold molar excess (relative to R-spondin1) of either murine IgG1 or α-LGR5 (FIG. 2G). No significant differences in Wnt/R-spondin1 potentiated activity between IgG 1 or α-LGR5 treated cells were observed indicating antibody binding to LGR5 does not interfere with R-spondin1 binding.

Taken together, the α-LGR5 antibody binds with high affinity to the N-terminus of human and cynomolgus LGR5 at a site that does not interfere with binding of R-spondin ligands.

### α-LGR5 Antibodies Discriminate Cells Expressing Human LGR5 but not Other LGR Family Members

To determine antibody specificity in detecting cellular LGR5 expression, the inventors overexpressed the LGR transgenes in HEK293T cells and probed expression with murine a-LGR5 clone 2 (α-LGR5) coupled to the Alexa647 fluorophore (FI-a-LGR5) by immune fluorescence. HEK293T cells do not express endogenous LGR5 at steady state and thus the inventors did not observe fluorescence signal from FI-a-LGR5 in the absence of transgenic expression. The inventors observed complete co-localisation of FI-a-LGR5 and overexpressed human LGR5-GFP with the signal from the antibody abrogated by pre-incubation with the Fragment 1A peptide (FIG. 1C). While FI-a-LGR5 was able to detect the overexpressed cynoLGR5 transgene (FIG. 1C), there was no signal in HEK293T cells over-expressing human LGR4 or LGR6 transgenes or the murine LGRs (FIG. 1C; FIG. 2H).

To determine whether fluorescent a-LGR5 specifically detects native LGR5 expressed at the cell surface, we carried out flow cytometric analysis of live HEK293T cells overexpressing human, murine, and cyno LGR versions. Cells expressing either human or cynoLGR5-eGFP were detected with the fluorescent a-LGR5 antibody. By contrast, HEK293T cells over-expressing human LGR4-eGFP, LGR6-eGFP or any of the murine LGRs were not detected by flow cytometry using FI-a-LGR5 (FIG. 1D). Importantly, the fluorescent signal observed in LGR5-eGFP-expressing cells was attenuated by pre-incubation with supra-stoichiometric levels of the Fragment 1A or the blocking peptide based on this sequence (10:1; FIG. 1E).

### Census of LGR5 Expression Levels in Healthy Tissues and Cancers

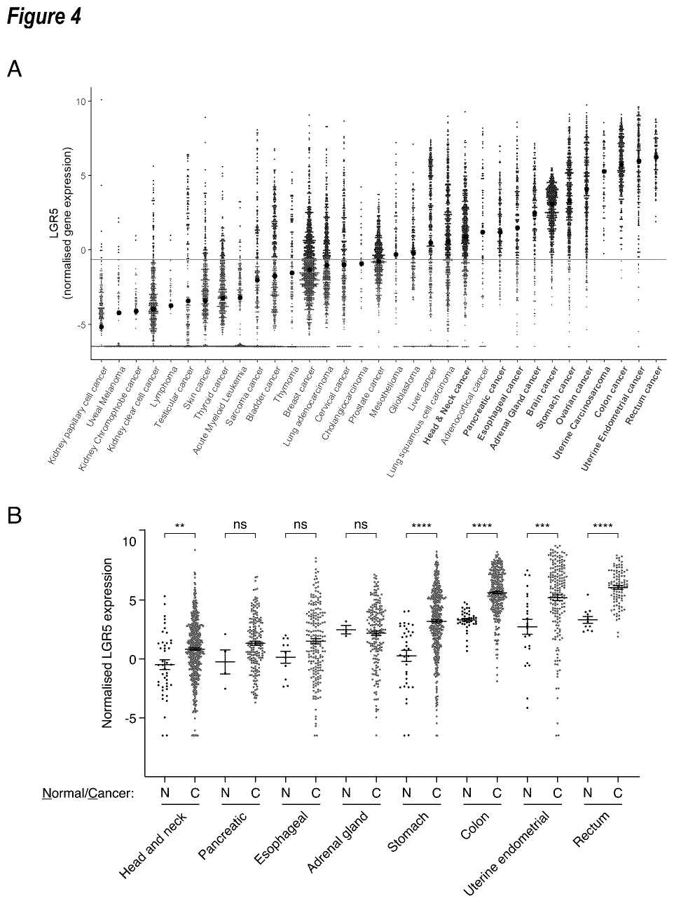

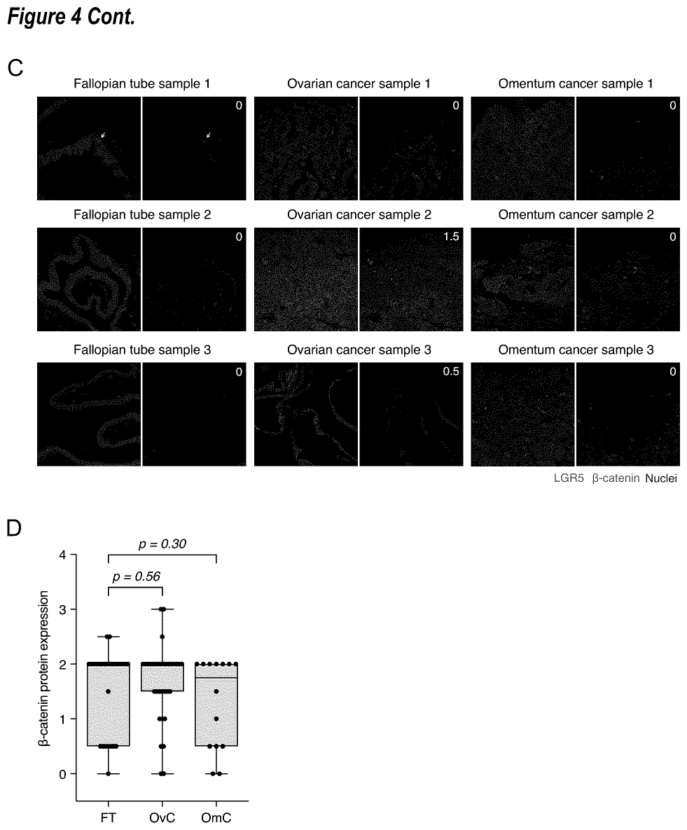

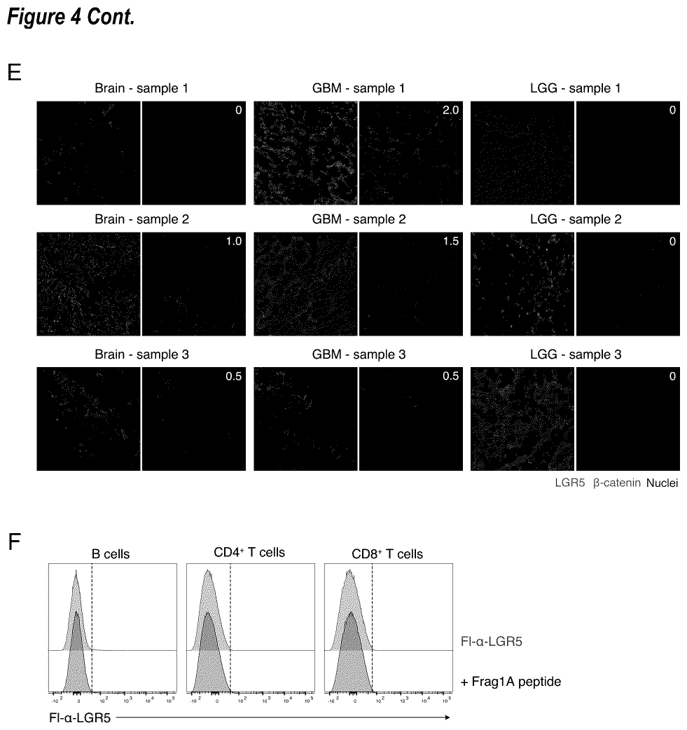

Previous studies have quantified LGR5 transcript levels in a number of cancer types and have established increased LGR5 mRNA expression some cancers relative to matched normal tissues. This work raises the intriguing possibility of discriminating and targeting cancer cells using a-LGR5-based therapeutic strategies. To investigate this in more detail the inventors carried out a comprehensive census of LGR5 transcript levels across 33 cancer types using datasets extracted from the TCGA database. Read counts were quantile normalized across the genome for direct comparison amongst cancer subtypes and sample sets and median expression levels for extracted LGR5 data determined across the entire dataset (FIG. 4A). The inventors triaged 12 of the cancer types as ‘high LGR5 expressors’ where greater than 70% of the component cases harboured LGR5 expression levels greater than the median. Brain cancer, ovarian cancer and uterine carcinosarcoma were excluded from this analysis since no normal tissue samples were available. For these high LGR5 expressing cancers the inventors compared LGR5 expression to matched healthy tissue. In all cases median LGR5 mRNA expression was higher in the cancer compared to matched healthy tissue with the exception of cancers of the adrenal gland. Highly significant increases in LGR5 expression was observed in head and neck, uterine endometrial, stomach, colon and rectal cancers (FIG. 4B).

Human expression data was also analysed by the inventors, see Hung-Chang Chen, et al, LGR5 targeting molecules as therapeutic agents for multiple cancer types Preprint, BioRxiv, https://doi.org/10.1101/2022.09.01.506182.

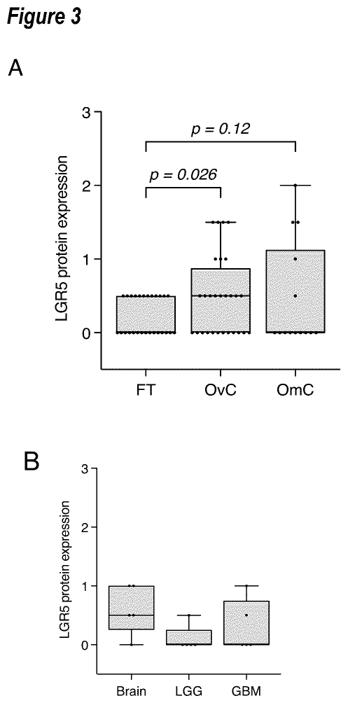

The transcription data ranked ovarian cancer as one of the highest LGR5 expressing cancers. However, owing to lack of transcriptomic data for fallopian tubes in the TCGA database, the presumptive tissue of origin for ovarian cancer, we were unable to determine whether this represents malignancy-specific LGR5 overexpression. The inventors therefore probed 24 fallopian tube biopsies with a-LGR5 and a-b-catenin alongside a TMA containing 28 ovarian cancer and 14 omentum cancer cases. b-catenin was expressed at the cortex of all epithelial cells; however, in all cancer cases the inventors did not detect appreciable LGR5 protein levels in the b-catenin positive fallopian tube epithelia except in very rare instances where we observed individual cells expressing LGR5 localized to intracellular puncta (less than <0.1% cells; FIG. 4C). Comparing LGR5 expression between fallopian tube samples and the ovarian and omentum cancer cases, the inventors did not observe a significant increase in protein levels (FIG. 3A), nor did the inventors observe significant differences in b-catenin protein expression (FIG. 4D).

The inventors next used a-LGR5 to determine whether LGR5 was upregulated in brain cancers (glioblastoma and low-grade glioma; LGG) using a TMA containing malignant and normal resected brain biopsies. No LGR5 expression was detected in healthy brain tissue and amongst the cancer cases we investigated, there was no significant upregulation of LGR5 protein levels compared to resected non-malignant brain controls (FIG. 3B; FIG. 4H) Finally, the inventors examined LGR5 protein expression in immune cells from healthy donors.

Flow cytometric analysis of human peripheral blood mononuclear cells (PBMCs) with FI-α-LGR5 did not detect any B cells, CD4+ T cells or CD8+ T cells expressing LGR5 protein (FIG. 4F). In line with a previous study, the inventors determined whether LGR5 transcript levels in tumour cells from acute lymphoblastic leukaemias (ALL) patients was upregulated relative to non-malignant controls. F or further human expression data and analysis by the inventors, see Hung-Chang Chen et al, LGR5 targeting molecules as therapeutic agents for multiple cancer types Preprint, BioRxiv, https://doi.org/10.1101/2022.09.01.506182

The transcript and protein data sets obtained establish that: (i) LGR5 overexpression is endemic to a number of cancer types, and (ii) there is a substantial therapeutic window of LGR5 expression between normal tissues and cancers. Together, the data supports therapeutic intervention in CRC, HCC and acute lymphoblastic leukaemia by strategies that target cellular LGR5 overexpression.

### α-LGR5 Detects Endogenous Expression of LGR5

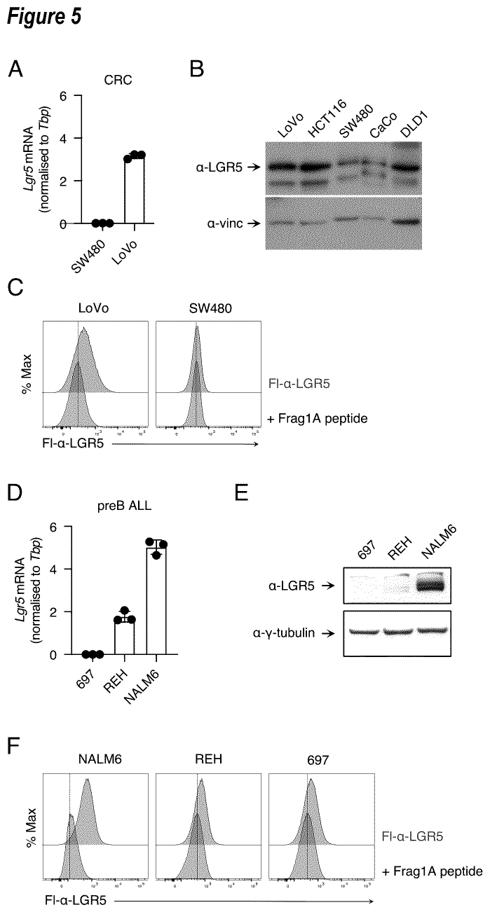

Based on the inventors' LGR5 expression data with CRC and ALL tumours, they sought to determine whether endogenous cellular LGR5 expression could be detected in cell line models of the disease. The LoVo colon cancer cell line has previously been shown to express sufficient levels of LGR5 for antibody detection and indeed, the inventors found that amongst a panel of five colorectal cancer cell lines, LoVo cells expressed at least 10-fold higher LGR5 transcript compared to the SW480 cell line (FIG. 5A). The inventors were also able to detect high LGR5 protein levels in lysates of LoVo, HCT116 and DLD1 cells, but low levels of the protein in lysates from SW480 and CaCo cells cells (FIG. 5B).

Cell surface expressed LGR5 was detected on LoVo cells using FI-α-LGR5 which could be abrogated by pre-incubation of the antibody with the Fragment1A peptide (FIG. 5C). No LGR5 was detected on SW480 cells by flow cytometry.

The inventors further investigated LGR5 protein expression in pre-B-ALL cell lines and identified NALM6 cells as expressing highest transcript levels of LGR5, followed by intermediate levels in REH cells, and lowest levels in 697 cells (FIG. 5D). LGR5 transcript levels in the pre-B-ALL cell lines are consistent with the relative levels of LGR5 protein detected by western blot (FIG. 5E) and flow cytometry (FIG. 5F).

Lgr5 Associates with a Unique Intracellular Compartment

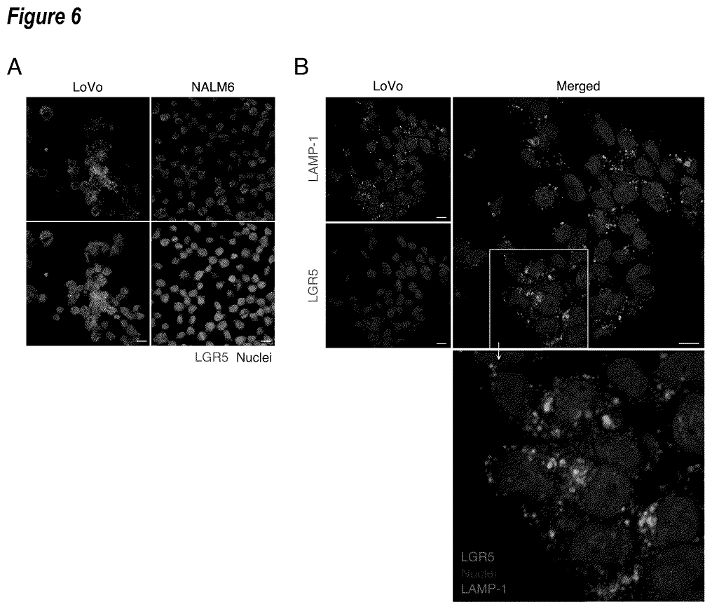

Endogenous LGR5 expression in NALM6 and LoVo cells allowed the inventors to determine its cellular localization using FI-α-LGR5. Consistent with the localization pattern of overexpressed transgenic LGR5-eGFP, the vast majority of endogenous LGR5 localizes to internal puncta (FIG. 6A). Previous work using transgenic LGR5 over-expression in HEK293T determined the association of LGR5 puncta with the LAMP 1 positive compartment and the inventors tested whether the endogenous protein behaved accordingly in LoVo cells. In both cases, antibodies to LAMP1 and LGR5 proteins detected puncta; however, these were never co-incident, indicating localisation to distinct cellular compartment. (FIG. 6B).

Rapid Internalization of a-LGR5 Antibodies by LGR5-Overexpressing Cell Lines

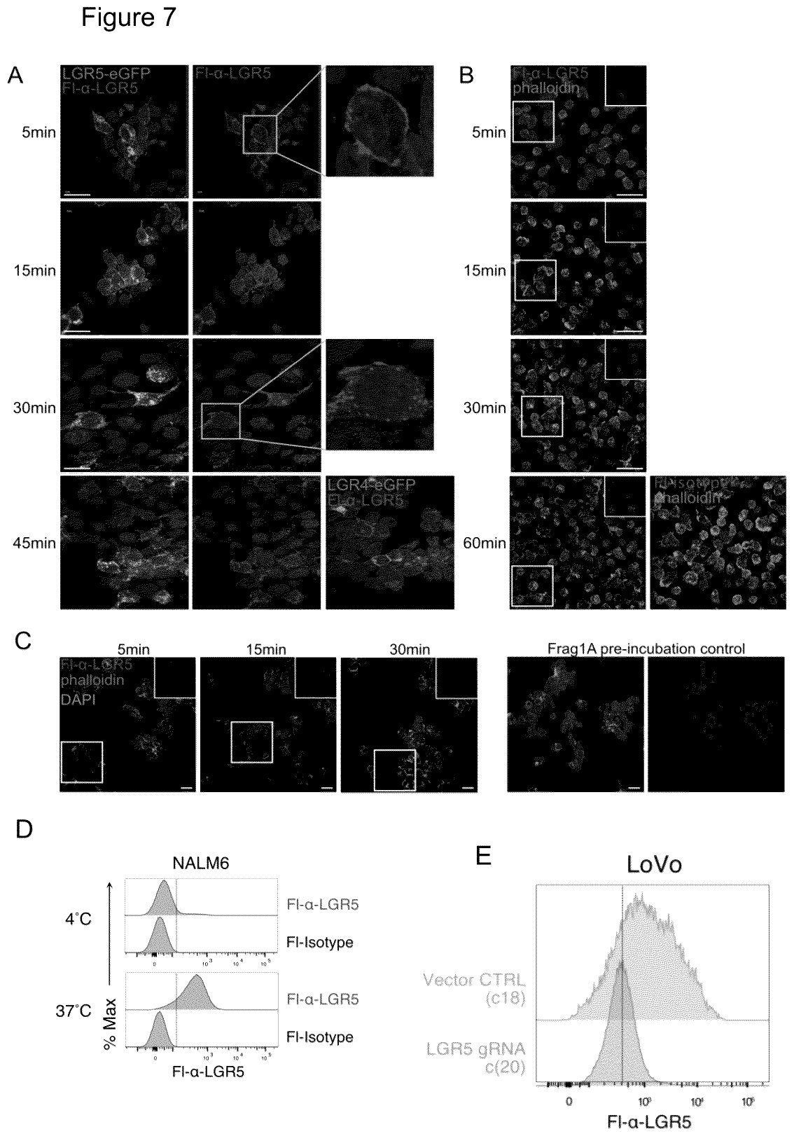

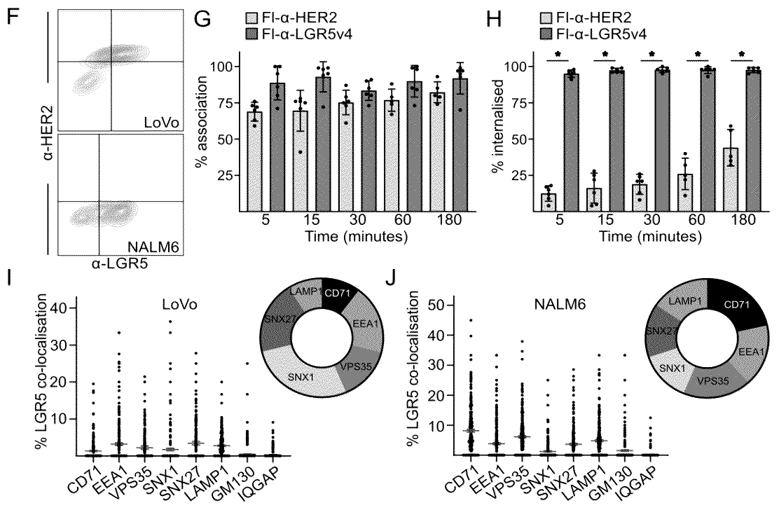

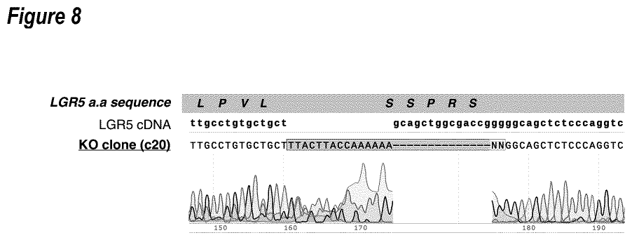

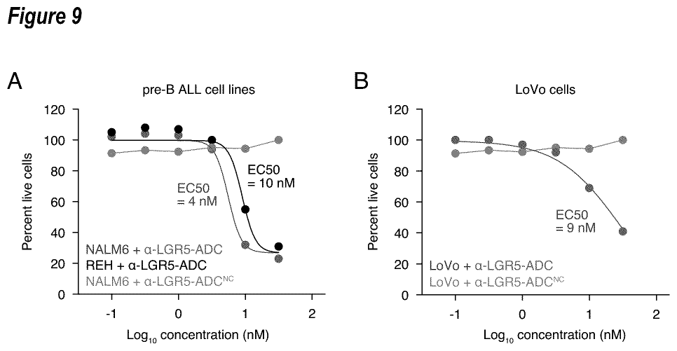

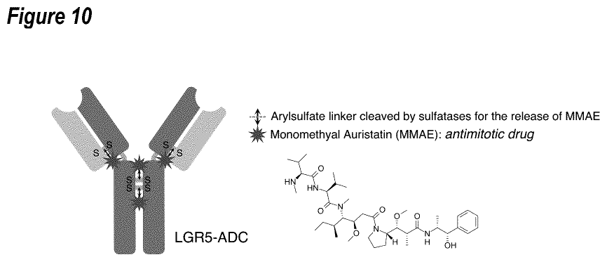

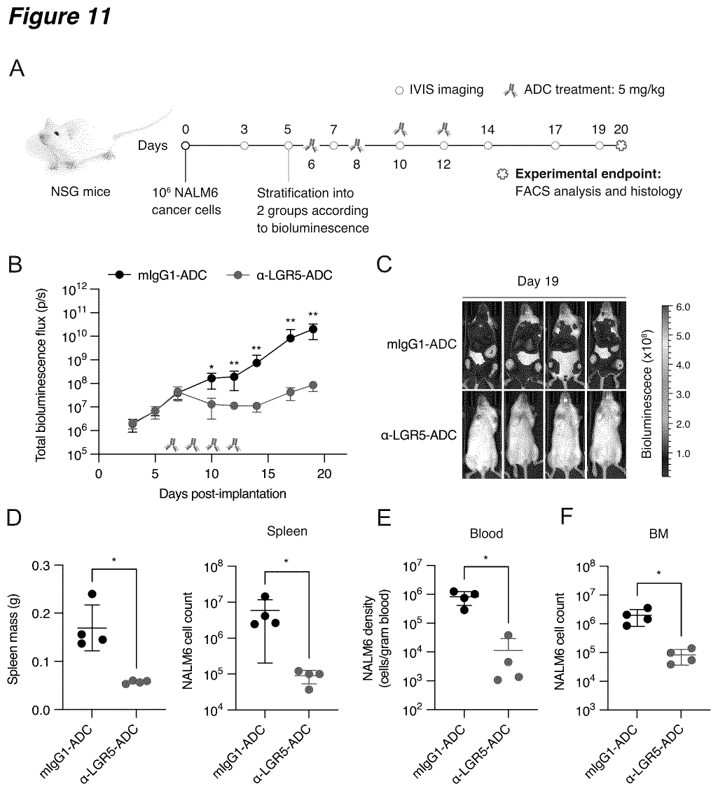

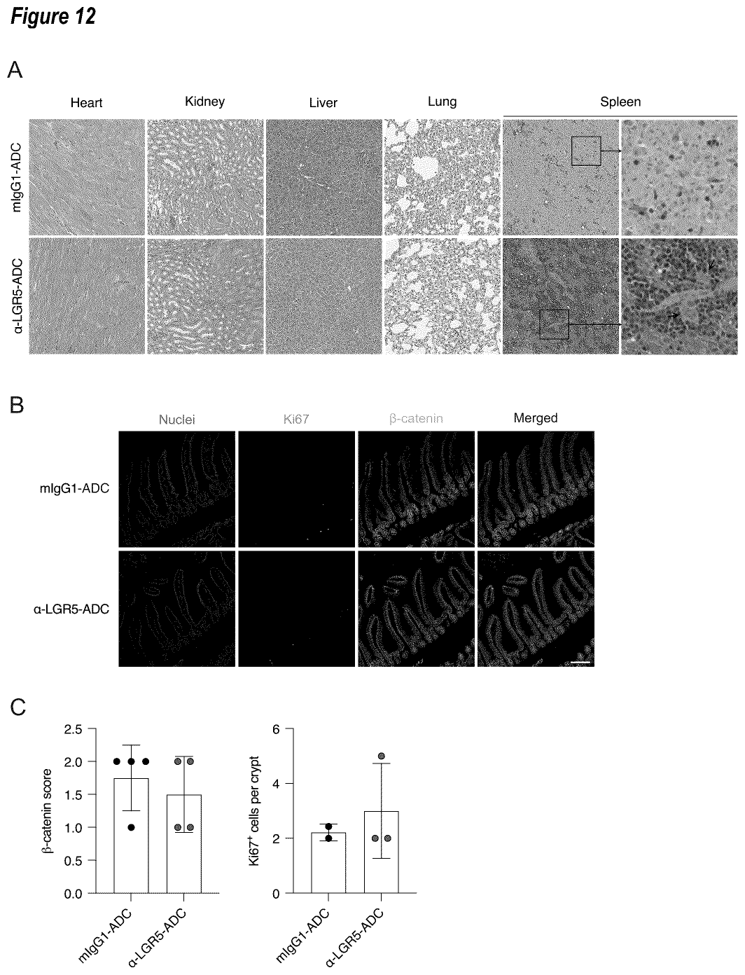

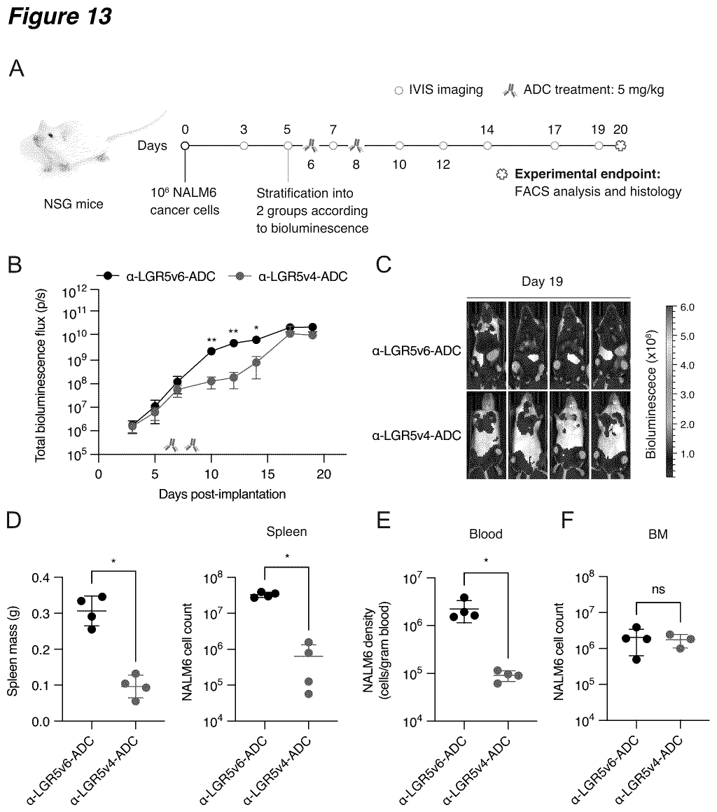

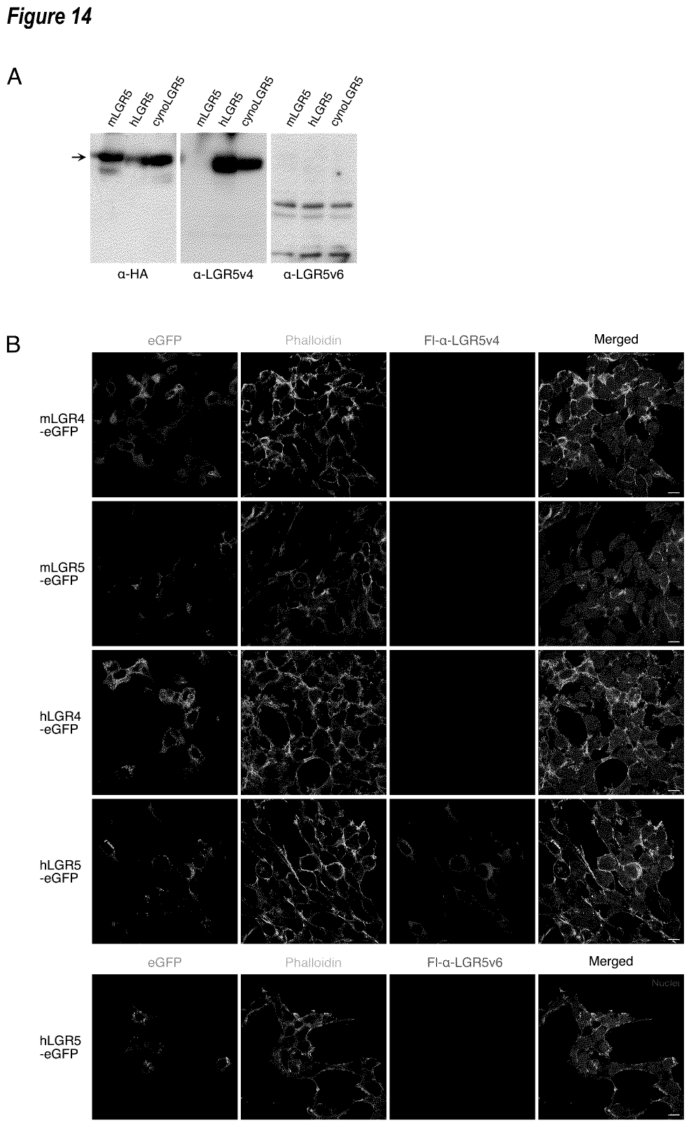

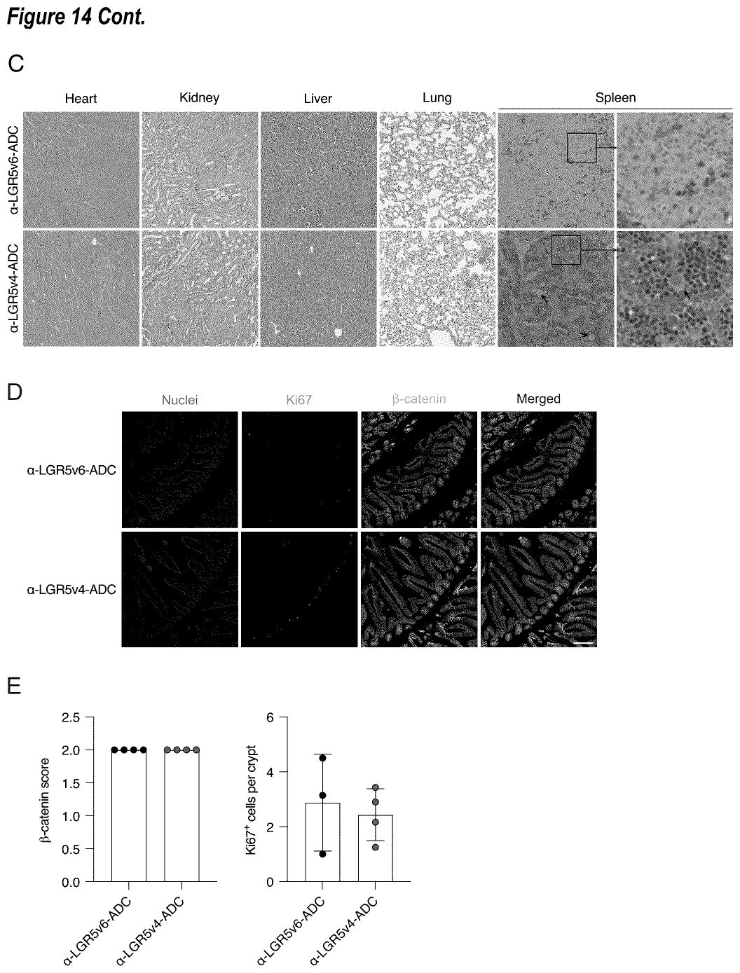

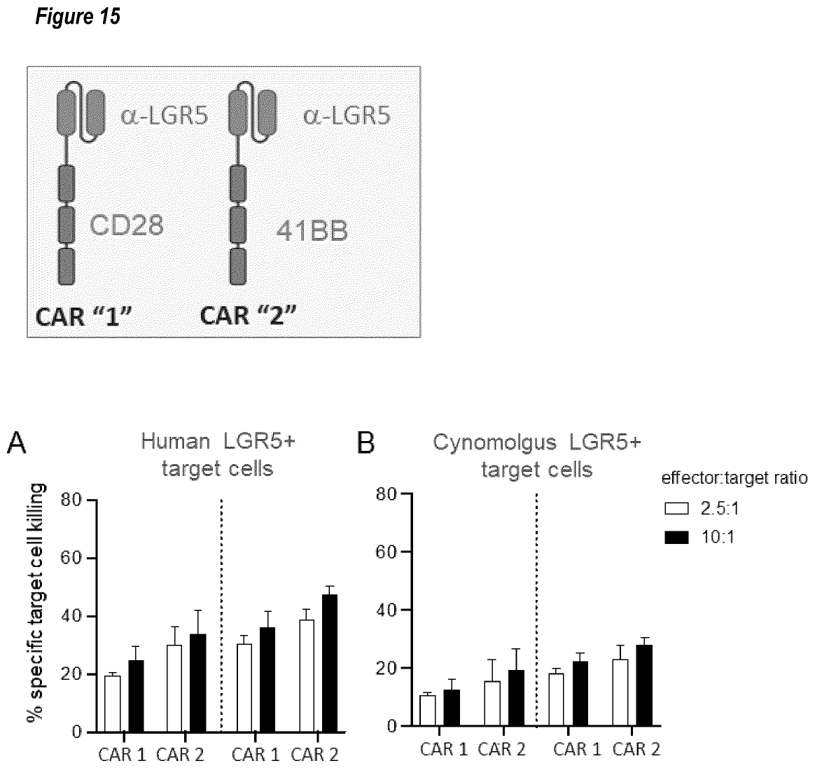

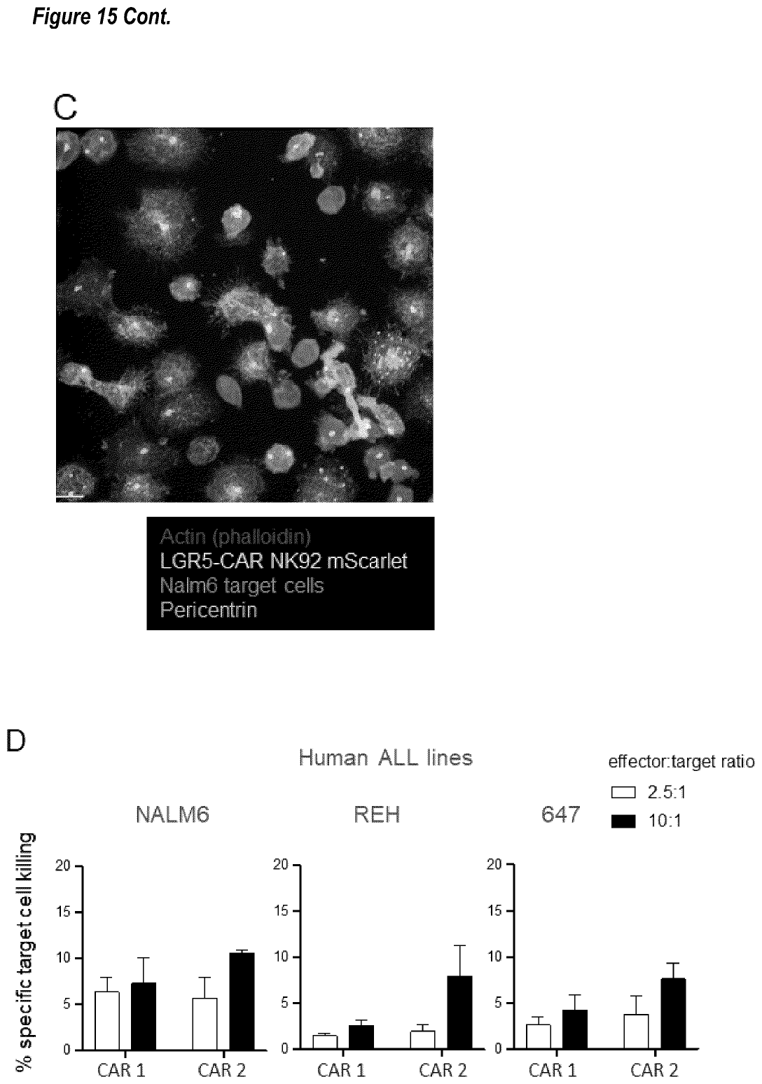

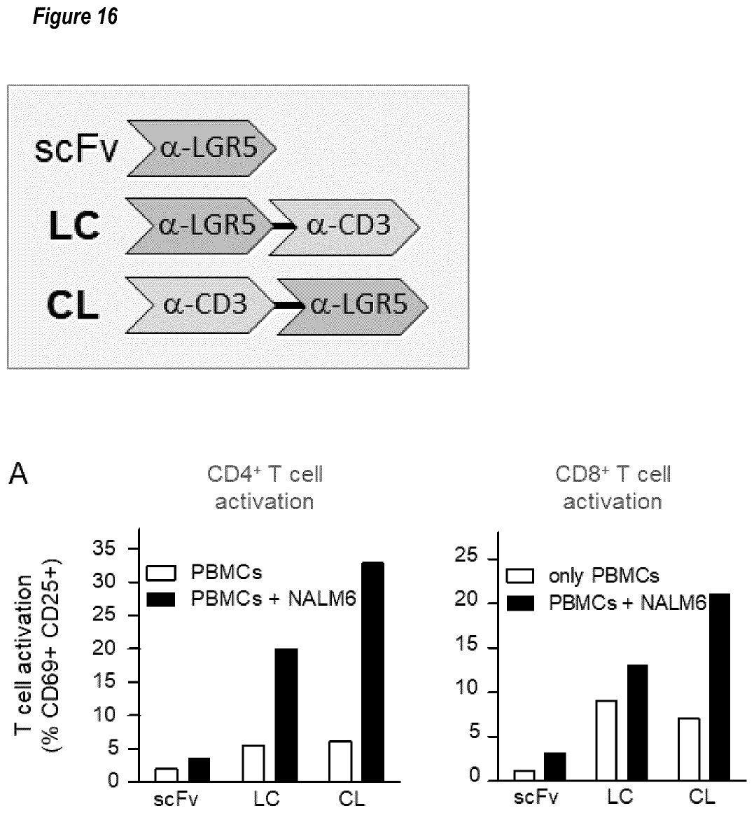

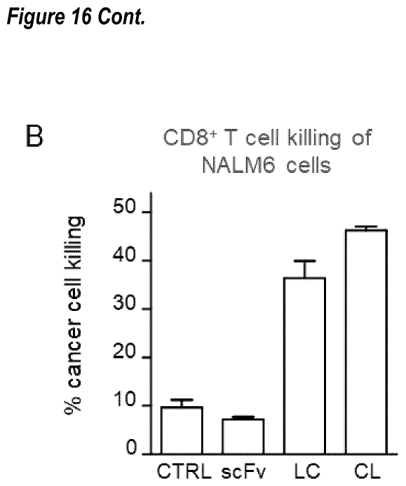

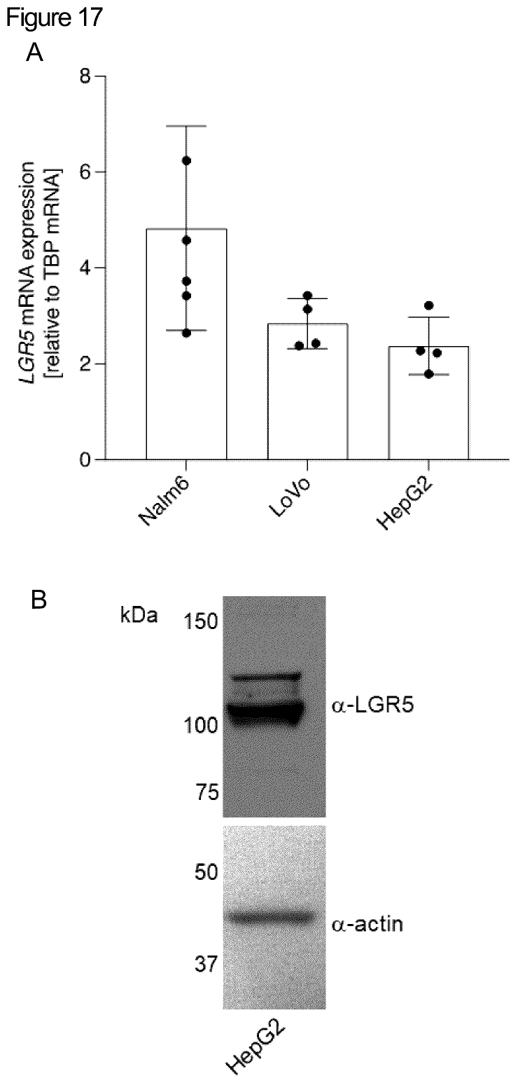

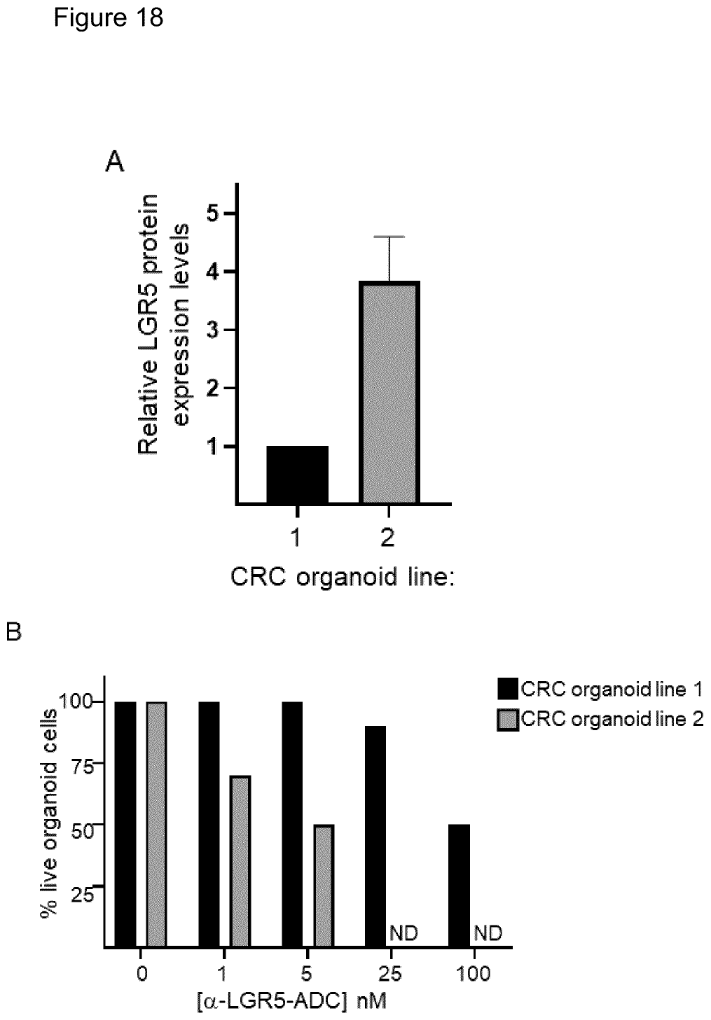

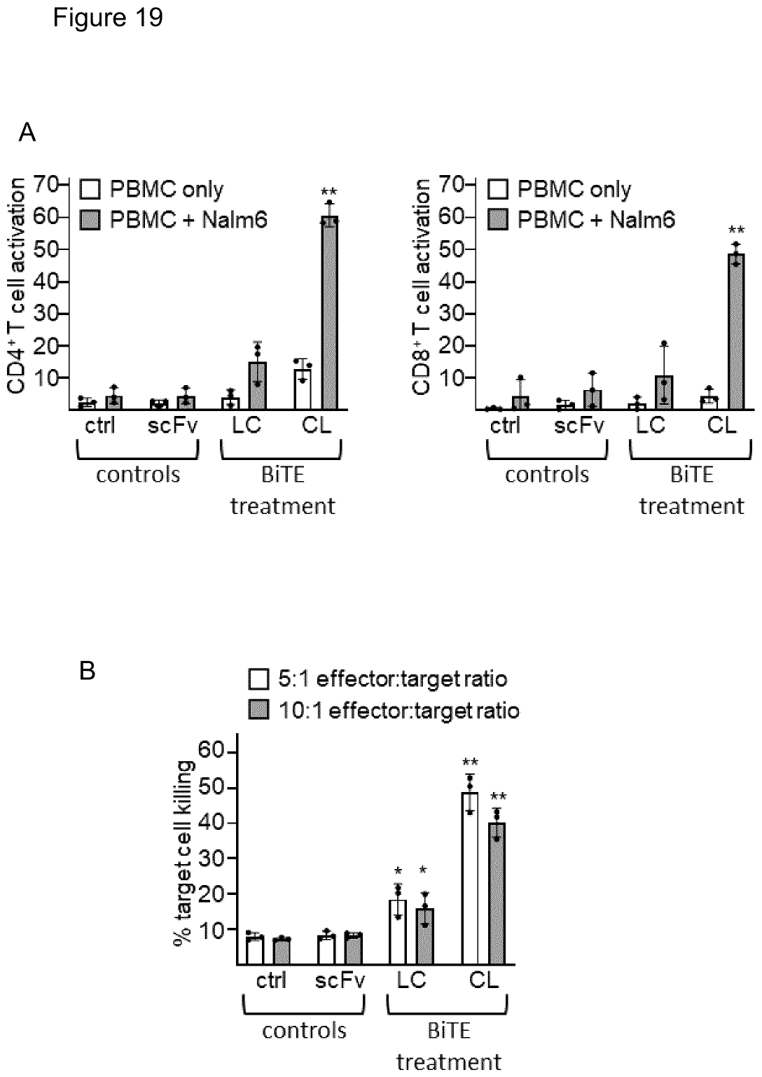

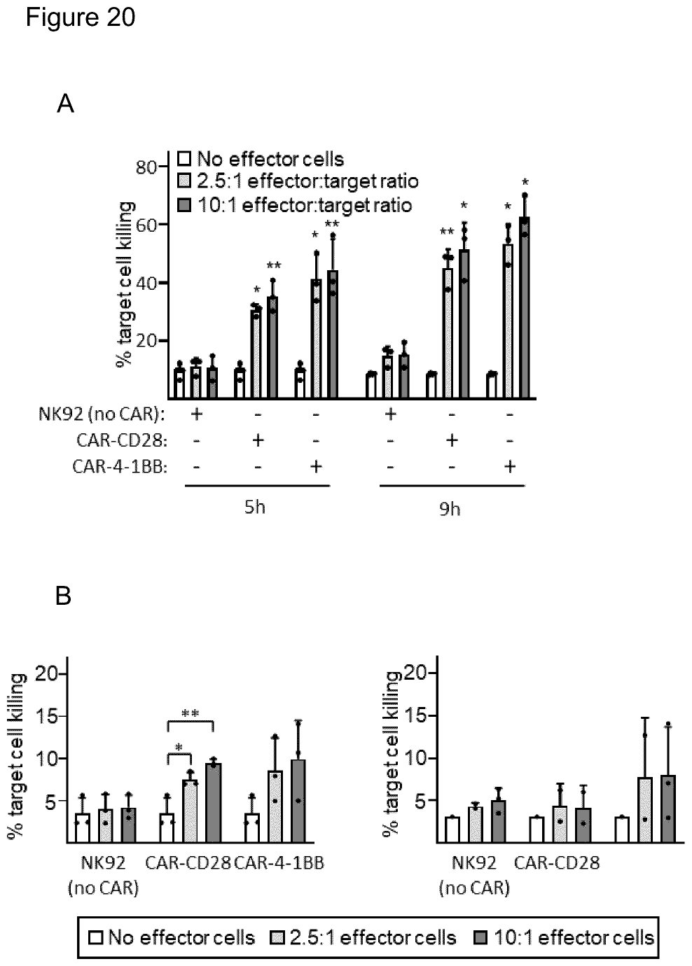

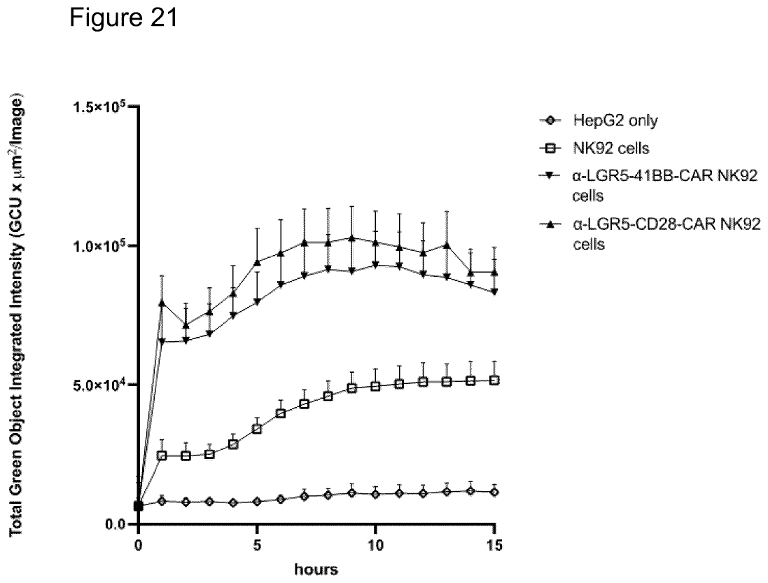

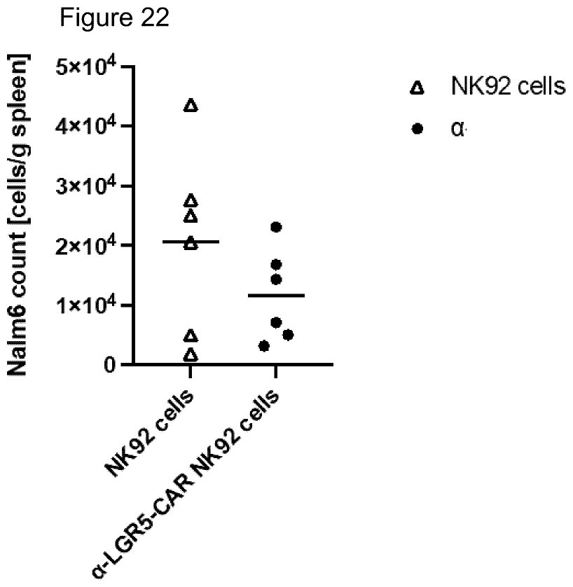

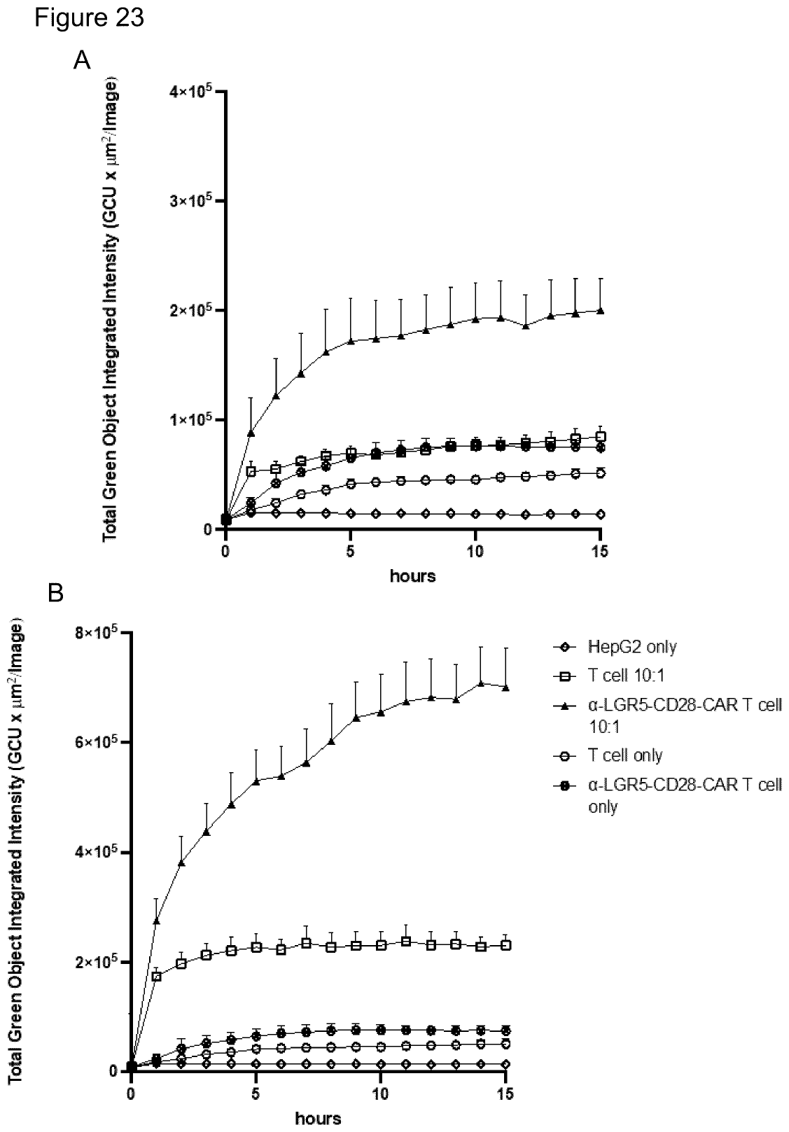

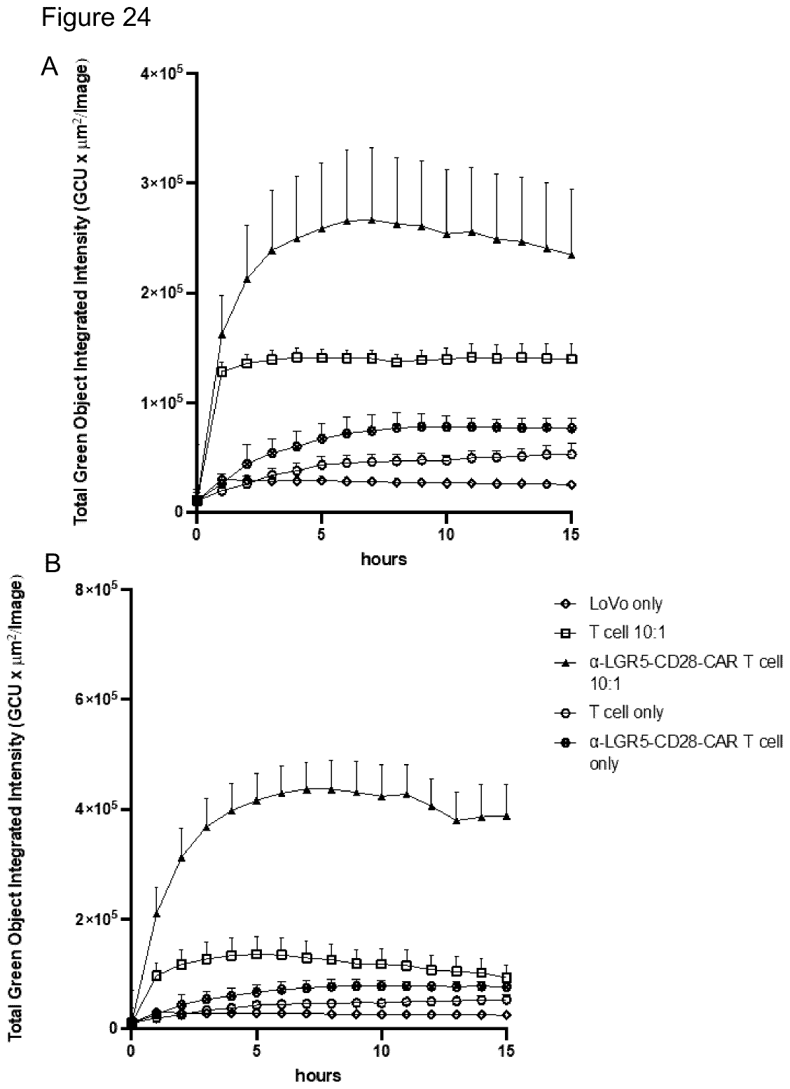

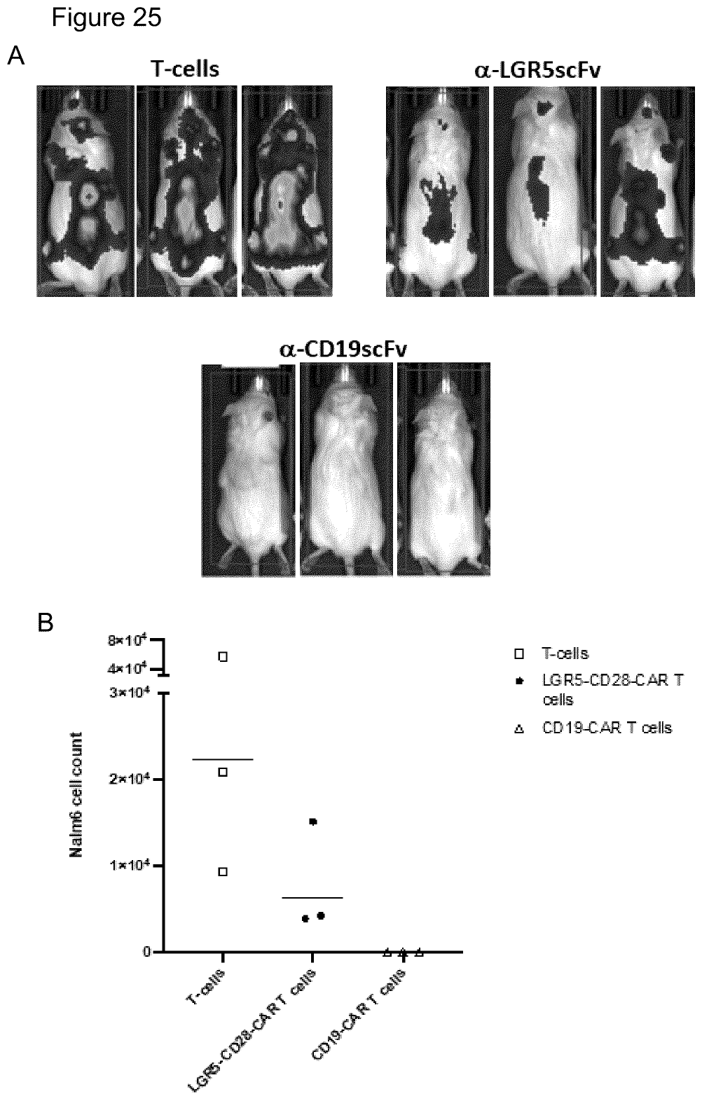

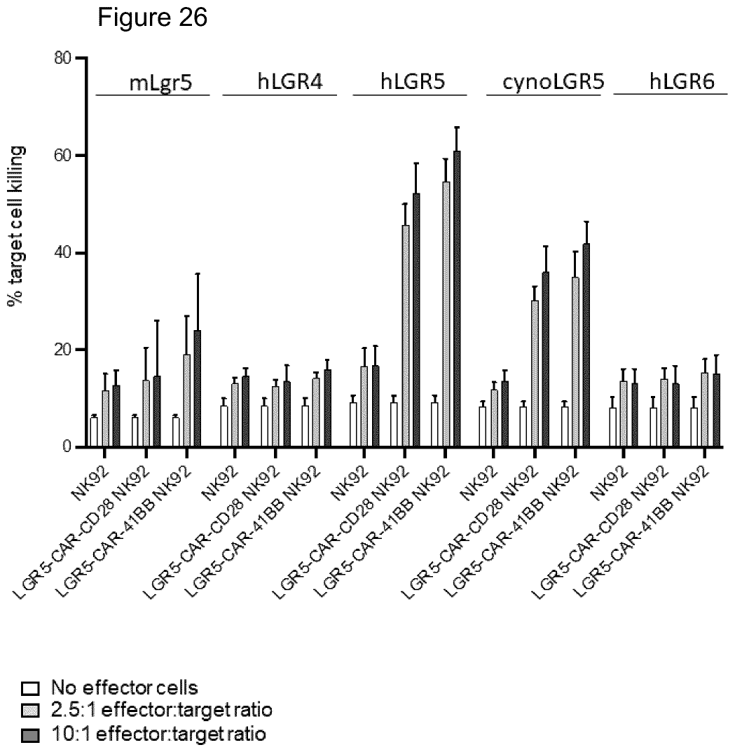

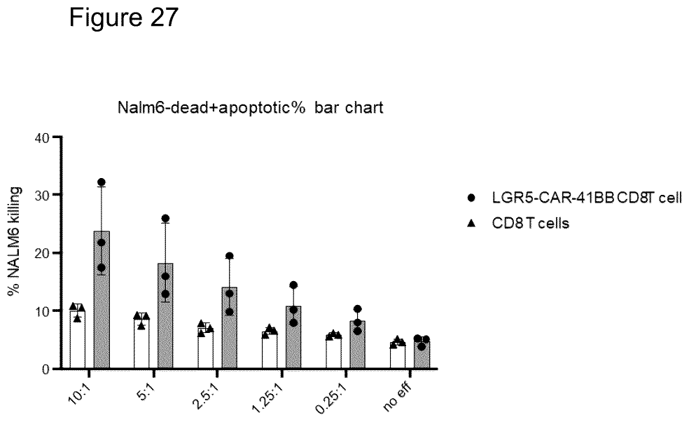

The inventors next sought to determine whether they could target exogenous cargo such as FL-α-LGR5 to the LGR5 decorated intracellular puncta. Treatment of HEK293T cells overexpressing LGR5-eGFP with FL-α-LGR5 at 37° C. led to specific internalization of the antibody (FIG. 7A). The kinetics of internalization were rapid; within 5 minutes FI-a-LGR5 was internalised, localising to puncta juxtaposed to the cell surface that ultimately associated entirely with the intracellular LGR5-eGFP puncta over the course of 45 minutes. HEK293T cells that were transfected with LGR4-eGFP failed to bind or internalize FL-α-LGR5 (FIG. 7A).

To determine whether endogenous LGR5 expression could support FI-α-LGR5 internalisation, the inventors repeated the internalisation assay using NALM6 cells. Similar to HEK293T cells overexpressing hLGR5, NALM6 cells rapidly internalised FI-α-LGR5, within 15 minutes, but not when the antibody was pre- incubated with the Fragment 1A peptide (FIG. 7B).

To distinguish between cell surface binding of FI-α-LGR5 and its internalisation in NALM6 cells, the inventors carried out the assay at 4° C. and 37° C. respectively. While the inventors detected antibody binding to only a small proportion of NALM6 cells (approx. 1%), the cells uniformly internalised FI-α-LGR5 after 30 minutes of incubation (FIG. 7C).

LoVo cells also rapidly internalised FI-α-LGR5, within 5 minutes, unless the antibody was pre-incubated with the Fragment 1A peptide (FIG. 7D).

To examine specific LGR5 internalisation by FI-α-LGR5 uptake in LoVo cells, the inventors generated a control line in which they inactivated LGR5 by targeting the first exon of the LGR5 gene using CRISPR/Cas9. Interestingly, some of the targeted clonal lines harboured lesions in both LGR5 alleles that code for a protein lacking the 7 amino acids of the signal sequence (Suppl. FIG. 5). The mutant protein was expressed at in the targeted cell line at comparable levels to the parental line (FIG. 5E); however, flow cytometry analysis using FI-α-LGR5 to probe for cell surface LGR5 did not detect the mutant protein in the LGR5-targeted line (FIG. 5F). The data suggests that the mutant LGR5 protein lacking a complete signal peptide is not translated into the endoplasmic reticulum for incorporation into the secretory pathway and cell surface expression.

Together, the internalization data indicates that α-LGR5 binds to and is rapidly internalized by cells with low and transient levels of cell-surface LGR5.

### Validation of α-LGR5 Antibody-Drug Conjugates

The inventors next engineered versions of α-LGR5 fused to the microtubule poison MMAE through a divinyl pyrimidine disulfide bridging linker inserted within the heavy chain-light chain disulfide linkages for precise 4:1 stoichiometry (FIG. 10). Two versions of the α-LGR5-MMAE conjugate were generated for in vitro studies: a sulphatase cleavable version (α-LGR5-ADC; (Walsh et al; Bargh et al)) and a non-cleavable version (a-LGR5-ADCNC; FIG. 10). The inventors also generated the control IgG1 conjugated to MMAE via the cleavable linker. Importantly, the α-LGR5-ADC demonstrated similar epitope binding affinities to the parental antibody (Table 5).

Single dose treatment of NALM6 cells with α-LGR5-ADC showed effective cell killing over three days with an EC50 of 4 nM (FIG. 9A). α-LGR5-ADC was slightly less effective against the REH pre-B-All cell line that expresses lower LGR5 levels with an EC50 of 10 nM. The inventors found no effect when treating NALM6 cells with α-LGR5-ADCNC consistent with a previous study that found a non-cleavable version of an LGR5 antibody-based ADC was ineffective at cell killing. In vitro killing assays using LoVo cells showed similar results with effective cell killing at an EC50 value of 9 nM.

### Targeting NALM6 Tumours in Vivo

To test in vivo efficacy of the α-LGR5-ADC the inventors implanted NALM6 cells constitutively expressing the luciferase transgene into NSG mice. On day 5 post-implantation IVIS imaging was performed and mice were stratified into two groups with identical overall tumour burden. On day 6, 8, 10, and 12 post implantation mice were treated with 5 mg/kg α-LGR5-ADC via tail vein injection (FIG. 11A). The control cohort of mice received injections of 5 mg/kg IgG1-ADC control on these days. Tumour burden was monitored at 2-3 day intervals by IVIS imaging. While NALM6 tumours treated with IgG1-ADC grew at a logarithmic rate, the inventors observed tumour regression within 4 days of the initial day 6 α-LGR5-ADC treatment (FIG. 11A). Tumour regression persisted throughout the course of treatment at which tumour growth resumed, with a four-day latency period, on day 16 p.i. At experimental endpoint on day 20, the a-LGR5-ADC treatment group had less than 0.5% of the tumour burden of control mice (FIG. 11A, B). The inventors also noted a marked reduction in splenic mass (approximately 2-fold) and residual NALM6 cells (approximately 100-fold; (FIG. 11C) as well as absolute numbers of NALM6 cells in the blood (100-fold) and bone marrow (50-fold) of α-LGR5-ADC treated mice (FIG. 11D, E). No tissue toxicity was observed and proliferation of stem cells in the intestinal crypts was unaffected by α-LGR5-ADC treatment (FIG. 12).

Targeting NALM6 Tumours with a Humanized ADC Version of α-LGR5

In order to generate an ADC product for potential clinical use, the inventors humanised a-LGR5 antibody leading to the generation of 16 variants based on the human IgG scaffold. The inventors proceeded with one of the humanized clones, variant 4 (α-LGR5v4) that displayed specific immune reactivity towards expressed human or cynoLGR5 (FIG. 14A) and high-affinity binding to the LGR5 epitope, Kd=2 nM, comparable to the parental murine antibody (Table 5). As a negative control for the experiments, the inventors used humanized variant 6 (a-LGR5v6) which lost all reactivity and did not bind to either human or cyno LGR5 proteins (FIG. 14A) or the LGR5 epitope (Table 5). Moreover, we observed essentially the same specificities as α-LGR5 when using a-LGR5v4 as an immune fluorescent probe; detection of the human and cyno over-expressed transgenic LGR5 proteins but none of the other LGR proteins (FIG. 14B).

The inventors generated ADC versions of α-LGR5v4 and the α-LGR5v6 control for treatment of NALM6 tumour bearing mice with a two-dose treatment regime on days 6 and 8 p.i. Consistent with the previous in vivo trial, the inventors observed a significant reduction in tumour growth after a four-day latency period from the first treatment day and persistence of the reduction in tumour growth for at least two days after the last α-LGR5v4 treatment (FIG. 13A). Overall NALM tumour burden, measured by IVIS imaging, was about half of control values at experimental endpoint (FIG. 13A, B). As with treatment with the α-LGR5-ADC, the inventors noted a reduction in splenic mass (3-fold) and residual NALM6 cells (approximately 50-fold) as well as an approximately 10-fold reduction NALM6 cells in the blood. Interestingly, this treatment regime saw the maintenance of the NALM6 tumour burden within the bone marrow where there were no significant differences in total tumour cell number. No tissue toxicity was observed and proliferation of stem cells in the intestinal crypts was unaffected by α-LGR5-ADC treatment (FIG. 14).

Together, the in vivo trial data obtained by the inventors indicates that the α-LGR5 ADCs are effective at suppressing tumour growth but require persistent treatment for durable response.

Binding Affinity Constant of scFv-α-LGR5

The inventors next sought to develop an LGR5 antibody fragment and determine whether such fragments exhibited binding activity equivalent to the α-LGR5 clones. The inventors prepared a single chain variable chain fragment (scFv) by transfecting an expression plasmid containing the 6×His-tagged scFv-α-LGR5 transgene into HEK293T cells and purifying the protein from the conditioned media. Binding affinity was determined by biolayer interferometry on the Octet platform. The inventors observed extremely high affinity binding between the scFv-α-LGR5 and the peptide with a Kd value 770 μM (table 5).

### Generation of CARs and BiTEs

The inventors next developed a chimeric antigen receptor (CAR) and bispecific T-cell engager (BiTE) using the antibodies of the present invention.

A humanized scFv fragment of the LGR5 antibody (scFv-α-LGR5) was engineered for use as a Bispecific T cell Engager(BiTE). The α-LGR5-BiTEs are composed of the polypeptide chain of scFv-α-LGR5 fused to the scFv fragment of an antibody binding to the CD3ε subunit of the T cell receptor (TCR). The two BiTE formats are either scFv-α-LGR5 or scFv-α-CD3ε as the N-terminal moiety and are compatible with either bacterial expression or expression in mammalian cell lines prior to purification and use as a BiTE.

A scFv-α-LGR5 was engineered for use as a chimeric antigen receptor (CAR). The α-LGR5-CAR was expressed from lentivirally-transduced cells as a single polypeptide chain consisting of the scFv-α-LGR5 fused to a CD8alpha stalk and transmembrane domain followed by either a CD28 or a 4-1BB costimulatory domain and ending with the CD3zeta signalling domain.

The α-LGR5-CAR is compatible for expression in T cells, NK cells and other immune cells.

As shown in FIG. 15, α-LGR5-CAR NK cells are efficient killers with favorable killing kinetics. 2nd generation CAR constructs were generated using the humanized α-LGR5 scFv. CAR constructs were stably introduced into NK92 cells by lentiviral delivery. (A,B) HEK293 cells were transfected with human (A) and cynomolgus (B) LGR5 and served as target cells in killing assays. α-LGR5 CAR NK92 cells were co-cultured with modified HEK target cells at the indicated effector to target ratios for 5 and 9 hours, respectively. (C) a-LGR5 CAR NK92 cells were incubated for 50 min with NALM6 target cells labelled with blue cell dye and immunological synapse formation was assessed by confocal microscopy showing that a-LGR5 CAR NK92 cells form productive synapses with tumour target cells. (D) a-LGR5 CAR NK92 cells were incubated with Acute Lymphoblastic Leukemia (ALL) human tumour cell lines at the indicated effector to target ratios and specific tumour cell killing was assessed after 12 hours.

As shown in FIG. 16, α-LGR5 Bispecific T cell engagers (BiTEs) are activating T cells and lead to efficient tumour cell destruction. BiTE constructs were generated using the humanized a-LGR5 scFv and a humanized a-CD3 scFv in both directions (LC and CL, respectively). (A) PBMCs were isolated from healthy donors and incubated with NALM6 tumour cells in the presence of α-LGR5 scFv control, α-LGR5LC or CL BiTE, respectively. CD4+ and CD8+ T cell activation was determined by flow cytometric analysis of CD69 and CD25 expression after 24 hours. (B) NALM6 tumour cells were co-cultured with cytotoxic CD8+ T cells generated from healthy donor PBMC in the presence of α-LGR5 scFv control, α-LGR5LC or CL BiTE, respectively. Tumour cell killing was assessed at an effector to target ratio of 5 to 1 after 5 hours.

Sequences used are shown below:

α-LGR5 scFv fragment used in the experiments relating to CAR and BiTE constructs, see FIGS. 15 to 27:

The human variant 4 (clone 2.4 as shown in Tables 1 and 2) was used to make the scFv fragment and therefore is incorporated in the CAR and BiTE constructs. Thus, the CAR and BiTE referred to in FIGS. 17 to 27 use the human variant 4 (clone 2.4, see also sequences in Tables 2 and 3).

peptide:
SEQ ID NO: 211
MPLLLLLPLLWAGALAEIVMTQSPATLSLSPGERATLSCRASQDISNRL
NWYQQKPGQAVRLLISYRSRRHTGIPARFSGSGSGTDYTLTISSLEPED
FAVYYCQQGNSLPPTFGGGTKLEIKGGGGSGGGGSGGGGSGGGGSQVQL
VQSGAEVKKPGASVKVSCKASGYTFTNYWMQWVRQAPGQGLEWIGEIDP
SDSYTNYNQKFQGRVTLTVDTSTSTAYMELSSLRSEDTAVYYCARSLSG
YVDYWGQGTTVTVSS

The CDRs of the light and heavy chains are underlined and are as follows (see also sequences in Table 2):

CDR1 light chain
SEQ ID NO: 212
RASQDISNRLN
CDR2 light chain
SEQ ID NO: 213
YRSRRHT
CDR3 light chain
SEQ ID NO: 214
QQGNSLPPT
CDR1 heavy chain
SEQ ID NO: 215
NYWMQ
CDR2 heavy chain
SEQ ID NO: 216
EIDPSDSYTNYNQKFQG
CDR3 heavy chain
SEQ ID NO: 217
SLSGYVDY
nucleic acid:
SEQ ID NO: 218
ATGCCGCTGCTGCTACTGCTGCCCCTGCTGTGGGCAGGGGCGCTAGCTG
AGATCGTGATGACTCAAAGCCCCGCCACACTGAGCCTATCTCCCGGCGA
GAGAGCCACGCTGAGCTGTAGAGCAAGCCAAGACATCAGCAACAGACTG
AACTGGTATCAGCAGAAGCCCGGCCAAGCCGTGAGACTGCTGATCAGCT
ACAGAAGCAGAAGACACACCGGCATCCCCGCTAGATTCAGCGGCAGCGG
CAGCGGCACCGACTACACCCTGACCATCAGCAGCCTGGAGCCTGAGGAC
TTCGCTGTATACTACTGCCAACAAGGCAACAGCCTGCCGCCAACCTTCG
GAGGCGGCACGAAGCTGGAGATCAAGGGCGGTGGTGGATCGGGCGGCGG
GGGCAGCGGGGGTGGTGGTTCTGGTGGAGGGGGCTCACAAGTGCAGCTG
GTGCAGAGCGGGGCCGAGGTGAAGAAGCCCGGCGCAAGCGTGAAGGTGA
GCTGCAAGGCAAGCGGCTACACCTTCACCAACTACTGGATGCAGTGGGT
GAGACAAGCCCCCGGCCAAGGCCTGGAGTGGATCGGCGAGATCGACCCT
AGCGACAGCTACACCAACTACAATCAGAAGTTCCAAGGCAGAGTGACCC
TGACCGTGGACACAAGCACAAGCACCGCCTACATGGAGCTGAGCAGCCT
GAGAAGCGAGGACACCGCGGTTTACTACTGCGCTAGAAGCCTGAGCGGC
TACGTGGACTACTGGGGCCAAGGCACCACCGTGACCGTGAGCAGC

### SUMMARY

Our census of human cancers revealed preB-ALL, CRC and HCC as indicator diseases for our portfolio of a-LGR5 therapeutics (FIG. 3).

Human preB-ALL cells (NALM6), CRC cells (LoVo), and HCC cells (HepG2) express high levels of LGR5 transcript and protein (FIG. 5, 17).

The a-LGR5 ADC shows high efficacy in vitro targeting NALM, REH and LoVo cells (FIG. 9) and importantly preferentially kills LGR5HIGH patient-derived CRC organoids (FIG. 18). Murine and humanised α-LGR5 ADC show pre-clinical efficacy in an NSG model of ALL (NALM6) (FIG. 11).

The a-LGR5 BiTE efficiently activated human CD4+ and CD8+ T cells and importantly efficiently leads to specific tumour cell death (NALM6) when incubated in the presence of tumour and human CD8+ cytotoxic T cells (FIG. 19).

α-LGR5-CAR NK92 cells efficiently and specifically target HEK293T cells overexpressing human and cynomolgus LGR5 but not HEK293T cells overexpressing human LGR4, human LGR6, and to a much lesser extend mouse Lgr5 (FIGS. 20 and 26). α-LGR5-CAR NK92 cells also specifically target LGR5HIGH NALM6 cells and to a much lesser extent LGR5LOW R E H cells in vitro (FIG. 20). Taken together, these experiments indicate exquisite specificity of the a-LGR5-CAR and a favourable therapeutic window by not targeting LGR5 low-expressing cells (potential stem cells). Furthermore, a-LGR5-CAR NK92 cells efficiently kill HepG2 cells (FIG. 21) and show moderate pre-clinical efficacy in an NSG model of ALL (NALM6) in vivo (FIG. 27).

α-LGR5-CART cells efficiently kill NALM6 (FIG. 23), HepG2 (FIG. 24), and LoVo tumour cells (PP FIG. 13) in vitro. Strikingly, our α-LGR5-CART cells show exquisite pre-clinical efficacy in an NSG model of ALL (NALM6) in vivo (FIG. 25).

### Discussion

The inventors have carried out extensive validation and characterisation of a novel antibody against the extracellular domain of LGR5 with a view to establishing it as a research tool and for determining its potential therapeutic applications. α-LGR5 is distinct from previously reported anti-LGR5 therapeutic antibodies owing to its target epitope on the LGR5 protein, unique variable heavy and light chains, and particularly high binding affinity to its epitope.

LGR5 has been established as a marker of stem cells in a number of epithelial tissues through extensive expression analysis with genetically engineered mouse models (GEMMs). Moreover, it has received a great deal of attention as a marker of certain malignancies through functional studies in GEMMs as well as detailed transcription analysis of cancer patient datasets.

The inventors have shown that the α-LGR5 antibodies developed are sensitive and specific tools for determining cellular levels and localisation of LGR5 protein in healthy and malignant tissue. The inventors used transcriptomic analysis of LGR5 expression and characterised LGR5 protein expression in a wide range of clinical samples from various cancers and matched healthy tissues. The inventors thereby established elevated LGR5 expression as a characteristic of various cancers including CRC, HCC and pre-B-ALL. Importantly, the tissue census, both transcriptional and at the level of LGR5 protein indicates that normal tissues, in general, harbour very low LGR5 levels, paving the way for therapeutic targeting of malignancies that overexpress the protein.

The inventors observed differences between the results from their and previous transcript analyses in relation to the LGR5 protein levels in brain and ovarian cancers; both malignancies consistently score high for LGR5 transcript levels yet the inventors were only able to detect very low LGR5 protein levels in few cells in less than 10% of the tumours tested. While this disparity may, in part, be due to different case samples used in the generation of the datasets, it highlights the need for establishing LGR5 protein levels as a prognostic indicator targeting of cancers based LGR5 overexpression.

The antibodies developed by the present inventors specifically recognise native LGR5 on the plasma membrane but also faithfully detect LGR5 expression under various fixation conditions.

This makes the antibodies developed by the inventors an excellent research tool and the inventors have used a-LGR5 to characterise the subcellular localisation of endogenous LGR5.

All detectable LGR5 protein in NALM6 and LoVo cells as well as in colon epithelial, CRC tumours, HCC and some of the ovarian cancer cases examined is localised to intracellular puncta. The ability to access the LGR5 positive intracellular puncta through rapid internalisation of FI-α-LGR5 supports dynamic shuttling between highly transitory population of LGR5 at the cell surface and an LGR5-associated vesicular compartment. Indeed, this hypothesis is corroborated by the inventors' comparative studies of LoVo cells and the LGR5-targeted in which LGR5 protein, lacking the signal peptide was not trafficked to the plasma membrane along the secretory pathway; these cells do not internalise LGR5.

Overexpression of LGR5 protein by some cancers and the highly dynamic nature of its internalisation makes LGR5 an ideal cellular target for ADC-based therapeutics. The inventors used a recently developed protocol for linking the MMAE payload to a-LGR5 resulting in an ADC displaying little change in Kd values for epitope binding indicating no loss in binding affinity. In vitro validation showed low nanomolar EC50 values, on par with Kd values for epitope binding, in targeting LGR5-expressing NALM6 and LoVo cells.

In vivo experiments targeting NALM6 tumours with α-LGR5-ADC are consistent with previous studies targeting LoVo tumours; after a two-day refractory period, the inventors observed tumour attrition that persisted during the course of treatment. Withdrawal of αLGR5-ADC restored tumour growth indicating the possibility that during the course of treatment, NALM6 cells may localise, reversibly, to niches that are less accessible to the drug. This notion is reinforced with the trial using the humanized versions of α-LGR5-ADC—while treatment over four days effectively targeted the NALM6 tumour, tumour growth was eventually restored. In this instance, the bone marrow was one of the compartments examined that contained substantial levels of NALM6 cells that were not eliminated by the low dose targeting regime.

Importantly, no detectable off-target toxicities of the α-LGR5-ADC was observed in the treated mice paving the way for future in vivo trials with increased treatment doses of α-LGR5-ADC that may provide increased efficacy in targeting potential tumour cell niches.

The present inventors have developed and validated novel antibodies and antibody fragments against human and cynoLGR5. The antibodies are highly specific, with high affinity and are taken up rapidly by LGR5 expressing cancer cells. They represent excellent research tools but also show great efficacy when used as ADCs in a model of ALL in vivo and so represent therapeutic tools which could be used to treat cancer, particularly cancers in which LGR5 is overexpressed. For example, the present inventors have designed and developed a chimeric antigen receptor (CAR) and bispecific T-cell engager (BiTE) using the antibodies of the present invention which could be used in the treatment of cancer, for example.

### REFERENCES

M. Rossmann, S. J. Greive, T. Moschetti, M. Dinan, M. Hyvönen, Development of a multipurpose scaffold for the display of peptide loops. Protein Eng. Des. Sel. 30, 419-430 (2017).
S. J. Walsh, et al., A general approach for the site-selective modification of native proteins, enabling the generation of stable and functional antibody-drug conjugates. Chem. Sci. 10, 694-700 (2019).
J. D. Bargh, et al., Sulfatase-cleavable linkers for antibody-drug conjugates. Chem. Sci. 11, 2375-2380 (2020).
W. C. Peng, et al., Structure of Stem Cell Growth Factor R-spondin 1 in Complex with the Ectodomain of Its Receptor LGR5. Cell Rep. 3, 1885-1892 (2013).
M. De La Roche, A. E. K. Ibrahim, J. Mieszczanek, M. Bienz, LEF1 and B9L shield b-catenin from inactivation by axin, desensitizing colorectal cancer cells to tankyrase inhibitors. Cancer Res. 74, 1495-1505 (2014).

1. An antibody or fragment thereof which binds to LGR5, wherein the antibody binds an epitope located within amino acids 22-37 of SEQ ID NO.1.

2. The antibody or fragment thereof of claim 1, wherein the antibody binds an epitope which consists of amino acids 22-37 of SEQ ID NO.1.

3. The antibody or fragment thereof of claim 1 or 2 wherein the V_{H} of the antibody comprises the following CDR1, CDR2 and CDR3:
a) a CDR1 of SEQ ID No. 50 or a sequence with at least 90% homology thereto, a CDR2 of SEQ ID No. 51 or a sequence with at least 90% homology thereto, a CDR3 of SEQ ID No. 52 or a sequence with at least 90% homology thereto; or
b) a CDR1 of SEQ ID No. 2 or a sequence with at least 90% homology thereto, a CDR2 of SEQ ID No. 3 or a sequence with at least 90% homology thereto, a CDR3 of SEQ ID No. 4 or a sequence with at least 90% homology thereto; or
c) a CDR1 of SEQ ID No. 8 or a sequence with at least 90% homology thereto, a CDR2 of SEQ ID No. 9 or a sequence with at least 90% homology thereto, a CDR3 of SEQ ID No. 10 or a sequence with at least 90% homology thereto; or
d) a CDR1 of SEQ ID No. 14 or a sequence with at least 90% homology thereto, a CDR2 of SEQ ID No. 15 or a sequence with at least 90% homology thereto, a CDR3 of SEQ ID No. 16 or a sequence with at least 90% homology thereto; or
e) a CDR1 of SEQ ID No. 20 or a sequence with at least 90% homology thereto, a CDR2 of SEQ ID No. 21 or a sequence with at least 90% homology thereto, a CDR3 of SEQ ID No. 22 or a sequence with at least 90% homology thereto; or
f) a CDR1 of SEQ ID No. 26 or a sequence with at least 90% homology thereto, a CDR2 of SEQ ID No. 27 or a sequence with at least 90% homology thereto, a CDR3 of SEQ ID No. 28 or a sequence with at least 90% homology thereto; or
g) a CDR1 of SEQ ID No. 32 or a sequence with at least 90% homology thereto, a CDR2 of SEQ ID No. 33 or a sequence with at least 90% homology thereto, a CDR3 of SEQ ID No. 34 or a sequence with at least 90% homology thereto; or
h) a CDR1 of SEQ ID No. 38 or a sequence with at least 90% homology thereto, a CDR2 of SEQ ID No. 39 or a sequence with at least 90% homology thereto, a CDR3 of SEQ ID No. 40 or a sequence with at least 90% homology thereto; or
i) a CDR1 of SEQ ID No. 44 or a sequence with at least 90% homology thereto, a CDR2 of SEQ ID No. 45 or a sequence with at least 90% homology thereto, a CDR3 of SEQ ID No. 46 or a sequence with at least 90% homology thereto; or
j) a CDR1 of SEQ ID No. 56 or a sequence with at least 90% homology thereto, a CDR2 of SEQ ID No. 57 or a sequence with at least 90% homology thereto, a CDR3 of SEQ ID No. 58 or a sequence with at least 90% homology thereto; or
k) a CDR1 of SEQ ID No. 62 or a sequence with at least 90% homology thereto, a CDR2 of SEQ ID No. 63 or a sequence with at least 90% homology thereto, a CDR3 of SEQ ID No. 64 or a sequence with at least 90% homology thereto; or
l) a CDR1 of SEQ ID No. 68 or a sequence with at least 90% homology thereto, a CDR2 of SEQ ID No. 69 or a sequence with at least 90% homology thereto, a CDR3 of SEQ ID No. 70 or a sequence with at least 90% homology thereto; or
m) a CDR1 of SEQ ID No. 74 or a sequence with at least 90% homology thereto, a CDR2 of SEQ ID No. 75 or a sequence with at least 90% homology thereto, a CDR3 of SEQ ID No. 76 or a sequence with at least 90% homology thereto.

4. The antibody or fragment thereof according to any preceding claim wherein the V_{L} of the antibody comprises the following CDR1, CDR2 and CDR3:
a) a CDR1 of SEQ ID No. 53 or a sequence with at least 90% homology thereto, a CDR2 of SEQ ID No. 54 or a sequence with at least 90% homology thereto, a CDR3 of SEQ ID No. 55 or a sequence with at least 90% homology thereto; or
b) a CDR1 of SEQ ID No. 5 or a sequence with at least 90% homology thereto, a CDR2 of SEQ ID No. 6 or a sequence with at least 90% homology thereto, a CDR3 of SEQ ID No. 7 or a sequence with at least 90% homology thereto; or
c) a CDR1 of SEQ ID No. 11 or a sequence with at least 90% homology thereto, a CDR2 of SEQ ID No. 12 or a sequence with at least 90% homology thereto, a CDR3 of SEQ ID No. 13 or a sequence with at least 90% homology thereto; or
d) a CDR1 of SEQ ID No. 17 or a sequence with at least 90% homology thereto, a CDR2 of SEQ ID No. 18 or a sequence with at least 90% homology thereto, a CDR3 of SEQ ID No. 19 or a sequence with at least 90% homology thereto; or
e) a CDR1 of SEQ ID No. 23 or a sequence with at least 90% homology thereto, a CDR2 of SEQ ID No. 24 or a sequence with at least 90% homology thereto, a CDR3 of SEQ ID No. 25 or a sequence with at least 90% homology thereto; or
f) a CDR1 of SEQ ID No. 29 or a sequence with at least 90% homology thereto, a CDR2 of SEQ ID No. 30 or a sequence with at least 90% homology thereto, a CDR3 of SEQ ID No. 31 or a sequence with at least 90% homology thereto; or
g) a CDR1 of SEQ ID No. 35 or a sequence with at least 90% homology thereto, a CDR2 of SEQ ID No. 36 or a sequence with at least 90% homology thereto, a CDR3 of SEQ ID No. 37 or a sequence with at least 90% homology thereto; or
h) a CDR1 of SEQ ID No. 41 or a sequence with at least 90% homology thereto, a CDR2 of SEQ ID No. 42 or a sequence with at least 90% homology thereto, a CDR3 of SEQ ID No. 43 or a sequence with at least 90% homology thereto; or
i) a CDR1 of SEQ ID No. 47 or a sequence with at least 90% homology thereto, a CDR2 of SEQ ID No. 48 or a sequence with at least 90% homology thereto, a CDR3 of SEQ ID No. 49 or a sequence with at least 90% homology thereto; or
j) a CDR1 of SEQ ID No. 59 or a sequence with at least 90% homology thereto, a CDR2 of SEQ ID No. 60 or a sequence with at least 90% homology thereto, a CDR3 of SEQ ID No. 61 or a sequence with at least 90% homology thereto; or
k) a CDR1 of SEQ ID No. 65 or a sequence with at least 90% homology thereto, a CDR2 of SEQ ID No. 66 or a sequence with at least 90% homology thereto, a CDR3 of SEQ ID No. 67 or a sequence with at least 90% homology thereto; or
l) a CDR1 of SEQ ID No. 71 or a sequence with at least 90% homology thereto, a CDR2 of SEQ ID No. 72 or a sequence with at least 90% homology thereto, a CDR3 of SEQ ID No. 73 or a sequence with at least 90% homology thereto; or
m) a CDR1 of SEQ ID No. 77 or a sequence with at least 90% homology thereto, a CDR2 of SEQ ID No. 78 or a sequence with at least 90% homology thereto, a CDR3 of SEQ ID No. 79 or a sequence with at least 90% homology thereto.

5. The antibody or fragment thereof according to any preceding claim, comprising a V_{H} sequence selected from SEQ ID NOs: 96, 80, 82, 84, 86, 88, 90, 92, 94, 98, 100, 102, 104.

6. The antibody or fragment thereof according to any preceding claim, comprising a V_{L} sequence selected from SEQ ID NOs: 97, 81, 83, 85, 87, 89, 91, 93, 95, 99, 101, 103, 105.

7. The antibody or fragment thereof according to any preceding claim comprising:
i) a V_{H} sequence of SEQ ID NO: 96 and a V_{L} sequence of SEQ ID NO: 97;
b) a V_{H} sequence of SEQ ID NO: 80 and a V_{L} sequence of SEQ ID NO: 81;
c) a V_{H} sequence of SEQ ID NO: 82 and a V_{L} sequence of SEQ ID NO: 83;
d) a V_{H} sequence of SEQ ID NO: 84 and a V_{L} sequence of SEQ ID NO: 85;
e) a V_{H} sequence of SEQ ID NO: 86 and a V_{L} sequence of SEQ ID NO: 87;
f) a V_{H} sequence of SEQ ID NO: 88 and a V_{L} sequence of SEQ ID NO: 89;
g) a V_{H} sequence of SEQ ID NO: 90 and a V_{L} sequence of SEQ ID NO: 91;
h) a V_{H} sequence of SEQ ID NO: 92 and a V_{L} sequence of SEQ ID NO: 93;
i) a V_{H} sequence of SEQ ID NO: 94 and a V_{L} sequence of SEQ ID NO: 95;
j) a V_{H} sequence of SEQ ID NO: 98 and a V_{L} sequence of SEQ ID NO: 99;
k) a V_{H} sequence of SEQ ID NO: 100 and a V_{L} sequence of SEQ ID NO: 101
l) a V_{H} sequence of SEQ ID NO: 102 and a V_{L} sequence of SEQ ID NO: 103; or
m) a V_{H} sequence of SEQ ID NO: 104 and a V_{L} sequence of SEQ ID NO: 105.

8. The antibody or fragment thereof according to any preceding claim comprising a sequence of an antibody clone as shown in Table 3 or a sequence with at least 70%, 80% or 90% homology thereto.

9. The antibody or fragment thereof according to claim 1 or claim 2 wherein the fragment is an scFV comprising SEQ ID NO. 211 or a sequence with at least 70%, 80% or 90% homology thereto.

10. The antibody or fragment thereof according to any preceding claim wherein the antibody binds to human LGR5.

11. The antibody or fragment thereof according to any preceding claim wherein the antibody is a monoclonal antibody.

12. The antibody or fragment thereof of any preceding claim, wherein the antibody is a human, humanized, or chimeric antibody.

13. The antibody or fragment thereof of any preceding claim, wherein the antibody is conjugated to a toxin, enzyme, radioisotope, label, therapeutic agent or other chemical moiety.

14. The antibody or fragment thereof of any preceding claim, wherein the antibody is capable of binding LGR5 with a Kd of less than around 4 nM.

15. The antibody or fragment thereof according to any preceding claim wherein the fragment comprises a Fab, scFv or single domain antibody.

16. An immunoconjugate comprising an antibody or fragment thereof according to any one of claims 1-15 linked to a therapeutic agent.

17. The immunoconjugate according to claim 16 wherein said therapeutic agent is a toxin, enzyme, radioisotope or other chemical moiety.

18. A pharmaceutical composition comprising an antibody according to any one of claims 1-15 or an immunoconjugate according to claim 16 or 17 and a pharmaceutical carrier.

19. An antibody or fragment thereof according to any one of claims 1-15, an immunoconjugate according to claim 16 or 17 or a pharmaceutical composition according to claim 18 for use as a medicament.

20. An antibody or fragment thereof according to any one of claims 1-15, an immunoconjugate according to claim 16 or 17 or a pharmaceutical composition according to claim 18 for use in the treatment or diagnosis of a cancer or an inflammatory disease.

21. A method for treating a cancer comprising administering a therapeutically effective amount of an antibody or fragment thereof according to any one of claims 1 to 15, an immunoconjugate according to claims 16 to 17 or a pharmaceutical composition according to claim 18.

22. The antibody or fragment thereof for use of claim 20 or the method of claim 21 wherein the cancer is an LGR5-positive cancer.

23. The antibody or fragment thereof for use of claim 22 or the method of claim 22 wherein the cancer overexpresses LGR5.

24. The antibody or fragment thereof for use of any of claims 20, 22 or 23 or the method of any of claims 21 to 23 wherein the cancer is selected from cancer of the head or neck, uterine cancer, colorectal cancer, stomach cancer, carcinoma of the endometrium, cancer of the esophagus, leukemia, such as acute lymphoblastic leukemia (ALL), liver cancer, such as hepatocellular carcinoma, or pancreatic cancer.

25. An isolated nucleic acid molecule comprising a nucleotide sequence encoding an antibody or fragment thereof according to any of claims 1-15.

26. A vector comprising a nucleic acid according to claim 26.

27. A host cell comprising a nucleic acid according to claim 25 or a vector according to claim 26.

28. A method of detecting LGR5 in a biological sample comprising contacting the biological sample with the antibody according to any of claims 1-15 under conditions permissive for binding of the antibody to LGR5, and detecting whether a complex is formed between the antibody and LGR5.

29. The method of claim 28 wherein the biological sample is a cancer sample selected from cancer of the head or neck, uterine cancer, colorectal cancer, stomach cancer, carcinoma of the endometrium, cancer of the esophagus, leukemia, such as acute lymphoblastic leukemia (ALL), liver cancer, such as hepatocellular carcinoma or pancreatic cancer.

30. An antibody or fragment thereof which binds to LGR5 wherein the VH of the antibody comprises the following CDR1, CDR2 and CDR3:
a) a CDR1 of SEQ ID No. 50 or a sequence with at least 90% homology thereto, a CDR2 of SEQ ID No. 51 or a sequence with at least 90% homology thereto, a CDR3 of SEQ ID No. 52 or a sequence with at least 90% homology thereto; or
b) a CDR1 of SEQ ID No. 2 or a sequence with at least 90% homology thereto, a CDR2 of SEQ ID No. 3 or a sequence with at least 90% homology thereto, a CDR3 of SEQ ID No. 4 or a sequence with at least 90% homology thereto; or
c) a CDR1 of SEQ ID No. 8 or a sequence with at least 90% homology thereto, a CDR2 of SEQ ID No. 9 or a sequence with at least 90% homology thereto, a CDR3 of SEQ ID No. 10 or a sequence with at least 90% homology thereto; or
d) a CDR1 of SEQ ID No. 14 or a sequence with at least 90% homology thereto, a CDR2 of SEQ ID No. 15 or a sequence with at least 90% homology thereto, a CDR3 of SEQ ID No. 16 or a sequence with at least 90% homology thereto; or
e) a CDR1 of SEQ ID No. 20 or a sequence with at least 90% homology thereto, a CDR2 of SEQ ID No. 21 or a sequence with at least 90% homology thereto, a CDR3 of SEQ ID No. 22 or a sequence with at least 90% homology thereto; or
f) a CDR1 of SEQ ID No. 26 or a sequence with at least 90% homology thereto, a CDR2 of SEQ ID No. 27 or a sequence with at least 90% homology thereto, a CDR3 of SEQ ID No. 28 or a sequence with at least 90% homology thereto; or
g) a CDR1 of SEQ ID No. 32 or a sequence with at least 90% homology thereto, a CDR2 of SEQ ID No. 33 or a sequence with at least 90% homology thereto, a CDR3 of SEQ ID No. 34 or a sequence with at least 90% homology thereto; or
h) a CDR1 of SEQ ID No. 38 or a sequence with at least 90% homology thereto, a CDR2 of SEQ ID No. 39 or a sequence with at least 90% homology thereto, a CDR3 of SEQ ID No. 40 or a sequence with at least 90% homology thereto; or
i) a CDR1 of SEQ ID No. 44 or a sequence with at least 90% homology thereto, a CDR2 of SEQ ID No. 45 or a sequence with at least 90% homology thereto, a CDR3 of SEQ ID No. 46 or a sequence with at least 90% homology thereto; or
j) a CDR1 of SEQ ID No. 56 or a sequence with at least 90% homology thereto, a CDR2 of SEQ ID No. 57 or a sequence with at least 90% homology thereto, a CDR3 of SEQ ID No. 58 or a sequence with at least 90% homology thereto; or
k) a CDR1 of SEQ ID No. 62 or a sequence with at least 90% homology thereto, a CDR2 of SEQ ID No. 63 or a sequence with at least 90% homology thereto, a CDR3 of SEQ ID No. 64 or a sequence with at least 90% homology thereto; or
l) a CDR1 of SEQ ID No. 68 or a sequence with at least 90% homology thereto, a CDR2 of SEQ ID No. 69 or a sequence with at least 90% homology thereto, a CDR3 of SEQ ID No. 70 or a sequence with at least 90% homology thereto; or
m) a CDR1 of SEQ ID No. 74 or a sequence with at least 90% homology thereto, a CDR2 of SEQ ID No. 75 or a sequence with at least 90% homology thereto, a CDR3 of SEQ ID No. 76 or a sequence with at least 90% homology thereto.

31. An antibody or fragment thereof according to claim 30 which binds to LGR5 wherein the VL of the antibody comprises the following CDR1, CDR2 and CDR3:
a) a CDR1 of SEQ ID No. 53 or a sequence with at least 90% homology thereto, a CDR2 of SEQ ID No. 54 or a sequence with at least 90% homology thereto, a CDR3 of SEQ ID No. 55 or a sequence with at least 90% homology thereto; or
b) a CDR1 of SEQ ID No. 5 or a sequence with at least 90% homology thereto, a CDR2 of SEQ ID No. 6 or a sequence with at least 90% homology thereto, a CDR3 of SEQ ID No. 7 or a sequence with at least 90% homology thereto; or
c) a CDR1 of SEQ ID No. 11 or a sequence with at least 90% homology thereto, a CDR2 of SEQ ID No. 12 or a sequence with at least 90% homology thereto, a CDR3 of SEQ ID No. 13 or a sequence with at least 90% homology thereto; or
d) a CDR1 of SEQ ID No. 17 or a sequence with at least 90% homology thereto, a CDR2 of SEQ ID No. 18 or a sequence with at least 90% homology thereto, a CDR3 of SEQ ID No. 19 or a sequence with at least 90% homology thereto; or
e) a CDR1 of SEQ ID No. 23 or a sequence with at least 90% homology thereto, a CDR2 of SEQ ID No. 24 or a sequence with at least 90% homology thereto, a CDR3 of SEQ ID No. 25 or a sequence with at least 90% homology thereto; or
f) a CDR1 of SEQ ID No. 29 or a sequence with at least 90% homology thereto, a CDR2 of SEQ ID No. 30 or a sequence with at least 90% homology thereto, a CDR3 of SEQ ID No. 31 or a sequence with at least 90% homology thereto; or
g) a CDR1 of SEQ ID No. 35 or a sequence with at least 90% homology thereto, a CDR2 of SEQ ID No. 36 or a sequence with at least 90% homology thereto, a CDR3 of SEQ ID No. 37 or a sequence with at least 90% homology thereto; or
h) a CDR1 of SEQ ID No. 41 or a sequence with at least 90% homology thereto, a CDR2 of SEQ ID No. 42 or a sequence with at least 90% homology thereto, a CDR3 of SEQ ID No. 43 or a sequence with at least 90% homology thereto; or
i) a CDR1 of SEQ ID No. 47 or a sequence with at least 90% homology thereto, a CDR2 of SEQ ID No. 48 or a sequence with at least 90% homology thereto, a CDR3 of SEQ ID No. 49 or a sequence with at least 90% homology thereto; or
j) a CDR1 of SEQ ID No. 59 or a sequence with at least 90% homology thereto, a CDR2 of SEQ ID No. 60 or a sequence with at least 90% homology thereto, a CDR3 of SEQ ID No. 61 or a sequence with at least 90% homology thereto; or
k) a CDR1 of SEQ ID No. 65 or a sequence with at least 90% homology thereto, a CDR2 of SEQ ID No. 66 or a sequence with at least 90% homology thereto, a CDR3 of SEQ ID No. 67 or a sequence with at least 90% homology thereto; or
l) a CDR1 of SEQ ID No. 71 or a sequence with at least 90% homology thereto, a CDR2 of SEQ ID No. 72 or a sequence with at least 90% homology thereto, a CDR3 of SEQ ID No. 73 or a sequence with at least 90% homology thereto; or
m) a CDR1 of SEQ ID No. 77 or a sequence with at least 90% homology thereto, a CDR2 of SEQ ID No. 78 or a sequence with at least 90% homology thereto, a CDR3 of SEQ ID No. 79 or a sequence with at least 90% homology thereto.

32. An antibody or fragment thereof which binds to LGR5 according to claim 30 or 31, comprising a VH sequence selected from SEQ ID NOs: 96, 80, 82, 84, 86, 88, 90, 92, 94, 98, 100, 102, 104.

33. An antibody or fragment thereof which binds to LGR5 according to claim 30 to 32, comprising a VL sequence selected from SEQ ID NOs: 97, 81, 83, 85, 87, 89, 91, 93, 95, 99, 101, 103, 105.

34. A chimeric antigen receptor comprising an antibody or fragment according to any of claims 1 to 15 or 30 to 33.

35. A CAR comprising SEQ ID NO. 211 or a sequence with at least 70%, 80% or 90% homology thereto.

36. A cell or cell population expressing a CAR of claim 34 or 35.

37. The cell or cell population according to claim 36 wherein the cell is an immune cell.

38. The cell or cell population according to claim 37 wherein the immune cell is selected from the group consisting of a T cell, a Natural Killer (NK) cell, a cytotoxic T lymphocyte (CTL), tumor infiltrating lymphocyte (TIL), TCR-expressing cell, dendritic cell, or NK-T cell and a regulatory T cell.

39. The cell or cell population according to claims 36 to 38 wherein the cell or is an autologous T cell or an allogeneic T cell.

40. A cell or population of cells according to any of claims 36 to 39 for use in adaptive immunotherapy.

41. An immune cell engager, such as a BiTE or TriKE, comprising an antibody or fragment according to any of claims 1 to 15 or 30 to 33.

42. A BiTE according to claim 41 comprising SEQ ID NO. 211 or a sequence with at least 70%, 80% or 90% homology thereto.

43. A method of producing an antibody comprising culturing the host cell according to claims 1 to 15 or 30 to 33 under conditions suitable for expression of the polynucleotide encoding the antibody and isolating the antibody.

44. An isolated synthetic or recombinant peptide comprising an epitope, the peptide consisting of residues 22 to 37 of SEQ ID NO: 1.

45. A method of diagnosing or assessing progression of cancer comprising assessing expression of LGR5 and/or protein levels of LGR5.

46. The method according to claim 45 further comprising comparing the level of expression to a threshold level.

47. The method according to claim 46 wherein said threshold level is the level of expression in normal tissue.

48. The method according to claim 47 further comprising selecting a treatment if the level of expression is above the threshold.

49. The method according to claim 48 further comprising administering said treatment.

50. The method according to claim 49 wherein the treatment is an antibody or antibody fragment of any of claims 1 to 15 or 30 to 33.

51. The method according to claim wherein the protein level is assessed using an antibody or antibody fragment of any of claims 1 to 15 or 30 to 33.


SEQUENCE LISTING (224 sequences)

SEQ ID NO: 1 - protein, 907 aa, Homo sapiens
MDTSRLGVLLSLPVLLQLATGGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRHVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSLISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIRIGVWTIAVLALTCNALVTSTVFRSPLYISPIKLLIGVIAAVNMLTGVSSAVLAGVDAFTFGSFARHGAWWENGVGCHVIGFLSIFASESSVFLLTLAALERGFSVKYSAKFETKAPFSSLKVIILLCALLALTMAAVPLLGGSKYGASPLCLPLPFGEPSTMGYMVALILLNSLCFLMMTIAYTKLYCNLDKGDLENIWDCSMVKHIALLLFTNCILNCPVAFLSFSSLINLTFISPEVIKFILLVVVPLPACLNPLLYILFNPHFKEDLVSLRKQTYVWTRSKHPSLMSINSDDVEKQSCDSTQALVTFTSSSITYDLPPSSVPSPAYPVTESCHLSSVAFVPCL

SEQ ID NO: 2 - protein, 5 aa, synthetic construct
  REGION 1..5: note = VH CDR1
SYWMQ

SEQ ID NO: 3 - protein, 17 aa, synthetic construct
  REGION 1..17: note = VH CDR2
EIDPSDYYTNYNQKFKG

SEQ ID NO: 4 - protein, 8 aa, synthetic construct
  REGION 1..8: note = VH CDR3
SLSGYVDY

SEQ ID NO: 5 - protein, 11 aa, synthetic construct
  REGION 1..11: note = VL CDR1
RASQDIRNRLN

SEQ ID NO: 6 - protein, 7 aa, synthetic construct
  REGION 1..7: note = VL CDR2
YRSRLQS

SEQ ID NO: 7 - protein, 9 aa, synthetic construct
  REGION 1..9: note = VL CDR3
QQGNTLPPT

SEQ ID NO: 8 - protein, 5 aa, synthetic construct
  REGION 1..5: note = VH CDR1
NYWMQ

SEQ ID NO: 9 - protein, 17 aa, synthetic construct
  REGION 1..17: note = VH CDR2
EIDPSDSYTNYNQKFKG

SEQ ID NO: 10 - protein, 8 aa, synthetic construct
  REGION 1..8: note = VH CDR3
SLSGYVDY

SEQ ID NO: 11 - protein, 11 aa, synthetic construct
  REGION 1..11: note = VL CDR1
RASQDISNRLN

SEQ ID NO: 12 - protein, 7 aa, synthetic construct
  REGION 1..7: note = VL CDR2
YRSRLHS

SEQ ID NO: 13 - protein, 9 aa, synthetic construct
  REGION 1..9: note = VL CDR3
QQGNSLPPT

SEQ ID NO: 14 - protein, 5 aa, synthetic construct
  REGION 1..5: note = VH CDR1
SYWMQ

SEQ ID NO: 15 - protein, 17 aa, synthetic construct
  REGION 1..17: note = VH CDR2
EIDPSDSYSNYNQKFKG

SEQ ID NO: 16 - protein, 8 aa, synthetic construct
  REGION 1..8: note = VH CDR3
SLSGYVDY

SEQ ID NO: 17 - protein, 11 aa, synthetic construct
  REGION 1..11: note = VL CDR1
RASQDISNRLN

SEQ ID NO: 18 - protein, 7 aa, synthetic construct
  REGION 1..7: note = VL CDR2
YRSRLQS

SEQ ID NO: 19 - protein, 9 aa, synthetic construct
  REGION 1..9: note = VL CDR3
QQGNTLPPT

SEQ ID NO: 20 - protein, 5 aa, synthetic construct
  REGION 1..5: note = VH CDR1
DYWMQ

SEQ ID NO: 21 - protein, 17 aa, synthetic construct
  REGION 1..17: note = VH CDR2
EIDPSDSYTNYNQKFQG

SEQ ID NO: 22 - protein, 8 aa, synthetic construct
  REGION 1..8: note = VH CDR3
SLSGSVDY

SEQ ID NO: 23 - protein, 11 aa, synthetic construct
  REGION 1..11: note = VL CDR1
RASQDISNRLN

SEQ ID NO: 24 - protein, 7 aa, synthetic construct
  REGION 1..7: note = VL CDR2
YRSRLQS

SEQ ID NO: 25 - protein, 9 aa, synthetic construct
  REGION 1..9: note = VL CDR3
QQGNTLPPT

SEQ ID NO: 26 - protein, 5 aa, synthetic construct
  REGION 1..5: note = VH CDR1
DYYMN

SEQ ID NO: 27 - protein, 17 aa, synthetic construct
  REGION 1..17: note = VH CDR2
DINPNNGGTIYNQKFKG

SEQ ID NO: 28 - protein, 6 aa, synthetic construct
  REGION 1..6: note = VH CDR3
GYLFDY

SEQ ID NO: 29 - protein, 17 aa, synthetic construct
  REGION 1..17: note = VL CDR1
KSSQSLLNSSNQKNYLA

SEQ ID NO: 30 - protein, 7 aa, synthetic construct
  REGION 1..7: note = VL CDR2
FASTRES

SEQ ID NO: 31 - protein, 9 aa, synthetic construct
  REGION 1..9: note = VL CDR3
QQHYSTPLT

SEQ ID NO: 32 - protein, 5 aa, synthetic construct
  REGION 1..5: note = VH CDR1
NYWMQ

SEQ ID NO: 33 - protein, 17 aa, synthetic construct
  REGION 1..17: note = VH CDR2
EIDPSDSYTNYNQKFQG

SEQ ID NO: 34 - protein, 8 aa, synthetic construct
  REGION 1..8: note = VH CDR3
SLSGYVDY

SEQ ID NO: 35 - protein, 11 aa, synthetic construct
  REGION 1..11: note = VL CDR1
RASQDISNRLN

SEQ ID NO: 36 - protein, 7 aa, synthetic construct
  REGION 1..7: note = VL CDR2
YRSRLHS

SEQ ID NO: 37 - protein, 9 aa, synthetic construct
  REGION 1..9: note = VL CDR3
QQGNSLPPT

SEQ ID NO: 38 - protein, 5 aa, synthetic construct
  REGION 1..5: note = VH CDR1
NYWMQ

SEQ ID NO: 39 - protein, 17 aa, synthetic construct
  REGION 1..17: note = VH CDR2
EIDPSDSYTNYNQKFQG

SEQ ID NO: 40 - protein, 8 aa, synthetic construct
  REGION 1..8: note = VH CDR3
SLSGYVDY

SEQ ID NO: 41 - protein, 11 aa, synthetic construct
  REGION 1..11: note = VL CDR1
RASQDISNRLN

SEQ ID NO: 42 - protein, 7 aa, synthetic construct
  REGION 1..7: note = VL CDR2
YRSRLHS

SEQ ID NO: 43 - protein, 9 aa, synthetic construct
  REGION 1..9: note = VL CDR3
QQGNSLPPT

SEQ ID NO: 44 - protein, 5 aa, synthetic construct
  REGION 1..5: note = VH CDR1
NYWMQ

SEQ ID NO: 45 - protein, 17 aa, synthetic construct
  REGION 1..17: note = VH CDR2
EIDPSDSYTNYNQKFQG

SEQ ID NO: 46 - protein, 8 aa, synthetic construct
  REGION 1..8: note = VH CDR3
SLSGYVDY

SEQ ID NO: 47 - protein, 11 aa, synthetic construct
  REGION 1..11: note = VL CDR1
RASQDISNRLN

SEQ ID NO: 48 - protein, 7 aa, synthetic construct
  REGION 1..7: note = VL CDR2
YRSRLHS

SEQ ID NO: 49 - protein, 9 aa, synthetic construct
  REGION 1..9: note = VL CDR3
QQGNSLPPT

SEQ ID NO: 50 - protein, 5 aa, synthetic construct
  REGION 1..5: note = VH CDR1
NYWMQ

SEQ ID NO: 51 - protein, 17 aa, synthetic construct
  REGION 1..17: note = VH CDR2
EIDPSDSYTNYNQKFQG

SEQ ID NO: 52 - protein, 8 aa, synthetic construct
  REGION 1..8: note = VH CDR3
SLSGYVDY

SEQ ID NO: 53 - protein, 11 aa, synthetic construct
  REGION 1..11: note = VL CDR1
RASQDISNRLN

SEQ ID NO: 54 - protein, 7 aa, synthetic construct
  REGION 1..7: note = VL CDR2
YRSRRHT

SEQ ID NO: 55 - protein, 9 aa, synthetic construct
  REGION 1..9: note = VL CDR3
QQGNSLPPT

SEQ ID NO: 56 - protein, 5 aa, synthetic construct
  REGION 1..5: note = VH CDR1
NYWMQ

SEQ ID NO: 57 - protein, 17 aa, synthetic construct
  REGION 1..17: note = VH CDR2
EIDPSDSYTNYNQGFTG

SEQ ID NO: 58 - protein, 8 aa, synthetic construct
  REGION 1..8: note = VH CDR3
SLSGYVDY

SEQ ID NO: 59 - protein, 11 aa, synthetic construct
  REGION 1..11: note = VL CDR1
RASQDISNRLN

SEQ ID NO: 60 - protein, 7 aa, synthetic construct
  REGION 1..7: note = VL CDR2
YRSRLHS

SEQ ID NO: 61 - protein, 9 aa, synthetic construct
  REGION 1..9: note = VL CDR3
QQGNSLPPT

SEQ ID NO: 62 - protein, 5 aa, synthetic construct
  REGION 1..5: note = VH CDR1
NYWMQ

SEQ ID NO: 63 - protein, 17 aa, synthetic construct
  REGION 1..17: note = VH CDR2
EIDPSDSYTNYNQGFTG

SEQ ID NO: 64 - protein, 8 aa, synthetic construct
  REGION 1..8: note = VH CDR3
SLSGYVDY

SEQ ID NO: 65 - protein, 11 aa, synthetic construct
  REGION 1..11: note = VL CDR1
RASQDISNRLN

SEQ ID NO: 66 - protein, 7 aa, synthetic construct
  REGION 1..7: note = VL CDR2
YRSRLHS

SEQ ID NO: 67 - protein, 9 aa, synthetic construct
  REGION 1..9: note = VL CDR3
QQGNSLPPT

SEQ ID NO: 68 - protein, 5 aa, synthetic construct
  REGION 1..5: note = VH CDR1
NYWMQ

SEQ ID NO: 69 - protein, 17 aa, synthetic construct
  REGION 1..17: note = VH CDR2
EIDPSDSYTNYNQGFTG

SEQ ID NO: 70 - protein, 8 aa, synthetic construct
  REGION 1..8: note = VH CDR3
SLSGYVDY

SEQ ID NO: 71 - protein, 11 aa, synthetic construct
  REGION 1..11: note = VL CDR1
RASQDISNRLN

SEQ ID NO: 72 - protein, 7 aa, synthetic construct
  REGION 1..7: note = VL CDR2
YRSRRHT

SEQ ID NO: 73 - protein, 9 aa, synthetic construct
  REGION 1..9: note = VL CDR3
QQGNSLPPT

SEQ ID NO: 74 - protein, 5 aa, synthetic construct
  REGION 1..5: note = VH CDR1
NYWMN

SEQ ID NO: 75 - protein, 17 aa, synthetic construct
  REGION 1..17: note = VH CDR2
EIDPSDSYTNYNQGFTG

SEQ ID NO: 76 - protein, 8 aa, synthetic construct
  REGION 1..8: note = VH CDR3
SLSGYVDY

SEQ ID NO: 77 - protein, 11 aa, synthetic construct
  REGION 1..11: note = VL CDR1
RASQDISNRLN

SEQ ID NO: 78 - protein, 7 aa, synthetic construct
  REGION 1..7: note = VL CDR2
YRSRRHT

SEQ ID NO: 79 - protein, 9 aa, synthetic construct
  REGION 1..9: note = VL CDR3
QQGNSLPPT

SEQ ID NO: 80 - protein, 117 aa, synthetic construct
  REGION 1..117: note = VH
QVQLQQPGAELVKPGASVKLSCKASGYTFTSYWMQWVKQRPGQGLEWIGEIDPSDYYTNYNQKFKGKATLTVDTSSSTAYMQLSSLTSEDSAVYYCARSLSGYVDYWGQGTTLTVSS

SEQ ID NO: 81 - protein, 107 aa, synthetic construct
  REGION 1..107: note = VL
DIQMTQSTSSLSASLGDRVTINCRASQDIRNRLNWYQQKPDGTVKLLISYRSRLQSGVPSRFSGSGSGTEYSLTISNLEQEDIATYFCQQGNTLPPTFGGGTKLEVR

SEQ ID NO: 82 - protein, 117 aa, synthetic construct
  REGION 1..117: note = VL
QVQLQQPGAELVKPGASVKLSCKASGYTFTNYWMQWVKQRPGQGLEWIGEIDPSDSYTNYNQKFKGKATLTVDTSSSTAYMQLSSLTSEDSAVYYCARSLSGYVDYWGQGTTLTVSS

SEQ ID NO: 83 - protein, 107 aa, synthetic construct
  REGION 1..107: note = VL
DIQMTQTTSSLSASLGDRVTISCRASQDISNRLNWYQQKPDGTVKLLISYRSRLHSGVPSRFSGSGSGTDYSLTISNLEQEDIATYFCQQGNSLPPTFGGGTKLEVR

SEQ ID NO: 84 - protein, 117 aa, synthetic construct
  REGION 1..117: note = VH
QVQLQQPGAELVKPGASVKLSCKASGYTFTSYWMQWVKQRPGQGLEWIGEIDPSDSYSNYNQKFKGKATLTVDTSSSTAYMQLSSLTSEDSAVYYCARSLSGYVDYWGQGTTLTVSS

SEQ ID NO: 85 - protein, 107 aa, synthetic construct
  REGION 1..107: note = VL
DIQMTQTTSSLSASLGDRVTISCRASQDISNRLNWYQQKPDGTVKLLISYRSRLQSGVPSRFSGSGSGTDYSLTISNLEEEDIATYFCQQGNTLPPTFGGGTKLEVR

SEQ ID NO: 86 - protein, 117 aa, synthetic construct
  REGION 1..117: note = VH
QVQLQQPGAELVKPGASVKLSCKASGYTFTDYWMQWVKQRPGQGLEWIGEIDPSDSYTNYNQKFQGKATLTVDTSSSTAYIQLSSLTSDDSAVYYCARSLSGSVDYWGQGTTLTVSS

SEQ ID NO: 87 - protein, 107 aa, synthetic construct
  REGION 1..107: note = VL
DIQMTQTTSSLSASLGDRVTISCRASQDISNRLNWYQQKPDGTVKLLISYRSRLQSGVPSRFSGSGSGTDYSLTITNLEQEDIATYFCQQGNTLPPTFGGGTKLEVR

SEQ ID NO: 88 - protein, 115 aa, synthetic construct
  REGION 1..115: note = VH
EVQLQQSGPELVKPGASVKISCKASGYTFTDYYMNWVKQSHGKSLEWIGDINPNNGGTIYNQKFKGKATLTVDKSSSTAYMELRSLTSEDSAVYYCASGYLFDYWGPGTTLTVSS

SEQ ID NO: 89 - protein, 113 aa, synthetic construct
  REGION 1..113: note = VL
DIVMTQSPSSLAMSVGQKVTMSCKSSQSLLNSSNQKNYLAWYQQKPGQSPKLLVYFASTRESGVPDRFIGSGSGTDFTLTISSVQAEDLAAYFCQQHYSTPLTFGAGTKLELK

SEQ ID NO: 90 - protein, 117 aa, synthetic construct
  REGION 1..117: note = VH
QVQLVQSGAEVKKPGASVKVSCKASGYTFTNYWMQWVRQAPGQGLEWIGEIDPSDSYTNYNQKFQGRVTLTVDTSTSTAYMELSSLRSEDTAVYYCARSLSGYVDYWGQGTTVTVSS

SEQ ID NO: 91 - protein, 107 aa, synthetic construct
  REGION 1..107: note = VL
DIQMTQSPSSLSASVGDRVTITCRASQDISNRLNWYQQKPGKAVKLLISYRSRLHSGVPSRFSGSGSGTDYTLTISSLQPEDFATYFCQQGNSLPPTFGGGTKLEIK

SEQ ID NO: 92 - protein, 117 aa, synthetic construct; identical to SEQ ID NO: 90
  REGION 1..117: note = VH

SEQ ID NO: 93 - protein, 107 aa, synthetic construct
  REGION 1..107: note = VL
DIQMTQSPSSLSASVGDRVTITCRASQDISNRLNWYQQKPGKAVKLLISYRSRLHSGVPSRFSGSGSGTDYTLTISSLQPEDFATYYCQQGNSLPPTFGGGTKLEIK

SEQ ID NO: 94 - protein, 117 aa, synthetic construct; identical to SEQ ID NO: 90
  REGION 1..117: note = VH

SEQ ID NO: 95 - protein, 107 aa, synthetic construct
  REGION 1..107: note = VL
DIVMTQSPATLSLSPGERATLSCRASQDISNRLNWYQQKPGQAVRLLISYRSRLHSGVPARFSGSGSGTDYTLTISSLEPEDFAVYFCQQGNSLPPTFGGGTKLEIK

SEQ ID NO: 96 - protein, 117 aa, synthetic construct; identical to SEQ ID NO: 90
  REGION 1..117: note = VH

SEQ ID NO: 97 - protein, 107 aa, synthetic construct
  REGION 1..107: note = VL
EIVMTQSPATLSLSPGERATLSCRASQDISNRLNWYQQKPGQAVRLLISYRSRRHTGIPARFSGSGSGTDYTLTISSLEPEDFAVYYCQQGNSLPPTFGGGTKLEIK

SEQ ID NO: 98 - protein, 117 aa, synthetic construct
  REGION 1..117: note = VH
QVQLVQSGSELKKPGASVKVSCKASGYTFTNYWMQWVRQAPGQGLEWIGEIDPSDSYTNYNQGFTGRFVLSVDTSVSTAYLQISSLKAEDTAVYYCARSLSGYVDYWGQGTTVTVSS

SEQ ID NO: 99 - protein, 107 aa, synthetic construct; identical to SEQ ID NO: 91
  REGION 1..107: note = VL

SEQ ID NO: 100 - protein, 117 aa, synthetic construct; identical to SEQ ID NO: 98
  REGION 1..117: note = VH

SEQ ID NO: 101 - protein, 107 aa, synthetic construct; identical to SEQ ID NO: 93
  REGION 1..107: note = VL

SEQ ID NO: 102 - protein, 117 aa, synthetic construct; identical to SEQ ID NO: 98
  REGION 1..117: note = VH

SEQ ID NO: 103 - protein, 107 aa, synthetic construct; identical to SEQ ID NO: 95
  REGION 1..107: note = VL

SEQ ID NO: 104 - protein, 117 aa, synthetic construct
  REGION 1..117: note = VH
QVQLVQSGSELKKPGASVKVSCKASGYTFTNYWMNWVRQAPGQGLEWMGEIDPSDSYTNYNQGFTGRFVFSVDTSVSTAYLQISSLKAEDTAVYYCARSLSGYVDYWGQGTTVTVSS

SEQ ID NO: 105 - protein, 107 aa, synthetic construct; identical to SEQ ID NO: 97
  REGION 1..107: note = VL

SEQ ID NO: 106 - protein, 117 aa, synthetic construct; identical to SEQ ID NO: 80
  REGION 1..117: note = VH

SEQ ID NO: 107 - protein, 324 aa, synthetic construct
  REGION 1..324: note = CH
AKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 108 - protein, 107 aa, synthetic construct; identical to SEQ ID NO: 81
  REGION 1..107: note = VL

SEQ ID NO: 109 - protein, 107 aa, synthetic construct
  REGION 1..107: note = CL
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 110 - protein, 117 aa, synthetic construct; identical to SEQ ID NO: 82
  REGION 1..117: note = VH

SEQ ID NO: 111 - protein, 336 aa, synthetic construct
  REGION 1..336: note = CH
AKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 112 - protein, 107 aa, synthetic construct; identical to SEQ ID NO: 83
  REGION 1..107: note = VL

SEQ ID NO: 113 - protein, 107 aa, synthetic construct; identical to SEQ ID NO: 109
  REGION 1..107: note = CL

SEQ ID NO: 114 - protein, 117 aa, synthetic construct; identical to SEQ ID NO: 84
  REGION 1..117: note = VH

SEQ ID NO: 115 - protein, 324 aa, synthetic construct; identical to SEQ ID NO: 107
  REGION 1..324: note = CH

SEQ ID NO: 116 - protein, 107 aa, synthetic construct; identical to SEQ ID NO: 85
  REGION 1..107: note = VL

SEQ ID NO: 117 - protein, 107 aa, synthetic construct; identical to SEQ ID NO: 109
  REGION 1..107: note = CL

SEQ ID NO: 118 - protein, 117 aa, synthetic construct; identical to SEQ ID NO: 86
  REGION 1..117: note = VH

SEQ ID NO: 119 - protein, 336 aa, synthetic construct; identical to SEQ ID NO: 111
  REGION 1..336: note = CH

SEQ ID NO: 120 - protein, 107 aa, synthetic construct; identical to SEQ ID NO: 87
  REGION 1..107: note = VL

SEQ ID NO: 121 - protein, 107 aa, synthetic construct; identical to SEQ ID NO: 109
  REGION 1..107: note = CL

SEQ ID NO: 122 - protein, 115 aa, synthetic construct; identical to SEQ ID NO: 88
  REGION 1..115: note = VH

SEQ ID NO: 123 - protein, 335 aa, synthetic construct
  REGION 1..335: note = CH
AKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 124 - protein, 113 aa, synthetic construct; identical to SEQ ID NO: 89
  REGION 1..113: note = VL

SEQ ID NO: 125 - protein, 107 aa, synthetic construct; identical to SEQ ID NO: 109
  REGION 1..107: note = CL

SEQ ID NO: 126 - protein, 117 aa, synthetic construct; identical to SEQ ID NO: 90
  REGION 1..117: note = VH

SEQ ID NO: 127 - protein, 330 aa, synthetic construct [sequence omitted]

SEQ ID NO: 128 - protein, 107 aa, synthetic construct; identical to SEQ ID NO: 91
  REGION 1..107: note = VL

SEQ ID NO: 129 - protein, 107 aa, synthetic construct [sequence omitted]

SEQ ID NO: 130 - protein, 117 aa, synthetic construct; identical to SEQ ID NO: 90
  REGION 1..117: note = VH

SEQ ID NO: 131 - protein, 330 aa, synthetic construct [sequence omitted]

SEQ ID NO: 132 - protein, 107 aa, synthetic construct; identical to SEQ ID NO: 93
  REGION 1..107: note = VL

SEQ ID NO: 133 - protein, 107 aa, synthetic construct [sequence omitted]

SEQ ID NO: 134 - protein, 117 aa, synthetic construct; identical to SEQ ID NO: 90
  REGION 1..117: note = VH

SEQ ID NO: 135 - protein, 330 aa, synthetic construct [sequence omitted]

SEQ ID NO: 136 - protein, 107 aa, synthetic construct; identical to SEQ ID NO: 95
  REGION 1..107: note = VL

SEQ ID NO: 137 - protein, 107 aa, synthetic construct [sequence omitted]

SEQ ID NO: 138 - protein, 117 aa, synthetic construct; identical to SEQ ID NO: 90
  REGION 1..117: note = VH

SEQ ID NO: 139 - protein, 330 aa, synthetic construct [sequence omitted]

SEQ ID NO: 140 - protein, 107 aa, synthetic construct; identical to SEQ ID NO: 97
  REGION 1..107: note = VL

SEQ ID NO: 141 - protein, 107 aa, synthetic construct [sequence omitted]

SEQ ID NO: 142 - protein, 117 aa, synthetic construct; identical to SEQ ID NO: 98
  REGION 1..117: note = VH

SEQ ID NO: 143 - protein, 330 aa, synthetic construct [sequence omitted]

SEQ ID NO: 144 - protein, 107 aa, synthetic construct; identical to SEQ ID NO: 91
  REGION 1..107: note = VL

SEQ ID NO: 145 - protein, 107 aa, synthetic construct [sequence omitted]

SEQ ID NO: 146 - protein, 117 aa, synthetic construct; identical to SEQ ID NO: 98
  REGION 1..117: note = VH

SEQ ID NO: 147 - protein, 330 aa, synthetic construct [sequence omitted]

SEQ ID NO: 148 - protein, 107 aa, synthetic construct; identical to SEQ ID NO: 95
  REGION 1..107: note = VL

SEQ ID NO: 149 - protein, 107 aa, synthetic construct [sequence omitted]

SEQ ID NO: 150 - protein, 117 aa, synthetic construct; identical to SEQ ID NO: 98
  REGION 1..117: note = VH

SEQ ID NO: 151 - protein, 330 aa, synthetic construct [sequence omitted]

SEQ ID NO: 152 - protein, 107 aa, synthetic construct; identical to SEQ ID NO: 97
  REGION 1..107: note = VL

SEQ ID NO: 153 - protein, 107 aa, synthetic construct [sequence omitted]

SEQ ID NO: 154 - protein, 117 aa, synthetic construct; identical to SEQ ID NO: 104
  REGION 1..117: note = VH

SEQ ID NO: 155 - protein, 330 aa, synthetic construct [sequence omitted]

SEQ ID NO: 156 - protein, 107 aa, synthetic construct; identical to SEQ ID NO: 97
  REGION 1..107: note = VL

SEQ ID NO: 157 - protein, 107 aa, synthetic construct [sequence omitted]

SEQ ID NO: 158 - DNA, 351 bp, synthetic construct
  misc_feature 1..351: note = VH
  source 1..351: mol_type = other DNA
caggtccaactgcagcagcctggggctgagcttgtgaagcctggggcttcagtgaaactgtcctgcaaggcttctggctacaccttcaccagctactggatgcagtgggtaaaacagaggcctggacagggccttgagtggatcggagagattgatccttctgattactatactaactacaatcaaaagttcaagggcaaggccacattgactgtagacacatcctccagcacagcctacatgcagctcagcagcctgacatctgaggactctgcggtctattactgtgcaagatccctctcaggctacgttgactactggggccaaggcaccactctcacagtctcctca

SEQ ID NO: 159 - DNA, 972 bp, synthetic construct
  misc_feature 1..972: note = CH
  source 1..972: mol_type = other DNA
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 160 - DNA, 321 bp, synthetic construct
  misc_feature 1..321: note = VL
  source 1..321: mol_type = other DNA
gatatccagatgacacagagtacatcctccctgtctgcctctctgggagacagagtcaccatcaattgcagggcaagtcaggacattagaaatcgtttaaactggtatcagcagaaaccagatggaactgttaaactcctgatcagctacagatcaagattacagtcaggagtcccatcaaggttcagtggcagtgggtctggcacagagtattctctcaccattagcaacctggagcaagaagacattgccacttatttttgccaacagggcaatacgcttcctcccacgttcggaggggggaccaagctggaagtgaga

SEQ ID NO: 161 - DNA, 321 bp, synthetic construct
  misc_feature 1..321: note = CL
  source 1..321: mol_type = other DNA
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 162 - DNA, 351 bp, synthetic construct
  misc_feature 1..351: note = VH
  source 1..351: mol_type = other DNA
caagtccaactgcagcagcctggggctgagcttgtgaagcctggggcttcagtgaagctgtcctgcaaggcttctggctacaccttcaccaactactggatgcagtgggtaaaacagaggcctggacagggccttgagtggatcggagagattgatccttctgatagttatactaactacaatcaaaagttcaagggcaaggccacattgactgtagacacatcctccagcacagcctacatgcagctcagcagcctgacatctgaggactctgcggtctattactgtgcaagatccctctcaggctacgttgactactggggccaaggcaccactctcacagtctcctca

SEQ ID NO: 163 - DNA, 1008 bp, synthetic construct
  misc_feature 1..1008: note = CH
  source 1..1008: mol_type = other DNA
gccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaa

SEQ ID NO: 164 - DNA, 321 bp, synthetic construct
  misc_feature 1..321: note = VL
  source 1..321: mol_type = other DNA
gatatccagatgacacagactacatcctccctgtctgcctctctgggagacagagtcaccatcagttgcagggcaagtcaggacattagcaatcgtttaaactggtatcagcagaaaccagatggaactgttaaactcctgatcagctacagatcaagattacactcaggagtcccatcaaggttcagtggcagtgggtctggcacagattattctctcaccattagcaacctggagcaagaagacattgccacttatttttgccaacagggtaattcgcttcctcccacgttcggaggggggaccaagctggaagtgaga

SEQ ID NO: 165 - DNA, 321 bp, synthetic construct; identical to SEQ ID NO: 161
  misc_feature 1..321: note = CL
  source 1..321: mol_type = other DNA

SEQ ID NO: 166 - DNA, 351 bp, synthetic construct
  misc_feature 1..351: note = VH
  source 1..351: mol_type = other DNA
caggtccaactgcagcagcctggggctgagcttgtgaagcctggggcttcagtgaagctgtcctgcaaggcttctggctacaccttcaccagctactggatgcagtgggtaaaacagaggcctggacagggccttgagtggatcggagagattgatccttctgatagctatagtaactacaatcaaaagttcaagggcaaggccacattgactgtagacacatcctccagcacagcctacatgcagctcagcagcctgacatctgaggactctgcggtctattactgtgcaagatccctctcaggctacgttgactactggggccaaggcaccactctcacagtctcctca

SEQ ID NO: 167 - DNA, 972 bp, synthetic construct; identical to SEQ ID NO: 159
  misc_feature 1..972: note = CH
  source 1..972: mol_type = other DNA

SEQ ID NO: 168 - DNA, 321 bp, synthetic construct
  misc_feature 1..321: note = VL
  source 1..321: mol_type = other DNA
gatatccagatgacacagactacatcctccctgtctgcctctctgggagacagagtcaccatcagttgcagggcaagtcaggacattagcaatcgtttaaactggtatcagcagaaaccagatggaactgttaaactcctgatcagctacagatcaagattacagtcaggagtcccatcaaggttcagtggcagtgggtctggcacagattattctctcaccattagcaacctggaggaagaagacattgccacctatttttgccaacagggtaatacccttcctcccacgttcggaggggggaccaagctggaagtgaga

SEQ ID NO: 169 - DNA, 321 bp, synthetic construct; identical to SEQ ID NO: 161
  misc_feature 1..321: note = CL
  source 1..321: mol_type = other DNA

SEQ ID NO: 170 - DNA, 351 bp, synthetic construct
  misc_feature 1..351: note = VH
  source 1..351: mol_type = other DNA
caggtccaactgcagcagcctggggctgagcttgtgaagcctggggcttcagtgaagctgtcctgcaaggcttctggctacaccttcaccgactactggatgcagtgggtaaaacagaggcctggacagggccttgagtggatcggagagattgatccttctgatagctatactaactacaatcaaaagttccagggcaaggccacattgactgtagacacatcctccagcacagcctacatccagctcagcagcctgacatctgacgactctgcggtctattactgtgcaagatccctctcaggctccgttgactactggggccaaggcaccactctcacagtctcctca

SEQ ID NO: 171 - DNA, 1008 bp, synthetic construct; identical to SEQ ID NO: 163
  misc_feature 1..1008: note = CH
  source 1..1008: mol_type = other DNA

SEQ ID NO: 172 - DNA, 321 bp, synthetic construct
  misc_feature 1..321: note = VL
  source 1..321: mol_type = other DNA
gatatccagatgacacagactacttcctccctgtctgcctctctgggagacagagtcaccatcagttgcagggcaagtcaggacattagcaatcgtttaaactggtatcagcagaaaccagatggaactgttaaactcctgatcagctacagatcaagattacagtcaggagtcccatcaaggttcagtggcagtgggtctggcacagattattctctcaccattaccaacctggagcaagaagacattgccacttatttttgccaacagggtaatacgcttcctcccacgttcggaggggggaccaagctggaagtgaga

SEQ ID NO: 173 - DNA, 321 bp, synthetic construct; identical to SEQ ID NO: 161
  misc_feature 1..321: note = CL
  source 1..321: mol_type = other DNA

SEQ ID NO: 174 - DNA, 345 bp, synthetic construct
  misc_feature 1..345: note = VH
  source 1..345: mol_type = other DNA
gaggtccagctgcaacaatctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggactctgcagtctattactgtgcaagcggatacctctttgactactggggcccaggcaccactctcacagtctcctca

SEQ ID NO: 175 - DNA, 1005 bp, synthetic construct
  misc_feature 1..1005: note = CH
  source 1..1005: mol_type = other DNA
gccaaaacaacagccccatcggtctatccactggcccctgtgtgtggaggtacaactggctcctcggtgactctaggatgcctggtcaagggttatttccctgagccagtgaccttgacctggaactctggatccctgtccagtggtgtgcacaccttcccagctctcctgcagtctggcctctacaccctcagcagctcagtgactgtaacctcgaacacctggcccagccagaccatcacctgcaatgtggcccacccggcaagcagcaccaaagtggacaagaaaattgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaa

SEQ ID NO: 176 - DNA, 339 bp, synthetic construct
  misc_feature 1..339: note = VL
  source 1..339: mol_type = other DNA
gacattgtgatgacacagtctccatcctccctggctatgtcagtaggacagaaggtcactatgagctgcaagtccagtcagagccttttaaatagtagcaatcaaaagaactatttggcctggtaccagcagaaaccaggacagtctcctaaacttctggtatactttgcatccactagggaatctggggtccctgatcgcttcataggcagtggatctgggacagatttcactcttaccatcagcagtgtgcaggctgaagacctggcagcttacttctgtcagcaacattatagcactccgctcacgttcggtgctgggaccaagctggagctgaaa

SEQ ID NO: 177 - DNA, 321 bp, synthetic construct; identical to SEQ ID NO: 161
  misc_feature 1..321: note = CL
  source 1..321: mol_type = other DNA

SEQ ID NO: 178 - DNA, 351 bp, synthetic construct
  misc_feature 1..351: note = VH
  source 1..351: mol_type = other DNA
caagtgcagctggtgcagagcggggccgaggtgaagaagcccggcgcaagcgtgaaggtgagctgcaaggcaagcggctacaccttcaccaactactggatgcagtgggtgagacaagcccccggccaaggcctggagtggatcggcgagatcgaccctagcgacagctacaccaactacaatcagaagttccaaggcagagtgaccctgaccgtggacacaagcacaagcaccgcctacatggagctgagcagcctgagaagcgaggacaccgccgtgtactactgcgctagaagcctgagcggctacgtggactactggggccaaggcaccaccgtgaccgtctcgagc

SEQ ID NO: 179 - DNA, 990 bp, synthetic construct
  misc_feature 1..990: note = CH
  source 1..990: mol_type = other DNA
gccagcaccaaaggtccatccgtgtttccgctcgccccgtcctcaaagtcgacctccggaggcactgccgccctgggctgccttgtcaaggactatttccccgaacctgtcacggtgtcctggaacagcggcgccctgacttccggagtgcacaccttccccgccgtcctgcaatccagcggcctgtactcactgtcatccgttgtgactgtcccgtcgtccagcctgggaacccaaacctacatttgcaacgtgaatcacaaaccatcgaataccaaggtcgataagaaagtcgagccgaagtcatgcgacaagactcacacctgtccgccttgcccagccccagaactgctcggcggcccttcggtgtttttgtttccgccgaagccgaaggacactctgatgatctcacgcactccagaggtgacttgcgtggtggtcgatgtttcgcacgaggacccggaagtgaaattcaactggtatgtcgacggggtggaagtgcataatgccaagacgaagccgagggaggaacagtacaactccacctacagagtggtttcagtccttaccgtcctccatcaagattggctgaacggaaaggagtacaaatgtaaggtgtcgaacaaagcgttgccggcccctatcgaaaagactatcagcaaggccaaaggacagccgcgggagccgcaagtgtacaccctcccgccttcgcgggacgagctgaccaagaatcaggtgtcccttacttgcctggtgaagggattctacccctcggatatcgcagtcgaatgggaatcgaatggacagccagaaaacaactacaagaccactcccccggtgctcgactccgacggttccttcttcctgtactcgaagctgaccgtggacaaatcacgctggcagcagggaaacgtgtttagctgcagcgtgatgcatgaggcgctgcataatcactacacccagaagtcactctcgctcagcccagggaag

SEQ ID NO: 180 - DNA, 321 bp, synthetic construct
  misc_feature 1..321: note = VL
  source 1..321: mol_type = other DNA
gacattcagatgacacagagccctagcagcctgagcgcaagcgtgggcgacagagtgaccatcacctgcagagcaagccaagacatcagcaacagactgaactggtatcagcagaagcccggcaaggccgtgaagctgctgatcagctacagaagcagactgcacagcggcgtgcctagcagattcagcggcagcggcagcggcaccgactacaccctgaccatcagcagcctgcagcctgaggacttcgccacctacttctgtcagcaaggcaacagcctgccccccaccttcggcggcggcaccaagctcgagatcaag

SEQ ID NO: 181 - DNA, 321 bp, synthetic construct
  misc_feature 1..321: note = CL
  source 1..321: mol_type = other DNA
agaactgtggccgcgccgtcagtgtttatcttccctccatcggatgaacagcttaagtccggcacggcgtctgtggtctgcctgctcaataacttttaccctagggaagctaaagtccaatggaaagtggataacgccctgcagtcaggaaacagccaggaatcggttaccgaacaggacagcaaggacagcacttactccttgtcgtcgactcttactctgagcaaggccgattacgagaagcacaaggtctacgcctgcgaggtcacccatcagggactctcgtccccggtgaccaaatccttcaatagaggcgaatgc

SEQ ID NO: 182 - DNA, 351 bp, synthetic construct; identical to SEQ ID NO: 178
  misc_feature 1..351: note = VH
  source 1..351: mol_type = other DNA

SEQ ID NO: 183 - DNA, 990 bp, synthetic construct; identical to SEQ ID NO: 179
  misc_feature 1..990: note = CH
  source 1..990: mol_type = other DNA

SEQ ID NO: 184 - DNA, 321 bp, synthetic construct
  misc_feature 1..321: note = VL
  source 1..321: mol_type = other DNA
gacattcagatgacacagagccctagcagcctgagcgcaagcgtgggcgacagagtgaccatcacctgcagagcaagccaagacatcagcaacagactgaactggtatcagcagaagcccggcaaggccgtgaagctgctgatcagctacagaagcagactgcacagcggcgtgcctagcagattcagcggcagcggcagcggcaccgactacaccctgaccatcagcagcctgcagcctgaggacttcgccacctactactgtcagcaaggcaacagcctgccccccaccttcggcggcggcaccaagctcgagatcaag

SEQ ID NO: 185 - DNA, 321 bp, synthetic construct; identical to SEQ ID NO: 181
  misc_feature 1..321: note = CL
  source 1..321: mol_type = other DNA

SEQ ID NO: 186 - DNA, 351 bp, synthetic construct; identical to SEQ ID NO: 178
  misc_feature 1..351: note = VH
  source 1..351: mol_type = other DNA

SEQ ID NO: 187 - DNA, 990 bp, synthetic construct; identical to SEQ ID NO: 179
  misc_feature 1..990: note = CH
  source 1..990: mol_type = other DNA

SEQ ID NO: 188 - DNA, 321 bp, synthetic construct
  misc_feature 1..321: note = VL
  source 1..321: mol_type = other DNA
gacatcgtaatgacacagagccccgcgaccctgagcctgtcccccggtgagagagctaccctgagctgcagagcaagccaagacatcagcaacagactgaactggtatcagcagaagcccggccaagccgtgagactgctgatcagctacagaagcagactgcacagcggcgtgcccgctagattcagcggcagcggcagcggcaccgactacaccctgaccatcagcagcctggagcctgaggacttcgccgtgtacttctgtcagcaaggcaacagcctgccccccaccttcggcggcggcaccaagctcgagatcaag

SEQ ID NO: 189 - DNA, 321 bp, synthetic construct; identical to SEQ ID NO: 181
  misc_feature 1..321: note = CL
  source 1..321: mol_type = other DNA

SEQ ID NO: 190 - DNA, 351 bp, synthetic construct; identical to SEQ ID NO: 178
  misc_feature 1..351: note = VH
  source 1..351: mol_type = other DNA

SEQ ID NO: 191 - DNA, 990 bp, synthetic construct; identical to SEQ ID NO: 179
  misc_feature 1..990: note = CH
  source 1..990: mol_type = other DNA

SEQ ID NO: 192 - DNA, 321 bp, synthetic construct
  misc_feature 1..321: note = VL
  source 1..321: mol_type = other DNA
gagatcgtaatgacacagagccccgcgaccctgagcctgtcccccggtgagagagctaccctgagctgcagagcaagccaagacatcagcaacagactgaactggtatcagcagaagcccggccaagccgtgagactgctgatcagctacagaagcagaagacacaccggcatccccgctagattcagcggcagcggcagcggcaccgactacaccctgaccatcagcagcctggagcctgaggacttcgccgtgtactactgtcagcaaggcaacagcctgccccccaccttcggcggcggcaccaagctcgagatcaag

SEQ ID NO: 193 - DNA, 321 bp, synthetic construct; identical to SEQ ID NO: 181
  misc_feature 1..321: note = CL
  source 1..321: mol_type = other DNA

SEQ ID NO: 194 - DNA, 351 bp, synthetic construct
  misc_feature 1..351: note = VH
  source 1..351: mol_type = other DNA
caagtgcagctggtgcagagcggcagcgagctgaagaagcccggcgcaagcgtgaaggtgagctgcaaggcaagcggctacaccttcaccaactactggatgcagtgggtgagacaagcccccggccaaggcctggagtggatcggcgagatcgaccctagcgacagctacaccaactacaaccaaggcttcaccggcagattcgtgctgagcgtggacacaagcgtgagcaccgcctacctgcagatcagcagcctgaaggccgaggacaccgccgtgtactactgcgctagaagcctgagcggctacgtggactactggggccaaggcaccaccgtgaccgtctcgagc

SEQ ID NO: 195 - DNA, 990 bp, synthetic construct; identical to SEQ ID NO: 179
  misc_feature 1..990: note = CH
  source 1..990: mol_type = other DNA

SEQ ID NO: 196 - DNA, 321 bp, synthetic construct; identical to SEQ ID NO: 180
  misc_feature 1..321: note = VL
  source 1..321: mol_type = other DNA

SEQ ID NO: 197 - DNA, 321 bp, synthetic construct; identical to SEQ ID NO: 181
  misc_feature 1..321: note = CL
  source 1..321: mol_type = other DNA

SEQ ID NO: 198 - DNA, 351 bp, synthetic construct; identical to SEQ ID NO: 194
  misc_feature 1..351: note = VH
  source 1..351: mol_type = other DNA

SEQ ID NO: 199 - DNA, 990 bp, synthetic construct; identical to SEQ ID NO: 179
  misc_feature 1..990: note = CH
  source 1..990: mol_type = other DNA

SEQ ID NO: 200 - DNA, 321 bp, synthetic construct; identical to SEQ ID NO: 188
  misc_feature 1..321: note = VL
  source 1..321: mol_type = other DNA

SEQ ID NO: 201 - DNA, 321 bp, synthetic construct; identical to SEQ ID NO: 181
  misc_feature 1..321: note = CL
  source 1..321: mol_type = other DNA

SEQ ID NO: 202 - DNA, 351 bp, synthetic construct; identical to SEQ ID NO: 194
  misc_feature 1..351: note = VH
  source 1..351: mol_type = other DNA

SEQ ID NO: 203 - DNA, 990 bp, synthetic construct; identical to SEQ ID NO: 179
  misc_feature 1..990: note = CH
  source 1..990: mol_type = other DNA

SEQ ID NO: 204 - DNA, 321 bp, synthetic construct; identical to SEQ ID NO: 192
  misc_feature 1..321: note = VL
  source 1..321: mol_type = other DNA

SEQ ID NO: 205 - DNA, 321 bp, synthetic construct; identical to SEQ ID NO: 181
  misc_feature 1..321: note = CL
  source 1..321: mol_type = other DNA

SEQ ID NO: 206 - DNA, 351 bp, synthetic construct
  misc_feature 1..351: note = VH
  source 1..351: mol_type = other DNA
caagtgcagctggtgcagagcggcagcgagctgaagaagcccggcgcaagcgtgaaggtgagctgcaaggcaagcggctacaccttcaccaactactggatgaactgggtgagacaagcccccggccaaggcctggagtggatgggcgagatcgaccctagcgacagctacaccaactacaaccaaggcttcaccggcagattcgtgttcagcgtggacacaagcgtgagcaccgcctacctgcagatcagcagcctgaaggccgaggacaccgccgtgtactactgcgctagaagcctgagcggctacgtggactactggggccaaggcaccaccgtgaccgtctcgagc

SEQ ID NO: 207 - DNA, 990 bp, synthetic construct; identical to SEQ ID NO: 179
  misc_feature 1..990: note = CH
  source 1..990: mol_type = other DNA

SEQ ID NO: 208 - DNA, 321 bp, synthetic construct; identical to SEQ ID NO: 192
  misc_feature 1..321: note = VL
  source 1..321: mol_type = other DNA

SEQ ID NO: 209 - DNA, 321 bp, synthetic construct; identical to SEQ ID NO: 181
  misc_feature 1..321: note = CL
  source 1..321: mol_type = other DNA

SEQ ID NO: 210 - protein, 18 aa, synthetic construct
  REGION 1..18: note = alpha-LGR5 epitope peptide
SSPRSGVLLRGCPTHCHC

SEQ ID NO: 211 - protein, 260 aa, synthetic construct
MPLLLLLPLLWAGALAEIVMTQSPATLSLSPGERATLSCRASQDISNRLNWYQQKPGQAVRLLISYRSRRHTGIPARFSGSGSGTDYTLTISSLEPEDFAVYYCQQGNSLPPTFGGGTKLEIKGGGGSGGGGSGGGGSGGGGSQVQLVQSGAEVKKPGASVKVSCKASGYTFTNYWMQWVRQAPGQGLEWIGEIDPSDSYTNYNQKFQGRVTLTVDTSTSTAYMELSSLRSEDTAVYYCARSLSGYVDYWGQGTTVTVSS

SEQ ID NO: 212 - protein, 11 aa, synthetic construct
RASQDISNRLN

SEQ ID NO: 213 - protein, 7 aa, synthetic construct
YRSRRHT

SEQ ID NO: 214 - protein, 9 aa, synthetic construct
QQGNSLPPT

SEQ ID NO: 215 - protein, 5 aa, synthetic construct
NYWMQ

SEQ ID NO: 216 - protein, 17 aa, synthetic construct
EIDPSDSYTNYNQKFQG

SEQ ID NO: 217 - protein, 8 aa, synthetic construct
SLSGYVDY

SEQ ID NO: 218 - DNA, 780 bp, synthetic construct
  source 1..780: mol_type = other DNA
atgccgctgctgctactgctgcccctgctgtgggcaggggcgctagctgagatcgtgatgactcaaagccccgccacactgagcctatctcccggcgagagagccacgctgagctgtagagcaagccaagacatcagcaacagactgaactggtatcagcagaagcccggccaagccgtgagactgctgatcagctacagaagcagaagacacaccggcatccccgctagattcagcggcagcggcagcggcaccgactacaccctgaccatcagcagcctggagcctgaggacttcgctgtatactactgccaacaaggcaacagcctgccgccaaccttcggaggcggcacgaagctggagatcaagggcggtggtggatcgggcggcgggggcagcgggggtggtggttctggtggagggggctcacaagtgcagctggtgcagagcggggccgaggtgaagaagcccggcgcaagcgtgaaggtgagctgcaaggcaagcggctacaccttcaccaactactggatgcagtgggtgagacaagcccccggccaaggcctggagtggatcggcgagatcgaccctagcgacagctacaccaactacaatcagaagttccaaggcagagtgaccctgaccgtggacacaagcacaagcaccgcctacatggagctgagcagcctgagaagcgaggacaccgcggtttactactgcgctagaagcctgagcggctacgtggactactggggccaaggcaccaccgtgaccgtgagcagc

SEQ ID NO: 219 - RNA, 21 nt, synthetic construct
  source 1..21: mol_type = other RNA
gctgctgcagctggcgaccgg

SEQ ID NO: 220 - RNA, 21 nt, synthetic construct
  source 1..21: mol_type = other RNA
gcgcggcgccgcctctctgcg

SEQ ID NO: 221 - DNA, 42 bp, synthetic construct
  source 1..42: mol_type = other DNA
gacagactgagtcgcccgggacgcgtcccaccatgccgctgc

SEQ ID NO: 222 - DNA, 27 bp, synthetic construct
  source 1..27: mol_type = other DNA
gcgtcgtggtgctgctcacggtcacgg

SEQ ID NO: 223 - DNA, 26 bp, synthetic construct
  source 1..26: mol_type = other DNA
cgtgagcagcaccacgacgccagcgc

SEQ ID NO: 224 - DNA, 39 bp, synthetic construct
  source 1..39: mol_type = other DNA
caccatggtggcgaccggtggatcccgagggggcagggc